SEQUENCE LISTING (288 sequences)

SEQ ID NO: 1 - DNA, 24 bp, Artificial Sequence
  Synthetic construct
accatggccagctaatttttgtat

SEQ ID NO: 2 - DNA, 24 bp, Artificial Sequence
  Synthetic construct
aaggcaaatccatagacacagaaa

SEQ ID NO: 3 - DNA, 1559 bp, Homo sapiens
gaagcacgcccgctgtgaacatgtccaggcgcggcgcttctccatcaattgaagaagacaccaccattctgaggccagggagcgagtgggggccagccacggggactcagcccttgtttctcttcctttctttttttaaaaaatagcagctttagccaagtgcagggcctgtaatcccagcattttgggaggccggggttggaggatcatttgagcccaggaattggaaagcagcctggccaacatagtgggaccctgtctctacaaaaaaaaaatttgccaagagcctgagtgacagagcaagaccccatctcaaaaaaaaaacaaacaaacaaaaaaaaaaccatatatatacatatatatatagcagctttatggagatataattcttatgccatataattcaccttcttttttttttttgtctgagacagaatctcagtctgtcacccaggttggagtgcagtggcgtgatctcagctcactgcaacctccacctcgcaggttcaagcaatcctcccacttcagcctcccaagcacctgggattacaagcatgagtcactacgcctggctgatttttgtagttttagtggagatggggtttcaccatgttggccaggcttgtctcgaactcctgaccccaagttatccacctgccttggcttcccaaagtcctgggattacaggtgtgagccaccacatccagcctaacttacattcttaaagtgtcgaatgacttctagtgtagaattgtgcaaccatcaccagaattaattttattattcttattatttttgagacagagtcttactctgttgccaggctggagtgcagtggcgcgatctcagctcactacaacctccgcctcccatgttcaagcgattctcctgcctcagcctcccgagtagctgggactatagatgcgccaccatggccagctaatttttgtatttttagtagagacgaggtttcactgtgttggccaggatggtctccatctcttgacctcgtgatccacccgcctcagcctcccaaagtgctgggattaacaggtatgaaccaccgcgcccagcctttttgtttttttttttttgagacagagtcttcctctgtctcctaagctggagtgcagtggcatcatctcagctcactgcaacctctgcctcccaggttcaagtgcttctccagcctcggcctcccaagtagctgagactacaggcacacaccaccacgcctggctaatttttgtatttttggtagagacgggtttcaccatgttggtcagactagtctcaaactcctgacctcaagtgatctgcccgcctcgacctctctcaaaatgctggcattacaggtgtgagccacggtgcccggcccacaattaattttagaacattttcatcacccctaaaagaaaccctgcacccattagcagtccctccacatttccccctagcctgcctcccctgcctcaccagccctggcaactgctaatctactttctgtgtctatggatttgccttctctaaacatttcatataaatggaattacacaatg

SEQ ID NO: 4 - DNA, 596 bp, Artificial Sequence
  Synthetic construct
accatggccagctaatttttgtatttttagtagagacgaggtttcactgtgttggccaggatggtctccatctcttgacctcgtgatccacccgcctcagcctcccaaagtgctgggattaacaggtatgaaccaccgcgcccagcctttttgtttttttttttttgagacagagtcttcctctgtctcctaagctggagtgcagtggcatcatctcagctcactgcaacctctgcctcccaggttcaagtgcttctccagcctcggcctcccaagtagctgagactacaggcacacaccaccacgcctggctaatttttgtatttttggtagagacgggtttcaccatgttggtcagactagtctcaaactcctgacctcaagtgatctgcccgcctcgacctctctcaaaatgctggcattacaggtgtgagccacggtgcccggcccacaattaattttagaacattttcatcacccctaaaagaaaccctgcacccattagcagtccctccacatttccccctagcctgcctcccctgcctcaccagccctggcaactgctaatctactttctgtgtctatggatttgcctt

SEQ ID NO: 5 - DNA, 24 bp, Artificial Sequence
  Synthetic construct
aagatgtctcaggtttgttttgtg

SEQ ID NO: 6 - DNA, 24 bp, Artificial Sequence
  Synthetic construct
ggtgtcccagtataagtaatgaga

SEQ ID NO: 7 - DNA, 3148 bp, Homo sapiens
caagccaaggaaacttgccaataagaagcaagaggcaagctgaaattttagaaatattcacatcttcggagatgaggagtaaaattcagtttttttccagttcctcttttgtgctgcttctcaattagcgtttaaggtgagcataaatcaactgtccatcaggtgaggtgtgctccatacccagcggttcttcatgagtagtgggctatgcaggagcttctgggagatttttttgagtcaaagacttaaagggcccaatgaattattatatacatactgcatcttggttatttctgaaggtagcattctttggagttaaaatgcacatatagacacatacacccaaacacttacaccaaactactgaatgaagaagtattttggtaaccaggccatttttggtgggaatccaagattggtctcccatatgcagaaatagacaaaaagtatattaaacaaagtttcagagtatattgttgaagagacagagacaagtaatttcagtgtaaagtgtgtgattgaaggtgataagggaaaagataaagaccagaaattcccttttcaccttttcaggaaaataacttagactctagtatttatgggtggatttatccttttgccttctggtatacttccttacttttaaggataaatcataaagtcagttgctcaaaaagaaatcaatagttgaattagtgagtatagtggggttccatgagttatcatgaattttaaagtatgcattattaaattgtaaaactccaaggtgatgttgtacctcttttgcttgccaaagtacagaatttgaattatcagcaaagaaaaaaaaaaaagccagccaagctttaaattatgtgaccataatgtactgatttcagtaagtctcataggttaaaaaaaaaagtcaccaaatagtgtgaaatatattacttaactgtccgtaagcagtatattagtattatcttgttcaggaaaaggttgaataatatatgccttgtgtaatattgaaaattgaaaagtacaactaacgcaaccaagtgtgctaaaaatgagcttgattaaatcaaccacctatttttgacatggaaatgaagcagggtttcttttcttcactcaaattttggcgaatctcaaaattagatcctaagatgtgttcttatttttataacatctttattgaaattctatttataatacagaatcttgttttgaaaataacctaattaatatattaaaattccaaattcatggcatgcttaaattttaactaaattttaaagccattctgattattgagttccagttgaagttagtggaaatctgaacattctcctgtggaaggcagagaaatctaagctgtgtctgcccaatgaataatggaaaatgccatgaattacctggatgttctttttacgaggtgacaagagttggggacagaactcccattacaactgaccaagtttctcttctagatgattttttgaaagttaacattaatgcctgctttttggaaagtcagaatcagaagatagtcttggaagctgtttggaaaagacagtggagatgaggtcagttgtgttttttaagatggcaattactttggtagctgggaaagcataaagctcaaatgaaatgtatgcattcacatttagaaaagtgaattgaagtttcaagttttaaagttcattgcaattaaacttccaaagaaagttctacagtgtcctaagtgctaagtgcttattacattttattaagctttttggaatctttgtaccaaaattttaaaaaagggagtttttgatagttgtgtgtatgtgtgtgtggggtggggggatggtaagagaaaagagagaaacactgaaaagaaggaaagatggttaaacattttcccactcattctgaattaattaatttggagcacaaaattcaaagcatggacatttagaagaaagatgtttggcgtagcagagttaaatctcaaataggctattaaaaaagtctacaacatagcagatctgttttgtggtttggaatattaaaaaacttcatgtaattttattttaaaatttcatagctgtacttcttgaatataaaaaatcatgccagtatttttaaaggcattagagtcaactacacaaagcaggcttgcccagtacatttaaattttttggcacttgccattccaaaatattatgccccaccaaggctgagacagtgaatttgggctgctgtagcctatttttttagattgagaaatgtgtagctgcaaaaataatcatgaaccaatctggatgcctcattatgtcaaccaggtccagatgtgctataatctgtttttacgtatgtaggcccagtcgtcatcagatgcttgcggcaaaagaaagctgtgtttatatggaagaaagtaaggtgcttggagtttacctggcttatttaatatgcttataacctagttaaagaaaggaaaagaaaacaaaaaacgaatgaaaataactgaatttggaggctggagtaatcagattactgctttaatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccattaccaaaataataaaaatcactttcataatcttgaaaaaa

SEQ ID NO: 8 - DNA, 405 bp, Artificial Sequence
  Synthetic construct
aagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacacc

SEQ ID NO: 9 - DNA, 24 bp, Artificial Sequence
  Synthetic construct
tgcttttgtgcccttttccatcgg

SEQ ID NO: 10 - DNA, 24 bp, Artificial Sequence
  Synthetic construct
tttccttcctctcatcccagctcg

SEQ ID NO: 11 - DNA, 551 bp, Homo sapiens
gtgctgagagaccccagtttcttctccaacccccaggacttcaatccccagcacttcctgaatgagaaggggcagtttaagaagagtgatgcttttgtgcccttttccatcggaaagcggaactgtttcggagaaggcctggccagaatggagctctttctcttcttcaccaccgtcatgcagaacttccgcctcaagtcctcccagtcacctaaggacattgacgtgtcccccaaacacgtgggctttgccacgatcccacgaaactacaccatgagcttcctgccccgctgagcgagggctgtgccggtgcaggtctggtgggcggggccagggaaagggcagggccaagaccgggcttgggagaggggcgcagctaagactgggggcaggatggcggaaaggaaggggcgtggtggctagagggaagagaagaaacagaagcggctcagttcaccttgataaggtgcttccgagctgggatgagaggaaggaaacccttacattatgctatgaagagtagtaataatagcagctcttatttcctgagc

SEQ ID NO: 12 - DNA, 408 bp, Artificial Sequence
  Syntethic construct
tgcttttgtgcccttttccatcggaaagcggaactgtttcggagaaggcctggccagaatggagctctttctcttcttcaccaccgtcatgcagaacttccgcctcaagtcctcccagtcacctaaggacattgacgtgtcccccaaacacgtgggctttgccacgatcccacgaaactacaccatgagcttcctgccccgctgagcgagggctgtgccggtgcaggtctggtgggcggggccagggaaagggcagggccaagaccgggcttgggagaggggcgcagctaagactgggggcaggatggcggaaaggaaggggcgtggtggctagagggaagagaagaaacagaagcggctcagttcaccttgataaggtgcttccgagctgggatgagaggaaggaaa

SEQ ID NO: 13 - DNA, 24 bp, Artificial Sequence
  Synthetic construct
gattctccagtctcagctcccaag

SEQ ID NO: 14 - DNA, 24 bp, Artificial Sequence
  Synthetic construct
tggggaggtcaggctttagagatg

SEQ ID NO: 15 - DNA, 1612 bp, Homo sapiens
caaaatacccccaacataccagatccgcttcctgccccgctgaaggggctgagggaagggggtcaaaggattccagggtcattcagtgtccccgcctctgtagacaatggctctgactccccgcaacttcctgcctctgagagacctgctacaagccagcttccttcccctccatggcaccagttgtctgaggtcacattgcaagtgagtgcaggagtgagattatcgaaaattataatatacaaaatcatatatatatatatgttcttgttttttgagacagagtctcacactgttgcccaggctggagtgcagtggcgtgatctcggctcactgcaacctccacccccggggatcaagcaactctcctgcctcagcctccctagtagctgggattacaggcatgcactaccacgcttggctaatttttgtatttttagtagagatggggtttcactgtgtaggccaggctggtctcgaactcctgaactcaagtgattcacccaccttagcctcccaaagtgctgggattacaggcgtgagtcaccgtgcccagccatgtatatatataattttaaaaattaagctgaaattcacataacataaaattagctgttttaaagtgtaaaatttagtggcgtgtggttcattcacaaagctgtacaaccaccaccatctagttccaaacattttctttttttctgagatggagtctcactctgtcacccaggttcgagttcagtggtgccatctctgtccactgcaacctccacatcctgggttcaagtgattctcctgcctcagcctctggaggagctggtatcacaggcgtcccccaccacgcctggctaaattttgtatttttaggtggtcttgaactcctgatgtcaggtgattctcctagctccaaatgttttcattatctctcccccaacaaaacccatacctatcaagctgtcactccccataccccattctctttttcatctcggcccctgtcaatctggtttttgtcactatggacttaccaattctgaatatttcccataaacagaatcatacaatatttgattttttttttttttttgaaactaagccttgctctgtctcccaggctggagtgctatggtgcaatttttgttcactgcaacctctgccttccaagatcaagagattctccagtctcagctcccaagtagctgggattacaggcatgtactaccatgcctggctaattttcttgtagttttagtagggacatgttggccaggctggtggtgagctcctggcctcaggtgatccacccacctcagtgttccaaagtgctgatattacaggcataatatgtgatcttttgtgtctggttgctttcatgttgaatgctatttttgaggttcatgcctgttgtagaccacagtcacacactgctgtagtcttccccagtcctcattcccagctgcctcttcctactgcttccgtctatcaaaaagcccccttggcccaggttccctgagctgtgggattctgcactggtgctttggattccctgatatgttccttcaaatctgctgagaattaaataaacatctctaaagcctgacctccccacgtc

SEQ ID NO: 16 - DNA, 436 bp, Artificial Sequence
  Synthetic construct
gattctccagtctcagctcccaagtagctgggattacaggcatgtactaccatgcctggctaattttcttgtagttttagtagggacatgttggccaggctggtggtgagctcctggcctcaggtgatccacccacctcagtgttccaaagtgctgatattacaggcataatatgtgatcttttgtgtctggttgctttcatgttgaatgctatttttgaggttcatgcctgttgtagaccacagtcacacactgctgtagtcttccccagtcctcattcccagctgcctcttcctactgcttccgtctatcaaaaagcccccttggcccaggttccctgagctgtgggattctgcactggtgctttggattccctgatatgttccttcaaatctgctgagaattaaataaacatctctaaagcctgacctcccca

SEQ ID NO: 17 - DNA, 24 bp, Artificial Sequence
  Synthetic construct
ttaaagaacctcaatactactgca

SEQ ID NO: 18 - DNA, 24 bp, Artificial Sequence
  Synthetic construct
tgaaccagcaattaataacacttt

SEQ ID NO: 19 - DNA, 651 bp, Homo sapiens
ccgtgctacatgatgacaaagaatttcctaatccaaatatctttgaccctggccactttctagataagaatggcaactttaagaaaagtgactacttcatgcctttctcagcaggaaaacgaatttgtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgctgatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactttttaatatgttatcactgtaaaacacagaaaagtgattaatgaatgataatttagtccatttcttttgtgaatgtgctaaataaaaagtgttattaattgctggttca

SEQ ID NO: 20 - DNA, 439 bp, Artificial Sequence
  Synthetic construct
ttaaagaacctcaatactactgcagttaccaaagggattgtttctctgccaccctcataccagatctgcttcatccctgtctgaagaatgctagcccatctggctgctgatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactttttaatatgttatcactgtaaaacacagaaaagtgattaatgaatgataatttagtccatttcttttgtgaatgtgctaaataaaaagtgttattaattgctggttca

SEQ ID NO: 21 - DNA, 24 bp, Artificial Sequence
  Synthetic construct
taaaactagggcacaaccataatg

SEQ ID NO: 22 - DNA, 24 bp, Artificial Sequence
  Synthetic construct
tgaaccagcaattaataacacttt

SEQ ID NO: 23 - DNA, 690 bp, Homo sapiens
cataaaactagggcacaaccataatggcattactgacttccgtgctacatgatgacaaagaatttcctaatccaaatatctttgaccctggccactttctagataagaatggcaactttaagaaaagtgactacttcatgcctttctcagcaggaaaacgaatttgtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgctgatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactttttaatatgttatcactgtaaaacacagaaaagtgattaatgaatgataatttagtccatttcttttgtgaatgtgctaaataaaaagtgttattaattgctggttca

SEQ ID NO: 24 - DNA, 688 bp, Artificial Sequence
  Synthetic construct
taaaactagggcacaaccataatggcattactgacttccgtgctacatgatgacaaagaatttcctaatccaaatatctttgaccctggccactttctagataagaatggcaactttaagaaaagtgactacttcatgcctttctcagcaggaaaacgaatttgtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgctgatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactttttaatatgttatcactgtaaaacacagaaaagtgattaatgaatgataatttagtccatttcttttgtgaatgtgctaaataaaaagtgttattaattgctggttca

SEQ ID NO: 25 - DNA, 24 bp, Artificial Sequence
  Synthetic construct
tttttattcctgacctccatttta

SEQ ID NO: 26 - DNA, 24 bp, Artificial Sequence
  Synthetic construct
gctttttatttagatcatgcagaa

SEQ ID NO: 27 - DNA, 635 bp, Homo sapiens
tttcccaacccagagatgtttgaccctcatcactttctggatgaaggtggcaattttaagaaaagtaaatacttcatgcctttctcagcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctgtgcttcattcctgtctgaagaagagcagatggcctggctgctgctgtgcagtccctgcagctctctttcctctggggcattatccatctttgcactatctgtaatgccttttctcacctgtcatctcacattttcccttccctgaagatctagtgaacattcgacctccattacggagagtttcctatgtttcactgtgcaaatatatctgctattctccatactctgtaacagttgcattgactgtcacataatgctcatacttatctaatgtagagtattaatatgttattattaaatagagaaatatgatttgtgtattataattcaaaggcatttcttttctgcatgatctaaataaaaagcattattatttgctg

SEQ ID NO: 28 - DNA, 486 bp, Artificial Sequence
  Synthetic construct
tttttattcctgacctccattttacagaactttaacctgaaatctctggttgacccaaagaaccttgacaccactccagttgtcaatggatttgcctctgtgccgcccttctaccagctgtgcttcattcctgtctgaagaagagcagatggcctggctgctgctgtgcagtccctgcagctctctttcctctggggcattatccatctttgcactatctgtaatgccttttctcacctgtcatctcacattttcccttccctgaagatctagtgaacattcgacctccattacggagagtttcctatgtttcactgtgcaaatatatctgctattctccatactctgtaacagttgcattgactgtcacataatgctcatacttatctaatgtagagtattaatatgttattattaaatagagaaatatgatttgtgtattataattcaaaggcatttcttttctgcatgatctaaataaaaagc

SEQ ID NO: 29 - DNA, 24 bp, Artificial Sequence
  Synthetic construct
gacatcaacaaccctcgggacttt

SEQ ID NO: 30 - DNA, 24 bp, Artificial Sequence
  Synthetic construct
atagaagggcgggacagaagcaaa

SEQ ID NO: 31 - DNA, 753 bp, Homo sapiens
gaaagtgatattttggagaaagtaaaagaacaccaagaatcgatggacatcaacaaccctcgggactttattgattgcttcctgatcaaaatggagaaggaaaagcaaaaccaacagtctgaattcactattgaaaacttggtaatcactgcagctgacttacttggagctgggacagagacaacaagcacaaccctgagatatgctctccttctcctgctgaagcacccagaggtcacagctaaagtccaggaagagattgaacgtgtcattggcagaaaccggagcccctgcatgcaggacaggggccacatgccctacacagatgctgtggtgcacgaggtccagagatacatcgacctcatccccaccagcctgccccatgcagtgacctgtgacgttaaattcagaaactacctcattcccaagggcacaaccatattaacttccctcacttctgtgctacatgacaacaaagaatttcccaacccagagatgtttgaccctcgtcactttctggatgaaggtggaaattttaagaaaagtaactacttcatgcctttctcagcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctatcagctgtgcttcattcctgtctga

SEQ ID NO: 32 - DNA, 684 bp, Artificial Sequence
  Synthetic construct
gacatcaacaaccctcgggactttattgattgcttcctgatcaaaatggagaaggaaaagcaaaaccaacagtctgaattcactattgaaaacttggtaatcactgcagctgacttacttggagctgggacagagacaacaagcacaaccctgagatatgctctccttctcctgctgaagcacccagaggtcacagctaaagtccaggaagagattgaacgtgtcattggcagaaaccggagcccctgcatgcaggacaggggccacatgccctacacagatgctgtggtgcacgaggtccagagatacatcgacctcatccccaccagcctgccccatgcagtgacctgtgacgttaaattcagaaactacctcattcccaagggcacaaccatattaacttccctcacttctgtgctacatgacaacaaagaatttcccaacccagagatgtttgaccctcgtcactttctggatgaaggtggaaattttaagaaaagtaactacttcatgcctttctcagcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctat

SEQ ID NO: 33 - DNA, 24 bp, Artificial Sequence
  Synthetic construct
ctgacctgttctctgccgggatgg

SEQ ID NO: 34 - DNA, 24 bp, Artificial Sequecne
  Synthetic construct
ttctagcggggcacagcacaaagc

SEQ ID NO: 35 - DNA, 815 bp, Homo sapiens
ggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtatgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctcctagcccagaggctctaatgtacaataaagcaatgtggtagttcc

SEQ ID NO: 36 - DNA, 598 bp, Artificial Sequence
  Synthetic construct
ctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtatgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaa

SEQ ID NO: 37 - DNA, 24 bp, Artificial Sequence
  Synthetic construct
agaagctccatgaagaaattgaca

SEQ ID NO: 38 - DNA, 24 bp, Artificial Sequence
  Synthetic construct
gtgatgatttatttatattctggg

SEQ ID NO: 39 - DNA, 829 bp, Homo sapiens
tgctcgtggaaatggagaaggaaaagcacagtgcagagcgcttgtacacaatggacggtatcaccgtgactgtggccgacctgttctttgcggggacagagaccaccagcacaactctgagatatgggctcctgattctcatgaaataccctgagatcgaagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaaccccccgctttcaaacaagttttcaaattgtttgaggtcaggatttctcaaactgattcctttctttgcatatgagtatttgaaaataaatattttcccagaatataaataaatcatcacatgattatttt

SEQ ID NO: 40 - DNA, 656 bp, Artificial Sequence
  Synthetic construct
agaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaaccccccgctttcaaacaagttttcaaattgtttgaggtcaggatttctcaaactgattcctttctttgcatatgagtatttgaaaataaatattttcccagaatataaataaatcatcac

SEQ ID NO: 41 - DNA, 24 bp, Artificial Sequence
  Synthetic construct
tttggtcattgtaatcactgttgg

SEQ ID NO: 42 - DNA, 24 bp, Artificial Sequence
  Synthetic construct
attaagtgttcattgcatcgagac

SEQ ID NO: 43 - DNA, 1208 bp, Homo sapiens
aaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctctgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagtattatcagatgctttcctgcacattaaggagaaatctatagaactgaatgagaaccaacaagtaaatatttttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaataaaaaacagtcgattggtc

SEQ ID NO: 44 - DNA, 607 bp, Artificial Sequence
  Synthetic construct
tttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaat

SEQ ID NO: 45 - DNA, 24 bp, Artificial Sequence
  Synthetic construct
atgatctgtctgtggcaaaagttt

SEQ ID NO: 46 - DNA, 24 bp, Artificial Sequence
  Synthetic construct
agttcctccattcatttgatttcc

SEQ ID NO: 47 - DNA, 1387 bp, Homo sapiens
ccaagaaactcagacaggtgtctggaacactagagaaggctggtcagtacctactctggagcatttctcatcagtagttcacatacaaatcatccatccttgccaatagtgtcatcctcacagtgaacactcagtggcccatggcattttataggcatacctcctatgggttgtcaccaagctaggtgctattggtcatctgctcctgttcacaccagagaaccaggctacaagagaaaaagcagaggccaagagtttgagggagaaatagtcggtgaagaaaccgtatccataaagacccgattccaccaaatgtgctttgagaaggataggccttcattaacaaaatgtatgtctggttccccagtagagctctactgcctcaacccaaggggatttttatgtctggggcagaaacactcaagttgattagaaagaccaggccaatgtcagggtacctggggccaaacccacctgctagtgtgaattaaagtactttaattttgttttctgtggaggtggaaaagcaacattcatagtctttggagaaatgcttagaaattcagcatttgacccttgctgtgaattaagcccaattaattcctgtttgtctacatatgatctgtctgtggcaaaagtttaatcagaggaaattctttcccagtctgtcgatttatgcctcagccacttgcctgtgctacaattcattgtgttacctgtagattcaggtaatacaaactatatataatcatcaagtaatacaaactaatttagtaatagcctgggttaagtattattagggccctgtgtctgctgtagaaaaaaaaattcacatgatgcacttcaaattcaaataaaaatccttttggcatgttcccatttttgcttagctcaattagtgtggctaaccaagagataactgtaaatgtgacattgatttgctcttactacagcttcagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctgaagtattttttaatcctacctcaaaatttaacagttacctgagagatttaacattatctagttcattgaatcattgtatgtggtcatggataaattgcacaccttggaattcgctttctaaaggaaatcaaatgaatggaggaactttccaaacaccactttacttgtgttatatagccaatataactatctctactgaatgtcattgaaaaactaaaaaattaaacttatttacaaataggtaaaaaaaaaaaaaaaaaaaaa

SEQ ID NO: 48 - DNA, 652 bp, Artificial Sequence
  Synthetic construct
atgatctgtctgtggcaaaagtttaatcagaggaaattctttcccagtctgtcgatttatgcctcagccacttgcctgtgctacaattcattgtgttacctgtagattcaggtaatacaaactatatataatcatcaagtaatacaaactaatttagtaatagcctgggttaagtattattagggccctgtgtctgctgtagaaaaaaaaattcacatgatgcacttcaaattcaaataaaaatccttttggcatgttcccatttttgcttagctcaattagtgtggctaaccaagagataactgtaaatgtgacattgatttgctcttactacagcttcagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctgaagtattttttaatcctacctcaaaatttaacagttacctgagagatttaacattatctagttcattgaatcattgtatgtggtcatggataaattgcacaccttggaattcgctttctaaaggaaatcaaatgaatggaggaact

SEQ ID NO: 49 - DNA, 24 bp, Artificial Sequence
  Synthetic construct
atgatctgtctgtggcaaaagttt

SEQ ID NO: 50 - DNA, 24 bp, Artificial Sequence
  Synthetic construct
agttcctccattcatttgatttcc

SEQ ID NO: 51 - DNA, 1376 bp, Homo sapiens
agacaggtgtctggaacactagagaaggctggtcagtacctactctggagcatttctcatcagtagttcacatacaaatcatccatccttgccaatagtgtcatcctcacagtgaacactcagtggcccatggcattttataggcatacctcctatgggttgtcaccaagctaggtgctattggtcatctgctcctgttcacaccagagaaccaggctacaagagaaaaagcagaggccaagagtttgagggagaaatagtcggtgaagaaaccgtatccataaagacccgattccaccaaatgtgctttgagaaggataggccttcattaacaaaatgtatgtctggttccccagtagagctctactgcctcaacccaaggggatttttatgtctggggcagaaacactcaagttgattagaaagaccaggccaatgtcagggtacctggggccaaacccacctgctagtgtgaattaaagtactttaattttgttttctgtggaggtggaaaagcaacattcatagtctttggagaaatgcttagaaattcagcatttgacccttgctgtgaattaagcccaattaattcctgtttgtctacatatgatctgtctgtggcaaaagtttaatcagaggaaattctttcccagtctgtcgatttatgcctcagccacttgcctgtgctacaattcattgtgttacctgtagattcaggtaatacaaactatatataatcatcaagtaatacaaactaatttagtaatagcctgggttaagtattattagggccctgtgtctgctgtagaaaaaaaaattcacatgatgcacttcaaattcaaataaaaatccttttggcatgttcccatttttgcttagctcaattagtgtggctaaccaagagataactgtaaatgtgacattgatttgctcttactacagcttcagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctgaagtattttttaatcctacctcaaaatttaacagttacctgagagatttaacattatctagttcattgaatcattgtatgtggtcatggataaattgcacaccttggaattcgctttctaaaggaaatcaaatgaatggaggaactttccaaacaccactttacttgtgttatatagccaatataactatctctactgaatgtcattgaaaaactaaaaaattaaacttatttacaaataggtaaaaaaaaaaaaaaaaaaaaa

SEQ ID NO: 52 - DNA, 652 bp, Artificial Sequence; identical to SEQ ID NO: 48
  Synthetic construct

SEQ ID NO: 53 - DNA, 24 bp, Artificial Sequence
  Synthetic construct
ccgggaccccactgccttctctga

SEQ ID NO: 54 - DNA, 24 bp, Artificial Sequence
  Synthetic construct
ttttatattctacccaaggacagc

SEQ ID NO: 55 - DNA, 619 bp, Homo sapiens
gatggcttcctcttccccaagaacacccagtttgtgttctgccactatgtggtgtcccgggaccccactgccttctctgagcctgaaagcttccagccccaccgctggctgagaaacagccagcctgctacccccaggatccagcacccatttggctctgtgccctttggctatggggtccgggcctgcctgggccgcaggattgcagagctggagatgcagctactcctcgcaaggctgatccagaagtacaaggtggtcctggccccggagacgggggagttgaagagtgtggcccgcattgtcctggttcccaataagaaagtgggcctgcagttcctgcagagacagtgctgagctgagtctccgccttgctggggcttgtcctagaggctccagctctggcacagtggttcctggctgctgccatgtctcagatgaggagggagagaaggaggccgccagactcgagaggtgggaggaactccttgcacacaccctgagcttttgccacttctatcatttttgagcaactccctctcagctaaaaggccacccctttatcgcattgctgtccttgggtagaatataaaataaagggacttttatttcttattgg

SEQ ID NO: 56 - DNA, 538 bp, Artificial Sequence
  Synthetic construct
ccgggaccccactgccttctctgagcctgaaagcttccagccccaccgctggctgagaaacagccagcctgctacccccaggatccagcacccatttggctctgtgccctttggctatggggtccgggcctgcctgggccgcaggattgcagagctggagatgcagctactcctcgcaaggctgatccagaagtacaaggtggtcctggccccggagacgggggagttgaagagtgtggcccgcattgtcctggttcccaataagaaagtgggcctgcagttcctgcagagacagtgctgagctgagtctccgccttgctggggcttgtcctagaggctccagctctggcacagtggttcctggctgctgccatgtctcagatgaggagggagagaaggaggccgccagactcgagaggtgggaggaactccttgcacacaccctgagcttttgccacttctatcatttttgagcaactccctctcagctaaaaggccacccctttatcgcattgctgtccttgggtagaatataaaa

SEQ ID NO: 57 - DNA, 24 bp, Artificial Sequence
  Synthetic construct
cttcccctaatgcctatctgacca

SEQ ID NO: 58 - DNA, 24 bp, Artificial Sequence
  Synthetic construct
cctgagaactaagtgatggggcaa

SEQ ID NO: 59 - DNA, 833 bp, Homo sapiens
atcaacctacagtttttggacagatagtcccatggaaagagactgtcatcatcaccctttcattcatcatagggataagattttttgtaggcacaagaccaaggtatacatcttcccctaatgcctatctgaccaaactggatagaaccaccatagtgaagtgtgaggcggccctgaccaatgtgtgaagtatgcacttggcctgactcaggaagccaggtgagaaaaccatggtctctctgcttgcttggcccttctgatcatgtatgcatcccccaaggatgaaatcagattttaactaataatgctggatggcctgaggaaagattcaactgcctctctttttgggctttcatagtgttcattgatgctgctggctaagcatttatcaaagcataagctcagtaactgtgcatctggtctgtacctggttggtccttcgtctttgcatgtaagctctttgagaggaagggtgaagccttatttgttttttatgtcccctgccagggcctgtctctgactaggtgtcaccatacacattcttagattgaatctgaaccatgtggcagaagggataagcagcttacttagtaggctctgtctacccccttccttctttgtcttgcccctaggaaggtgaatctgccctagcctggtttacggtttcttataactctcctttgctctctggccactattaagtgggtttgccccatcacttagttctcaggcagagacatctttgggcctgtccctgcccaggcctctggctttttatattgaaaatttttaaatattcacaaattttagaataaatcaaatattccattctt

SEQ ID NO: 60 - DNA, 620 bp, Artificial Sequence
  Synthetic construct
cttcccctaatgcctatctgaccaaactggatagaaccaccatagtgaagtgtgaggcggccctgaccaatgtgtgaagtatgcacttggcctgactcaggaagccaggtgagaaaaccatggtctctctgcttgcttggcccttctgatcatgtatgcatcccccaaggatgaaatcagattttaactaataatgctggatggcctgaggaaagattcaactgcctctctttttgggctttcatagtgttcattgatgctgctggctaagcatttatcaaagcataagctcagtaactgtgcatctggtctgtacctggttggtccttcgtctttgcatgtaagctctttgagaggaagggtgaagccttatttgttttttatgtcccctgccagggcctgtctctgactaggtgtcaccatacacattcttagattgaatctgaaccatgtggcagaagggataagcagcttacttagtaggctctgtctacccccttccttctttgtcttgcccctaggaaggtgaatctgccctagcctggtttacggtttcttataactctcctttgctctctggccactattaagtgggtttgccccatcacttagttctcagg

SEQ ID NO: 61 - DNA, 24 bp, Artificial Sequence
  Synthetic construct
caaacacaaaacttcctctgcggg

SEQ ID NO: 62 - DNA, 24 bp, Artificial Sequence
  Synthetic construct
tctttcattgcaaccactgggctc

SEQ ID NO: 63 - DNA, 736 bp, Homo sapiens
tgcccaccctggcccctgtgctgcctctggtcacacacttcgcagacatcaacactttcatggtactgcaagtcatcaagtttactaaggacctgcccgtcttccgttccctgcccattgaagaccagatctcccttctcaagggagcagctgtggaaatctgtcacatcgtactcaataccactttctgtctccaaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatgccgctgctccaggagatctgcagctgaggccatgctcacttccttccccagctcacctggaacaccctggatacactggagtgggaaaatgctgggaccaaagattgggccgggttcaaagggagcccagtggttgcaatgaaagactaaagcaaaac

SEQ ID NO: 64 - DNA, 530 bp, Artificial Sequence
  Synthetic construct
caaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatgccgctgctccaggagatctgcagctgaggccatgctcacttccttccccagctcacctggaacaccctggatacactggagtgggaaaatgctgggaccaaagattgggccgggttcaaagggagcccagtggttgcaatgaaaga

SEQ ID NO: 65 - DNA, 24 bp, Artificial Sequence
  Synthetic construct
ccagatagacaatacataaaggat

SEQ ID NO: 66 - DNA, 24 bp, Artificial Sequence
  Synthetic construct
ctgttgccattatgtttgctttat

SEQ ID NO: 67 - DNA, 718 bp, Homo sapiens
ctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtgatggggattacaggggaggggtctagctcctttttctctctcatattaatctgatgtataactttcctttatttcacttgtacccagtttcactcaagaaatcttgatgaatatttatgttgtaattacatgtgtaacttccacaactgtaaatattgggctagatagaacaactttctctacattgtgttttaaaaggctccagggaatcctgcattctaattggcaagccctgtttgcctaattaaattgattgttacttcaattctatctgttgaactagggaaaatctcattttgctcatcttaccatattgcatatattttattaaagagttgtattcaatcttggcaataaagcaaacataatggcaacagaaaaaaaaaaaaaaaaaaaaaaaaaaaaaaaaaaaaaaaaaaaaaaaaaaaaaaaaaaaaaaaaaaaaaa

SEQ ID NO: 68 - DNA, 619 bp, Artificial Sequence
  Synthetic construct
ccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtgatggggattacaggggaggggtctagctcctttttctctctcatattaatctgatgtataactttcctttatttcacttgtacccagtttcactcaagaaatcttgatgaatatttatgttgtaattacatgtgtaacttccacaactgtaaatattgggctagatagaacaactttctctacattgtgttttaaaaggctccagggaatcctgcattctaattggcaagccctgtttgcctaattaaattgattgttacttcaattctatctgttgaactagggaaaatctcattttgctcatcttaccatattgcatatattttattaaagagttgtattcaatcttggcaataaagcaaacataatggcaacag

SEQ ID NO: 69 - DNA, 24 bp, Artificial Sequence
  Synthetic construct
gctcatcgccatcaacatcttctc

SEQ ID NO: 70 - DNA, 24 bp, Artificial Sequence
  Synthetic construct
taaaagcagaggaagaggaaggcc

SEQ ID NO: 71 - DNA, 690 bp, Homo sapiens
catgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctagggtggaaggggccctgggcgagcctgtagacctatcggctctcatcccttgggataagccccagtccaggtccaggaggctccctccctgcccagcgagtcttccagaaggggtgaaagggttgcaggtcccgaccactgacccttcccggctgccctccctccccagcttacacctcaagcccagcacgcagcgtaccttgaacagagggaggggaggacccatggctctccccccctagcccgggagaccaggggccttcctcttcctctgcttttatttaataaaaataaaaacagaaa

SEQ ID NO: 72 - DNA, 628 bp, Artificial Sequence
  Synthetic construct
gctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctagggtggaaggggccctgggcgagcctgtagacctatcggctctcatcccttgggataagccccagtccaggtccaggaggctccctccctgcccagcgagtcttccagaaggggtgaaagggttgcaggtcccgaccactgacccttcccggctgccctccctccccagcttacacctcaagcccagcacgcagcgtaccttgaacagagggaggggaggacccatggctctccccccctagcccgggagaccaggggccttcctcttcctctgctttta

SEQ ID NO: 73 - DNA, 24 bp, Artificial Sequence
  Synthetic construct
aagcagaaagcagaaaccacagac

SEQ ID NO: 74 - DNA, 24 bp, Artificial Sequence
  Synthetic construct
cagtaggacatcccaaacacagaa

SEQ ID NO: 75 - DNA, 705 bp, Homo sapiens
cgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaatttt

SEQ ID NO: 76 - DNA, 582 bp, Artificial Sequence
  Synthetic construct
aagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactg

SEQ ID NO: 77 - DNA, 24 bp, Artificial Sequence
  Synthetic construct
cacacacacataagcactgaaatc

SEQ ID NO: 78 - DNA, 24 bp, Artificial Sequence
  Synthetic construct
aaagtttcgtcagtctgtgtacac

SEQ ID NO: 79 - DNA, 964 bp, Homo sapiens
actccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaa

SEQ ID NO: 80 - DNA, 914 bp, Artificial Sequence
  Synthetic construct
cacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaacttt

SEQ ID NO: 81 - DNA, 24 bp, Artificial Sequence
  Synthetic construct
aggaaagacaacagacaaatcacc

SEQ ID NO: 82 - DNA, 24 bp, Artificial Sequence
  Synthetic construct
ttaggtgtcagattttccctcaga

SEQ ID NO: 83 - DNA, 1043 bp, Homo sapiens
gaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 84 - DNA, 795 bp, Artificial Sequence
  Synthetic construct
aggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaa

SEQ ID NO: 85 - DNA, 24 bp, Artificial Sequence
  Synthetic construct
cagacagctttagccgttcccaat

SEQ ID NO: 86 - DNA, 24 bp, Artificial Sequence
  Synthetic construct
tctcctctcacacttctccctttg

SEQ ID NO: 87 - DNA, 659 bp, Homo sapiens
tgctttggagcagacagctttagccgttcccaatccttagcaatgccttagctgggacgcatagctaatactttagagaggatgacagatccataaagagagtaaagataagagaaaatgtctaaagcatctggaaaggtaaaaaaaaaaaatctatttttgtacaaatgtaattttatccctcatgtatacttggatatggcggggggagggctgggactgtttcgtttctgcttctagagattgaggtgaaagcttcgtccgagaaacgccaggacagacgatggcagaggagagggctcctgtgacggcggcgaggcttgggaggaaaccgccgcaatgggggtgtcttccctcggggcaggagggtgggcctgaggctttcaagggttttcttccctttcgagtaatttttaaagccttgctctgttgtgtcctgttgccggctctggccttcctgtgactgactgtgaagtggcttctccgtacgattgtctctgaaacatcgtggcctcaggtgccagggtttgatggacagtagcattagaattgtggaaaaggaacacgcaaagggagaagtgtgagaggagaaacaaaatatgagcgtttaaaatacatcgccattcagttcgttaaaaaaaaaaaaaaaaaaaaaaaaa

SEQ ID NO: 88 - DNA, 581 bp, Artificial Sequence
  Synthetic construct
cagacagctttagccgttcccaatccttagcaatgccttagctgggacgcatagctaatactttagagaggatgacagatccataaagagagtaaagataagagaaaatgtctaaagcatctggaaaggtaaaaaaaaaaaatctatttttgtacaaatgtaattttatccctcatgtatacttggatatggcggggggagggctgggactgtttcgtttctgcttctagagattgaggtgaaagcttcgtccgagaaacgccaggacagacgatggcagaggagagggctcctgtgacggcggcgaggcttgggaggaaaccgccgcaatgggggtgtcttccctcggggcaggagggtgggcctgaggctttcaagggttttcttccctttcgagtaatttttaaagccttgctctgttgtgtcctgttgccggctctggccttcctgtgactgactgtgaagtggcttctccgtacgattgtctctgaaacatcgtggcctcaggtgccagggtttgatggacagtagcattagaattgtggaaaaggaacacgcaaagggagaagtgtgagaggaga

SEQ ID NO: 89 - DNA, 24 bp, Artificial Sequence
  Synthetic construct
ccttgcttccttctcatcttgcct

SEQ ID NO: 90 - DNA, 24 bp, Artificial Sequence
  Synthetic construct
tatgtatgagaggggaaaggagcc

SEQ ID NO: 91 - DNA, 802 bp, Artificial Sequence
  Synthetic construct
ctccaggaccttgcttccttctcatcttgcctcattttgcttcccatctgaagagtggaaatggggaactcccccagaggtggatactggggggcaggcctcccaagctgatggacatgagagtagggccctgacaggccttcctcctctcaaacctggcagatgggggcctctctggaagagggaggggccctgtcactgtccagagtctctttttacacttcacctccttctgcagtcagactgaaatataaaaaaggtggtggtggtggtgaaggggctggtggagatgtaggaaccgatctgctatttttaatttcctgtgaggatagagacttgcagttagactcaaagaagtactgtactttcccaggttgactaagaaatgccagtggtggaggtgggtgtttgggaaaggcagggccctgaaatggcctgtccctagggctctccaagcactagccttcccagcttcccgccgccccccctatctcttcctgtctaacttggggaaggggcctgggctgtgaggacagggcccccacaggggatggtttcacgagtgtagtcccggaggccttccctttacagctctcctccagccctgggcacatagcataggctggggacacaggatcctggcctgagaattgaggggaggtggccagcccgcagaggtggggtgctggggctgcatgatttttgccctgcgtcccttctctttggggctcctttcccctctcatacataaaatcgctttcaaattaaaatcgctgttttctggaaaaaaaaaaaaaaaaaa

SEQ ID NO: 92 - DNA, 742 bp, Homo sapiens
ccttgcttccttctcatcttgcctcattttgcttcccatctgaagagtggaaatggggaactcccccagaggtggatactggggggcaggcctcccaagctgatggacatgagagtagggccctgacaggccttcctcctctcaaacctggcagatgggggcctctctggaagagggaggggccctgtcactgtccagagtctctttttacacttcacctccttctgcagtcagactgaaatataaaaaaggtggtggtggtggtgaaggggctggtggagatgtaggaaccgatctgctatttttaatttcctgtgaggatagagacttgcagttagactcaaagaagtactgtactttcccaggttgactaagaaatgccagtggtggaggtgggtgtttgggaaaggcagggccctgaaatggcctgtccctagggctctccaagcactagccttcccagcttcccgccgccccccctatctcttcctgtctaacttggggaaggggcctgggctgtgaggacagggcccccacaggggatggtttcacgagtgtagtcccggaggccttccctttacagctctcctccagccctgggcacatagcataggctggggacacaggatcctggcctgagaattgaggggaggtggccagcccgcagaggtggggtgctggggctgcatgatttttgccctgcgtcccttctctttggggctcctttcccctctcatacata

SEQ ID NO: 93 - DNA, 24 bp, Artificial Sequence
  Synthetic construct
ttgctgattgcctctttctcccac

SEQ ID NO: 94 - DNA, 24 bp, Artificial Sequence
  Synthetic construct
atcacattttggggacaggaaggg

SEQ ID NO: 95 - DNA, 672 bp, Homo sapiens
ttggaggaccaggtcattttgcttcgggcagggtggaatgaattgctgattgcctctttctcccaccgctcagtttccgtgcaggatggcatccttctggccacgggtttacatgtccaccggagcagtgcccacagtgctggggtcggctccatctttgacagagtcctaactgagctggtttccaaaatgaaagacatgcagatggacaagtcggaactgggatgcctgcgagccattgtactctttaacccagatgccaagggcctgtccaacccctctgaggtggagactctgcgagagaaggtttatgccacccttgaggcctacaccaagcagaagtatccggaacagccaggcaggtttgccaagctgctgctgcgcctcccagctctgcgttccattggcttgaaatgcctggagcacctcttcttcttcaagctcatcggggacacccccattgacaccttcctcatggagatgttggagaccccgctgcagatcacctgagccccaccagccacagcctccccacccaggatgacccctgggcaggtgtgtgtggacccccaccctgcactttcctccacctcccaccctgacccccttcctgtccccaaaatgtgatgcttataataaagaaaacctttctacaaaaaaaaaaaaaaaaaa

SEQ ID NO: 96 - DNA, 586 bp, Artificial Sequence
  Synthetic construct
ttgctgattgcctctttctcccaccgctcagtttccgtgcaggatggcatccttctggccacgggtttacatgtccaccggagcagtgcccacagtgctggggtcggctccatctttgacagagtcctaactgagctggtttccaaaatgaaagacatgcagatggacaagtcggaactgggatgcctgcgagccattgtactctttaacccagatgccaagggcctgtccaacccctctgaggtggagactctgcgagagaaggtttatgccacccttgaggcctacaccaagcagaagtatccggaacagccaggcaggtttgccaagctgctgctgcgcctcccagctctgcgttccattggcttgaaatgcctggagcacctcttcttcttcaagctcatcggggacacccccattgacaccttcctcatggagatgttggagaccccgctgcagatcacctgagccccaccagccacagcctccccacccaggatgacccctgggcaggtgtgtgtggacccccaccctgcactttcctccacctcccaccctgacccccttcctgtccccaaaatgtgat

SEQ ID NO: 97 - DNA, 24 bp, Artificial Sequence
  Synthetic construct
cacgtttgttcgcttcctgagtct

SEQ ID NO: 98 - DNA, 24 bp, Artificial Sequence
  Synthetic construct
caagtggctataaacaaggcaggc

SEQ ID NO: 99 - DNA, 926 bp, Homo sapiens
ctggggtctatgcccacatacccacgtttgttcgcttcctgagtcttttcattgctacctctaatagtcctgtctcccacttcccactcgttcccctcctcttccgagctgctttgtgggctccaggcctgtactcatcggcaggtgcatgagtatctgtgggagtcctctagagagatgagaagccaggaggcctgcaccaaatgtcagaagcttggcatgacctcattccggccacatcattctgtgtctctgcatccatttgaacacattattaagcaccgataataggtagcctgctgtggggtatacagcattgactcagatatagatcctgagctcacagagtttatagttaaaaaaacaaacagaaacacaaacaatttggatcaaaaggagaaatgataagtgacaaaagcagcacaaggaatttccctgtgtggatgctgagctgtgatggcgggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctaaaaaaaaaaaaaaaaaaaaaaaaaaaaaa

SEQ ID NO: 100 - DNA, 736 bp, Artificial Sequence
  Synthetic construct
cacgtttgttcgcttcctgagtcttttcattgctacctctaatagtcctgtctcccacttcccactcgttcccctcctcttccgagctgctttgtgggctccaggcctgtactcatcggcaggtgcatgagtatctgtgggagtcctctagagagatgagaagccaggaggcctgcaccaaatgtcagaagcttggcatgacctcattccggccacatcattctgtgtctctgcatccatttgaacacattattaagcaccgataataggtagcctgctgtggggtatacagcattgactcagatatagatcctgagctcacagagtttatagttaaaaaaacaaacagaaacacaaacaatttggatcaaaaggagaaatgataagtgacaaaagcagcacaaggaatttccctgtgtggatgctgagctgtgatggcgggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttg

SEQ ID NO: 101 - DNA, 24 bp, Artificial Sequence
  Synthetic construct
ccaccctgttctctacctcttcta

SEQ ID NO: 102 - DNA, 24 bp, Artificial Sequence
  Synthetic construct
cagaatctcactatgttgcccagg

SEQ ID NO: 103 - DNA, 1042 bp, Homo sapiens
ttgcagaggcactggggcccctgcagagcttccaggcccggcctgatgacctgctcatcagcacctaccccaagtccggcactacctgggtaagccagattctggacatgatctaccagggtggtgacctggagaagtgtcaccgagctcccatcttcatgcgggtgcccttccttgagttcaaagccccagggattccctcagggatggagactctgaaagacacaccggccccacgactcctgaagacacacctgcccctggctctgctcccccagactctgttggatcagaaggtcaaggtggtctatgttgcccgcaacgcaaaggatgtggcagtttcctactaccacttctaccacatggccaaggtgcaccctgagcctgggacctgggacagcttcctggagaagttcatggtcggagaagtgtcctacggatcctggtaccagcacgtgcaggagtggtgggagctgagccgcacccaccctgttctctacctcttctatgaagacatgaaggagaacccgaaaagggagattcaaaagatcctggagtttgtggggcgctccctgccagaggagaccgtggacttcgtggttcagcacacgtcgttcaaggagatgaagaagaaccctatgaccaactacaccaccgtcccccaggagttcatggaccacagcatctcccccttcatgaggaaaggcatggctggggactggaagaccaccttcaccgtggcgcagaatgagcgcttcgatgcggactatgcggagaagatggcaggctgcagcctcagcttccgctctgagctgtgagaggggctcctggagtcactgcagagggagtgtgcgaatcaaacctgaccaagcggctcaagaataaaatatgaattgagggcccgggacggtaggtcatgtctgtaatcccagcaatttggaggctgaggtgggaggatcatttgagcccaggagttcgagaccaacctgggcaacatagtgagattctgttaaaaaaataaaataaaataaaaccaattttt

SEQ ID NO: 104 - DNA, 525 bp, Artificial Sequence
  Synthetic construct
ccaccctgttctctacctcttctatgaagacatgaaggagaacccgaaaagggagattcaaaagatcctggagtttgtggggcgctccctgccagaggagaccgtggacttcgtggttcagcacacgtcgttcaaggagatgaagaagaaccctatgaccaactacaccaccgtcccccaggagttcatggaccacagcatctcccccttcatgaggaaaggcatggctggggactggaagaccaccttcaccgtggcgcagaatgagcgcttcgatgcggactatgcggagaagatggcaggctgcagcctcagcttccgctctgagctgtgagaggggctcctggagtcactgcagagggagtgtgcgaatcaaacctgaccaagcggctcaagaataaaatatgaattgagggcccgggacggtaggtcatgtctgtaatcccagcaatttggaggctgaggtgggaggatcatttgagcccaggagttcgagaccaacctgggcaacatagtgagattctg

SEQ ID NO: 105 - DNA, 24 bp, Artificial Sequence
  Synthetic construct
gctcgtaatgccaaggatgtttca

SEQ ID NO: 106 - DNA, 24 bp, Artificial Sequence
  Synthetic construct
gcccaaatcaattcataactgccc

SEQ ID NO: 107 - DNA, 1034 bp, Homo sapiens
tttccccaaaagatattctgcgaaaagatctgaagttggtccatggttatcccatgacctgtgcttttgcaagcaactgggaaaaaattgaacagttccatagcagaccagatgacattgtgatagccacttatcctaaatcaggtactacttgggttagtgaaattatagacatgattctaaatgatggagatattgaaaaatgtaagcgaggttttattactgaaaaagttccaatgttggaaatgactctccctggattaagaacatcaggtatagaacaattggagaagaatccatcaccccggattgtgaaaacacatctaccgactgatcttcttcctaaatctttctgggaaaacaattgcaagatgatttatctggctcgtaatgccaaggatgtttcagtctcatattaccattttgacttaatgaataatttacagccttttcctggtacctgggaagaatatctggagaaattcttaactggaaaagtggcctatggttcctggtttactcatgttaaaaactggtggaagaaaaaggaagaacacccaatactttttttgtactatgaagatatgaaagagaatccaaaggaggaaatcaagaagatcattagatttctagagaagaacctgaatgatgagatcttggataggatcatccatcacacctcatttgaagtgatgaaggacaatcctttggtaaattatacacatctaccaactacagtgatggatcatagcaaatccccttttatgcgtaaagggacggctggtgactggaagaattacttcaccgtggcccaaaatgagaaatttgatgctatttatgagacagaaatgtccaaaactgcacttcaattccgcacagagatttaaagtgtctaagtcacaaatctgaagaaataagagattgtctgtagttgattgaaacgagggcagttatgaattgatttgggcaatcaaatgaatttataaaggagaataatatgcttttaaaaaaaaaaaaaaaaaaaaaaaaaaaaa

SEQ ID NO: 108 - DNA, 585 bp, Artificial Sequence
  Synthetic construct
gctcgtaatgccaaggatgtttcagtctcatattaccattttgacttaatgaataatttacagccttttcctggtacctgggaagaatatctggagaaattcttaactggaaaagtggcctatggttcctggtttactcatgttaaaaactggtggaagaaaaaggaagaacacccaatactttttttgtactatgaagatatgaaagagaatccaaaggaggaaatcaagaagatcattagatttctagagaagaacctgaatgatgagatcttggataggatcatccatcacacctcatttgaagtgatgaaggacaatcctttggtaaattatacacatctaccaactacagtgatggatcatagcaaatccccttttatgcgtaaagggacggctggtgactggaagaattacttcaccgtggcccaaaatgagaaatttgatgctatttatgagacagaaatgtccaaaactgcacttcaattccgcacagagatttaaagtgtctaagtcacaaatctgaagaaataagagattgtctgtagttgattgaaacgagggcagttatgaattgatttgggc

SEQ ID NO: 109 - DNA, 24 bp, Artificial Sequence
  Synthetic construct
aaagcaatgccctctccacggata

SEQ ID NO: 110 - DNA, 24 bp, Artificial Sequence
  Synthetic construct
tctggctgggactgaaggattgaa

SEQ ID NO: 111 - DNA, 1020 bp, Homo sapiens
gcagggacaacgtggattcaggaaattgtggatatgattgaacagaatggggacgtggagaagtgccagcgagccatcatccaacaccgccatcctttcattgagtgggctcggccaccccaaccttctggtgtggaaaaagccaaagcaatgccctctccacggatactaaagactcacctttccactcagctgctgccaccgtctttctgggaaaacaactgcaagttcctttatgtagctcgaaatgccaaagactgtatggtttcctactaccatttccaaaggatgaaccacatgcttcctgaccctggtacctgggaagagtattttgaaaccttcatcaatggaaaagtggtttggggttcctggtttgaccacgtgaaaggatggtgggagatgaaagacagacaccagattctcttcctcttctatgaggacataaagagggacccaaagcatgaaattcggaaggtgatgcagttcatgggaaagaaggtggatgaaacagtgctagataaaattgtccaggagacgtcatttgagaaaatgaaagaaaatcccatgacaaatcgttctacagtttccaaatctatcttggaccagtcaatttcctccttcatgagaaaaggaactgtgggggattggaaaaaccacttcactgttgcccagaatgagaggtttgatgaaatctatagaagaaagatggaaggaacctccataaacttctgcatggaactctgagcaagatgtaaataaaattaaaaggtggatggcaagagtgcaaatactatcttcaatccttcagtcccagccagaagaatctctgaaagcatattgtgaatgtatacaatgtagtacaaacaatctctgtgatgattaacagtatgtcaccacttcattttttaaaaaggatcacgtctaatgcccattttcccaactattctttccaaagtaagatataaggtagcttaataaactaagtaaaacgtaaaaaaaaaaaaaaaaaaaaaaaaaaaa

SEQ ID NO: 112 - DNA, 675 bp, Artificial Sequence
  Synthetic construct
aaagcaatgccctctccacggatactaaagactcacctttccactcagctgctgccaccgtctttctgggaaaacaactgcaagttcctttatgtagctcgaaatgccaaagactgtatggtttcctactaccatttccaaaggatgaaccacatgcttcctgaccctggtacctgggaagagtattttgaaaccttcatcaatggaaaagtggtttggggttcctggtttgaccacgtgaaaggatggtgggagatgaaagacagacaccagattctcttcctcttctatgaggacataaagagggacccaaagcatgaaattcggaaggtgatgcagttcatgggaaagaaggtggatgaaacagtgctagataaaattgtccaggagacgtcatttgagaaaatgaaagaaaatcccatgacaaatcgttctacagtttccaaatctatcttggaccagtcaatttcctccttcatgagaaaaggaactgtgggggattggaaaaaccacttcactgttgcccagaatgagaggtttgatgaaatctatagaagaaagatggaaggaacctccataaacttctgcatggaactctgagcaagatgtaaataaaattaaaaggtggatggcaagagtgcaaatactatcttcaatccttcagtcccagccaga

SEQ ID NO: 113 - DNA, 24 bp, Artificial Sequence
  Synthetic construct
ccttgactcaattgatcctcccat

SEQ ID NO: 114 - DNA, 24 bp, Artificial Sequence
  Synthetic construct
cattcccataggttatagttgtgc

SEQ ID NO: 115 - DNA, 1025 bp, Homo sapiens
tacatcatacttcgttccaagagatgaagaacaatccatccacaaattacacaacactgccagacgaaattatgaaccagaaattgtcgcccttcatgagaaagggaattacaggagactggaaaaatcactttacagtagccctgaatgaaaaatttgataaacattatgagcagcaaatgaaggaatctacactgaagtttcgaactgagatctaagaaggtctttctttacttaacatatctgatattaaagatttcttttcattattctccactttttcttattttagattgctagaaaagacataatcatggattatgttgacattttctttttaaatttttgtttaactttttttttttttttttgagacagagtctcactctgttgcctaggctggaggacagtggcacaatcatggctgattgcagccttgacctccttgactcaattgatcctcccatctcagcctcccaagtagctaggactacagacatgtgcaaccatgtttggctaatttttttaatgtttttttgtagagatgaggtcttattatattgtccaggctggtcttgaattcctgggctcaagcttcccaagtagctgcaacaacaggcacacaccaccatgctcaactaattttatttctattttttgtatagacaggggcttgctatagtgtccaggctggtctgaaacccttgagctcaagtgatcttcccacaccagcctcccaaaatactgggattacaggcttgagcctccatgcctggcccaggtaacatgtttattgagctgtacatgcatatgagaaataagaaacttttttttcctactatcatctcttaaattttgttttctttttcttttgcttcctcttcttcttttctattttttataaatatcatgcacaactataacctatgggaatgatgtagtaacacagattattcatcttgttagagttgtattaaaaataaacaagcatttcaaattaaaaaaaaaaaaaaaaaaaaaaaaaa

SEQ ID NO: 116 - DNA, 492 bp, Artificial Sequence
  Synthetic construct
ccttgactcaattgatcctcccatctcagcctcccaagtagctaggactacagacatgtgcaaccatgtttggctaatttttttaatgtttttttgtagagatgaggtcttattatattgtccaggctggtcttgaattcctgggctcaagcttcccaagtagctgcaacaacaggcacacaccaccatgctcaactaattttatttctattttttgtatagacaggggcttgctatagtgtccaggctggtctgaaacccttgagctcaagtgatcttcccacaccagcctcccaaaatactgggattacaggcttgagcctccatgcctggcccaggtaacatgtttattgagctgtacatgcatatgagaaataagaaacttttttttcctactatcatctcttaaattttgttttctttttcttttgcttcctcttcttcttttctattttttataaatatcatgcacaactataacctatgggaatg

SEQ ID NO: 117 - DNA, 24 bp, Artificial Sequence
  Synthetic construct
gatgtccaattattccctcctgag

SEQ ID NO: 118 - DNA, 24 bp, Artificial Sequence
  Synthetic construct
atagggtttcatcatgttggccag

SEQ ID NO: 119 - DNA, 1059 bp, Homo sapiens
tctcaagaacagctcctttcagagcatgaaagaaaacaagatgtccaattattccctcctgagtgttgattatgtagtggacaaagcacaacttctgagaaaaggtgtatctggggactggaaaaatcacttcacagtggcccaagctgaagactttgataaattgttccaagagaagatggcagatcttcctcgagagctgttcccatgggaataacgtccaaaacactctggatcttatatggagaatgacattgattctcctgtccttgtacatgtacctgactggggtcattgtgtaagacttattattttatcctgaaaccttaaatatcaaacctctgcatctctgatcccttccttgttaaaagttaccacggttggccaggcgcggtggttcatgcctgtaatcccagcactatgggaggccgagacgggcggatcacgaggtcaggagactgagaccatcctggctaacacggtgaaaccccatctctactaaaaatacaaaaaacaaaaaaaattagccaggcgcattggctcatgtctgtaatcccagcactttgggaggtcgggggggtgggggaggatcacggggtcaggagatcgagaccatcctggccaacatgatgaaaccctatctctactaaaaatacaaaaattagccgggcatggtggtgcacgcctatagtcccagctactcgggaggctgaggtaggagaatcgtttgaactcaggaggcagaggttgcaatgagccaagatcgcgccactgcactccagcctgggtgacagagcgagaccgtctcaaaaagaaagaagtgactagggttcagagaaccagggttcaaagcccagggatgcaaaggttgcagtgagttgagtcatgggatcccagacttttttaaatgtttgcaatgtttcccgtttacagaatgctacaagaataatgtacgtactacctaaaaggatgtctaaatgtttgttaataaaaataagaaatagctacagtgacagattttagagcaaaaattagtaataaaaataagaaataaaatta

SEQ ID NO: 120 - DNA, 602 bp, Artificial Sequence
  Synthetic construct
gatgtccaattattccctcctgagtgttgattatgtagtggacaaagcacaacttctgagaaaaggtgtatctggggactggaaaaatcacttcacagtggcccaagctgaagactttgataaattgttccaagagaagatggcagatcttcctcgagagctgttcccatgggaataacgtccaaaacactctggatcttatatggagaatgacattgattctcctgtccttgtacatgtacctgactggggtcattgtgtaagacttattattttatcctgaaaccttaaatatcaaacctctgcatctctgatcccttccttgttaaaagttaccacggttggccaggcgcggtggttcatgcctgtaatcccagcactatgggaggccgagacgggcggatcacgaggtcaggagactgagaccatcctggctaacacggtgaaaccccatctctactaaaaatacaaaaaacaaaaaaaattagccaggcgcattggctcatgtctgtaatcccagcactttgggaggtcgggggggtgggggaggatcacggggtcaggagatcgagaccatcctggccaacatgatgaaaccctat

SEQ ID NO: 121 - DNA, 24 bp, Artificial Sequence
  Synthetic construct
tgcgggacgacgacatctttatca

SEQ ID NO: 122 - DNA, 24 bp, Artificial Sequence
  Synthetic construct
agttggacatggtgttggccttca

SEQ ID NO: 123 - DNA, 1048 bp, Homo sapiens
tacaagggcgtccccttccccgtcggcctgtactcgctcgagagcatcagcttggcggagaacacccaagatgtgcgggacgacgacatctttatcatcacctaccccaagtcaggcacgacctggatgatcgagatcatctgcttaatcctgaaggaaggggatccatcctggatccgctccgtgcccatctgggagcgggcaccctggtgtgagaccattgtgggtgccttcagcctcccggaccagtacagcccccgcctcatgagctcccatcttcccatccagatcttcaccaaggccttcttcagctccaaggccaaggtgatctacatgggccgcaacccccgggacgttgtggtctccctctatcattactccaagatcgccgggcagttaaaggacccgggcacacccgaccagttcctgagggacttcctcaaaggcgaagtgcagtttggctcctggttcgaccacattaagggctggcttcggatgaagggcaaagacaacttcctatttatcacctacgaggagctgcagcaggacttacagggctccgtggagcgcatctgtgggttcctgggccgtccgctgggcaaggaggcactgggctccgtcgtggcacactcaaccttcagcgccatgaaggccaacaccatgtccaactacacgctgctgcctcccagcctgctggaccaccgtcgcggggccttcctccggaaaggggtctgcggcgactggaagaaccacttcacggtggcccagagcgaagccttcgatcgtgcctaccgcaagcagatgcgggggatgccgaccttcccctgggatgaagacccggaggaggacggcagcccagatcctgagcccagccctgagcctgagcccaagcccagccttgagcccaacaccagcctggagcgtgagcccagacccaactccagccccagccccagccccggccaggcctctgagaccccgcacccacgaccctcataataaacacgtcgattctgtccaaaaaaaaaaaaaaaaaaaaaaaaaaaaa

SEQ ID NO: 124 - DNA, 597 bp, Artificial Sequence
  Synthetic construct
tgcgggacgacgacatctttatcatcacctaccccaagtcaggcacgacctggatgatcgagatcatctgcttaatcctgaaggaaggggatccatcctggatccgctccgtgcccatctgggagcgggcaccctggtgtgagaccattgtgggtgccttcagcctcccggaccagtacagcccccgcctcatgagctcccatcttcccatccagatcttcaccaaggccttcttcagctccaaggccaaggtgatctacatgggccgcaacccccgggacgttgtggtctccctctatcattactccaagatcgccgggcagttaaaggacccgggcacacccgaccagttcctgagggacttcctcaaaggcgaagtgcagtttggctcctggttcgaccacattaagggctggcttcggatgaagggcaaagacaacttcctatttatcacctacgaggagctgcagcaggacttacagggctccgtggagcgcatctgtgggttcctgggccgtccgctgggcaaggaggcactgggctccgtcgtggcacactcaaccttcagcgccatgaaggccaacaccatgtccaact

SEQ ID NO: 125 - DNA, 24 bp, Artificial Sequence
  Synthetic construct
actacgttatgtgagactatgggg

SEQ ID NO: 126 - DNA, 24 bp, Artificial Sequence
  Synthetic construct
tttaggttcacttccacagctgct

SEQ ID NO: 127 - DNA, 1106 bp, Homo sapiens
ctcaccgaccaaatgtctttcactgacagaataagaaatttcatctcctaccacctacaggactacatgtttgaaactctttggaaatcatgggattcatactatagtaaagctttaggaagacccactacgttatgtgagactatggggaaagctgaaatttggttaatccgaacatattgggattttgaatttcctcgtccatacttacctaattttgagtttgttggaggattgcactgcaaacctgccaaacctttacctaaggaaatggaagaatttatccagagctcaggtaaaaatggtgttgtggtgttttctctgggatcaatggtcaaaaaccttacagaagaaaaggccaatcttattgcctcagcccttgcccagattccacagaaggttttatggagatacaaaggaaagaaaccagccacattaggaaacaatactcagctctttgattggataccccagaatgatcttcttggacatcccaaaaccaaagcttttatcactcatggtggaactaatgggatctacgaagctatttaccacggagtccctatggtgggagttcccatgtttgctgatcagcctgataacattgctcacatgaaggccaaaggagcagctgtggaagtgaacctaaacacaatgacaagtgtggatttgcttagcgctttgagaacagtcattaatgaaccttcttataaagagaatgctatgaggttatcaagaattcaccatgatcaacctgtaaagcccctggatcgagcagtcttctggatcgagtttgtcatgcgccacaaaggagccaagcaccttcgggttgcagcccatgacctcacctggttccagtaccactctttggatgtaattgggttcttgctggtctgtgtgacaacggctatatttttggtcatacaatgttgtttgttttcctgtcaaaaatttggtaagataggaaagaagaaaaaaagagaataggtcaagaaaaagaggaaatatatatattcttaagtttggcaaaatcctgagtagtggaagtcctattaattccagacaaaagggagtttaacaaaaacacgtcttccatcctggttcca

SEQ ID NO: 128 - DNA, 524 bp, Artificial Sequence
  Synthetic construct
actacgttatgtgagactatggggaaagctgaaatttggttaatccgaacatattgggattttgaatttcctcgtccatacttacctaattttgagtttgttggaggattgcactgcaaacctgccaaacctttacctaaggaaatggaagaatttatccagagctcaggtaaaaatggtgttgtggtgttttctctgggatcaatggtcaaaaaccttacagaagaaaaggccaatcttattgcctcagcccttgcccagattccacagaaggttttatggagatacaaaggaaagaaaccagccacattaggaaacaatactcagctctttgattggataccccagaatgatcttcttggacatcccaaaaccaaagcttttatcactcatggtggaactaatgggatctacgaagctatttaccacggagtccctatggtgggagttcccatgtttgctgatcagcctgataacattgctcacatgaaggccaaaggagcagctgtggaagtgaacctaaa

SEQ ID NO: 129 - DNA, 24 bp, Artificial Sequence
  Synthetic construct
ccaatggcatctataaggcaatct

SEQ ID NO: 130 - DNA, 24 bp, Artificial Sequence
  Synthetic construct
ttccagcctcagacgtaattaatc

SEQ ID NO: 131 - DNA, 1013 bp, Homo sapiens
actcaatactcggctgtacaagtggataccccagaatgatcttcttggtcacccaaaaaccagagcttttataactcatggtggagccaatggcatctataaggcaatctctcctagaatccctatggtgggcgttccattgtttgcagatcaacctgataacattgcacacatgaaggccaagggagcagctgttagtttggacttccacacaatgtcgagtacagacttactcaatgcactgaagacagtaattaatgatcctttatataaagagaatgctatgaaattatcaagaattcatcatgatcaaccagtgaagccccttgatcgagcagtcttctggattgaatttgtcatgcgccataaaggagccaagcaccttcgggttgcagcccacgacctcacctggttccagtaccactctttggatgtgactgggttcctgctggcctgtgtggcaactgtgatattcatcatcacaaaatgtctgttttgtgtctggaagtttgttagaacaggaaagaaggggaaaagagattaattacgtctgaggctggaagctgggaaacccaataaatgaactcctttagtttattacaacaagaagacgttgtgatacaagagattcctttcttcttgtgacaaaacatctttcaaaacttaccttgtcaagtcaaaatttgttttagtacctgtttaaccattagaaatatttcatgtcaaggaggaaaacattagggaaaacaaaaatgatataaagccatatgaggttatattgaaatgtattgagcttatattgaaatttattgttccaattcacaggttacatgaaaaaaaatttactaagcttaactacatgtcacacattgtacatggaaacaagaacattaagaagtccgactgacagtatcagtactgttttgcaaatactcagcatactttggatccatttcatgcaggattgtgttgttttaactgttgttgaggaagctaataaataattaaattgt

SEQ ID NO: 132 - DNA, 476 bp, Artificial Sequence
  Synthetic construct
ccaatggcatctataaggcaatctctcctagaatccctatggtgggcgttccattgtttgcagatcaacctgataacattgcacacatgaaggccaagggagcagctgttagtttggacttccacacaatgtcgagtacagacttactcaatgcactgaagacagtaattaatgatcctttatataaagagaatgctatgaaattatcaagaattcatcatgatcaaccagtgaagccccttgatcgagcagtcttctggattgaatttgtcatgcgccataaaggagccaagcaccttcgggttgcagcccacgacctcacctggttccagtaccactctttggatgtgactgggttcctgctggcctgtgtggcaactgtgatattcatcatcacaaaatgtctgttttgtgtctggaagtttgttagaacaggaaagaaggggaaaagagattaattacgtctgaggctggaa

SEQ ID NO: 133 - DNA, 24 bp, Artificial Sequence
  Synthetic construct
tctggattgagtttgtcatgcgcc

SEQ ID NO: 134 - DNA, 24 bp, Artificial Sequence
  Synthetic construct
ttagggtacatgtgcacaacgaag

SEQ ID NO: 135 - DNA, 1010 bp, Homo sapiens
ctgtacaagtggttaccccagaatgaccttcttggtcatcccaaaaccaaagcttttataactcatggtggaaccaatggcatctatgaggcgatctaccatgggatccctatggtgggcattcccttgtttgcggatcaacatgataacattgctcacatgaaagccaagggagcagccctcagtgtggacatcaggaccatgtcaagtagagatttgctcaatgcattgaagtcagtcattaatgaccctgtctataaagagaatgtcatgaaattatcaagaattcatcatgaccaaccaatgaagcccctggatcgagcagtcttctggattgagtttgtcatgcgccacaaaggagccaagcaccttcgagtcgcagctcacaacctcacctggatccagtaccactctttggatgtgatagcattcctgctggcctgcgtggcaactgtgatatttatcatcacaaaattttgcctgttttgtttccgaaagcttgccaaaacaggaaagaagaagaaaagagattagttatatcaaaagcctgaagtggaatgactgaaagatgggactcctcctttatttcagcatggagggttttaaatggaggatttcctttttcctgtgacaaaacatcttttcacaacttaccttgttaagacaaaatttattttccagggatttaatacgtactttagttggaattattctatgtcaatgatttttaagctatgaaaaatacaatggggggaaggatagcatttggagatatacctaatgttaaatgacgagttactggatgcagcacgcaacatggcacatgtgtatacatatgtagctaacccttcgttgtgcacatgtaccctaaaacttaaagtataatttaaaaaaagcaaaaaaaaaaaataccaactcttttttttaaaccaggaaggaaaatgtgaacatggaaacaacttctagtattggatctgaaaataaagtgtcatccaagccataaaaaaaaa

SEQ ID NO: 136 - DNA, 543 bp, Artificial Sequence
  Synthetic cosntruct
tctggattgagtttgtcatgcgccacaaaggagccaagcaccttcgagtcgcagctcacaacctcacctggatccagtaccactctttggatgtgatagcattcctgctggcctgcgtggcaactgtgatatttatcatcacaaaattttgcctgttttgtttccgaaagcttgccaaaacaggaaagaagaagaaaagagattagttatatcaaaagcctgaagtggaatgactgaaagatgggactcctcctttatttcagcatggagggttttaaatggaggatttcctttttcctgtgacaaaacatcttttcacaacttaccttgttaagacaaaatttattttccagggatttaatacgtactttagttggaattattctatgtcaatgatttttaagctatgaaaaatacaatggggggaaggatagcatttggagatatacctaatgttaaatgacgagttactggatgcagcacgcaacatggcacatgtgtatacatatgtagctaacccttcgttgtgcacatgtaccctaa

SEQ ID NO: 137 - DNA, 24 bp, Artificial Sequence
  Synthetic construct
tcgagcagtcttctggattgagtt

SEQ ID NO: 138 - DNA, 24 bp, Artificial Sequence
  Synthetic construct
agctcagtaacttttgtgtggggt

SEQ ID NO: 139 - DNA, 1147 bp, Homo sapiens
ggtattgtggtgttttctctggggtcgatgatcagtaacatgtcagaagaaagtgccaacatgattgcatcagcccttgcccagatcccacaaaaggttctatggagatttgatggcaagaagccaaatactttaggttccaatactcgactgtataagtggttaccccagaatgaccttcttggtcatcccaaaaccaaagcttttataactcatggtggaaccaatggcatctatgaggcgatctaccatgggatccctatggtgggcattcccttgtttgcggatcaacatgataacattgctcacatgaaagccaagggagcagccctcagtgtggacatcaggaccatgtcaagtagagatttgctcaatgcattgaagtcagtcattaatgaccctatctataaagagaatatcatgaaattatcaagaattcatcatgatcaaccggtgaagcccctggatcgagcagtcttctggattgagtttgtcatgcgccataaaggagccaagcaccttcgggtcgcagcccacaacctcacctggatccagtaccactctttggatgtgatagcattcctgctggcctgcgtggcaactatgatatttatgatcacaaaatgttgcctgttttgtttccgaaagcttgccaaaacaggaaagaagaagaaaagggattagttatatcaaaagcctgaagtggaatgaccaaaagatgggactcctcctttattccagcatggagggttttaaatggaggatttcctttttcctgcgacaaaacgtcttttcacaacttaccctgttaagtcaaaatttattttccaggaatttaatatgtactttagttggaattattctatgtcaatgatttttaagctatgaaaaataataatataaaaccttatgggcttatattgaaatttattattctaatccaaaagttaccccacacaaaagttactgagcttccttatgtttcacacattgtatttgaacacaaaacattaacaactccactcatagtatcaacattgttttgcaaatactcagaatattttggcttcattttgagcagaatttttgtttttaattttgccaatgaaatcttcaataattaaaaaaaaaaaaaaaaaaaaaaaaa

SEQ ID NO: 140 - DNA, 506 bp, Artificial Sequence
  Synthetic construct
tcgagcagtcttctggattgagtttgtcatgcgccataaaggagccaagcaccttcgggtcgcagcccacaacctcacctggatccagtaccactctttggatgtgatagcattcctgctggcctgcgtggcaactatgatatttatgatcacaaaatgttgcctgttttgtttccgaaagcttgccaaaacaggaaagaagaagaaaagggattagttatatcaaaagcctgaagtggaatgaccaaaagatgggactcctcctttattccagcatggagggttttaaatggaggatttcctttttcctgcgacaaaacgtcttttcacaacttaccctgttaagtcaaaatttattttccaggaatttaatatgtactttagttggaattattctatgtcaatgatttttaagctatgaaaaataataatataaaaccttatgggcttatattgaaatttattattctaatccaaaagttaccccacacaaaagttactgagct

SEQ ID NO: 141 - DNA, 24 bp, Artificial Sequence
  Synthetic construct
tggagctggtgtcaagtatctgtc

SEQ ID NO: 142 - DNA, 24 bp, Artificial Sequence
  Synthetic construct
gatagttcgattgacagggtgacc

SEQ ID NO: 143 - DNA, 1054 bp, Homo sapiens
aggggtcagctttctggttcttcccaaatatgaaaggataatgcagaagtacaacctgctgccggagaagtccatgtatgatttggttcatgggtccagcctgtggatgctgtgtactgacgtagcactggaattcccaagacccactctgcctaatgttgtttatgtaggaggaatcctaaccaaaccagccagcccactaccagaagatctccaaagatgggtaaatggtgctaatgaacatggctttgtcttggtgtcttttggagctggtgtcaagtatctgtcagaagacattgctaacaaactggcaggagctctggggagattgcctcaaaaagtgatttggaggttttctggacccaaaccaaagaatctaggaaacaacactaaactcatagaatggttaccacaaaatgacctgcttgggcattcaaagattaaagccttcgtgagccatggtggtttgaacagtatttttgaaactatgtatcatggtgtgcctgtagtgggaattccagtctttggagaccattatgatactatgaccagagtacaggcaaaaggcatggggatattgctagaatggaagacagttactgaaaaagagctctatgaagcactagtgaaggttatcaataatcccagctaccgtcagagggctcagaagctttcggaaattcacaaggatcaacctggtcaccctgtcaatcgaactatctattggatagattatattattcgtcacaatggagcccatcacctacgtgccgctgtccatcagatctccttttgtcagtattttttactggatattgcctttgtgcttttgcttggtgctgccttgttatactttctcttgtcttgggtgacaaaatttatctacagaaaaatcaaaagtctgtggtctagaaataagcatagcacagttaatggacattaccacaatggaatcctcaatggcaagtacaaaagaaatggccatattaaacatgaaaagaaagtgaaatgagccgacagcccaggtgatagaaataaattggttcactcattg

SEQ ID NO: 144 - DNA, 457 bp, Artificial Sequence
  Synthetic construct
tggagctggtgtcaagtatctgtcagaagacattgctaacaaactggcaggagctctggggagattgcctcaaaaagtgatttggaggttttctggacccaaaccaaagaatctaggaaacaacactaaactcatagaatggttaccacaaaatgacctgcttgggcattcaaagattaaagccttcgtgagccatggtggtttgaacagtatttttgaaactatgtatcatggtgtgcctgtagtgggaattccagtctttggagaccattatgatactatgaccagagtacaggcaaaaggcatggggatattgctagaatggaagacagttactgaaaaagagctctatgaagcactagtgaaggttatcaataatcccagctaccgtcagagggctcagaagctttcggaaattcacaaggatcaacctggtcaccctgtcaatcgaactatc

SEQ ID NO: 145 - DNA, 24 bp, Artificial Sequence
  Synthetic construct
ccaagtttaggagggaggaaggag

SEQ ID NO: 146 - DNA, 24 bp, Artificial Sequence
  Synthetic construct
gctactgctgctgagggtcgtgtt

SEQ ID NO: 147 - DNA, 1058 bp, Homo sapiens
tctgaagtccacgttgtcatgaccggaggttacgccaccattgctggcagcctgctgggtgcctacatctcctttgggatcgatgccacctcgttgattgcagcctctgtgatggctgccccttgtgccttggccctctccaagctggtctacccggaggtggaggagtccaagtttaggagggaggaaggagtgaaactgacctatggagatgctcagaacctcatagaagcagccagcactggggccgccatctccgtgaaggtggtcgccaacatcgctgccaacctgattgcgttcctggctgtgctggactttatcaatgctgccctctcctggctgggagacatggtggacatccaggggctcagcttccagctcatctgctcctacatcctgcggcctgtagccttcttgatgggtgtggcgtgggaggactgcccagtggtagctgagctgctggggatcaagctgtttctgaacgagtttgtggcctatcaagacctctccaagtacaagcaacgccgcctggcaggggccgaggagtgggtcggcaacaggaagcagtggatctccgtcagagctgaagtcctcacgacgtttgccctctgtggatttgccaatttcagctccattgggatcatgctgggaggcttgacctccatggtcccccaacggaagagcgacttctcccagatagtgctccgggcgctcttcacgggagcctgtgtgtccctggtgaacgcctgtatggcagggatcctctacatgcccaggggggctgaagttgactgcatgtccctcttgaacacgaccctcagcagcagtagctttgagatttaccagtgctgccgtgaggccttccagagcgtcaatccagagttcagcccagaggccctggacaactgctgtcggttttacaaccacacgatctgtgcacagtgaggacagaacatgcttgtgcttctgcgcttctgagggctgttctcccccgggaaccatctgtccccaccttccctttcccagagccctcttcagggaagccacaggacttagat

SEQ ID NO: 148 - DNA, 662 bp, Artificial Sequence
  Synthetic construct
ccaagtttaggagggaggaaggagtgaaactgacctatggagatgctcagaacctcatagaagcagccagcactggggccgccatctccgtgaaggtggtcgccaacatcgctgccaacctgattgcgttcctggctgtgctggactttatcaatgctgccctctcctggctgggagacatggtggacatccaggggctcagcttccagctcatctgctcctacatcctgcggcctgtagccttcttgatgggtgtggcgtgggaggactgcccagtggtagctgagctgctggggatcaagctgtttctgaacgagtttgtggcctatcaagacctctccaagtacaagcaacgccgcctggcaggggccgaggagtgggtcggcaacaggaagcagtggatctccgtcagagctgaagtcctcacgacgtttgccctctgtggatttgccaatttcagctccattgggatcatgctgggaggcttgacctccatggtcccccaacggaagagcgacttctcccagatagtgctccgggcgctcttcacgggagcctgtgtgtccctggtgaacgcctgtatggcagggatcctctacatgcccaggggggctgaagttgactgcatgtccctcttgaacacgaccctcagcagcagtagc

SEQ ID NO: 149 - DNA, 24 bp, Artificial Sequence
  Synthetic construct
cataggaatcacacttggaggctt

SEQ ID NO: 150 - DNA, 24 bp, Artificial Sequence
  Synthetic construct
cctttagtagagacggggtttcac

SEQ ID NO: 151 - DNA, 1019 bp, Homo sapiens
atctcctaaggcccatggttttcatgatgggtgtagagtggacagactgtccaatggtggctgagatggtgggaatcaagttcttcataaatgagtttgtggcttatcagcaactgtctcaatacaagaacaaacgtctctctggaatggaggagtggattgagggagagaaacagtggatttctgtgagagctgaaatcattacaacattttcactctgtggatttgccaatcttagttccataggaatcacacttggaggcttgacatcaatagtacctcaccggaagagtgacttgtccaaggttgtggtcagggccctcttcacaggggcctgtgtatcccttatcagtgcctgtatggcaggaatcctctatgtccccaggggagctgaagctgactgtgtctccttcccaaacacaagtttcaccaatagaacctatgagacctacatgtgctgcagagggctctttcagagtacttctctgaatggcaccaaccctccttctttttctggtccctgggaagataaggagttcagtgctatggcccttactaactgctgtggattctacaacaataccgtctgtgcctaaggctgcttgatctatttctataacagttttgatcttaaaagctttgtgattgcaaaggtgtttatgtactcagggtgcccacaactcactcaccaagatgtttaacagtaagtaacagtaaatgtaaaagattcattttgggccgggctcagtggctcacgcctgtaatcccagcgctttgggaggccgaggcgggcggatcgcagggtcaggagatcgagaccatcctggctaacacggtgaaaccccgtctctactaaaggtacaaaaaattggccgggagtggtgtcgggcgactgtagtcccagctactcgcgagactgaggcaggagaatggcgtgaatccgggaggcggagcttgcagcgagccgggatcgcgccactgtactccagcctgggtgacagagcgagactctgtctcag

SEQ ID NO: 152 - DNA, 618 bp, Artificial Sequence
  Synethic construct
cataggaatcacacttggaggcttgacatcaatagtacctcaccggaagagtgacttgtccaaggttgtggtcagggccctcttcacaggggcctgtgtatcccttatcagtgcctgtatggcaggaatcctctatgtccccaggggagctgaagctgactgtgtctccttcccaaacacaagtttcaccaatagaacctatgagacctacatgtgctgcagagggctctttcagagtacttctctgaatggcaccaaccctccttctttttctggtccctgggaagataaggagttcagtgctatggcccttactaactgctgtggattctacaacaataccgtctgtgcctaaggctgcttgatctatttctataacagttttgatcttaaaagctttgtgattgcaaaggtgtttatgtactcagggtgcccacaactcactcaccaagatgtttaacagtaagtaacagtaaatgtaaaagattcattttgggccgggctcagtggctcacgcctgtaatcccagcgctttgggaggccgaggcgggcggatcgcagggtcaggagatcgagaccatcctggctaacacggtgaaaccccgtctctactaaagg

SEQ ID NO: 153 - DNA, 24 bp, Artificial Sequence
  Synthetic construct
cgtcattggctgctgctaaactct

SEQ ID NO: 154 - DNA, 24 bp, Artificial Sequence
  Synthetic construct
cagggaaagtggagttgaaggcat

SEQ ID NO: 155 - DNA, 1069 bp, Homo sapiens
catatttaccttacatcaccaagtctgaactccacgccatcatgaccgccgggttctctaccattgctggaagcgtgctaggtgcatacatttcttttggggttccatcctcccacttgttaacagcgtcagttatgtcagcacctgcgtcattggctgctgctaaactcttttggcctgagacagaaaaacctaaaataaccctcaagaatgccatgaaaatggaaagtggtgattcagggaatcttctagaagctgcaacacagggagcatcctcctccatctccctggtggccaacatcgctgtgaatctgattgccttcctggccctgctgtcttttatgaattcagccctgtcctggtttggaaacatgtttgactacccacagctgagttttgagctaatctgctcctacatcttcatgcccttttccttcatgatgggagtggaatggcaggacagctttatggttgccagactcataggttataagaccttcttcaatgaatttgtggcttatgagcacctctcaaaatggatccacttgaggaaagaaggtggacccaaatttgtaaacggtgtgcagcaatatatatcaattcgttctgagataatcgccacttacgctctctgtggttttgccaatatcgggtccctaggaatcgtgatcggcggactcacatccatggctccttccagaaagcgtgatatcgcctcgggggcagtgagagctctgattgcggggaccgtggcctgcttcatgacagcctgcatcgcaggcatactctccagcactcctgtggacatcaactgccatcacgttttagagaatgccttcaactccactttccctggaaacacaaccaaggtgatagcttgttgccaaagtctgttgagcagcactgttgccaagggtcctggtgaagtcatcccaggaggaaaccacagtctgtattctttgaagggctgctgcacattgttgaatccatcgacctttaactgcaatgggatctctaatacattttgaggtcagccacttctccagtggaactctgaagtacagatgct

SEQ ID NO: 156 - DNA, 708 bp, Artificial Sequence
  Synthetic construct
cgtcattggctgctgctaaactcttttggcctgagacagaaaaacctaaaataaccctcaagaatgccatgaaaatggaaagtggtgattcagggaatcttctagaagctgcaacacagggagcatcctcctccatctccctggtggccaacatcgctgtgaatctgattgccttcctggccctgctgtcttttatgaattcagccctgtcctggtttggaaacatgtttgactacccacagctgagttttgagctaatctgctcctacatcttcatgcccttttccttcatgatgggagtggaatggcaggacagctttatggttgccagactcataggttataagaccttcttcaatgaatttgtggcttatgagcacctctcaaaatggatccacttgaggaaagaaggtggacccaaatttgtaaacggtgtgcagcaatatatatcaattcgttctgagataatcgccacttacgctctctgtggttttgccaatatcgggtccctaggaatcgtgatcggcggactcacatccatggctccttccagaaagcgtgatatcgcctcgggggcagtgagagctctgattgcggggaccgtggcctgcttcatgacagcctgcatcgcaggcatactctccagcactcctgtggacatcaactgccatcacgttttagagaatgccttcaactccactttccctg

SEQ ID NO: 157 - DNA, 24 bp, Artificial Sequence
  Synthetic construct
tgtttgtgccactgctgctgctgt

SEQ ID NO: 158 - DNA, 24 bp, Artificial Sequence
  Synthetic construct
ggggagaatggagtatatcaggtc

SEQ ID NO: 159 - DNA, 1022 bp, Homo sapiens
cagcacctgggaacgttacttcattcctgtgtcctgtttcttgactttcaatatctttgactggttgggccggagcctcacagctgtattcatgtggcctgggaaggacagccgctggctgccaagcctggtgctggcccggctggtgtttgtgccactgctgctgctgtgcaacattaagccccgccgctacctgactgtggtcttcgagcacgatgcctggttcatcttcttcatggctgcctttgccttctccaacggctacctcgccagcctctgcatgtgcttcgggcccaagaaagtgaagccagctgaggcagagaccgcaggagccatcatggccttcttcctgtgtctgggtctggcactgggggctgttttctccttcctgttccgggcaattgtgtgacaaaggatggacagaaggactgcctgcctccctccctgtctgcctcctgccccttccttctgccaggggtgatcctgagtggtctggcggttttttcttctaactgacttctgctttccacggcgtgtgctgggcccggatctccaggccctggggagggagcctctggacggacagtggggacattgtgggtttggggctcagagtcgagggacggggtgtagcctcggcatttgcttgagtttctccactcttggctctgactgatccctgcttgtgcaggccagtggaggctcttgggcttggagaacacgtgtgtctctgtgtatgtgtctgtgtgtctgcgtccgtgtctgtcagactgtctgcctgtcctggggtggctaggagctgggtctgaccgttgtatggtttgacctgatatactccattctcccctgcgcctcctcctctgtgttttttccatgtccccctcccaactccccatgcccagtttttacccatcatgcaccctgtacagttgccacgttactgccttttttaaaaatatatttgacagaaaccaggtgccttcagaggctctctgatttaaataaacctttcttgttttttt

SEQ ID NO: 160 - DNA, 701 bp, Artificial Sequence
  Synthetic construct
tgtttgtgccactgctgctgctgtgcaacattaagccccgccgctacctgactgtggtcttcgagcacgatgcctggttcatcttcttcatggctgcctttgccttctccaacggctacctcgccagcctctgcatgtgcttcgggcccaagaaagtgaagccagctgaggcagagaccgcaggagccatcatggccttcttcctgtgtctgggtctggcactgggggctgttttctccttcctgttccgggcaattgtgtgacaaaggatggacagaaggactgcctgcctccctccctgtctgcctcctgccccttccttctgccaggggtgatcctgagtggtctggcggttttttcttctaactgacttctgctttccacggcgtgtgctgggcccggatctccaggccctggggagggagcctctggacggacagtggggacattgtgggtttggggctcagagtcgagggacggggtgtagcctcggcatttgcttgagtttctccactcttggctctgactgatccctgcttgtgcaggccagtggaggctcttgggcttggagaacacgtgtgtctctgtgtatgtgtctgtgtgtctgcgtccgtgtctgtcagactgtctgcctgtcctggggtggctaggagctgggtctgaccgttgtatggtttgacctgatatactccattctcccc

SEQ ID NO: 161 - DNA, 24 bp, Artificial Sequence
  Synthetic construct
cccagtagtccccagaaagtagct

SEQ ID NO: 162 - DNA, 24 bp, Artificial Sequence
  Synthetic construct
acgtcgagaagaggctgccaaaga

SEQ ID NO: 163 - DNA, 1036 bp, Homo sapiens
ctgtccatggccagtggcgtggacgccgagacctctgccctggggtactttatcacgccctatgtgggcatcctcatgtccatcgtgtgttacctgagcctgcctcacctgaagtttgcccgctactacctggccaataaatcatcccaggcccaagctcaggagctggagaccaaagctgagctcctccagtctgatgagaacgggattcccagtagtccccagaaagtagctctgaccctggatcttgacctggagaaggagccggaatcagagccagatgagccccagaagccaggaaaaccttcagtcttcactgtcttccagaagatctggctgacagcgctgtgccttgtgttggtcttcacagtcaccctgtccgtcttccccgccatcacagccatggtgaccagctccaccagtcctgggaagtggagtcagttcttcaaccccatctgctgcttcctcctcttcaacatcatggactggctgggacggagcctgacctcttacttcctgtggccagacgaggacagccggctgctgcccctgctggtctgcctgcggttcctgttcgtgcccctcttcatgctgtgccacgtgccccagaggtcccggctgcccatcctcttcccacaggatgcctacttcatcaccttcatgctgctctttgccgtttctaatggctacctggtgtccctcaccatgtgcctggcgcccaggcaggtgctgccacacgagagggaggtggccggcgccctcatgaccttcttcctggccctgggactttcctgtggagcctccctctccttcctcttcaaggcgctgctctgaagtggcccctccaggctctttggcagcctcttctcgacgtctccttccggagctgagatccagcccagggcgaatggcgagcttggctcaggcctctgcggggtggaggcccctgggcctgaggctgccagcagcgggcaggagctgctcttcatccacttggagtgctgcggggaagaaatcaccaccggtcattctaacc

SEQ ID NO: 164 - DNA, 664 bp, Artificial Sequence
  Synthetic construct
cccagtagtccccagaaagtagctctgaccctggatcttgacctggagaaggagccggaatcagagccagatgagccccagaagccaggaaaaccttcagtcttcactgtcttccagaagatctggctgacagcgctgtgccttgtgttggtcttcacagtcaccctgtccgtcttccccgccatcacagccatggtgaccagctccaccagtcctgggaagtggagtcagttcttcaaccccatctgctgcttcctcctcttcaacatcatggactggctgggacggagcctgacctcttacttcctgtggccagacgaggacagccggctgctgcccctgctggtctgcctgcggttcctgttcgtgcccctcttcatgctgtgccacgtgccccagaggtcccggctgcccatcctcttcccacaggatgcctacttcatcaccttcatgctgctctttgccgtttctaatggctacctggtgtccctcaccatgtgcctggcgcccaggcaggtgctgccacacgagagggaggtggccggcgccctcatgaccttcttcctggccctgggactttcctgtggagcctccctctccttcctcttcaaggcgctgctctgaagtggcccctccaggctctttggcagcctcttctcgacgt

SEQ ID NO: 165 - DNA, 24 bp, Artificial Sequence
  Synthetic construct
cttcagcagcagcatctacggcat

SEQ ID NO: 166 - DNA, 24 bp, Artificial Sequence
  Synthetic construct
ggtagttacagagcacgaagaggg

SEQ ID NO: 167 - DNA, 1073 bp, Homo sapiens
tggtggccaacttcctgcttgtcaacagggttgcagtccacatccgtgtcctggcctcactgacggtcatcctggccatcttcatggtgataactgcactggtgaaggtggacactttctcctggacccgtggcttttttgcggtcaccattgtctgcatggtgatcctcagcggtgcctccactgtcttcagcagcagcatctacggcatgaccggctcctttcctatgaggaactcccaggcactgatatcaggaggagccatgggcgggacggtcagcgccgtggcctcattggtggacttggctgcatccagtgatgtgaggaacagcgccctggccttcttcctgacggccaccatcttcctcgtgctctgcatgggactctacctgctgctgtccaggctggagtatgccaggtactacatgaggcctgttcttgcggcccatgtgttttctggtgaagaggagcttccccaggactccctcagtgccccttcggtggcctccagattcattgattcccacacaccccctctccgccccatcctgaagaagacggccagcctgggcttctgtgtcacctacgtcttcttcatcaccagcctcatctaccccgccgtctgcaccaacatcgagtccctcaacaagggctcgggctcactgtggaccaccaagtttttcatccccctcactaccttcctcctgtacaactttgctgacctatgtggccggcagctcaccgcctggatccaggtgccagggcccaatagcaaggcgctcccagggttcgtgctcctccggacctgcctcatccccctcttcgtgctctgtaactaccagccccgcgtccacctgaagactgtggtcttccagtccgatgtgtaccccgcactcctcagctccctgctggggctcagcaacggctacctcagcaccctggccctcctctacgggcctaagattgtgcccagggagctggctgaggccacgggagtggtgatgtccttttatgtgtgcttgggcttaacactgggctcagcctgctctaccctcctggtgcacctcatctag

SEQ ID NO: 168 - DNA, 653 bp, Artificial Sequence
  Synthetic construct
cttcagcagcagcatctacggcatgaccggctcctttcctatgaggaactcccaggcactgatatcaggaggagccatgggcgggacggtcagcgccgtggcctcattggtggacttggctgcatccagtgatgtgaggaacagcgccctggccttcttcctgacggccaccatcttcctcgtgctctgcatgggactctacctgctgctgtccaggctggagtatgccaggtactacatgaggcctgttcttgcggcccatgtgttttctggtgaagaggagcttccccaggactccctcagtgccccttcggtggcctccagattcattgattcccacacaccccctctccgccccatcctgaagaagacggccagcctgggcttctgtgtcacctacgtcttcttcatcaccagcctcatctaccccgccgtctgcaccaacatcgagtccctcaacaagggctcgggctcactgtggaccaccaagtttttcatccccctcactaccttcctcctgtacaactttgctgacctatgtggccggcagctcaccgcctggatccaggtgccagggcccaatagcaaggcgctcccagggttcgtgctcctccggacctgcctcatccccctcttcgtgctctgtaactacc

SEQ ID NO: 169 - DNA, 24 bp, Artificial Sequence
  Synthetic construct
gagcaacagtatggtcagccttca

SEQ ID NO: 170 - DNA, 24 bp, Artificial Sequecne
  Synthetic ocnstruct
cagagccccaaaatatataggagc

SEQ ID NO: 171 - DNA, 1056 bp, Homo sapiens
tttgtgcttttgacgttgttacaagtaagcagctatattggtgcttttacttatgtcttcaaatacgtagagcaacagtatggtcagccttcatctaaggctaacatcttattgggagtcataaccatacctatttttgcaagtggaatgtttttaggaggatatatcattaaaaaattcaaactgaacaccgttggaattgccaaattctcatgttttactgctgtgatgtcattgtccttttacctattatattttttcatactctgtgaaaacaaatcagttgccggactaaccatgacctatgatggaaataatccagtgacatctcatagagatgtaccactttcttattgcaactcagactgcaattgtgatgaaagtcaatgggaaccagtctgtggaaacaatggaataacttacatctcaccctgtctagcaggttgcaaatcttcaagtggcaataaaaagcctatagtgttttacaactgcagttgtttggaagtaactggtctccagaacagaaattactcagcccatttgggtgaatgcccaagagatgatgcttgtacaaggaaattttacttttttgttgcaatacaagtcttgaatttatttttctctgcacttggaggcacctcacatgtcatgctgattgttaaaattgttcaacctgaattgaaatcacttgcactgggtttccactcaatggttatacgagcactaggaggaattctagctcctatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaa

SEQ ID NO: 172 - DNA, 693 bp, Artificial Sequence
  Synthetic construct
gagcaacagtatggtcagccttcatctaaggctaacatcttattgggagtcataaccatacctatttttgcaagtggaatgtttttaggaggatatatcattaaaaaattcaaactgaacaccgttggaattgccaaattctcatgttttactgctgtgatgtcattgtccttttacctattatattttttcatactctgtgaaaacaaatcagttgccggactaaccatgacctatgatggaaataatccagtgacatctcatagagatgtaccactttcttattgcaactcagactgcaattgtgatgaaagtcaatgggaaccagtctgtggaaacaatggaataacttacatctcaccctgtctagcaggttgcaaatcttcaagtggcaataaaaagcctatagtgttttacaactgcagttgtttggaagtaactggtctccagaacagaaattactcagcccatttgggtgaatgcccaagagatgatgcttgtacaaggaaattttacttttttgttgcaatacaagtcttgaatttatttttctctgcacttggaggcacctcacatgtcatgctgattgttaaaattgttcaacctgaattgaaatcacttgcactgggtttccactcaatggttatacgagcactaggaggaattctagctcctatatattttggggctctg

SEQ ID NO: 173 - DNA, 24 bp, Artificial Sequence
  Synthetic construct
cctaaccttgacctatgatggaaa

SEQ ID NO: 174 - DNA, 24 bp, Artificial Sequecne
  Synthetic construct
tatagataagcccaagtagaccct

SEQ ID NO: 175 - DNA, 1029 bp, Homo sapiens
aaatatatggagcaacagtacggtcagtctgcatctcatgctaactttttgttgggaatcataaccattcctacggttgcaactggaatgtttttaggaggatttatcattaaaaaattcaaattgtctttagttggaattgccaaattttcatttcttacttcgatgatatccttcttgtttcaacttctatatttccctctaatctgcgaaagcaaatcagttgccggcctaaccttgacctatgatggaaataattcagtggcatctcatgtagatgtaccactttcttattgcaactcagagtgcaattgtgatgaaagtcagtgggaaccagtctgtgggaacaatggaataacttacctgtcaccttgtctagcaggatgcaaatcctcaagtggtattaaaaagcatacagtgttttataactgtagttgtgtggaagtaactggtctccagaacagaaattactcagcacacttgggtgaatgcccaagagataatacttgtacaaggaaatttttcatctatgttgcaattcaagtcataaactctttgttctctgcaacaggaggtaccacatttatcttgttgactgtgaagattgttcaacctgaattgaaagcacttgcaatgggtttccagtcaatggttataagaacactaggaggaattctagctccaatatattttggggctctgattgataaaacatgtatgaagtggtccaccaacagctgtggagcacaaggagcttgtaggatatataattccgtattttttggaagggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaa

SEQ ID NO: 176 - DNA, 580 bp, Artificial Sequence
  Synthetic construct
cctaaccttgacctatgatggaaataattcagtggcatctcatgtagatgtaccactttcttattgcaactcagagtgcaattgtgatgaaagtcagtgggaaccagtctgtgggaacaatggaataacttacctgtcaccttgtctagcaggatgcaaatcctcaagtggtattaaaaagcatacagtgttttataactgtagttgtgtggaagtaactggtctccagaacagaaattactcagcacacttgggtgaatgcccaagagataatacttgtacaaggaaatttttcatctatgttgcaattcaagtcataaactctttgttctctgcaacaggaggtaccacatttatcttgttgactgtgaagattgttcaacctgaattgaaagcacttgcaatgggtttccagtcaatggttataagaacactaggaggaattctagctccaatatattttggggctctgattgataaaacatgtatgaagtggtccaccaacagctgtggagcacaaggagcttgtaggatatataattccgtattttttggaagggtctacttgggcttatctata

SEQ ID NO: 177 - DNA, 24 bp, Artificial Sequence
  Synthetic construct
gggctctgattgataaaacatgta

SEQ ID NO: 178 - DNA, 24 bp, Artificial Sequence
  Synthetic construct
tgaaaaatatacaacttaacatga

SEQ ID NO: 179 - DNA, 1032 bp, Homo sapiens
gcacacttgggtgaatgcccaagagataatacttgtacaaggaaatttttcatctatgttgcaattcaagtcataaactctttgttctctgcaacaggaggtaccacatttatcttgttgactgtgaagattgttcaacctgaattgaaagcacttgcaatgggtttccagtcaatggttataagaacactaggaggaattctagctccaatatattttggggctctgattgataaaacatgtatgaagtggtccaccaacagctgtggagcacaaggagcttgtaggatatataattccgtattttttggaagggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaacattgcattgattcattaagatgttatttttgaggtgttcctggtctttcactgacaattccaacattctttacttacagtggaccaatggataagtctatgcatctataataaactataaaaaatgggagtacccatggttaggatatagctatgcctttatggttaagattagaatatatgatccataaaaatttaaagtgagaggcatggttagtgtgtgatacaataaaaagtaattgtttggtagttgtaactgctaataaaaccagtgactagaatataagggaggtaaaaaggacaagatagattaatagcctaaataaagagaaaagcctgatgcctttaaaaaaaatgaaacactttggatgtattacttaggccaaaatctggcctggatttatgctataatatatattttcatgttaagttgtatatttttcagaaattataaatattattaatttaaaatttgaa

SEQ ID NO: 180 - DNA, 780 bp, Artificial Sequence
  Synthetic construct
ggggctctgattgataaaacatgtatgaagtggtccaccaacagctgtggagcacaaggagcttgtaggatatataattccgtattttttggaagggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaacattgcattgattcattaagatgttatttttgaggtgttcctggtctttcactgacaattccaacattctttacttacagtggaccaatggataagtctatgcatctataataaactataaaaaatgggagtacccatggttaggatatagctatgcctttatggttaagattagaatatatgatccataaaaatttaaagtgagaggcatggttagtgtgtgatacaataaaaagtaattgtttggtagttgtaactgctaataaaaccagtgactagaatataagggaggtaaaaaggacaagatagattaatagcctaaataaagagaaaagcctgatgcctttaaaaaaaatgaaacactttggatgtattacttaggccaaaatctggcctggatttatgctataatatatattttcatgttaagttgtatatttttca

SEQ ID NO: 181 - DNA, 24 bp, Artificial Sequence
  Synthetic construct
ccatgacaccactcttgattgcca

SEQ ID NO: 182 - DNA, 24 bp, Artificial Sequence
  Synthetic construct
tttagagatcccagcaagaggcag

SEQ ID NO: 183 - DNA, 1040 bp, Homo sapiens
ggtgccctataaaggcatcgtgatatcactggtcctggttctcattccttgcaccatagggatcgtcctcaaatccaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagcccttcccctggtggcctggattctggtcccaaagcaattctgaaagccagtgtggtaaactagagagagcagcaaaaacaccagtcttgcctgagtctttctccagcatttccagtacatctatcagaatcatcaagtcttggccgggaacacagacagggtgtctacccaagaagcctcacctatccccaacttagaatttgctacttattttaaagacttgttcagtgactgtaaactctatgaaaccagaaaccgaatctgcctcttgctgggatctctaaaagtgtctgataagcatcttaaagtcactcaattcctgaactaatcaatatatatgtttaacccattactcaaatacccaaatcccattccaagttttgtgacccaaaagagaaataaatgctcacaagtgctgtagaattaaacttcagaagttctaacctt

SEQ ID NO: 184 - DNA, 688 bp, Artificial Sequence
  Synthetic construct
gccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagcccttcccctggtggcctggattctggtcccaaagcaattctgaaagccagtgtggtaaactagagagagcagcaaaaacaccagtcttgcctgagtctttctccagcatttccagtacatctatcagaatcatcaagtcttggccgggaacacagacagggtgtctacccaagaagcctcacctatccccaacttagaatttgctacttattttaaagacttgttcagtgactgtaaactctatgaaaccagaaaccgaatctgcctcttgctgggatctctaaa

SEQ ID NO: 185 - DNA, 24 bp, Artificial Sequence
  Synthetic construct
ttctgcttttcaaattcataacat

SEQ ID NO: 186 - DNA, 24 bp, Artificial Sequence
  Synthetic construct
tcattttcatttatttaagccttt

SEQ ID NO: 187 - DNA, 659 bp, Homo sapiens
acccaagcattatgggaacaggaactcaacttagctcttccagtagaggggtgagggattctgcttttcaaattcataacattgatctttttatgcaagatttccatttacagttgaataagtacttcatatttttccatcattagacaaatacaaaatggactaaataattttaagagatagtggaggcagcagggggtacagacttccttcttagagagtgtcagagaatatgctcccaatggtggaaaggaagatttacagtctagcggctaagtacctcctacacatttcccatcaatcagaaaatagacaggtacactaaagggacctgagaactcctcttgtaatttcaacacacccaaaatcaagggcctggatgccagcagctgcagcaagcaggtttttcctccctgttgagcaagacaggtgaggcaagataggacttggctttcttacatgatgcggtaacttgtgacttgagtctttttccctaatttgctagtgggaagaaaaatagctgagctttctaaaatgatagctctctatttttaaatgaatttgaaaagtcgattaaattatgtattttattgcctctgagtatcatattaaatgaatattttattttaaaggcttaaataaatgaaaatgatttttgt

SEQ ID NO: 188 - DNA, 594 bp, Artificial Sequence
  Synthetic cosntruct
ttctgcttttcaaattcataacattgatctttttatgcaagatttccatttacagttgaataagtacttcatatttttccatcattagacaaatacaaaatggactaaataattttaagagatagtggaggcagcagggggtacagacttccttcttagagagtgtcagagaatatgctcccaatggtggaaaggaagatttacagtctagcggctaagtacctcctacacatttcccatcaatcagaaaatagacaggtacactaaagggacctgagaactcctcttgtaatttcaacacacccaaaatcaagggcctggatgccagcagctgcagcaagcaggtttttcctccctgttgagcaagacaggtgaggcaagataggacttggctttcttacatgatgcggtaacttgtgacttgagtctttttccctaatttgctagtgggaagaaaaatagctgagctttctaaaatgatagctctctatttttaaatgaatttgaaaagtcgattaaattatgtattttattgcctctgagtatcatattaaatgaatattttattttaaaggcttaaataaatgaaaatga

SEQ ID NO: 189 - DNA, 24 bp, Artificial Sequence
  Synthetic construct
atcaatgggaagcgggaagaagga

SEQ ID NO: 190 - DNA, 24 bp, Artificial Sequence
  Synthetic construct
cacaggaacagcaccgtagatgaa

SEQ ID NO: 191 - DNA, 1071 bp, Homo sapiens
gcccgctggcactcctcctccgggaggctggacctcaccctgagggccctgcagagagtcgcccggatcaatgggaagcgggaagaaggagccaaattgagtatggaggtactccgggccagtctgcagaaggagctgaccatgggcaaaggccaggcatcggccatggagctgctgcgctgccccaccctccgccacctcttcctctgcctctccatgctgtggtttgccactagctttgcatactatgggctggtcatggacctgcagggctttggagtcagcatctacctaatccaggtgatctttggtgctgtggacctgcctgccaagcttgtgggcttccttgtcatcaactccctgggtcgccggcctgcccagatggctgcactgctgctggcaggcatctgcatcctgctcaatggggtgataccccaggaccagtccattgtccgaacctctcttgctgtgctggggaagggttgtctggctgcctccttcaactgcatcttcctgtatactggggaactgtatcccacaatgatccggcagacaggcatgggaatgggcagcaccatggcccgagtgggcagcatcgtgagcccactggtgagcatgactgccgagctctacccctccatgcctctcttcatctacggtgctgttcctgtggccgccagcgctgtcactgtcctcctgccagagaccctgggccagccactgccagacacggtgcaggacctggagagcaggtgggcccccactcagaaagaagcagggatatatcccaggaaagggaaacagacgcgacagcaacaagagcaccagaagtatatggtcccactgcaggcctcagcacaagagaagaatggactctgaggactgagaaggggccttacagaaccctaaagggagggaaggtcctacaggtctccggccacccacacaaggaggaggaagaggaaatggtgacccaagtgtgggggttgtggttcaggaaagcatcttcccaggggtccacctccctttataaaccccaccagaaccacatcattaaaaggtttgactgcg

SEQ ID NO: 192 - DNA, 606 bp, Artificial Sequence
  Synthetic construct
atcaatgggaagcgggaagaaggagccaaattgagtatggaggtactccgggccagtctgcagaaggagctgaccatgggcaaaggccaggcatcggccatggagctgctgcgctgccccaccctccgccacctcttcctctgcctctccatgctgtggtttgccactagctttgcatactatgggctggtcatggacctgcagggctttggagtcagcatctacctaatccaggtgatctttggtgctgtggacctgcctgccaagcttgtgggcttccttgtcatcaactccctgggtcgccggcctgcccagatggctgcactgctgctggcaggcatctgcatcctgctcaatggggtgataccccaggaccagtccattgtccgaacctctcttgctgtgctggggaagggttgtctggctgcctccttcaactgcatcttcctgtatactggggaactgtatcccacaatgatccggcagacaggcatgggaatgggcagcaccatggcccgagtgggcagcatcgtgagcccactggtgagcatgactgccgagctctacccctccatgcctctcttcatctacggtgctgttcctgtg

SEQ ID NO: 193 - DNA, 24 bp, Artificial Sequence
  Synthetic construct
accttcatacctagacctgttccg

SEQ ID NO: 194 - DNA, 24 bp, Artificial Sequence
  Synthetic construct
cacttagttctggacctgcttcat

SEQ ID NO: 195 - DNA, 1056 bp, Homo sapiens
tccgaagaccttcatacctagacctgttccgcacaccacggctccgacacatctcactgtgctgcgtggtggtgtggttcggagtgaacttctcctattacggcctgagtctggatgtgtcggggctggggctgaacgtgtaccagacacagctgttgttcggggctgtggaactgccctccaagctgctggtctacttgtcggtgcgctacgcaggacgccgcctcacgcaagccgggacactgctgggcacggccctggcgttcggcactagactgctagtgtcctccgatatgaagtcctggagcactgtcctggcagtgatggggaaagctttttctgaagctgccttcaccactgcttacctgttcacttcagagttgtaccctacggtgctcagacagacagggatggggctgactgcactggtgggccggctggggggctctttggccccactggcggccttgctagatggagtgtggctgtcactgcccaagcttacttatggggggatcgccctgctggctgccggcaccgccctcctgctgccagagacgaggcaggcacagctgccagagaccatccaggacgtggagagaaagagtgccccaaccagtcttcaggaggaagagatgcccatgaagcaggtccagaactaagtgggagtggaggcaggccctccacagaagctctgcagcaggggctgggagagcagaagggcaggcccttcaactcaggctgggagagcagaagggcaggccctgcaactcaggctgggagtatcgaaccctctgcctagggccggagttgctgccagtacccgctccctctgctcatccatccttgattatttggcttctaggaacagttgacttcccagaatgcagtgggctgctgggcacccctctcacggttggggaggattctgtaaataaaggtgccccttgggttggggcagtggtgacgagctgtgggaagagccctggataggaagccactgagtctgccctgggctctgataaaacttcaccattaaaaaaaaaaaaaaaaaaa

SEQ ID NO: 196 - DNA, 658 bp, Artificial Sequence
  Synthetic construct
accttcatacctagacctgttccgcacaccacggctccgacacatctcactgtgctgcgtggtggtgtggttcggagtgaacttctcctattacggcctgagtctggatgtgtcggggctggggctgaacgtgtaccagacacagctgttgttcggggctgtggaactgccctccaagctgctggtctacttgtcggtgcgctacgcaggacgccgcctcacgcaagccgggacactgctgggcacggccctggcgttcggcactagactgctagtgtcctccgatatgaagtcctggagcactgtcctggcagtgatggggaaagctttttctgaagctgccttcaccactgcttacctgttcacttcagagttgtaccctacggtgctcagacagacagggatggggctgactgcactggtgggccggctggggggctctttggccccactggcggccttgctagatggagtgtggctgtcactgcccaagcttacttatggggggatcgccctgctggctgccggcaccgccctcctgctgccagagacgaggcaggcacagctgccagagaccatccaggacgtggagagaaagagtgccccaaccagtcttcaggaggaagagatgcccatgaagcaggtccagaactaagtg

SEQ ID NO: 197 - DNA, 24 bp, Artificial Sequence
  Synthetic construct
aagtgacctgttccggatacccat

SEQ ID NO: 198 - DNA, 24 bp, Artificial Sequence
  Synthetic construct
ccagttttccaggtcttcgatcgt

SEQ ID NO: 199 - DNA, 1047 bp, Homo sapiens
cagttcattctgcccggcctggcctacgccatcccccagtggcgttggctgcagttaactgtgtccattcccttcttcgtcttcttcctatcatcctggtggacaccagagtccatacgctggtggtcttgtctggaagtcctcgaaggccctgaagatactccggcgggtggctgtcttcaatggcaagaagagggagaaaggctcagcttggaggagctcaaactcaacctgcagaaggagatctccttggccaaggccaagtacaccgcaagtgacctgttccggatacccatcggtgcgccgcatgaccttctgctttccctggcctggtttgctaccggttttgcctactatagtttggctatgggtgtggaagaatttggagtcaacctctacatcctccagatcatctttggtggggtcgatgtcccagccaagttcatcaccatcctctccttaagctacctgggccggcataccactcaggggcgctgccctgctcctggcagaggggccatcttggctctcacctttgtgcccttggacttgcagaccgtggagacagtattggctgtgtttgggaagggatgcctatccagctccttcagctgcctcttcctctacacaagtgaattataccccacagtcatcaggcaaacaggtatgggcgtaagtaacctgtggacccgcgtgggaagcatggtgtccccgctggtgaaaatcacgggtgaggtacagcccttcatccccaatatcatctttacgggatctaccgccctcctcgggggcagtgctgccctcttcctgcctgagaccctgaacagcccttgccagagacgatcgaagacctggaaaactggtcagtcactgcctctggccccatcagtgctcctccctggggaagcaggtctgggcccagggcttttccttagctctctgtccctaggtctgcgggcaaagaagccaaagcaggagccagaggtggaaaaggcctcccagaggatccctctacagcctcacggaccaggcctgggctccagctga

SEQ ID NO: 200 - DNA, 591 bp, Artificial Sequence
  Synthetic construct
aagtgacctgttccggatacccatcggtgcgccgcatgaccttctgctttccctggcctggtttgctaccggttttgcctactatagtttggctatgggtgtggaagaatttggagtcaacctctacatcctccagatcatctttggtggggtcgatgtcccagccaagttcatcaccatcctctccttaagctacctgggccggcataccactcaggggcgctgccctgctcctggcagaggggccatcttggctctcacctttgtgcccttggacttgcagaccgtggagacagtattggctgtgtttgggaagggatgcctatccagctccttcagctgcctcttcctctacacaagtgaattataccccacagtcatcaggcaaacaggtatgggcgtaagtaacctgtggacccgcgtgggaagcatggtgtccccgctggtgaaaatcacgggtgaggtacagcccttcatccccaatatcatctttacgggatctaccgccctcctcgggggcagtgctgccctcttcctgcctgagaccctgaacagcccttgccagagacgatcgaagacctggaaaactgg

SEQ ID NO: 201 - DNA, 24 bp, Artificial Sequence
  Synthetic construct
gcctaacctgcctcaccatctaca

SEQ ID NO: 202 - DNA, 24 bp, Artificial Sequecne
  Synthetic construct
gtctcgttattggttgggcatggc

SEQ ID NO: 203 - DNA, 1070 bp, Homo sapiens
ggtcttcgacctgcagagcctgggccgtgacatcttcctcctccaggccctcttcggggccgtggacttcctgggccgggccaccactgccctcttgctcagtttccttggccgccgcaccatccaggcgggttcccaggccatggccggcctcgccattctagccaacatgctggtgccgcaagatttgcagaccctgcgtgtggtctttgctgtgctgggaaagggatgttttgggataagcctaacctgcctcaccatctacaaggctgaactctttccaacgccagtgcggatgacagcagatggcattctgcatacagtgggccggctgggggctatgatgggtcccctgatcctgatgagccgccaagccctgcccctgctgcctcctctcctctatggcgttatctccattgcttccagcctggttgtgctgttcttcctcccggagacccagggacttccgctccctgacactatccaggacctggagagccagaaatcaacagcagcccagggcaaccggcaagaggccgtcactgtggaaagtacctcgctctagaaattgtgcctgcatggagcccctttagtcaaagactcctggaaaggagttgcctcttctccaatcagagcgtggaggcgagttgggcgacttcaagggcctggcatggcagaggccaggcagccgtggccgagtggacagcgtggccgtctgctgtggctgaaggcagcttccacagctcactcctcttctccctgccctgatcagattccccaccttacccgggccctacaggagcctgtgcagatggccatgcccaaccaataacgagacggttcccctccctttccctgccaggctcatgtctttacaccttcactcagccacgccaaccagagactgggttccaatctcaccccaccacatacagagccctcatctgtgaaatgagaatgatcacgtgacccaccccccagggcaggtatcagggtgaactgatcttagcaccggccaaataaatggaacctgctgagagagctgccagataaaaaaaaaaaaaaaaaaa

SEQ ID NO: 204 - DNA, 596 bp, Artificial Sequence
  Synthetic construct
gcctaacctgcctcaccatctacaaggctgaactctttccaacgccagtgcggatgacagcagatggcattctgcatacagtgggccggctgggggctatgatgggtcccctgatcctgatgagccgccaagccctgcccctgctgcctcctctcctctatggcgttatctccattgcttccagcctggttgtgctgttcttcctcccggagacccagggacttccgctccctgacactatccaggacctggagagccagaaatcaacagcagcccagggcaaccggcaagaggccgtcactgtggaaagtacctcgctctagaaattgtgcctgcatggagcccctttagtcaaagactcctggaaaggagttgcctcttctccaatcagagcgtggaggcgagttgggcgacttcaagggcctggcatggcagaggccaggcagccgtggccgagtggacagcgtggccgtctgctgtggctgaaggcagcttccacagctcactcctcttctccctgccctgatcagattccccaccttacccgggccctacaggagcctgtgcagatggccatgcccaaccaataacgagac

SEQ ID NO: 205 - DNA, 24 bp, Artificial Sequence
  Synthetic construct
aagaaggcaacacatggcacgctg

SEQ ID NO: 206 - DNA, 24 bp, Artificial Sequence
  Synthetic construct
tgggtaggagtttcacgggcatct

SEQ ID NO: 207 - DNA, 1038 bp, Homo sapiens
ggcccctggctgcccttgctggtgtatgggacggtgccagtgctgagtggcctggccgcactgcttctgcccgagacccagagcttgccgctgcccgacaccatccaagatgtgcagaaccaggcagtaaagaaggcaacacatggcacgctggggaactctgtcctaaaatccacacagttttagcctcctggggaacctgcgatgggacggtcagaggaagagacttcttctgttctctggagaaggcaggaggaaagcaaagacctccatttccagaggcccagaggctgccctctgaggtccccactctcccccagggctgcccctccaggtgagccctgcccctctcacagtccaaggggcccccttcaatactgaaggggaaaaggacagtttgattggcaggaggtgacccagtgcaccatcaccctgccctgccctcgtggcttcggagagcagaggggtcaggcccaggggaacgagctggccttgccaaccctctgcttgactccgcactgccacttgtccccccacacccgtccacctgcccagagctcagagctaaccaccatccatggtcaagacctctcctagctccacacaagcagtagagtctcagctccacagctttacccagaagccctgtaagcctggcccctggcccctccccatgtccctccaggcctcagccacctgcccgccacatcctctgcctgctgtccccttcccaccctcatccctgaccgactccacttaacccccaaacccagccccccttccaggggtccagggccagcctgagatgcccgtgaaactcctacccacagttacagccacaagcctgcctcctcccaccctgccagcctatgagttcccagagggttggggcagtcccatgaccccatgtcccagctccccacacagcgctgggccagagaggcattggtgcgagggattgaataaagaaacaaatgaatggcaaaaaaaaaaaaaaaaaaaaaaaaaaaaaaaaaaaaaaaaaaaaaaaaaaaaaaa

SEQ ID NO: 208 - DNA, 698 bp, Artificial Sequence
  Synthetic construct
aagaaggcaacacatggcacgctggggaactctgtcctaaaatccacacagttttagcctcctggggaacctgcgatgggacggtcagaggaagagacttcttctgttctctggagaaggcaggaggaaagcaaagacctccatttccagaggcccagaggctgccctctgaggtccccactctcccccagggctgcccctccaggtgagccctgcccctctcacagtccaaggggcccccttcaatactgaaggggaaaaggacagtttgattggcaggaggtgacccagtgcaccatcaccctgccctgccctcgtggcttcggagagcagaggggtcaggcccaggggaacgagctggccttgccaaccctctgcttgactccgcactgccacttgtccccccacacccgtccacctgcccagagctcagagctaaccaccatccatggtcaagacctctcctagctccacacaagcagtagagtctcagctccacagctttacccagaagccctgtaagcctggcccctggcccctccccatgtccctccaggcctcagccacctgcccgccacatcctctgcctgctgtccccttcccaccctcatccctgaccgactccacttaacccccaaacccagccccccttccaggggtccagggccagcctgagatgcccgtgaaactcctaccca

SEQ ID NO: 209 - DNA, 24 bp, Artificial Sequence
  Synthetic construct
cctgcacctatatattttggcgct

SEQ ID NO: 210 - DNA, 24 bp, Artificial Sequence
  Synthetic construct
ctttagggggctgttattgatgtc

SEQ ID NO: 211 - DNA, 1049 bp, Homo sapiens
atctgaagagaagtcccttggtgtgggattacatacattttgcacaagagtatttgctggcattcctgcacctatatattttggcgctttaatggattccacatgtttacactggggaactttgaaatgtggtgagtcaggggcatgcaggatatatgattccaccaccttcagatacatctacctcggattgccggcagcactaagaggatcaagctttgttccagccttaatcatcttaattcttttgaggaagtgtcatctacctggtgaaaatgcctcttcaggaacagagcttatagagacaaaagtcaaagggaaggaaaatgagtgcaaagatatataccaaaagtccacggttttgaaagatgatgaattgaaaactaaattgtaattgtcctattatattacttttttcagaattagagaacatgctgtacaacttaattgttttaaaaatcagtagagatataatagataactttttcttgtctttaagaacctaaaaaacctcttaactcaaaataataaaatgttcactaatgatatttctaaggtatcagtgacacttgagttttcctaggagggacatcaataacagccccctaaagaagattcttagagccagctttatttttatgttgaaacagcaatttcccttaattcatcgaagtaagggtgtacttcctacatctccttctactaatacttctaaaaattttctgttatgaaaacctatttaattccactaaatttgttctttgatattggaattattcagatgcctaaattctcattctgttatgtgaagatttaaatattttattcaagtttatcgcttccatgtgagagaagcctacatcttcttattctatttaggaatcgttctttaactcttcttattcattctaggcatgactcctatataatagattactcataaatataccctcctactttcaattttttcttttctttattactcatacatttgctcaatttgtacagaatactgacaaacttaagcaggttattaaacatcatgaggc

SEQ ID NO: 212 - DNA, 547 bp, Artificial Sequence
  Synthetic construct
cctgcacctatatattttggcgctttaatggattccacatgtttacactggggaactttgaaatgtggtgagtcaggggcatgcaggatatatgattccaccaccttcagatacatctacctcggattgccggcagcactaagaggatcaagctttgttccagccttaatcatcttaattcttttgaggaagtgtcatctacctggtgaaaatgcctcttcaggaacagagcttatagagacaaaagtcaaagggaaggaaaatgagtgcaaagatatataccaaaagtccacggttttgaaagatgatgaattgaaaactaaattgtaattgtcctattatattacttttttcagaattagagaacatgctgtacaacttaattgttttaaaaatcagtagagatataatagataactttttcttgtctttaagaacctaaaaaacctcttaactcaaaataataaaatgttcactaatgatatttctaaggtatcagtgacacttgagttttcctaggagggacatcaataacagccccctaaag

SEQ ID NO: 213 - DNA, 24 bp, Artificial Sequence
  Synthetic construct
ttcagacaaacacacactcagcgc

SEQ ID NO: 214 - DNA, 24 bp, Artificial Sequence
  Synthetic construct
ctgggaaacaagagggatgaagga

SEQ ID NO: 215 - DNA, 1068 bp, Homo sapiens
tcaggagtgggacacccagacttggcagggccttcaagaggcctgtgtgggggccccaggaatccttagctgaagcggggagactcactctccatctcaggaaattctagcccttgccctcagggagccacggttgagggtgaggcccaacacctgccttagggccctgggtgggcaagtctgggccctggggtagggagggagactcaggcccacacttgggtattttctaatttcagacaaacacacactcagcgcgcactcactgattcctacacattgccaagatttcacacatgtgaccaggggccaccaaagtccctgtgacctttgtgactaggatcctaatttctctattttctcctgggtgcctgggtctgtgtcacctggggcagtgtggataatgtttagttctgtgacactgttttttgggggtggcacctggttctccgatgcctgggctggtgtcaggcccaggactgtagtgctgggagcagtaaagctcagctctgtgtaatgagtgatgctatggcttgctcgtgtcttatgatccaatccttttctacatcagcccttgttttgttttatggctagtcttatctggcctggttatttccttgcggggaggagagggtttgctaatctgctcccagcccaacctattaccaccccacctcgctgggacctactgctcgggaggcagcagacagggagccaccagcagtggcttcctggccctgtgctgggggtggggggaagctgggggcacatgtggcccttgccttctgagcagctcccagtgccagggctttgagactttcccacatgataaaagaaaagggaggtacagaagttccaattccctttttattttgctggttggtatctgtaaatgtttaataaatatctgagcatgtatctatcaacgccaagaatttcaaagtctccttcaacaatatgaggcttttaggatgtttatattccttcatccctcttgtttcccaggttttgcagggaaaaaaagtctggaattatagatacagcttattattaaatttgttcttgcat

SEQ ID NO: 216 - DNA, 771 bp, Artificial Sequence
  Synthetic construct
ttcagacaaacacacactcagcgcgcactcactgattcctacacattgccaagatttcacacatgtgaccaggggccaccaaagtccctgtgacctttgtgactaggatcctaatttctctattttctcctgggtgcctgggtctgtgtcacctggggcagtgtggataatgtttagttctgtgacactgttttttgggggtggcacctggttctccgatgcctgggctggtgtcaggcccaggactgtagtgctgggagcagtaaagctcagctctgtgtaatgagtgatgctatggcttgctcgtgtcttatgatccaatccttttctacatcagcccttgttttgttttatggctagtcttatctggcctggttatttccttgcggggaggagagggtttgctaatctgctcccagcccaacctattaccaccccacctcgctgggacctactgctcgggaggcagcagacagggagccaccagcagtggcttcctggccctgtgctgggggtggggggaagctgggggcacatgtggcccttgccttctgagcagctcccagtgccagggctttgagactttcccacatgataaaagaaaagggaggtacagaagttccaattccctttttattttgctggttggtatctgtaaatgtttaataaatatctgagcatgtatctatcaacgccaagaatttcaaagtctccttcaacaatatgaggcttttaggatgtttatattccttcatccctcttgtttcccag

SEQ ID NO: 217 - DNA, 24 bp, Artificial Sequence
  Synthetic construct
gaattgaaatcacttgcactgggt

SEQ ID NO: 218 - DNA, 24 bp, Artificial Sequence
  Synthetic construct
gaatctagctcctcctttttaacc

SEQ ID NO: 219 - DNA, 1030 bp, Homo sapiens
tcatgctgattgttaaaattgttcaacctgaattgaaatcacttgcactgggtttccactcaatggttatacgagcactaggaggaattctagctccaatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaaggggagaaaaaaagccacttctgcttctgtgtttccaaacagcattgcattgattcagtaagatgttatttttgaggagttcctggtcctttcactaagaatttccacatcttttatggtggaagtataaataagcctatgaacttataataaaacaaactgtaggtagaaaaaatgagagtactcattgttacattatagctacatatttgtggttaaggttagactatatgatccatacaaattaaagtgagagacatggttactgtgtaataaaagaaaaaatacttgttcaggtaattctaattcttaataaaacaaatgagtatcatacaggtagaggttaaaaaggaggagctagattcatatcctaagtaaagagaaatgcctagtgtctattttattaaacaaacaaacacagagtttgaactataatactaaggcctgaagtctagcttggatatatgctacaataatatctgttactcacataaaattatatatttcacagactttatcaatgtataattaacaattatcttgtttaagtaaatttagaatacatttaagtattgtggaagaaataaagacattccaatatttgcaaaaaaaaaaaaaaa

SEQ ID NO: 220 - DNA, 746 bp, Artificial Sequence
  Synthetic construct
gaattgaaatcacttgcactgggtttccactcaatggttatacgagcactaggaggaattctagctccaatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaaggggagaaaaaaagccacttctgcttctgtgtttccaaacagcattgcattgattcagtaagatgttatttttgaggagttcctggtcctttcactaagaatttccacatcttttatggtggaagtataaataagcctatgaacttataataaaacaaactgtaggtagaaaaaatgagagtactcattgttacattatagctacatatttgtggttaaggttagactatatgatccatacaaattaaagtgagagacatggttactgtgtaataaaagaaaaaatacttgttcaggtaattctaattcttaataaaacaaatgagtatcatacaggtagaggttaaaaaggaggagctagattc

SEQ ID NO: 221 - DNA, 24 bp, Artificial Sequence
  Synthetic construct
tcaagatcttcctggtgtccgagt

SEQ ID NO: 222 - DNA, 24 bp, Artificial Sequence
  Synthetic construct
ccaaataccagcatcgtgaacagg

SEQ ID NO: 223 - DNA, 1022 bp, Homo sapiens
cggcgggggaaggatgcaggggaagaagccgggcggttcgtcgggcggcggccggagcggcgagctgcagggggacgaggcgcagaggaacaagaaaaagaaaaagaaggtgtcctgcttttccaacatcaagatcttcctggtgtccgagtgcgccctgatgctggcgcagggcacggtgggcgcctacctggtgagcgtcctgaccaccctggagcgtaggttcaacctgcagagcgctgacgtgggtgtgatcgctagcagcttcgagatcgggaacctggcgctcatcctcttcgtgagctacttcggggcacgcgggcaccggccgcgcctgatcggctgcggcggcatcgtcatggcgctgggcgcgctgctgtcggcgctgcccgagttcctgacccaccagtacaagtacgaggcgggcgagatccgctggggcgccgagggccgcgacgtctgcgcagccaacggctcgggcggcgacgaggggcccgaccccgacctcatctgccgcaaccggacggctaccaacatgatgtacttgctgctcattggggcccaggtgctcctgggcatcggtgctacccctgtgcagcccctgggcgtctcctacatcgacgaccacgtgcggaggaaggactcctcgctctatataggaatcctgttcacgatgctggtatttggaccagcctgcgggtttatcctgggctctttctgtaccaaaatctacgtggatgcggtcttcattgacacaagtaacctggacatcactccggacgacccccgctggatcggagcctggtggggtggctttctgctctgcggtgccttactcttcttctcttccctcttgatgtttgggtttccacagtccctgcccccgcactcagagcccgccatggaaagcgagcaggccatgctctccgaaagagaatacgagagacccaagcccagcaacggggtcctgaggcaccccctggagccagacagcagtgcctcctgtttccagcagctgagag

SEQ ID NO: 224 - DNA, 559 bp, Artificial Sequence
  Synthetic construct
tcaagatcttcctggtgtccgagtgcgccctgatgctggcgcagggcacggtgggcgcctacctggtgagcgtcctgaccaccctggagcgtaggttcaacctgcagagcgctgacgtgggtgtgatcgctagcagcttcgagatcgggaacctggcgctcatcctcttcgtgagctacttcggggcacgcgggcaccggccgcgcctgatcggctgcggcggcatcgtcatggcgctgggcgcgctgctgtcggcgctgcccgagttcctgacccaccagtacaagtacgaggcgggcgagatccgctggggcgccgagggccgcgacgtctgcgcagccaacggctcgggcggcgacgaggggcccgaccccgacctcatctgccgcaaccggacggctaccaacatgatgtacttgctgctcattggggcccaggtgctcctgggcatcggtgctacccctgtgcagcccctgggcgtctcctacatcgacgaccacgtgcggaggaaggactcctcgctctatataggaatcctgttcacgatgctggtatttgg

SEQ ID NO: 225 - DNA, 24 bp, Artificial Sequence
  Synthetic construct
acggcctcatgtacttctcactgt

SEQ ID NO: 226 - DNA, 24 bp, Artificial Sequence
  Synthetic construct
gcaggtcaaatagaagttcccgtg

SEQ ID NO: 227 - DNA, 1070 bp, Homo sapiens
tgcctgcagctgccagccagaacactacagccctgtgtgcggctcggacggcctcatgtacttctcactgtgccacgcagggtgccctgcagccacggagacgaatgtggacggccagaaggtgtaccgagactgtagctgtatccctcagaatctttcctctggttttggccatgccactgcagggaaatgcacttcaacttgtcagagaaagcccctccttctggttttcatattcgttgtaattttctttacattcctcagcagcattcctgcactaacggcaactctacgatgtgtccgtgaccctcagagatcctttgccctgggaatccagtggattgtagttagaatactagggggcatcccggggcccatcgccttcggctgggtgatcgacaaggcctgtctgctgtggcaggaccagtgtggccagcagggctcctgcttggtgtaccagaattcggccatgagccgctacatactcatcatggggctcctgtacaaggtgctgggcgtcctcttctttgccatagcctgcttcttatacaagcccctgtcggagtcttcagatggcctggaaacttgtctgcccagccagtcctcagcccctgacagtgccacagatagccagctccagagcagcgtctgaccaccgcccgcgcccacccggccacggcgggcactcagcatttcctgatgacagaacagtgccgttgggtgatgcaatcacacgggaacttctatttgacctgcaaccttctacttaacctgtggtttaaagtcggctgtgacctcctgtccccagagctgtacggccctgcagtgggtgggaggaacttgcataaatatatatttatggacacacagtttgcatcagaacgtgtttatagaatgtgttttatacccgatcgtgtgtggtgtgcgtgaggacaaactccgcaggggctgtgaatcccactgggagggcggcgggcctgcagcccgaggaaggcttgtgtgtcctcagttaaaactgtgcatatcgaaatatattttgttatttaagcctgaaaaaaaaaaaaaaaaaa

SEQ ID NO: 228 - DNA, 709 bp, Artificial Sequence
  Synthetic construct
acggcctcatgtacttctcactgtgccacgcagggtgccctgcagccacggagacgaatgtggacggccagaaggtgtaccgagactgtagctgtatccctcagaatctttcctctggttttggccatgccactgcagggaaatgcacttcaacttgtcagagaaagcccctccttctggttttcatattcgttgtaattttctttacattcctcagcagcattcctgcactaacggcaactctacgatgtgtccgtgaccctcagagatcctttgccctgggaatccagtggattgtagttagaatactagggggcatcccggggcccatcgccttcggctgggtgatcgacaaggcctgtctgctgtggcaggaccagtgtggccagcagggctcctgcttggtgtaccagaattcggccatgagccgctacatactcatcatggggctcctgtacaaggtgctgggcgtcctcttctttgccatagcctgcttcttatacaagcccctgtcggagtcttcagatggcctggaaacttgtctgcccagccagtcctcagcccctgacagtgccacagatagccagctccagagcagcgtctgaccaccgcccgcgcccacccggccacggcgggcactcagcatttcctgatgacagaacagtgccgttgggtgatgcaatcacacgggaacttctatttgacctgc

SEQ ID NO: 229 - DNA, 24 bp, Artificial Sequence
  Synthetic construct
tgggactaactgtgatactgggca

SEQ ID NO: 230 - DNA, 24 bp, Artificial Sequence
  Synthetic construct
cacagatgaagacagctatgggag

SEQ ID NO: 231 - DNA, 1097 bp, Homo sapiens
acatatatatctgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaacatataatggaagatgcagatgataaaactaattttgaactttttaatttatataaattattttatatcacttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtgtattttaataatgatcttaggatggagcagaacatggagaggaagatttcattttaagctcctccttttctttgaaatacaataatttatatagaaatgtgtagcagcaaattatattggggattagaattttgaattaatagctctcctactattaatttacatgtgctttttgtgtggcgctataagtgactatggttgtaaagtaataaaattgatgttaacatgcccaattattgttcttttatgaattcaatgaatttaaaactattgttaaatataatactgccccactttaatatatgtaagcaacttcctacttatacacgacgtgttcctaaaacatgtttgaaaggtgaatttctgaaagtctacaataaatgtaggtgttacaacaggaaaaaaaaaaaaaaaaaa

SEQ ID NO: 232 - DNA, 669 bp, Artificial Sequence
  Synthetic construct
tgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaacatataatggaagatgcagatgataaaactaattttgaactttttaatttatataaattattttatatcacttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtg

SEQ ID NO: 233 - DNA, 24 bp, Artificial Sequence
  Synthetic construct
acggcctcatgtacttctcactgt

SEQ ID NO: 234 - DNA, 24 bp, Artificial Sequence
  Synthetic construct
gcaggtcaaatagaagttcccgtg

SEQ ID NO: 235 - DNA, 1083 bp, Homo sapiens
agaacactacagccctgtgtgcggctcggacggcctcatgtacttctcactgtgccacgcagggtgccctgcagccacggagacgaatgtggacggccagaaggtgtaccgagactgtagctgtatccctcagaatctttcctctggttttggccatgccactgcagggaaatgcacttcaacttgtcagagaaagcccctccttctggttttcatattcgttgtaattttctttacattcctcagcagcattcctgcactaacggcaactctacgatgtgtccgtgaccctcagagatcctttgccctgggaatccagtggattgtagttagaatactagggggcatcccggggcccatcgccttcggctgggtgatcgacaaggcctgtctgctgtggcaggaccagtgtggccagcagggctcctgcttggtgtaccagaattcggccatgagccgctacatactcatcatggggctcctgtacaaggtgctgggcgtcctcttctttgccatagcctgcttcttatacaagcccctgtcggagtcttcagatggcctggaaacttgtctgcccagccagtcctcagcccctgacagtgccacagatagccagctccagagcagcgtctgaccaccgcccgcgcccacccggccacggcgggcactcagcatttcctgatgacagaacagtgccgttgggtgatgcaatcacacgggaacttctatttgacctgcaaccttctacttaacctgtggtttaaagtcggctgtgacctcctgtccccagagctgtacggccctgcagtgggtgggaggaacttgcataaatatatatttatggacacacagtttgcatcagaacgtgtttatagaatgtgttttatacccgatcgtgtgtggtgtgcgtgaggacaaactccgcaggggctgtgaatcccactgggagggcggcgggcctgcagcccgaggaaggcttgtgtgtcctcagttaaaactgtgcatatcgaaatatattttgttatttaagcctgcgaaaaaaaaaaaaaaaaaaaaaaaaaaaaaaaaaaaaaaaaaaaaaaa

SEQ ID NO: 236 - DNA, 709 bp, Artificial Sequence; identical to SEQ ID NO: 228
  Synthetic construct

SEQ ID NO: 237 - DNA, 24 bp, Artificial Sequence
  Synthetic construct
cgctcttctttatcggctgctcca

SEQ ID NO: 238 - DNA, 24 bp, Artificial Sequence
  Synthetic construct
ttgcctctttgtcctgctgcctca

SEQ ID NO: 239 - DNA, 1122 bp, Homo sapiens
catcacagcctcctacgccaacctgctcatcggctgcctctccttcccttcggtcatcgtgggcatcgtggtgggtggcgtcctggtcaagcggctccacctgggccctgtgggatgcggtgccctttgcctgctggggatgctgctgtgcctcttcttcagcctgccgctcttctttatcggctgctccagccaccagattgcgggcatcacacaccagaccagtgcccaccctgggctggagctgtctccaagctgcatggaggcctgctcctgcccattggacggctttaaccctgtctgcgaccccagcactcgtgtggaatacatcacaccctgccacgcaggctgctcaagctgggtggtccaggatgctctggacaacagccaggttttctacaccaactgcagctgcgtggtggagggcaaccccgtgctggcaggatcctgcgactcaacgtgcagccatctggtggtgcccttcctgctcctggtcagcctgggctcggccctggcctgtctcacccacacaccctccttcatgctcatcctaagaggagtgaagaaagaagacaagactttggctgtgggcatccagttcatgttcctgaggattttggcctggatgcccagccccgtgatccacggcagcgccatcgacaccacctgtgtgcactgggccctgagctgtgggcgtcgagctgtctgtcgctactacaataatgacctgctccgaaaccggttcatcggcctccagttcttcttcaaaacaggttctgtgatctgcttcgccttagttttggctgtcctgaggcagcaggacaaagaggcaaggaccaaagagagcagatccagccctgccgtagagcagcaattgctagtgtcggggccagggaagaagccagaggattcccgagtgtgagctgtcttggggccccacctggccaagagtagcagccacagcagtacctcctctgagtcctttgcccaagattgggtgtcaagagccctgtgttccattctggctcctccactaaattgctgtgtgacttcaggcaaaaaaaaaaaaaaaaaaaaaaaaaaaaaaaaaaaaaaaaaaaaaaaaaaaaaaaaaaaaaaaaa

SEQ ID NO: 240 - DNA, 666 bp, Artificial Sequence
  Synthetic construct
cgctcttctttatcggctgctccagccaccagattgcgggcatcacacaccagaccagtgcccaccctgggctggagctgtctccaagctgcatggaggcctgctcctgcccattggacggctttaaccctgtctgcgaccccagcactcgtgtggaatacatcacaccctgccacgcaggctgctcaagctgggtggtccaggatgctctggacaacagccaggttttctacaccaactgcagctgcgtggtggagggcaaccccgtgctggcaggatcctgcgactcaacgtgcagccatctggtggtgcccttcctgctcctggtcagcctgggctcggccctggcctgtctcacccacacaccctccttcatgctcatcctaagaggagtgaagaaagaagacaagactttggctgtgggcatccagttcatgttcctgaggattttggcctggatgcccagccccgtgatccacggcagcgccatcgacaccacctgtgtgcactgggccctgagctgtgggcgtcgagctgtctgtcgctactacaataatgacctgctccgaaaccggttcatcggcctccagttcttcttcaaaacaggttctgtgatctgcttcgccttagttttggctgtcctgaggcagcaggacaaagaggcaa

SEQ ID NO: 241 - DNA, 24 bp, Artificial Sequence
  Synthetic construct
ggagagaccttttgcactgggaat

SEQ ID NO: 242 - DNA, 24 bp, Artificial Sequence
  Synthetic construct
ccctcaatgaatagcggctgtgta

SEQ ID NO: 243 - DNA, 1112 bp, Homo sapiens
tttcatagtcaccttcatcacagcatgtgcccaaccatcagctatcatagtaacactcaggtccgtagaagatgaggagagaccttttgcactgggaatgcagtttgttttgttgcgaacacttgcatacattcctactccaatctactttggagcagtcattgacaccacctgcatgctctggcaacaggaatgtggtgtgcagggttcttgctgggagtacaacgtgacgtcgtttcgttttgtgtattttggtttggctgccggcctcaaattcgttgggtttatttttatttttctggcctggtactccataaaatacaaggaggatggactgcagaggcggaggcagagagaatttcccctgagcaccgtgagtgagagagtgggacaccccgacaatgcccggactagatcttgcccagctttcagcacccagggagaattccacgaagagactggcctgcaaaaagggatccagtgcgcagcacagacctacccggggcccttcccagaagcaataagttcctctgcggacccggggctggaagagagccccgctgccttggagccgccctcctgaagcttgaaaatggaagaatttagttttgttggttgaattgaaaatggcgacttgagaaacaactgtgccttcttttctttctttcttttttttaacctctacagacacaatcctcaaaccaacaaaactcagtatacacagccgctattcattgagggctggatacctcaacaagactgagagcctttccccgcttctctccaagaaggagacgttcagctagatttgttcccatttccgttgtgttaattcaaagctcatgctcccctacggtacaggctgaggtacacggttagcaaaaccatgggaaggggaatggcggtgcatatcattaactaacactccaaacaaaggtgagcttgcccaggacttggcatttccaaatcaaagtttttagatatgaacacctactgtgagttctgctacaaagcacaaatgaatttgtctcaactatgcaatttgattggaaaaatgtatgtgcagcatgttacatttactttcacggaataaagcagatatgtttctgaaa

SEQ ID NO: 244 - DNA, 666 bp, Artificial Sequence
  Synthetic construct
ggagagaccttttgcactgggaatgcagtttgttttgttgcgaacacttgcatacattcctactccaatctactttggagcagtcattgacaccacctgcatgctctggcaacaggaatgtggtgtgcagggttcttgctgggagtacaacgtgacgtcgtttcgttttgtgtattttggtttggctgccggcctcaaattcgttgggtttatttttatttttctggcctggtactccataaaatacaaggaggatggactgcagaggcggaggcagagagaatttcccctgagcaccgtgagtgagagagtgggacaccccgacaatgcccggactagatcttgcccagctttcagcacccagggagaattccacgaagagactggcctgcaaaaagggatccagtgcgcagcacagacctacccggggcccttcccagaagcaataagttcctctgcggacccggggctggaagagagccccgctgccttggagccgccctcctgaagcttgaaaatggaagaatttagttttgttggttgaattgaaaatggcgacttgagaaacaactgtgccttcttttctttctttcttttttttaacctctacagacacaatcctcaaaccaacaaaactcagtatacacagccgctattcattgaggg

SEQ ID NO: 245 - DNA, 24 bp, Artificial Sequence
  Synthetic construct
gggcacagtgtcaattctcctaag

SEQ ID NO: 246 - DNA, 24 bp, Artificial Sequence
  Synthetic construct
cacagatgaagacagctatgggag

SEQ ID NO: 247 - DNA, 1101 bp, Homo sapiens
ttcagacatatatatttgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaatatataatggaagatgcagatgataaaactaattttgaactttttaatttatataaattattttatatcatttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtgtattttaataatgatcttaggatggagcagaacatggagaggaagatttcattttaagctcctccttttccttgaaatacaataatttatatagaaatgtgtagcagcaaattatattggggattagaattttgaattaatagctctcctactattaatttacatgtgctttttgtgtggcgctataagtgactatggttgtaaagtaataaaattgatgttaacatgcccaattattgttcttttatgaattcaatgaatttaaaactattgttaaatataatactgccccactttaatatatgtaagcaacttcctacttatacacgacgtgttcctaaaacatgtttgaaaggtgaatttctgaaagtctcccataaatgtaggtgttacaacaggaaaaaaaaaaaaaaaaa

SEQ ID NO: 248 - DNA, 650 bp, Artificial Sequence
  Synthetic contruct
gggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaatatataatggaagatgcagatgataaaactaattttgaactttttaatttatataaattattttatatcatttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtg

SEQ ID NO: 249 - DNA, 24 bp, Artificial Sequence
  Synthetic construct
agggtctacttgggcttatctata

SEQ ID NO: 250 - DNA, 24 bp, Artificial Sequence
  Synthetic construct
ggcctaagtaatacatccaaagtg

SEQ ID NO: 251 - DNA, 1026 bp, Homo sapiens
gagataatacttgtacaaggaaatttttcatctatgttgcaattcaagtcataaactctttgttctctgcaacaggaggtaccacatttatcttgttgactgtgaagattgttcaacctgaattgaaagcacttgcaatgggtttccagtcaatggttataagaacactaggaggaattctagctccaatatattttggggctctgattgataaaacatgtatgaagtggtccaccaacagctgtggagcacaaggagcttgtaggatatataattccgtattttttggaagggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaacattgcattgattcattaagatgttatttttgaggtgttcctggtctttcactgacaattccaacattctttacttacagtggaccaatggataagtctatgcatctataataaactataaaaaatgggagtacccatggttaggatatagctatgcctttatggttaagattagaatatatgatccataaaatttaaagtgagaggcatggttagtgtgtgatacaataaaaagtaattgtttggtagttgtaactgctaataaaaccagtgactagaatataagggaggtaaaaaggacaagatagattaatagcctaaataaagagaaaagcctgatgcctttaaaaaatgaaacactttggatgtattacttaggccaaaatctggcctggatttatgctataatatatattttcatgttaagttgtatatttttcagaaattataaatattattaatttaaaattcgaaaaaaaaaaaaaaaaaaaaa

SEQ ID NO: 252 - DNA, 624 bp, Artificial Sequence
  Synthetic construct
agggtctacttgggcttatctatagctttaagattcccagcacttgttttatatattgttttcatttttgctatgaagaaaaaatttcaaggaaaagataccaaggcatcggacaatgaaagaaaagtaatggatgaagcaaacttagaattcttaaataatggtgaacattttgtaccttctgctggaacagatagtaaaacatgtaatttggacatgcaagacaatgctgctgccaactaacattgcattgattcattaagatgttatttttgaggtgttcctggtctttcactgacaattccaacattctttacttacagtggaccaatggataagtctatgcatctataataaactataaaaaatgggagtacccatggttaggatatagctatgcctttatggttaagattagaatatatgatccataaaatttaaagtgagaggcatggttagtgtgtgatacaataaaaagtaattgtttggtagttgtaactgctaataaaaccagtgactagaatataagggaggtaaaaaggacaagatagattaatagcctaaataaagagaaaagcctgatgcctttaaaaaatgaaacactttggatgtattacttaggcc

SEQ ID NO: 253 - DNA, 24 bp, Artificial Sequence
  Synthetic construct
agcccttcatttgcagacctgttc

SEQ ID NO: 254 - DNA, 24 bp, Artificial Sequence
  Synthetic construct
actccatcttcatccctccaacac

SEQ ID NO: 255 - DNA, 1081 bp, Homo sapiens
gtggggctggtggcgcttaccgggctggcctacgccctgcctcactggcgctggctgcagctggcagtctccctgcccaccttcctcttcctgctctactactggtgtgtgccggagtcccctcggtggctgttatcacaaaaaagaaacactgaagcaataaagataatggaccacatcgctcaaaagaatgggaagttgcctcctgctgatttaaagatgctttccctcgaagaggatgtcaccgaaaagctgagcccttcatttgcagacctgttccgcacgccgcgcctgaggaagcgcaccttcatcctgatgtacctgtggttcacggactctgtgctctatcaggggctcatcctgcacatgggcgccaccagcgggaacctctacctggatttcctttactccgctctggtcgaaatcccgggggccttcatagccctcatcaccattgaccgcgtgggccgcatctaccccatggccatgtcaaatttgttggcgggggcagcctgcctcgtcatgatttttatctcacctgacctgcactggttaaacatcataatcatgtgtgttggccgaatgggaatcaccattgcaatacaaatgatctgcctggtgaatgctgagctgtaccccacattcgtcaggaacctcggagtgatggtgtgttcctccctgtgtgacataggtgggataatcacccccttcatagtcttcaggctgagggaggtctggcaagccttgcccctcattttgtttgcggtgttgggcctgcttgccgcgggagtgacgctacttcttccagagaccaagggggtcgctttgccagagaccatgaaggacgccgagaaccttgggagaaaagcaaagcccaaagaaaacacgatttaccttaaggtccaaacctcagaaccctcgggcacctgagagagatgttttgcggcgatgtcgtgttggagggatgaagatggagttatcctctgcagaaattcctagacgccttcacttctctgtattcttcctcatacttgcctacccccaaattaatatcagtcctaaagaaaaaaaaaaaaaaaaa

SEQ ID NO: 256 - DNA, 722 bp, Artificial Sequence
  Synthetic construct
agcccttcatttgcagacctgttccgcacgccgcgcctgaggaagcgcaccttcatcctgatgtacctgtggttcacggactctgtgctctatcaggggctcatcctgcacatgggcgccaccagcgggaacctctacctggatttcctttactccgctctggtcgaaatcccgggggccttcatagccctcatcaccattgaccgcgtgggccgcatctaccccatggccatgtcaaatttgttggcgggggcagcctgcctcgtcatgatttttatctcacctgacctgcactggttaaacatcataatcatgtgtgttggccgaatgggaatcaccattgcaatacaaatgatctgcctggtgaatgctgagctgtaccccacattcgtcaggaacctcggagtgatggtgtgttcctccctgtgtgacataggtgggataatcacccccttcatagtcttcaggctgagggaggtctggcaagccttgcccctcattttgtttgcggtgttgggcctgcttgccgcgggagtgacgctacttcttccagagaccaagggggtcgctttgccagagaccatgaaggacgccgagaaccttgggagaaaagcaaagcccaaagaaaacacgatttaccttaaggtccaaacctcagaaccctcgggcacctgagagagatgttttgcggcgatgtcgtgttggagggatgaagatggagt

SEQ ID NO: 257 - DNA, 24 bp, Artificial Sequence
  Synthetic construct
attcctggtctaccggctcactaa

SEQ ID NO: 258 - DNA, 24 bp, Artificial Sequence
  Synthetic construct
gatgctcctctcccaactttactg

SEQ ID NO: 259 - DNA, 1132 bp, Homo sapiens
gttacccttgggctgcatcaaatatggttgcaggggcagcctgtctggcctcagtttttatacctggtgatctacaatggctaaaaattattatctcatgcttgggaagaatggggatcacaatggcctatgagatagtctgcctggtcaatgctgagctgtaccccacattcattaggaatcttggcgtccacatctgttcctcaatgtgtgacattggtggcatcatcacgccattcctggtctaccggctcactaacatctggcttgagctcccgctgatggttttcggcgtgcttggcttggttgctggaggtctggtgctgttgcttccagaaactaaagggaaagctttgcctgagaccatcgaggaagccgaaaatatgcaaagaccaagaaaaaataaagaaaagatgatttacctccaagttcagaaactagacattccattgaactaagaagagagaccgttgctgctgtcatgacctagctttgatggcagcaagaccaaaagtagaaatccctgcactcatcacaaagcccatacaactcaaccaaacttacccctgagccctatcaacctaggtctacagccagtggagtctattgtacactgtggaaaaatacccatgggaccagatcctgccaaattcttccagctcactttattctcagcattcctaggacattggacattggttttctggagggttttttttccatctttgtatttttttaaatttgattcttttctttgcaatgctatctaaccagaatacataggggaactgtgggctaggcaaacaaaatagaaaaaagtgtgaaaaacagtaaagttgggagaggagcatctattttcttaaagaaataaaacacccaaaacaatataaagttgtccagaatgtatgtcaagaattttagataggcctttcagtaacacaggtgaagaaatttttaaaaatacattgattattatctaggttagacttaaagtgaatctcaaataaaagaatcaggaatacaacttaagtgatcatgaggtccttccatatttagattgggtaagcatgaatgtgtattttctacaaaagaccttgagaagagttcaataaaaaatgttagcattataaaa

SEQ ID NO: 260 - DNA, 617 bp, Artificial Sequence
  Synthetic construct
attcctggtctaccggctcactaacatctggcttgagctcccgctgatggttttcggcgtgcttggcttggttgctggaggtctggtgctgttgcttccagaaactaaagggaaagctttgcctgagaccatcgaggaagccgaaaatatgcaaagaccaagaaaaaataaagaaaagatgatttacctccaagttcagaaactagacattccattgaactaagaagagagaccgttgctgctgtcatgacctagctttgatggcagcaagaccaaaagtagaaatccctgcactcatcacaaagcccatacaactcaaccaaacttacccctgagccctatcaacctaggtctacagccagtggagtctattgtacactgtggaaaaatacccatgggaccagatcctgccaaattcttccagctcactttattctcagcattcctaggacattggacattggttttctggagggttttttttccatctttgtatttttttaaatttgattcttttctttgcaatgctatctaaccagaatacataggggaactgtgggctaggcaaacaaaatagaaaaaagtgtgaaaaacagtaaagttgggagaggagcatc

SEQ ID NO: 261 - DNA, 24 bp, Artificial Sequence
  Synthetic construct
ttgctgctatggatgctgacctca

SEQ ID NO: 262 - DNA, 24 bp, Artificial Sequence
  Synthetic construct
ctgcatctgctctaaggtttctgg

SEQ ID NO: 263 - DNA, 1115 bp, Homo sapiens
aggctgaagatatcatccaaaaagctgcaaaaatgaacaacacagctgtaccagcagtgatatttgattctgtggaggagctaaatcccctgaagcagcagaaagctttcattctggacctgttcaggactcggaatattgccataatgaccattatgtctttgctgctatggatgctgacctcagtgggttactttgctctgtctctggatgctcctaatttacatggagatgcctacctgaactgtttcctctctgccttgattgaaattccagcttacattacagcctggctgctattgcgaacgctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcttcaccctttttttccctgaaagtttgggaatgactcttccagaaaccttagagcagatgcagaaagtgaaatggttcagatctgggaaaaaaacaagagactcaatggagacagaagaaaatcccaaggttctaataactgcattctgaaaaaatatctaccccatttggtgaagtgaaaaacagaaaaataagaccctgtggagaaattcgttgttcccactgaaatggactgactgtaacgattgacaccaaaatgaaccttgctatcaagaaatgctcgtcatacagtaaactctggatgattcttccagataatgtccttgctttacaaaccaaccatttctagagagtctccttactcattaattcaatgaaatggattggtaagatgtcttgaaaacatgttagtcaaggactggtaaaatacatataaagattaacactcatttccaatcatacaaatactatccaaataaaaat

SEQ ID NO: 264 - DNA, 534 bp, Artificial Sequence
  Synthetic construct
ttgctgctatggatgctgacctcagtgggttactttgctctgtctctggatgctcctaatttacatggagatgcctacctgaactgtttcctctctgccttgattgaaattccagcttacattacagcctggctgctattgcgaacgctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcttcaccctttttttccctgaaagtttgggaatgactcttccagaaaccttagagcagatgcag

SEQ ID NO: 265 - DNA, 24 bp, Artificial Sequence
  Synthetic construct
actgatgtgtgagctcttaagacc

SEQ ID NO: 266 - DNA, 24 bp, Artificial Sequence
  Synthetic construct
gaggcatatgctttaggagtacca

SEQ ID NO: 267 - DNA, 1092 bp, Homo sapiens
gcagttaatttttcactagaaccagtgagatctggaggaatgtgagaagcatatgctaaatgtacattttaattttagactacttgaaaaggcccctaataaggctagaggtctaagtcccccacccctttccccactcccctctagtggtgaactttagaggaaaaggaagtaattgcacaaggagtttgattcttaccttttctcagttacagaggacattaactggatcattgcttccccagggcaggagagcgcagagctagggaaagtgaaaggtaatgaagatggagcagaatgagcagatgcagatcaccagcaaagtgcactgatgtgtgagctcttaagaccactcagcatgacgactgagtagacttgtttacatctgatcaaagcactgggcttgtccaggctcataataaatgctccattgaatctactattcttgttttccactgctgtggaaacctccttgctactatagcgtcttatgtatggtttaaaggaaatttatcaggtgagagagatgagcaacgttgtcttttctctcaaagctgtaatgtgggttttgttttactgtttatttgtttgttgttgtatccttttctccttgttatttgcccttcagaatgcacttgggaaaggctggttccttagcctcctggtttgtgtctttttttttttttttttaaacacagaatcactctggcaattgtctgcagctgccactggtgcaaggccttaccagccctagcctctagcacttctctaagtgccaaaaacagtgtcattgtgtgtgttcctttcttgatacttagtcatgggaggatattacaaaaaagaaatttaaattgtgttcatagtctttcagagtagctcactttagtcctgtaactttattgggtgatattttgtgttcagtgtaattgtcttctctttgctgattatgttaccatggtactcctaaagcatatgcctcacctggttaaaaaagaacaaacatgtttttgtgaaagctactgaagtgccttgggaaatgagaaagttttaataagtaaaatgattttttaaatatcaaaaaaaaaaaaaaaa

SEQ ID NO: 268 - DNA, 652 bp, Artificial Sequence
  Synthetic construct
actgatgtgtgagctcttaagaccactcagcatgacgactgagtagacttgtttacatctgatcaaagcactgggcttgtccaggctcataataaatgctccattgaatctactattcttgttttccactgctgtggaaacctccttgctactatagcgtcttatgtatggtttaaaggaaatttatcaggtgagagagatgagcaacgttgtcttttctctcaaagctgtaatgtgggttttgttttactgtttatttgtttgttgttgtatccttttctccttgttatttgcccttcagaatgcacttgggaaaggctggttccttagcctcctggtttgtgtctttttttttttttttttaaacacagaatcactctggcaattgtctgcagctgccactggtgcaaggccttaccagccctagcctctagcacttctctaagtgccaaaaacagtgtcattgtgtgtgttcctttcttgatacttagtcatgggaggatattacaaaaaagaaatttaaattgtgttcatagtctttcagagtagctcactttagtcctgtaactttattgggtgatattttgtgttcagtgtaattgtcttctctttgctgattatgttaccatggtactcctaaagcatatgcctc

SEQ ID NO: 269 - DNA, 24 bp, Artificial Sequence
  Syntheric construct
tgcctaaacacctccttggatatg

SEQ ID NO: 270 - DNA, 24 bp, Artificial Sequence
  Synthetic construct
tgggccatctttgaagtgaacaca

SEQ ID NO: 271 - DNA, 1027 bp, Homo sapiens
ctgctgtgcaccctgctgccagagacccatggccagggcctgaaagacaccctccaggacctggagctggggcctcacccacggtcccccaaatcagtgccctcagagaaggaaacagaggccaagggaagaacttccagcccgggagtggcctttgtgagcagcacatacttctgattgaggtctctaagagctggaccatcagcagcagggagctgcctaaacacctccttggatatggccaggacccacagggacacagggcaagaccagccttgcttatggaggcaggacaccacaatctggcccatggctgtcacctcctgccgagtccaatcccagactgggaaccaccatctgagacaggacctcccggcctccttcacctttctcatctccagagccctgcccccaatactctgtctgggttaggatcttgggtatgtcttggaattaacttgtcctctaacaatcttcatggggtatggctctcttgatctcctcaatctggagtcccctgccctcaaaacacagtgatgttcagaacagaacacaaggtaagccctttccaatttgtgggaacaggaggggagaggaaacaaatgtgaagttgtggactctacccaggcaggtggatgaaaatgctgtggataaaaggaaggttatgattccttctagcggatggaccagattcctctggctaacgtatggccccataggtcactgggtcatacagagagaagattcagttcagcctaaatcaaaacttccaccttgtgttcacttcaaagatggcccaacccccgccctacactcagctcatgcctaacctatgtgtggctcagggaccagcttggggaaggaaaggaggtttgttctgctccccgcctcaccccgcctcctcctgctcatgctcagctgcttctggaccttccagggcccatgcagggtgagggaaagggtagaggtcttttcaccgagctgctgctggttgcagttctttctggtgcacattggctaatgccag

SEQ ID NO: 272 - DNA, 583 bp, Artificial Sequence
  Synthetic construct
tgcctaaacacctccttggatatggccaggacccacagggacacagggcaagaccagccttgcttatggaggcaggacaccacaatctggcccatggctgtcacctcctgccgagtccaatcccagactgggaaccaccatctgagacaggacctcccggcctccttcacctttctcatctccagagccctgcccccaatactctgtctgggttaggatcttgggtatgtcttggaattaacttgtcctctaacaatcttcatggggtatggctctcttgatctcctcaatctggagtcccctgccctcaaaacacagtgatgttcagaacagaacacaaggtaagccctttccaatttgtgggaacaggaggggagaggaaacaaatgtgaagttgtggactctacccaggcaggtggatgaaaatgctgtggataaaaggaaggttatgattccttctagcggatggaccagattcctctggctaacgtatggccccataggtcactgggtcatacagagagaagattcagttcagcctaaatcaaaacttccaccttgtgttcacttcaaagatggccca

SEQ ID NO: 273 - DNA, 24 bp, Artificial Sequence
  Synthetic construct
ccacagagctgaaatccatgacga

SEQ ID NO: 274 - DNA, 24 bp, Artificial Sequence
  Synthetic construct
ggccactcaattccaacccaagat

SEQ ID NO: 275 - DNA, 1120 bp, Homo sapiens
aaggaggccaagcaggtgctgtgctacgccgcaagtgtgaacaagaagaccattccttcaaatctgctggacgagctgcagctgcccagaaagaaggtgactcgggcctctgtcctggacttctgtaagaataggcagctctgcaaggtgaccttggtgatgagctgtgtgtggtttaccgtcagttacacctattttacgttgagcctgagaatgagagagctgggcgtgagcgtccacttcagacacgtggtccccagcatcatggaggtgcctgcccggctgtgctgcatctttctcctccagcagattgggaggaagtggagcctggctgtgactctcctccaagccatcatctggtgcttgcttctccttttcctccctgaaggggaggatggcctcagactcaagtggccacgttgtccggccacagagctgaaatccatgacgatcttggtgctcatgctcagagagttcagcctggccgccactgtcactgtgttcttcctctacaccgctgagctcctccccactgtgctcagggcgacaggtctggggctggtgtctctggcctcggtggctggagccatcttgtccctgacaatcatcagccagaccccctccctcctgcccatctttctctgctgcgtcttagccatcgtggccttttccctctcctccctgctgccggaaacgcgagatcagcccctctccgagagcctgaaccactcctcacagataaggaataaggtcaaggacatgaagactaaggaaacatcatctgatgatgtctgaggaagcggccaagaatgtcattctcaatgcccagatcctgagattggacccataccctgtctccaaccctgccttgaagcaattcaataaagaggaagcaaacagccaggctccctgagggccaggcccccagaccatcttgggttggaattgagtggccaagtatggggtcatggattccaggccacaaattccaggcctagttcagtttgggggcagggtcagtcctgctcccaggccagcccttgacattaaaaaaaaatgccccctccttctgcaggagctctgctgtgattcattccaataaaggtacaatgttggtctt

SEQ ID NO: 276 - DNA, 528 bp, Artificial Sequence
  Synthetic construct
ccacagagctgaaatccatgacgatcttggtgctcatgctcagagagttcagcctggccgccactgtcactgtgttcttcctctacaccgctgagctcctccccactgtgctcagggcgacaggtctggggctggtgtctctggcctcggtggctggagccatcttgtccctgacaatcatcagccagaccccctccctcctgcccatctttctctgctgcgtcttagccatcgtggccttttccctctcctccctgctgccggaaacgcgagatcagcccctctccgagagcctgaaccactcctcacagataaggaataaggtcaaggacatgaagactaaggaaacatcatctgatgatgtctgaggaagcggccaagaatgtcattctcaatgcccagatcctgagattggacccataccctgtctccaaccctgccttgaagcaattcaataaagaggaagcaaacagccaggctccctgagggccaggcccccagaccatcttgggttggaattgagtggcc

SEQ ID NO: 277 - DNA, 24 bp, Artificial Sequence
  Synthetic construct
ggttgagagacacagctgctacgt

SEQ ID NO: 278 - DNA, 24 bp, Artificial Sequence
  Synthetic construct
aaagaccagggttagttgcagggc

SEQ ID NO: 279 - DNA, 1040 bp, Homo sapiens
tgtcagcaaagcaagtgatgaagcagagtggatgtccactgtcaccaagctggatggcaagctgcggcccacaaaacagccagtcaggttggctttcctggtttcagacatgctcataccattcccattttctcagcctcttctctgcctccagagaggtggatgcctgggttgagagacacagctgctacgtgatagatgttgagagacagaagccaacgaaggaggtcattcatcaacaaatatatttattggagaccgactttgtgcaaagcaatgctaatcagggttctccatggagcttccctcagctcttacctcacctccctccatttacattagggccttctcccagggtgtgctcggtgggcagtgtgggactgggggtgtgggagttggtgagagcaggaggagaggtggggacagcaagaagccacagattggcatgaaggatcctgacctgactatccatgccatccatggcccccagactgactctgcacctggccctttgccagacagctctgtctccccatgtcctctggaacagctgggcatgggtcatggccattcatgacccttaagtgccacccttcttggaagaccccctccagaagcatactggaagccacctctggaaaagcctcatatggtgatatgccaaaatatttatgtcaatgtccaaacaaagtccaatgccatgagactgaagtctttgtggaaaccactgttacagacaagcttatttccaaagccacctcatttccaaacatctcactcaggaagggaggctcaatgtaacctcaggggccagttttagcatttgaaatggttctgcttggaaaatgatgccctgcaactaaccctggtctttcccatggcaatttaaccacatttggaaggcactgccttcagctgagtttatgaacaatgaatgccaaccttcaggttctagaagattggttgcactcccaaacctttattctattatattactattaaaatattctaattttgctattgaggtaaaaaaaaaaaaa

SEQ ID NO: 280 - DNA, 705 bp, Artificial Sequence
  Synthetic construct
ggttgagagacacagctgctacgtgatagatgttgagagacagaagccaacgaaggaggtcattcatcaacaaatatatttattggagaccgactttgtgcaaagcaatgctaatcagggttctccatggagcttccctcagctcttacctcacctccctccatttacattagggccttctcccagggtgtgctcggtgggcagtgtgggactgggggtgtgggagttggtgagagcaggaggagaggtggggacagcaagaagccacagattggcatgaaggatcctgacctgactatccatgccatccatggcccccagactgactctgcacctggccctttgccagacagctctgtctccccatgtcctctggaacagctgggcatgggtcatggccattcatgacccttaagtgccacccttcttggaagaccccctccagaagcatactggaagccacctctggaaaagcctcatatggtgatatgccaaaatatttatgtcaatgtccaaacaaagtccaatgccatgagactgaagtctttgtggaaaccactgttacagacaagcttatttccaaagccacctcatttccaaacatctcactcaggaagggaggctcaatgtaacctcaggggccagttttagcatttgaaatggttctgcttggaaaatgatgccctgcaactaaccctggtcttt

SEQ ID NO: 281 - DNA, 24 bp, Artificial Sequence
  Synthetic construct
agcaccaaaggggccaaaactgac

SEQ ID NO: 282 - DNA, 24 bp, Artificial Sequence
  Synthetic construct
gagttcggagcagtggttgtacag

SEQ ID NO: 283 - DNA, 1050 bp, Homo sapiens
ttcctggctgccttggcgctctacctgctcctggcggccgcctccagcccggccctgcccggggtctacctgctcttcgcctcgcgcctgcccggagcgctcatgcacacgctgccagccgcccagatggtcatcacggacctgtcggcacccgaggagcggcccgcggccctgggccggctgggcctctgcttcggcgtcggagtcatcctcggctccctgctgggcgggaccctggtctccgcgtacgggattcagtgcccggccatcctggctgccctggccaccctcctgggagctgtcctcagcttcacctgcatccccgccagcaccaaaggggccaaaactgacgcccaggctccactgccaggcggcccccgggccagtgtgttcgacctgaaggccatcgcctccctgctgcggctgccagacgtcccgaggatcttcctggtgaaggtggcctccaactgccccacagggctcttcatggtcatgttctccatcatctccatggacttcttccagctggaggccgcccaagctggctacctcatgtccttcttcgggctcctccagatggtgacccagggcctggtcatcgggcagctgagcagccacttctcggaggaggtgctgctccgggccagcgtgctggtcttcatcgtggtgggcctggccatggcctggatgtccagcgtcttccacttctgcctcctggtgcccggcctggtgttcagcctctgcaccctcaacgtggtcaccgacagcatgctgatcaaggctgtctccacctcggacacagggaccatgctgggcctctgcgcctctgtacaaccactgctccgaactctgggacccacggtcggcggcctcctgtaccgcagctttggcgtccccgtcttcggccacgtgcaggttgctatcaatacccttgtcctcctggtcctctggaggaaacctatgccccagaggaaggacaaagtccggtgaccgctgcccagacacagactggcaataaactccttccgaaaaaaaaaaaaaaaaaaaaaa

SEQ ID NO: 284 - DNA, 523 bp, Artificial Sequence
  Synthetic construct
agcaccaaaggggccaaaactgacgcccaggctccactgccaggcggcccccgggccagtgtgttcgacctgaaggccatcgcctccctgctgcggctgccagacgtcccgaggatcttcctggtgaaggtggcctccaactgccccacagggctcttcatggtcatgttctccatcatctccatggacttcttccagctggaggccgcccaagctggctacctcatgtccttcttcgggctcctccagatggtgacccagggcctggtcatcgggcagctgagcagccacttctcggaggaggtgctgctccgggccagcgtgctggtcttcatcgtggtgggcctggccatggcctggatgtccagcgtcttccacttctgcctcctggtgcccggcctggtgttcagcctctgcaccctcaacgtggtcaccgacagcatgctgatcaaggctgtctccacctcggacacagggaccatgctgggcctctgcgcctctgtacaaccactgctccgaactc

SEQ ID NO: 285 - DNA, 24 bp, Artificial Sequence
  Synthetic construct
ttcatcaaatctggtcaagggact

SEQ ID NO: 286 - DNA, 24 bp, Artificial Sequence
  Synthetic construct
gttccacatttcaaaagcctcgat

SEQ ID NO: 287 - DNA, 1113 bp, Homo sapiens
ttcatcaaatctggtcaagggactaagctcctagctgaccattcattctgaagattgcatggaggatgaacatctgggaatcctgttaatgagaaggctgaatcacaggcacctgggccaaagggtgtgagcattcatgttctctgctcaccttggtttccgcacaccttcgcaatgtgaacaggtcaggagtccctcccgtccacctcctctgtaacagctggggttccaggcatggtttaggccctgttccagcaataagaaccaatctgctgtacaatctgaggacttggctgtgttatttacaaaatgatgctgtggttctgagattatttgggacatttttggctctcctttagtggacacctagagccacagattcccttctttactaaacaaatcccatggattctgatttctgggtcttaggattttaaaagtgaagggatatttttcttatatttgtgagttcagttccgatggtgcccgtggtcaaaagcgaaaaacatggacaattcctattcattcttagcactttgacatgtcttggggaaaagctttacattttaatttaaaagaaagatcaattatatccatgcttaacaggatcagcaggagctttataaatgactttacagagactaataagggattgatctttctttttttgttatcgaggcttttgaaatgtggaacttgtgtgttctgctttatatgttatattcaatatcttttcagatgcagtctatattttatgctgagttttaaaaatgaaatactttatgcaaacaggcaaaattggtaccaaagggaaacattaaccatgaggaagagcatttttctaaggagaacaggtgacaatatacacatgtcgcgtaatcgtaaaatgagcatcttagtctttaaaacacatcagaattgaatacgaataatctatttgtcgatgaaataaacacaactctttgaggatttgagactacattcaccctttattcacagtcacttgcagttttgcttttctctccatttctctgctgtaagatgactgttgcattgttgaattgtattttgagtggatatttttgtttggtaacaattaaaattttaaatcgt

SEQ ID NO: 288 - DNA, 696 bp, Artificial Sequence
  Synthetic construct
ttcatcaaatctggtcaagggactaagctcctagctgaccattcattctgaagattgcatggaggatgaacatctgggaatcctgttaatgagaaggctgaatcacaggcacctgggccaaagggtgtgagcattcatgttctctgctcaccttggtttccgcacaccttcgcaatgtgaacaggtcaggagtccctcccgtccacctcctctgtaacagctggggttccaggcatggtttaggccctgttccagcaataagaaccaatctgctgtacaatctgaggacttggctgtgttatttacaaaatgatgctgtggttctgagattatttgggacatttttggctctcctttagtggacacctagagccacagattcccttctttactaaacaaatcccatggattctgatttctgggtcttaggattttaaaagtgaagggatatttttcttatatttgtgagttcagttccgatggtgcccgtggtcaaaagcgaaaaacatggacaattcctattcattcttagcactttgacatgtcttggggaaaagctttacattttaatttaaaagaaagatcaattatatccatgcttaacaggatcagcaggagctttataaatgactttacagagactaataagggattgatctttctttttttgttatcgaggcttttgaaatgtggaac

(19) United States
(12) Patent Application Publication
(10) Pub. No.: US 2010/0304984 A1
(43) Pub. Date: Dec. 2, 2010

(54) PREDICTION OF POTENTIAL DRUG-DRUG INTERACTIONS USING GENE EXPRESSION PROFILING OF DRUG TRANSPORTERS, CYTOCHROME P450S AND NUCLEAR X RECEPTORS

(75) Inventors: Robert Shipman, Mississauga (CA); Jodi A Morrison, Mississauga (CA); Ines De Lannoy, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: NOAB BIODISCOVERIES INC., Mississauga, ON (CA)

(21) Appl. No.: 12/513,962

(22) PCT Filed: Nov. 8, 2007
(86) PCT No.: PCT/CA07/01996

§ 371 Date: Aug. 13, 2010

Related U.S. Application Data
(60) Provisional application No. 60/865,266, filed on Nov. 10, 2006.

(51) Int. Cl. C40B 30/02; C40B 40/08; C40B 30/04; C07H 21/04

(52) U.S. Cl. 506/8; 506/17; 506/9; 536/23.1; 536/24.5

(57) ABSTRACT

The invention provides materials and methods for detecting the expression of genes encoding cytochrome p450, nuclear X receptors, phase H transferases, and solute carrier family uptake pumps. The materials include sets of primers, PCR amplicons and arrays. The methods of the invention include hybridization assays. Kits and assays for the detection of the expression of the genes are also provided by the invention. In addition, the invention provides the use of the materials and methods of the invention in drug screening assays.

### FIELD OF THE INVENTION

The invention relates to materials and methods for detecting gene expression, particularly genes encoding cytochrome p450, nuclear X receptors, phase II transferases, and solute carrier family uptake pumps.

### BACKGROUND OF THE INVENTION

Adverse effects of drugs, as well as other xenobiotics, represent a significant public health problem. The variation in the degree of response to drug between patients is well documented and poses a serious problem in medicine. At present, there are no reliable biomarkers that can predict which group of patients will respond positively, adversely or not at all to a particular medication and dose. Adverse drug effects account for more than 2,000,000 hospitalizations and 100,000 deaths per year in the US. The variability in drug response is due to multiple factors including disease determinants, genetic, environmental, pharmacokinetic and pharmacodynamic factors. All these factors influence drug absorption, distribution, metabolism and excretion. An understanding of how these factors contribute to the variability in efficacy and toxicity of prescribed medications may provide safer and more efficient drug therapy.

Cytochrome P450s and other drug sensing, transport and metabolism systems play a major role in the potentiation of adverse drug effects. All these genes are strongly expressed in liver cells. The interplay between drug metabolism, detoxification and toxicity depends not only on the drug itself but also on the coordinated regulation and expression of the CYPs and other genes in the drug sensing, transport and metabolism systems.

### Transporters

Membrane transporters are critical facilitators of the uptake (e.g. solute carrier family (SLC) transporters) and export (e.g. ABC transporters) of drugs. Transporters can alter drug disposition and distribution in several important ways. First, drug uptake can be enhanced by members of the SLC family of transporters. Second, significant and adverse drug-drug interactions can occur when one of the co-administered drugs induces or suppresses transporter gene expression or protein function. Third, drug efflux can be enhanced by members of the ABC family of transporters. Fourth, food-drug interactions can influence both uptake and efflux transporter levels.

Many of these transporters play key roles in pharmacology affecting both the uptake and efflux of administered drugs. As such, these transporters play critical roles in mediating both the chemo-sensitivity and chemo-resistance of cancer cells to cancer chemotherapeutics. ABC transporters are frequently associated with decreased intracellular concentration of chemotherapeutic agents and acquired multi-drug resistance of tumor cells. SLC transporters, including anion, cation, nucleoside and amino acid transporters, are associated with increased sensitivity of tumor cells to chemotherapeutic agents since these transporters facilitate the cellular uptake of hydrophilic compounds.

Membrane transporters can be classified as either passive or active transporters. The active transporters can be further divided into primary or secondary active transporters based on the process of energy coupling and facilitated transport.

The ABC transporters are primary active transporters which export compounds against a chemical gradient driven by ATP and an inherent ATPase activity.

The majority of passive transporters, which permit compounds to equilibrate along a concentration gradient, ion pumps, secondary active transporters and exchangers belong to the SLC transporter family.

Understanding the role and function of membrane transporters in both normal cells and cancer cells should prove valuable in “predicting” chemotherapeutic drug response as well as indicating which transporters might serve as potential therapeutic targets for “preventing” acquired drug resistance.

### CYPs

Drug metabolism is a major determinant of drug clearance and is the factor most frequently responsible for pharmacokinetic differences in drug responses between individuals. These differences in drug response between individuals are due primarily to the inducible expression of, and polymorphisms in, the drug metabolizing cytochrome P450 enzymes (CYPs).

Many drug-drug interactions are metabolism-based and most involve induction of CYPs. Of the eleven xenobiotic metabolizing CYPs expressed in the human liver, a specific group of six CYPs appear to be responsible for the metabolism of most drugs and their associated drug-drug interactions. This is likely due to the ability of these CYPs to bind and metabolize chemical structures common to many drugs and to the mass abundance of these CYPs in human liver.

An increase in the level of a specific CYP following drug exposure usually raises concerns of potential toxicity, dosage limitations or possible drug-drug interactions should the drug be used in a clinical setting. Consequently, CYP induction following treatment with novel therapeutic agents can be used as a potential marker of adverse drug response.

### NXRs

A complex signaling network exists to protect cells against the potential toxic effects of xenobiotics (exogenous compounds). This system includes the nuclear xenoreceptors (NXRs) and functions in concert with other signaling pathways involved in the metabolism of endogenous compounds. The expression of the CYPs and other genes in the drug sensing, transport and metabolism systems is not only regulated by drugs but is also influenced by physiopathological (e.g. steroids, lipids, salts, etc.) and environmental (e.g. nutrients) factors. In addition to regulating CYP expression, the NXRs interact with other nuclear receptors controlling various facets of endogenous metabolism. The clinical consequences of this xenoreceptor:nuclear receptor cross-signaling has yet to be established.

The expression of cytochrome p450, nuclear X receptors, phase II transferases and solute carrier family uptake pumps in a cell may significantly influence the efficacy of drugs. Thus, an integrated approach to the analysis of cytochrome P450, uptake transporter and nuclear xenoreceptor gene expression, with respect to drug transport and metabolism, will better define and predict the pharmacokinetics, pharmacodynamics and potential toxic effects of new or existing drugs. For example, gene expression data of genes encoding these proteins can be used to design drug treatment protocols to specific cell types, tissues, diseases or cancers. In addition, the information on gene expression can be used in candidate population profiling, such as the pre-screening of patients for inclusion or exclusion from clinical trials.

There is a need for tools that reveal the impact of drug compounds and other stimuli on the expression of genes encoding cytochrome p450, nuclear X receptors, phase II transferases and solute carrier family uptake pumps. The need for such assays is accentuated by the fact that (i) adverse drug effects account for more than 2,000,000 hospitalizations and 100,000 deaths per year in the US and (ii) half of the drugs withdrawn from the US market between 1997 and 2002 exhibited significant drug-drug interactions.

### SUMMARY OF THE INVENTION

The inventors provide materials and methods to determine a change in the gene expression profile of a subject in response to a drug or combination of drugs. In particular, the materials and methods can be used to determine a change in the gene expression profile in a test sample of genes involved in drug transport, drug metabolism or regulators of the expression of these genes or function of the proteins encoded by these genes. In a specific embodiment, the materials and methods can be used to determine the gene expression of cytochrome P450 enzymes, uptake transporters and/or nuclear xenoreceptors.

Accordingly, the inventors provide an array, which can be used for the convenient and collective analysis of the effects of different stimuli on the coordinated gene expression of cytochrome P450 enzymes, phase II metabolic enzymes, uptake transporters and/or nuclear xenoreceptors. The array provides a screening process for the evaluation of potential drug-drug interactions or adverse effects prior to administration to humans. For example, the array could be used to pre-screen or pre-select patients for inclusion or exclusion from clinical trials for a new drug or new formulation of an existing drug.

The inventors have prepared primer pairs for nucleic acids encoding cytochrome p450, nuclear X receptors, phase II transferases and solute carrier family uptake pumps. These primers were used to generate nucleic acid molecules, also referred to herein as amplicons, that can be used as probes in assays, such as array-based assays, to screen for the expression of genes encoding these proteins in test samples.

Accordingly, one aspect of the invention is a primer pair selected from:
(a) the following pairs of nucleic acid sequences:
   SEQ ID NO:1 and SEQ ID NO:2;
   SEQ ID NO:5 and SEQ ID NO:6;
   SEQ ID NO:9 and SEQ ID NO:10;
   SEQ ID NO:13 and SEQ ID NO:14;
   SEQ ID NO:17 and SEQ ID NO:18;
   SEQ ID NO:21 and SEQ ID NO:22;
   SEQ ID NO:25 and SEQ ID NO:26;
   SEQ ID NO:29 and SEQ ID NO:30;
   SEQ ID NO:33 and SEQ ID NO:34;
   SEQ ID NO:37 and SEQ ID NO:38;
   SEQ ID NO:41 and SEQ ID NO:42;
   SEQ ID NO:45 and SEQ ID NO:46;
   SEQ ID NO:49 and SEQ ID NO:50;
   SEQ ID NO:53 and SEQ ID NO:54;
   SEQ ID NO:57 and SEQ ID NO:58;
   SEQ ID NO:61 and SEQ ID NO:62;
   SEQ ID NO:65 and SEQ ID NO:66;
   SEQ ID NO:69 and SEQ ID NO:70;
   SEQ ID NO:73 and SEQ ID NO:74;
   SEQ ID NO:77 and SEQ ID NO:78;
   SEQ ID NO:81 and SEQ ID NO:82;
   SEQ ID NO:85 and SEQ ID NO:86;
   SEQ ID NO:89 and SEQ ID NO:90;
   SEQ ID NO:93 and SEQ ID NO:94;
   SEQ ID NO:97 and SEQ ID NO:98;
   SEQ ID NO:101 and SEQ ID NO:102;
   SEQ ID NO:105 and SEQ ID NO:106;
   SEQ ID NO:109 and SEQ ID NO:110;
   SEQ ID NO:113 and SEQ ID NO:114;
   SEQ ID NO:117 and SEQ ID NO:118;
   SEQ ID NO:121 and SEQ ID NO:122;
   SEQ ID NO:125 and SEQ ID NO:126;
   SEQ ID NO:129 and SEQ ID NO:130;
   SEQ ID NO:133 and SEQ ID NO:134;
   SEQ ID NO:137 and SEQ ID NO: 138;
   SEQ ID NO:141 and SEQ ID NO:142;
   SEQ ID NO:145 and SEQ ID NO:146;
   SEQ ID NO:149 and SEQ ID NO:150;
   SEQ ID NO:153 and SEQ ID NO:154;
   SEQ ID NO:157 and SEQ ID NO:158;
   SEQ ID NO:161 and SEQ ID NO:162;
   SEQ ID NO:165 and SEQ ID NO:166;
   SEQ ID NO:169 and SEQ ID NO:170;
   SEQ ID NO:173 and SEQ ID NO:174;
   SEQ ID NO:177 and SEQ ID NO:178;
   SEQ ID NO:181 and SEQ ID NO:182;
   SEQ ID NO:185 and SEQ ID NO:186;
   SEQ ID NO:189 and SEQ ID NO:190;
   SEQ ID NO:193 and SEQ ID NO:194;
   SEQ ID NO:197 and SEQ ID NO:198;
   SEQ ID NO:201 and SEQ ID NO:202;
   SEQ ID NO:205 and SEQ ID NO:206;
   SEQ ID NO:209 and SEQ ID NO:210;
   SEQ ID NO:213 and SEQ ID NO:214;
   SEQ ID NO:217 and SEQ ID NO:218;
   SEQ ID NO:221 and SEQ ID NO:222;
   SEQ ID NO:225 and SEQ ID NO:226;
   SEQ ID NO:229 and SEQ ID NO:230;
   SEQ ID NO:233 and SEQ ID NO:234;
   SEQ ID NO:237 and SEQ ID NO:238;
   SEQ ID NO:241 and SEQ ID NO:242;
   SEQ ID NO:245 and SEQ ID NO:246;
   SEQ ID NO:249 and SEQ ID NO:250;
   SEQ ID NO:253 and SEQ ID NO:254;
   SEQ ID NO:257 and SEQ ID NO:258;
   SEQ ID NO:261 and SEQ ID NO:262;
   SEQ ID NO:265 and SEQ ID NO:266;
   SEQ ID NO:269 and SEQ ID NO:270;
   SEQ ID NO:273 and SEQ ID NO:274;
   SEQ ID NO:277 and SEQ ID NO:278;
   SEQ ID NO:281 and SEQ ID NO:282; or
   SEQ ID NO:285 and SEQ ID NO:286;
(b) the nucleic acid sequences in (a) wherein T can also be U;
(c) nucleic acid sequences complementary to (a) or (b); or
(d) nucleic acid sequences that have substantial sequence homology to (a), (b) or (c).

Another aspect of the invention includes isolated nucleic acid molecules prepared using any known amplification method, such as polymerase chain reaction (PCR), and the primer pairs of the invention.

Accordingly, a further aspect of the invention is an isolated nucleic acid molecule having a nucleic acid sequence consisting of:
(a) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288,
(b) a nucleic acid sequence in (a) wherein T can also be U;
(c) a nucleic acid sequence complementary to (a) or (b);
(d) a nucleic acid sequence that has substantial sequence homology to (a), (b) or (c); or
(e) a fragment of (a) to (d).

These primer pairs and isolated nucleic acid molecules can be used in assays, such as arrays, to detect the expression of genes encoding cytochrome p450, nuclear X receptors, phase II transferases and solute carrier family uptake pumps. Accordingly, one aspect of the invention is an array comprising two or more nucleic acid molecules of the invention immobilized on a substrate. The array can be used to determine a change in the gene expression profile of a subject in response to a drug or a combination of drugs. In addition, the array can be used to detect the presence of drug-drug interactions in a subject.

In addition, the invention includes methods for detecting the expression of genes encoding cytochrome p450, nuclear X receptors, phase II transferases and solute carrier family uptake pumps. Accordingly, one aspect of the invention is a method of detecting the expression of two or more genes, comprising the steps:
(a) providing two or more nucleic acid molecules of the invention;
(b) providing transcription indicators from a test sample;
(c) allowing the transcription indicators to hybridize with said two or more nucleic acid molecules; and
(d) detecting hybridization of said transcription indicators with said two or more nucleic acid molecules, wherein hybridization is indicative of the expression of the genes.

Additionally, the invention provides a method of detecting the expression of two or more genes in a test sample using the arrays of the invention.

The gene expression data generated using the materials and methods of the invention can be contained in a database. Accordingly, the invention includes a computer system comprising (a) a database containing information identifying the expression level of two or more genes; and (b) a user interface to view the information, wherein the information identifying the expression level of two or more genes is obtained using the methods and/or arrays of the invention.

The materials and methods of the present invention can be used to perform drug-associated gene expression profiling of genes encoding cytochrome p450, nuclear X receptors, phase II transferases and solute carrier family uptake pumps. Such profiling can be used to identify potential modulators of gene expression. Accordingly, another aspect of the invention is a method for screening a compound for its effect on the expression of two or more genes, comprising the steps:
a) providing a transcription indicator from a test sample from a subject exposed to the compound;
b) providing two or more nucleic acid molecules of the invention,
c) allowing said transcription indicator to hybridize with said two or more nucleic acid molecules; and
d) detecting hybridization of said transcription indicator with said two or more nucleic acid molecules, wherein hybridization is indicative of the expression of the two or more genes.

Additionally, the invention provides a method for screening a compound for its effect on the expression of two or more genes using the array and/or methods of the invention to prepare gene expression profiles of a test sample from a subject that has been exposed to the compound.

A further aspect of the invention is a method of assessing the toxicity and/or efficacy of a compound in a subject using the array and/or methods of the invention.

The array and methods of the invention can also be used to analyze the presence of drug-drug interactions. Accordingly, one aspect of the invention is a method for determining a change in gene expression profile for a compound in the presence of one or more different compounds using the array and/or methods of the invention.

The drug screening methods of the invention can be used to generate information useful when designing drug or chemical therapy for the treatment of disease.

The invention also includes kits comprising the nucleic acids molecules and/or arrays of the invention.

An additional aspect of the invention is a relational database comprising gene expression profiles of genes encoding cytochrome p450, nuclear X receptors, phase II transferases and solute carrier family uptake pumps that are generated using the arrays and/or methods of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the upper and lower primer sequences (SEQ ID NOS:1-2) and PCR conditions; the nucleic acid sequence of a portion of CYP1A2 (SEQ ID NO:3); and the PCR product obtained using the primers is shown underlined (SEQ ID NO:4).

FIG. 2 shows the upper and lower primer sequences (SEQ ID NOS:5-6) and PCR conditions; the nucleic acid sequence of a portion of CYP1B1 (SEQ ID NO:7); and the PCR product obtained using the primers is shown underlined (SEQ ID NO:8).

FIG. 3 shows the upper and lower primer sequences (SEQ ID NOS:9-10) and PCR conditions; the nucleic acid sequence of a portion of CYP2A6 (SEQ ID NO:11); and the PCR product obtained using the primers is shown underlined (SEQ ID NO:12).

FIG. 4 shows the upper and lower primer sequences (SEQ ID NOS:13-14) and PCR conditions; the nucleic acid sequence of a portion of CYP2B6 (SEQ ID NO:15); and the PCR product obtained using the primers is shown underlined (SEQ ID NO:16).

FIG. 5 shows the upper and lower primer sequences (SEQ ID NOS:17-18) and PCR conditions; the nucleic acid sequence of a portion of CYP2C8 variant 1 (SEQ ID NO:19); and the PCR product obtained using the primers is shown underlined (SEQ ID NO:20).

FIG. 6 shows the upper and lower primer sequences (SEQ ID NOS:21-22) and PCR conditions; the nucleic acid sequence of a portion of CYP2C8 variant 2 (SEQ ID NO:23); and the PCR product obtained using the primers is shown underlined (SEQ ID NO:24).

FIG. 7 shows the upper and lower primer sequences (SEQ ID NOS:25-26) and PCR conditions; the nucleic acid sequence of a portion of CYP2C9 (SEQ ID NO:27); and the PCR product obtained using the primers is shown underlined (SEQ ID NO:28).

FIG. 8 shows the upper and lower primer sequences (SEQ ID NOS:29-30) and PCR conditions; the nucleic acid sequence of a portion of CYP2C19 (SEQ ID NO:31); and the PCR product obtained using the primers is shown underlined (SEQ ID NO:32).

FIG. 9 shows the upper and lower primer sequences (SEQ ID NOS:33-34) and PCR conditions; the nucleic acid sequence of a portion of CYP2D6 (SEQ ID NO:35); and the PCR product obtained using the primers is shown underlined (SEQ ID NO:36).

FIG. 10 shows the upper and lower primer sequences (SEQ ID NOS:37-38) and PCR conditions; the nucleic acid sequence of a portion of CYP2E1 (SEQ ID NO:39); and the PCR product obtained using the primers is shown underlined (SEQ ID NO:40).

FIG. 11 shows the upper and lower primer sequences (SEQ ID NOS:41-42) and PCR conditions; the nucleic acid sequence of a portion of CYP3A4 (SEQ ID NO:43); and the PCR product obtained using the primers is shown underlined (SEQ ID NO:44).

FIG. 12 shows the upper and lower primer sequences (SEQ ID NOS:45-46) and PCR conditions; the nucleic acid sequence of a portion of CYP19A variant 1 (SEQ ID NO:47); and the PCR product obtained using the primers is shown underlined (SEQ ID NO:48).

FIG. 13 shows the upper and lower primer sequences (SEQ ID NOS:49-50) and PCR conditions; the nucleic acid sequence of a portion of CYP19A variant 2 (SEQ ID NO:51); and the PCR product obtained using the primers is shown underlined (SEQ ID NO:52).

FIG. 14 shows the upper and lower primer sequences (SEQ ID NOS:53-54) and PCR conditions; the nucleic acid sequence of a portion of CYP27A1 (SEQ ID NO:55); and the PCR product obtained using the primers is shown underlined (SEQ ID NO:56).

FIG. 15 shows the upper and lower primer sequences (SEQ ID NOS:57-58) and PCR conditions; the nucleic acid sequence of a portion of CYP27B1 (SEQ ID NO:59); and the PCR product obtained using the primers is shown underlined (SEQ ID NO:60).

FIG. 16 shows the upper and lower primer sequences (SEQ ID NOS:61-62) and PCR conditions; the nucleic acid sequence of a portion of CAR1 (SEQ ID NO:63); and the PCR product obtained using the primers is shown underlined (SEQ ID NO:64).

FIG. 17 shows the upper and lower primer sequences (SEQ ID NOS:65-66) and PCR conditions; the nucleic acid sequence of a portion of FXR (SEQ ID NO:67); and the PCR product obtained using the primers is shown underlined (SEQ ID NO:68).

FIG. 18 shows the upper and lower primer sequences (SEQ ID NOS:69-70) and PCR conditions; the nucleic acid sequence of a portion of LXR (SEQ ID NO:71); and the PCR product obtained using the primers is shown underlined (SEQ ID NO:72).

FIG. 19 shows the upper and lower primer sequences (SEQ ID NOS:73-74) and PCR conditions; the nucleic acid sequence of a portion of PPARA (SEQ ID NO:75); and the PCR product obtained using the primers is shown underlined (SEQ ID NO:76).

FIG. 20 shows the upper and lower primer sequences (SEQ ID NOS:77-78) and PCR conditions; the nucleic acid sequence of a portion of PPARD-B (SEQ ID NO:79); and the PCR product obtained using the primers is shown underlined (SEQ ID NO:80).

FIG. 21 shows the upper and lower primer sequences (SEQ ID NOS:81-82) and PCR conditions; the nucleic acid sequence of a portion of PPARG (SEQ ID NO:83); and the PCR product obtained using the primers is shown underlined (SEQ ID NO:84).

FIG. 22 shows the upper and lower primer sequences (SEQ ID NOS:85-86) and PCR conditions; the nucleic acid sequence of a portion of RXRA (SEQ ID NO:87); and the PCR product obtained using the primers is shown underlined (SEQ ID NO:88).

FIG. 23 shows the upper and lower primer sequences (SEQ ID NOS:89-90) and PCR conditions; the nucleic acid sequence of a portion of RXRB (SEQ ID NO:91); and the PCR product obtained using the primers is shown underlined (SEQ ID NO:92).

FIG. 24 shows the upper and lower primer sequences (SEQ ID NOS:93-94) and PCR conditions; the nucleic acid sequence of a portion of RXRG (SEQ ID NO:95); and the PCR product obtained using the primers is shown underlined (SEQ ID NO:96).

FIG. 25 shows the upper and lower primer sequences (SEQ ID NOS:97-98) and PCR conditions; the nucleic acid sequence of a portion of SXR (PXR) transcript variant 1 (SEQ ID NO:99); and the PCR product obtained using the primers is shown underlined (SEQ ID NO:100).

FIG. 26 shows the upper and lower primer sequences (SEQ ID NOS:101-102) and PCR conditions; the nucleic acid sequence of a portion of SULT1A1 (SEQ ID NO:103); and the PCR product obtained using the primers is shown underlined (SEQ ID NO:104).

FIG. 27 shows the upper and lower primer sequences (SEQ ID NOS:105-106) and PCR conditions; the nucleic acid sequence of a portion of SULT1B1 (SEQ ID NO:107); and the PCR product obtained using the primers is shown underlined (SEQ ID NO:108).

FIG. 28 shows the upper and lower primer sequences (SEQ ID NOS:109-110) and PCR conditions; the nucleic acid sequence of a portion of SULT1C1 (SEQ ID NO:111); and the PCR product obtained using the primers is shown underlined (SEQ ID NO:112).

FIG. 29 shows the upper and lower primer sequences (SEQ ID NOS:113-114) and PCR conditions; the nucleic acid sequence of a portion of SULT1E1 (SEQ ID NO:115); and the PCR product obtained using the primers is shown underlined (SEQ ID NO:116).

FIG. 30 shows the upper and lower primer sequences (SEQ ID NOS:117-118) and PCR conditions; the nucleic acid sequence of a portion of SULT2A1 (SEQ ID NO:119); and the PCR product obtained using the primers is shown underlined (SEQ ID NO:120).

FIG. 31 shows the upper and lower primer sequences (SEQ ID NOS:121-122) and PCR conditions; the nucleic acid sequence of a portion of SULT2B1b (SEQ ID NO:123); and the PCR product obtained using the primers is shown underlined (SEQ ID NO:124).

FIG. 32 shows the upper and lower primer sequences (SEQ ID NOS:125-126) and PCR conditions; the nucleic acid sequence of a portion of UGT2A1 (SEQ ID NO:127); and the PCR product obtained using the primers is shown underlined (SEQ ID NO:128).

FIG. 33 shows the upper and lower primer sequences (SEQ ID NOS:129-130) and PCR conditions; the nucleic acid sequence of a portion of UGT2B4 (SEQ ID NO:131); and the PCR product obtained using the primers is shown underlined (SEQ ID NO:132).

FIG. 34 shows the upper and lower primer sequences (SEQ ID NOS:133-134) and PCR conditions; the nucleic acid sequence of a portion of UGT2B15 (also known as UGT2B8) (SEQ ID NO:135); and the PCR product obtained using the primers is shown underlined (SEQ ID NO:136).

FIG. 35 shows the upper and lower primer sequences (SEQ ID NOS:137-138) and PCR conditions; the nucleic acid sequence of a portion of UGT2B17 (SEQ ID NO:139); and the PCR product obtained using the primers is shown underlined (SEQ ID NO:140).

FIG. 36 shows the upper and lower primer sequences (SEQ ID NOS:141-142) and PCR conditions; the nucleic acid sequence of a portion of UGT8 (SEQ ID NO:143); and the PCR product obtained using the primers is shown underlined (SEQ ID NO:144).

FIG. 37 shows the upper and lower primer sequences (SEQ ID NOS:145-146) and PCR conditions; the nucleic acid sequence of a portion of CNT1 (also known as SLC28A1) (SEQ ID NO:147); and the PCR product obtained using the primers is shown underlined (SEQ ID NO:148).

FIG. 38 shows the upper and lower primer sequences (SEQ ID NOS:149-150) and PCR conditions; the nucleic acid sequence of a portion of CNT2 (also known as SLC28A2) (SEQ ID NO:151); and the PCR product obtained using the primers is shown underlined (SEQ ID NO:152).

FIG. 39 shows the upper and lower primer sequences (SEQ ID NOS:153-154) and PCR conditions; the nucleic acid sequence of a portion of CNT3 (also known as SLC28A3) (SEQ ID NO:155); and the PCR product obtained using the primers is shown underlined (SEQ ID NO:156).

FIG. 40 shows the upper and lower primer sequences (SEQ ID NOS:157-158) and PCR conditions; the nucleic acid sequence of a portion of ENT1 (also known as SLC29A1) (SEQ ID NO:159); and the PCR product obtained using the primers is shown underlined (SEQ ID NO:160).

FIG. 41 shows the upper and lower primer sequences (SEQ ID NOS:161-162) and PCR conditions; the nucleic acid sequence of a portion of ENT2 (also known as SLC29A2) (SEQ ID NO:163); and the PCR product obtained using the primers is shown underlined (SEQ ID NO:164).

FIG. 42 shows the upper and lower primer sequences (SEQ ID NOS:165-166) and PCR conditions; the nucleic acid sequence of a portion of ENT3 (SEQ ID NO:167); and the PCR product obtained using the primers is shown underlined (SEQ ID NO:168).

FIG. 43 shows the upper and lower primer sequences (SEQ ID NOS:169-170) and PCR conditions; the nucleic acid sequence of a portion of LST1 (SEQ ID NO:171); and the PCR product obtained using the primers is shown underlined (SEQ ID NO:172).

FIG. 44 shows the upper and lower primer sequences (SEQ ID NOS:173-174) and PCR conditions; the nucleic acid sequence of a portion of LST2 (SEQ ID NO:175); and the PCR product obtained using the primers is shown underlined (SEQ ID NO:176).

FIG. 45 shows the upper and lower primer sequences (SEQ ID NOS:177-178) and PCR conditions; the nucleic acid sequence of a portion of LST3 (SEQ ID NO:179); and the PCR product obtained using the primers is shown underlined (SEQ ID NO:180).

FIG. 46 shows the upper and lower primer sequences (SEQ ID NOS:181-182) and PCR conditions; the nucleic acid sequence of a portion of NTCP (also known as SLC10A1) (SEQ ID NO:183); and the PCR product obtained using the primers is shown underlined (SEQ ID NO:184).

FIG. 47 shows the upper and lower primer sequences (SEQ ID NOS:185-186) and PCR conditions; the nucleic acid sequence of a portion of NTCP2 (SEQ ID NO:187); and the PCR product obtained using the primers is shown underlined (SEQ ID NO:188).

FIG. 48 shows the upper and lower primer sequences (SEQ ID NOS:189-190) and PCR conditions; the nucleic acid sequence of a portion of OAT1 (also known as SLC22A6) (SEQ ID NO:191); and the PCR product obtained using the primers is shown underlined (SEQ ID NO:192).

FIG. 49 shows the upper and lower primer sequences (SEQ ID NOS:193-194) and PCR conditions; the nucleic acid sequence of a portion of OAT2 (also known as SLC22A7) (SEQ ID NO:195); and the PCR product obtained using the primers is shown underlined (SEQ ID NO:196).

FIG. 50 shows the upper and lower primer sequences (SEQ ID NOS:197-198) and PCR conditions; the nucleic acid sequence of a portion of OAT3 (also known as SLC22A8) (SEQ ID NO:199); and the PCR product obtained using the primers is shown underlined (SEQ ID NO:200).

FIG. 51 shows the upper and lower primer sequences (SEQ ID NOS:201-202) and PCR conditions; the nucleic acid sequence of a portion of OAT4 (also known as SLC22A11) (SEQ ID NO:203); and the PCR product obtained using the primers is shown underlined (SEQ ID NO:204).

FIG. 52 shows the upper and lower primer sequences (SEQ ID NOS:205-206) and PCR conditions; the nucleic acid sequence of a portion of OAT4L (also known as SLC22A12) (SEQ ID NO:207); and the PCR product obtained using the primers is shown underlined (SEQ ID NO:208).

FIG. 53 shows the upper and lower primer sequences (SEQ ID NOS:209-210) and PCR conditions; the nucleic acid sequence of a portion of OATP-A (also known as SLC21A3) (SEQ ID NO:211); and the PCR product obtained using the primers is shown underlined (SEQ ID NO:212).

FIG. 54 shows the upper and lower primer sequences (SEQ ID NOS:213-214) and PCR conditions; the nucleic acid sequence of a portion of OATP-B (also known as SLC21A9) (SEQ ID NO:215); and the PCR product obtained using the primers is shown underlined (SEQ ID NO:216).

FIG. 55 shows the upper and lower primer sequences (SEQ ID NOS:217-218) and PCR conditions; the nucleic acid sequence of a portion of OATP-C (also known as SLC21A6) (SEQ ID NO:219); and the PCR product obtained using the primers is shown underlined (SEQ ID NO:220).

FIG. 56 shows the upper and lower primer sequences (SEQ ID NOS:221-222) and PCR conditions; the nucleic acid sequence of a portion of OATP-D (also known as SLC21A11) (SEQ ID NO:223); and the PCR product obtained using the primers is shown underlined (SEQ ID NO:224).

FIG. 57 shows the upper and lower primer sequences (SEQ ID NOS:225-226) and PCR conditions; the nucleic acid sequence of a portion of OATP-E (also known as SLC21A12) (SEQ ID NO:227); and the PCR product obtained using the primers is shown underlined (SEQ ID NO:228).

FIG. 58 shows the upper and lower primer sequences (SEQ ID NOS:229-230) and PCR conditions; the nucleic acid sequence of a portion of OATP-F (also known as SLC21A14) (SEQ ID NO:231); and the PCR product obtained using the primers is shown underlined (SEQ ID NO:232).

FIG. 59 shows the upper and lower primer sequences (SEQ ID NOS:233-234) and PCR conditions; the nucleic acid sequence of a portion of OATP-RP1 (SEQ ID NO:235); and the PCR product obtained using the primers is shown underlined (SEQ ID NO:236).

FIG. 60 shows the upper and lower primer sequences (SEQ ID NOS:237-238) and PCR conditions; the nucleic acid sequence of a portion of OATP-RP2 (SEQ ID NO:239); and the PCR product obtained using the primers is shown underlined (SEQ ID NO:240).

FIG. 61 shows the upper and lower primer sequences (SEQ ID NOS:241-242) and PCR conditions; the nucleic acid sequence of a portion of OATP-RP4 (SEQ ID NO:243); and the PCR product obtained using the primers is shown underlined (SEQ ID NO:244).

FIG. 62 shows the upper and lower primer sequences (SEQ ID NOS:245-246) and PCR conditions; the nucleic acid sequence of a portion of OATP-RP5 (SEQ ID NO:247); and the PCR product obtained using the primers is shown underlined (SEQ ID NO:248).

FIG. 63 shows the upper and lower primer sequences (SEQ ID NOS:249-250) and PCR conditions; the nucleic acid sequence of a portion of OATP8 (also known as SLC21A8, SLC01B3, OATP1B3) (SEQ ID NO:251); and the PCR product obtained using the primers is shown underlined (SEQ ID NO:252).

FIG. 64 shows the upper and lower primer sequences (SEQ ID NOS:253-254) and PCR conditions; the nucleic acid sequence of a portion of OCT1 (also known as SLC22A1) (SEQ ID NO:255); and the PCR product obtained using the primers is shown underlined (SEQ ID NO:256).

FIG. 65 shows the upper and lower primer sequences (SEQ ID NOS:257-258) and PCR conditions; the nucleic acid sequence of a portion of OCT2 (also known as SLC22A2) (SEQ ID NO:259); and the PCR product obtained using the primers is shown underlined (SEQ ID NO:260).

FIG. 66 shows the upper and lower primer sequences (SEQ ID NOS:261-262) and PCR conditions; the nucleic acid sequence of a portion of OCTN1 (also known as SLC22A4) (SEQ ID NO:263); and the PCR product obtained using the primers is shown underlined (SEQ ID NO:264).

FIG. 67 shows the upper and lower primer sequences (SEQ ID NOS:265-266) and PCR conditions; the nucleic acid sequence of a portion of OCTN2 (also known as SLC22A5) (SEQ ID NO:267); and the PCR product obtained using the primers is shown underlined (SEQ ID NO:268).

FIG. 68 shows the upper and lower primer sequences (SEQ ID NOS:269-270) and PCR conditions; the nucleic acid sequence of a portion of ORCTL3 (SEQ ID NO:271); and the PCR product obtained using the primers is shown underlined (SEQ ID NO:272).

FIG. 69 shows the upper and lower primer sequences (SEQ ID NOS:273-274) and PCR conditions; the nucleic acid sequence of a portion of ORCTL4 (SEQ ID NO:275); and the PCR product obtained using the primers is shown underlined (SEQ ID NO:276).

FIG. 70 shows the upper and lower primer sequences (SEQ ID NOS:277-278) and PCR conditions; the nucleic acid sequence of a portion of PGT (also known as SLC21A2) (SEQ ID NO:279); and the PCR product obtained using the primers is shown underlined (SEQ ID NO:280).

FIG. 71 shows the upper and lower primer sequences (SEQ ID NOS:281-282) and PCR conditions; the nucleic acid sequence of a portion of SLC22A1L (SEQ ID NO:283); and the PCR product obtained using the primers is shown underlined (SEQ ID NO:284).

FIG. 72 shows the upper and lower primer sequences (SEQ ID NOS:285-286) and PCR conditions; the nucleic acid sequence of a portion of SLC22A3 (SEQ ID NO:287); and the PCR product obtained using the primers is shown underlined (SEQ ID NO:288).

FIG. 73 shows the CYP, NXR, SLC transporter or SULT/UGT gene RT-PCR amplification products from various total RNA sources including cell lines (Caco-2, HEK293, HepG2) and human tissues (colon, kidney, liver).

FIG. 74 shows the fluorescence intensity matrix plot for the relative levels of CYP, NXR, SLC transporter or SULT/UGT gene expression in normal colon, normal liver, the Caco-2 cell line and Caco-2 treated with doxorubicin.

FIG. 75 shows the fluorescence intensity duster plot the relative levels of CYP, NXR, SLC transporter or SULT/UGT gene expression in the HepG2 cell line treated with doxorubicin at various time intervals.

FIG. 76 shows the fluorescence intensity cluster plot for the relative levels of CYP, NXR, SLC transporter or SULT/UGT gene expression in the HepG2 cell line treated with vinblastine at various time intervals.

FIG. 77 shows the fluorescence intensity matrix plot for the relative levels of drug transporter, drug metabolising enzyme and nuclear receptor-transcription factor gene expression in Caco-2 cell monolayers treated with dimethylsulfoxide, dexamethasone and rifampicin for 7, 14 and 21 days.

FIG. 78 shows the fluorescence intensity matrix plot for the relative levels of drug transporter, drug metabolizing enzymes and nuclear receptor-transcription factor gene expression in fresh human hepatocytes treated with dimethylsulfoxide, dexamethasone and rifampicin for 2 and 4 hours.

### DETAILED DESCRIPTION OF THE INVENTION

The present invention provides materials and methods for detecting the gene expression of cytochrome p450, nuclear X receptors, phase II transferases, and solute carrier family uptake pumps.

### (I) Abbreviations

The following standard abbreviations for the nucleic acid residues are used throughout the specification: A-adenine; C-cytosine; G-guanine; T-thymine; and U-uracil.

### (II) Definitions

The term “nucleic acids”, “nucleic acid molecules”, “nucleic acid sequences”, “nucleotide sequences” and “nucleotide molecules” are used interchangeably herein and refer to a polymer of ribonucleic acids or deoxyribonucleic acids, including RNA, mRNA, rRNA, tRNA, small nuclear RNAs, cDNA, DNA, PNA, or RNA/DNA copolymers. Nucleic acid may be obtained from a cellular extract, genomic or extragenomic DNA, viral RNA or DNA, or artificially/chemically synthesized molecules. The term can include double stranded or single stranded ribonucleic acids or deoxyribonucleic acids.

The term “cDNA” refers to complementary or “copy” DNA. Generally, cDNA is synthesized by a DNA polymerase using any type of RNA molecule as a template. Alternatively, the cDNA can be obtained by direct chemical synthesis.

The term “RNA” refers to a polymer of ribonucleic acids, including RNA, mRNA, rRNA, tRNA and small nuclear RNAS, as well as to RNAs that comprise ribonucleotide analogues to natural ribonucleic acid residues, such as 2-O-methylated residues.

The term “PCR amplicon” or “amplicon” refers to a nucleic acid generated by nucleic acid amplification, particularly PCR amplification.

“Amplification” is defined as the production of additional copies of a nucleic acid sequence and is generally carried out using polymerase chain reaction technologies well known in the art (Dieffenbach C W and G S Dveksler (1995) PCR Primer, a Laboratory Manual, Cold Spring Harbor Press, Plainview N.Y.). As used herein, the term “polymerase chain reaction” (PCR) refers to the method of K. B. Mullis U.S. Pat. Nos. 4,683,195 and 4,683,202, hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as PCR. Because the desired amplified segments of the target sequence become the predominant sequences (in terms of concentration) in the mixture, they are said to be “PCR amplified”.

Amplification in PCR requires “PCR reagents” or “PCR materials”, which herein are defined as all reagents necessary to carry out amplification except the polymerase, primers and template. PCR reagents normally include nucleic acid precursors (dCTP, dTTP etc.) and buffer.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer can be single stranded for maximum efficiency in amplification, but may alternatively be double stranded. If double stranded, the primer is first treated to separate its strands before being used to prepare extension products. In one embodiment, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “pair(s) of primers” refers to an upper primer and a lower primer. The primers can be categorized as upper or lower primers, depending upon the relative orientation of the primer versus the polarity of the nucleic acid sequence of interest (e.g., whether the primer binds to the coding strand or a complementary (noncoding) strand of the sequence of interest).

The term “transcription” refers to the process of copying a DNA sequence of a gene into an RNA product, generally conducted by a DNA-directed RNA polymerase using the DNA as a template.

The term “isolated”, when used in relation to a nucleic acid molecule or sequence, refers to a nucleic acid sequence that is identified and separated from at least one contaminant nucleic acid with which it is ordinarily associated in its natural source. Isolated nucleic acid is nucleic acid present in a form or setting that is different from that in which it is found in nature. In a preferred embodiment, an isolated nucleic acid is substantially free of cellular material or culture medium when produced by recombinant DNA techniques, or chemical precursors, or other chemicals when chemically synthesized.

As used herein, the term “purified” or “to purify” refers to the removal of undesired components from a sample.

### (III) Nucleic Acid Molecules

The inventors have prepared primer pairs for nucleic acids encoding cytochrome p450, nuclear X receptors, phase II transferases and solute carrier family uptake pumps, which can be used, for example, to prepare probes for gene expression screening analysis. For example, the primer pairs of the invention can be used to generate PCR amplicons. Each of these PCR amplicons specifically hybridizes to a different cytochrome p450, nuclear X receptor, phase II transferase or a solute carrier family uptake pump gene expression product. By “specifically hybridizes to” it is meant that the subject PCR amplicon will bind, duplex or hybridize substantially to or only with a particular nucleic acid sequence with minimum cross-hybridization with other nucleic acid sequences. In other words, the PCR amplicon represents a probe to detect the expression of a specific gene, preferably a cytochrome p450 gene, nuclear X receptor gene, phase II transferase gene or solute carrier family uptake pump gene.

Accordingly, one aspect of the invention is a primer pair selected from:
(a) the following pairs of nucleic acid sequences:
   SEQ ID NO:1 and SEQ ID NO:2;
   SEQ ID NO:5 and SEQ ID NO:6;
   SEQ ID NO:9 and SEQ ID NO:10;
   SEQ ID NO:13 and SEQ ID NO:14;
   SEQ ID NO:17 and SEQ ID NO:18;
   SEQ ID NO:21 and SEQ ID NO:22;
   SEQ ID NO:25 and SEQ ID NO:26;
   SEQ ID NO:29 and SEQ ID NO:30;
   SEQ ID NO:33 and SEQ ID NO:34;
   SEQ ID NO:37 and SEQ ID NO:38;
   SEQ ID NO:41 and SEQ ID NO:42;
   SEQ ID NO:45 and SEQ ID NO:46;
   SEQ ID NO:49 and SEQ ID NO:50;
   SEQ ID NO:53 and SEQ ID NO:54;
   SEQ ID NO:57 and SEQ ID NO:58;
   SEQ ID NO:61 and SEQ ID NO:62;
   SEQ ID NO:65 and SEQ ID NO:66;
   SEQ ID NO:69 and SEQ ID NO:70;
   SEQ ID NO:73 and SEQ ID NO:74;
   SEQ ID NO:77 and SEQ ID NO:78;
   SEQ ID NO:81 and SEQ ID NO:82;
   SEQ ID NO:85 and SEQ ID NO:86;
   SEQ ID NO:89 and SEQ ID NO:90;
   SEQ ID NO:93 and SEQ ID NO:94;
   SEQ ID NO:97 and SEQ ID NO:98;
   SEQ ID NO:101 and SEQ ID NO:102;
   SEQ ID NO:105 and SEQ ID NO:106;
   SEQ ID NO:109 and SEQ ID NO:110;
   SEQ ID NO:113 and SEQ ID NO:114;
   SEQ ID NO:117 and SEQ ID NO:118;
   SEQ ID NO:121 and SEQ ID NO:122;
   SEQ ID NO:125 and SEQ ID NO:126;
   SEQ ID NO:129 and SEQ ID NO:130;
   SEQ ID NO:133 and SEQ ID NO:134;
   SEQ ID NO:137 and SEQ ID NO: 138;
   SEQ ID NO:141 and SEQ ID NO:142;
   SEQ ID NO:145 and SEQ ID NO:146;
   SEQ ID NO:149 and SEQ ID NO:150;
   SEQ ID NO:153 and SEQ ID NO:154;
   SEQ ID NO:157 and SEQ ID NO:158;
   SEQ ID NO:161 and SEQ ID NO:162;
   SEQ ID NO:165 and SEQ ID NO:166;
   SEQ ID NO:169 and SEQ ID NO:170;
   SEQ ID NO:173 and SEQ ID NO:174;
   SEQ ID NO:177 and SEQ ID NO:178;
   SEQ ID NO:181 and SEQ ID NO:182;
   SEQ ID NO:185 and SEQ ID NO:186;
   SEQ ID NO:189 and SEQ ID NO:190;
   SEQ ID NO:193 and SEQ ID NO:194;
   SEQ ID NO:197 and SEQ ID NO:198;
   SEQ ID NO:201 and SEQ ID NO:202;
   SEQ ID NO:205 and SEQ ID NO:206;
   SEQ ID NO:209 and SEQ ID NO:210;
   SEQ ID NO:213 and SEQ ID NO:214;
   SEQ ID NO:217 and SEQ ID NO:218;
   SEQ ID NO:221 and SEQ ID NO:222;
   SEQ ID NO:225 and SEQ ID NO:226;
   SEQ ID NO:229 and SEQ ID NO:230;
   SEQ ID NO:233 and SEQ ID NO:234;
   SEQ ID NO:237 and SEQ ID NO:238;
   SEQ ID NO:241 and SEQ ID NO:242;
   SEQ ID NO:245 and SEQ ID NO:246;
   SEQ ID NO:249 and SEQ ID NO:250;
   SEQ ID NO:253 and SEQ ID NO:254;
   SEQ ID NO:257 and SEQ ID NO:258;
   SEQ ID NO:261 and SEQ ID NO:262;
   SEQ ID NO:265 and SEQ ID NO:266;
   SEQ ID NO:269 and SEQ ID NO:270;
   SEQ ID NO:273 and SEQ ID NO:274;
   SEQ ID NO:277 and SEQ ID NO:278;
   SEQ ID NO:281 and SEQ ID NO:282; or
   SEQ ID NO:285 and SEQ ID NO:286;
(b) the nucleic acid sequences in (a) wherein T can also be U;
(c) nucleic acid sequences complementary to (a) or (b); or
(d) nucleic acid sequences that have substantial sequence homology to (a), (b) or (c).

In one embodiment, the primer pairs disclosed herein are used to prepare probes to detect the expression of genes encoding cytochrome P450 enzymes, uptake transporters and/or nuclear xenoreceptors.

The term “complementary” as used herein refers to nucleic acid sequences capable of base-pairing according to the standard Watson-Crick complementary rules, or being capable of hybridizing to a particular nucleic acid segment under stringent conditions.

The term “hybridization” refers to duplex formation between two or more polynucleotides to form, for example a double-stranded nucleic acid, via base pairing. The ability of two regions of complementarity to hybridize and remain together depends on the length and continuity of the complementary regions, and the stringency of the hybridization conditions.

The term “substantial sequence homology” as used herein refers to nucleic acid sequences which have slight or inconsequential sequence variations from the nucleic acid sequences of the invention (i.e. the nucleic acid sequences of (a), (b) or (c)), and function in substantially the same manner of the nucleic acid sequences of the invention. Nucleic acid sequences having substantial homology include nucleic acid sequences having at least 70%, more preferably at least 80%, even more preferably at least 90%, and most preferably at least 95% sequence identity with the nucleic acid sequences of the invention.

The term “sequence identity” as used herein refers to the percentage of sequence identity between two nucleic acid sequences. In order to determine the percentage of identity between two nucleic sequences, the nucleic acid sequences of such two sequences are aligned. Sequence identity is most preferably assessed by the algorithms of BLAST (References to BLAST Searches include: Altschul, S. F., Gish, W., Miller, W., Myers, E. W. & Lipman, D. J. (1990) “Basic local alignment search tool.” J. Mol. Biol. 215:403.sub.-410; Madden, T. L., Tatusov, R. L. & Zhang, J. (1996) “Applications of network BLAST server” Meth. Enzymol. 266:131.sub.-141; Zhang, J. & Madden, T. L. (1997) “PowerBLAST: A new network BLAST application for interactive or automated sequence analysis and annotation.” Genome Res. 7:649.sub.-656).

Another aspect of the invention includes the isolated nucleic acid molecule, such as a PCR amplicon, generated using the primer pairs of the invention. Accordingly, the invention includes isolated nucleic acid molecules prepared using any known amplification method, such as PCR, and the primer pairs of the invention. A further aspect of the invention is an isolated nucleic acid molecule having a nucleic acid sequence consisting of:
(a) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288,
(b) a nucleic acid sequence in (a) wherein T can also be U;
(c) a nucleic acid sequence complementary to (a) or (b); or
(d) a nucleic acid sequence that has substantial sequence homology to (a), (b) or (c); or
(e) a fragment of (a) to (d).

The term “fragment” as used herein refers to a contiguous portion or part of a reference sequence and has the same function as the reference sequence. For example, SEQ ID NO:4 is a probe to detect the expression of CYP1A2. Thus, it is able to specifically hybridize to a nucleic acid sequence that encodes CYP1A2 with minimum cross-hybridization to other nucleic acid sequences. Thus, a fragment of SEQ ID NO:4 is a contiguous portion or part of SEQ ID NO:4 and is able to specifically hybridize to a nucleic acid sequence that encodes CYP1A2 with minimum cross-hybridization to other nucleic acid sequences. In one embodiment, the fragment is 400 to 1000 nucleotides in length.

The invention also includes primer pairs for preparing the isolated nucleic acid molecules disclosed herein.

### (IV) Arrays

The nucleic acid of the invention, such as the PCR amplicons generated using the primer pairs of the invention, can be used in assays, such as arrays to detect the expression of genes encoding cytochrome p450, nuclear X receptors, phase II transferases, and solute carrier family uptake pumps. Arrays, such as microarrays, have the benefit of assaying gene expression in a high throughput fashion.

Accordingly, one aspect of the invention is an array comprising two or more nucleic acid molecules of the invention immobilized to a substrate (i.e. target). The term “immobilized” includes attaching or directly chemically synthesizing the nucleic acid molecules of the invention on the substrate. The term “array” refers to a substrate with at least two target nucleic acid molecules, such as a nucleic acid molecule of the invention, immobilized to said substrate. The target nucleic acid molecules are typically immobilized in prearranged patterns so that their locations are known or determinable. Nucleic acids in a sample can be detected by contacting the sample with the microarray; allowing the target nucleic acid molecule and nucleic acids in the sample to hybridize; and analyzing the extent of hybridization.

The substrate may be, for example, a membrane, a glass support, a filter, a tissue culture dish, a polymeric material, a bead or a silica support. For example, the substrate can be NoAb BioDiscoveries Inc. activated covalent-binding epoxy slide [UAS0005E].

In a preferred embodiment, the array is a microarray.

In embodiments of the invention, the two or more nucleic acid molecules are arranged in distinct spots on the substrate that are known or on determinable locations within the array. A spot refers to a region where the target nucleic acid molecule is attached to the substrate, for example, as a result of contacting a solution comprising target nucleic acid molecule with the substrate. Each spot can be sufficiently separated from each other spot on the substrate such that they are distinguishable from each other during the hybridization analysis.

In an embodiment, there are at least 72 spots on the array; one spot for each of the 72 PCR amplicons generated by the 72 sets of primers disclosed herein which are used as target nucleic acid molecules. In another embodiment, the array additionally includes at least one spot for an expression level control.

When the nucleic acid molecule is immobilized on the substrate, a conventionally known technique can be used. For example, the surface of the substrate can be treated with polycations such as polylysines to electrostatically bind the target molecules through their charges on the surface of the substrate, and techniques to covalently bind the 5′-end of the target DNA to the substrate may be used. Also, a substrate that has linkers on its surface can be produced, and functional groups that can form covalent bonds with the linkers can be introduced at the end of the DNA to be immobilized. Then, by forming a covalent bond between the linker and the functional group, the DNA and such can be immobilized.

Other methods of forming arrays of oligonucleotides, peptides and other polymer sequences with a minimal number of synthetic steps are known and may be used in the present invention. These methods include, but are not limited to, light-directed chemical coupling and mechanically directed coupling. See Pirrung et al., U.S. Pat. No. 5,143,854 and PCT Application No. WO 90/15070, Fodor et al., PCT Publication Nos. WO 92/10092 and WO 93/09668, which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPSTM procedures. Using the VLSIPSTM approach, one heterogeneous array of polymers is converted, through simultaneous coupling at a number of reaction sites, into a different heterogeneous array.

Accordingly, the invention includes an array comprising two or more nucleic acid molecules immobilized on a substrate, wherein at least two of the nucleic acid molecules have a nucleic acid sequence consisting of:
(a) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288;
(b) a nucleic acid sequence prepared using amplification and primer pairs, wherein the primer pairs are selected from the following pairs of nucleic acid sequences:
   SEQ ID NO:1 and SEQ ID NO:2;
   SEQ ID NO:5 and SEQ ID NO:6;
   SEQ ID NO:9 and SEQ ID NO:10;
   SEQ ID NO:13 and SEQ ID NO:14;
   SEQ ID NO:17 and SEQ ID NO:18;
   SEQ ID NO:21 and SEQ ID NO:22;
   SEQ ID NO:25 and SEQ ID NO:26;
   SEQ ID NO:29 and SEQ ID NO:30;
   SEQ ID NO:33 and SEQ ID NO:34;
   SEQ ID NO:37 and SEQ ID NO:38;
   SEQ ID NO:41 and SEQ ID NO:42;
   SEQ ID NO:45 and SEQ ID NO:46;
   SEQ ID NO:49 and SEQ ID NO:50;
   SEQ ID NO:53 and SEQ ID NO:54;
   SEQ ID NO:57 and SEQ ID NO:58;
   SEQ ID NO:61 and SEQ ID NO:62;
   SEQ ID NO:65 and SEQ ID NO:66;
   SEQ ID NO:69 and SEQ ID NO:70;
   SEQ ID NO:73 and SEQ ID NO:74;
   SEQ ID NO:77 and SEQ ID NO:78;
   SEQ ID NO:81 and SEQ ID NO:82;
   SEQ ID NO:85 and SEQ ID NO:86;
   SEQ ID NO:89 and SEQ ID NO:90;
   SEQ ID NO:93 and SEQ ID NO:94;
   SEQ ID NO:97 and SEQ ID NO:98;
   SEQ ID NO:101 and SEQ ID NO:102;
   SEQ ID NO:105 and SEQ ID NO:106;
   SEQ ID NO:109 and SEQ ID NO:110;
   SEQ ID NO:113 and SEQ ID NO:114;
   SEQ ID NO:117 and SEQ ID NO:118;
   SEQ ID NO:121 and SEQ ID NO:122;
   SEQ ID NO:125 and SEQ ID NO:126;
   SEQ ID NO:129 and SEQ ID NO:130;
   SEQ ID NO:133 and SEQ ID NO:134;
   SEQ ID NO:137 and SEQ ID NO: 138;
   SEQ ID NO:141 and SEQ ID NO:142;
   SEQ ID NO:145 and SEQ ID NO:146;
   SEQ ID NO:149 and SEQ ID NO:150;
   SEQ ID NO:153 and SEQ ID NO:154;
   SEQ ID NO:157 and SEQ ID NO:158;
   SEQ ID NO:161 and SEQ ID NO:162;
   SEQ ID NO:165 and SEQ ID NO:166;
   SEQ ID NO:169 and SEQ ID NO:170;
   SEQ ID NO:173 and SEQ ID NO:174;
   SEQ ID NO:177 and SEQ ID NO:178;
   SEQ ID NO:181 and SEQ ID NO:182;
   SEQ ID NO:185 and SEQ ID NO:186;
   SEQ ID NO:189 and SEQ ID NO:190;
   SEQ ID NO:193 and SEQ ID NO:194;
   SEQ ID NO:197 and SEQ ID NO:198;
   SEQ ID NO:201 and SEQ ID NO:202;
   SEQ ID NO:205 and SEQ ID NO:206;
   SEQ ID NO:209 and SEQ ID NO:210;
   SEQ ID NO:213 and SEQ ID NO:214;
   SEQ ID NO:217 and SEQ ID NO:218;
   SEQ ID NO:221 and SEQ ID NO:222;
   SEQ ID NO:225 and SEQ ID NO:226;
   SEQ ID NO:229 and SEQ ID NO:230;
   SEQ ID NO:233 and SEQ ID NO:234;
   SEQ ID NO:237 and SEQ ID NO:238;
   SEQ ID NO:241 and SEQ ID NO:242;
   SEQ ID NO:245 and SEQ ID NO:246;
   SEQ ID NO:249 and SEQ ID NO:250;
   SEQ ID NO:253 and SEQ ID NO:254;
   SEQ ID NO:257 and SEQ ID NO:258;
   SEQ ID NO:261 and SEQ ID NO:262;
   SEQ ID NO:265 and SEQ ID NO:266;
   SEQ ID NO:269 and SEQ ID NO:270;
   SEQ ID NO:273 and SEQ ID NO:274;
   SEQ ID NO:277 and SEQ ID NO:278;
   SEQ ID NO:281 and SEQ ID NO:282; or
   SEQ ID NO:285 and SEQ ID NO:286;
(c) a nucleic acid sequence in (a) or (b) wherein T can also be U;
(d) a nucleic acid sequence complementary to (a), (b) or (c);
(e) a nucleic acid sequence that has substantial sequence homology to (a), (b), (c) or (d); or
(f) a fragment of (a) to (e).

Another aspect provided is an array for screening a sample for the presence of nucleic acid molecules that encode cytochrome P450 enzymes, uptake transporters and/or nuclear xenoreceptors, the array comprising a substrate having immobilized in distinct spots thereon at least 2 nucleic acid probes selected from the group consisting of:
1) a probe that specifically hybridizes to a nucleic acid sequence encoding CYP1A2, wherein the nucleic acid sequence of the probe is selected from the group consisting of:
   (a) a nucleic acid sequence consisting of SEQ ID NO:4,
   (b) a nucleic acid sequence prepared using amplification and primer pairs having the nucleic acid sequence of SEQ ID NO:1 and SEQ ID NO:2,
   (c) a nucleic acid sequence of (a) or (b) wherein T can be U, and
   (d) a fragment of (a), (b) or (c);
2) a probe that specifically hybridizes to a nucleic acid sequence encoding CYP1B1, wherein the nucleic acid sequence of the probe is selected from the group consisting of:
   (a) a nucleic acid sequence consisting of SEQ ID NO:8,
   (b) a nucleic acid sequence prepared using amplification and primer pairs having the nucleic acid sequence of SEQ ID NO:5 and SEQ ID NO:6,
   (c) a nucleic acid sequence of (a) or (b) wherein T can be U, and
   (d) a fragment of (a), (b) or (c);
3) a probe that specifically hybridizes to a nucleic acid sequence encoding CYP2A6, wherein the nucleic acid sequence of the probe is selected from the group consisting of:
   (a) a nucleic acid sequence consisting of SEQ ID NO:12,
   (b) a nucleic acid sequence prepared using amplification and primer pairs having the nucleic acid sequence of SEQ ID NO:9 and SEQ ID NO:10,
   (c) a nucleic acid sequence of (a) or (b) wherein T can be U, and
   (d) a fragment of (a), (b) or (c);
4) a probe that specifically hybridizes to a nucleic acid sequence encoding CYP2B6, wherein the nucleic acid sequence of the probe is selected from the group consisting of:
   (a) a nucleic acid sequence consisting of SEQ ID NO:16,
   (b) a nucleic acid sequence prepared using amplification and primer pairs having the nucleic acid sequence of SEQ ID NO:13 and SEQ ID NO:14,
   (c) a nucleic acid sequence of (a) or (b) wherein T can be U, and
   (d) a fragment of (a), (b) or (c);
5) a probe that specifically hybridizes to a nucleic acid sequence encoding CYP2C8 variant 1, wherein the nucleic acid sequence of the probe is selected from the group consisting of:
   (a) a nucleic acid sequence consisting of SEQ ID NO:20,
   (b) a nucleic acid sequence prepared using amplification and primer pairs having the nucleic acid sequence of SEQ ID NO:17 and SEQ ID NO:18,
   (c) a nucleic acid sequence of (a) or (b) wherein T can be U, and
   (d) a fragment of (a), (b) or (c);
6) a probe that specifically hybridizes to a nucleic acid sequence encoding CYP2C8 variant 2, wherein the nucleic acid sequence of the probe is selected from the group consisting of:
   (a) a nucleic acid sequence consisting of SEQ ID NO:24,
   (b) a nucleic acid sequence prepared using amplification and primer pairs having the nucleic acid sequence of SEQ ID NO:21 and SEQ ID NO:22,
   (c) a nucleic acid sequence of (a) or (b) wherein T can be U, and
   (d) a fragment of (a), (b) or (c);
7) a probe that specifically hybridizes to a nucleic acid sequence encoding CYP2C9, wherein the nucleic acid sequence of the probe is selected from the group consisting of:
   (a) a nucleic acid sequence consisting of SEQ ID NO:28,
   (b) a nucleic acid sequence prepared using amplification and primer pairs having the nucleic acid sequence of SEQ ID NO:25 and SEQ ID NO:26,
   (c) a nucleic acid sequence of (a) or (b) wherein T can be U, and
   (d) a fragment of (a), (b) or (c);
8) a probe that specifically hybridizes to a nucleic acid sequence encoding CYP2C19, wherein the nucleic acid sequence of the probe is selected from the group consisting of:
   (a) a nucleic acid sequence consisting of SEQ ID NO:32,
   (b) a nucleic acid sequence prepared using amplification and primer pairs having the nucleic acid sequence of SEQ ID NO:29 and SEQ ID NO:30,
   (c) a nucleic acid sequence of (a) or (b) wherein T can be U, and
   (d) a fragment of (a), (b) or (c);
9) a probe that specifically hybridizes to a nucleic acid sequence encoding CYP2D6, wherein the nucleic acid sequence of the probe is selected from the group consisting of:
   (a) a nucleic acid sequence consisting of SEQ ID NO:36,
   (b) a nucleic acid sequence prepared using amplification and primer pairs having the nucleic acid sequence of SEQ ID NO:33 and SEQ ID NO:34,
   (c) a nucleic acid sequence of (a) or (b) wherein T can be U, and
   (d) a fragment of (a), (b) or (c);
10) a probe that specifically hybridizes to a nucleic acid sequence encoding CYP2E1, wherein the nucleic acid sequence of the probe is selected from the group consisting of:
   (a) a nucleic acid sequence consisting of SEQ ID NO:40,
   (b) a nucleic acid sequence prepared using amplification and primer pairs having the nucleic acid sequence of SEQ ID NO:37 and SEQ ID NO:38,
   (c) a nucleic acid sequence of (a) or (b) wherein T can be U, and
   (d) a fragment of (a), (b) or (c);
11) a probe that specifically hybridizes to a nucleic acid sequence encoding CYP3A4, wherein the nucleic acid sequence of the probe is selected from the group consisting of:
   (a) a nucleic acid sequence consisting of SEQ ID NO:44,
   (b) a nucleic acid sequence prepared using amplification and primer pairs having the nucleic acid sequence of SEQ ID NO:41 and SEQ ID NO:42,
   (c) a nucleic acid sequence of (a) or (b) wherein T can be U, and
   (d) a fragment of (a), (b) or (c);
12) a probe that specifically hybridizes to a nucleic acid sequence encoding CYP19A variant 1, wherein the nucleic acid sequence of the probe is selected from the group consisting of:
   (a) a nucleic acid sequence consisting of SEQ ID NO:48,
   (b) a nucleic acid sequence prepared using amplification and primer pairs having the nucleic acid sequence of SEQ ID NO:45 and SEQ ID NO:46,
   (c) a nucleic acid sequence of (a) or (b) wherein T can be U, and
   (d) a fragment of (a), (b) or (c);
13) a probe that specifically hybridizes to a nucleic acid sequence encoding CYP19A variant 2, wherein the nucleic acid sequence of the probe is selected from the group consisting of:
   (a) a nucleic acid sequence consisting of SEQ ID NO:52,
   (b) a nucleic acid sequence prepared using amplification and primer pairs having the nucleic acid sequence of SEQ ID NO:49 and SEQ ID NO:50,
   (c) a nucleic acid sequence of (a) or (b) wherein T can be U, and
   (d) a fragment of (a), (b) or (c);
14) a probe that specifically hybridizes to a nucleic acid sequence encoding CYP27A1, wherein the nucleic acid sequence of the probe is selected from the group consisting of:
   (a) a nucleic acid sequence consisting of SEQ ID NO:56,
   (b) a nucleic acid sequence prepared using amplification and primer pairs having the nucleic acid sequence of SEQ ID NO:53 and SEQ ID NO:54,
   (c) a nucleic acid sequence of (a) or (b) wherein T can be U, and
   (d) a fragment of (a), (b) or (c);
15) a probe that specifically hybridizes to a nucleic acid sequence encoding CYP27B1, wherein the nucleic acid sequence of the probe is selected from the group consisting of:
   (a) a nucleic acid sequence consisting of SEQ ID NO:60,
   (b) a nucleic acid sequence prepared using amplification and primer pairs having the nucleic acid sequence of SEQ ID NO:57 and SEQ ID NO:58,
   (c) a nucleic acid sequence of (a) or (b) wherein T can be U, and
   (d) a fragment of (a), (b) or (c);
16) a probe that specifically hybridizes to a nucleic acid sequence encoding CAR1, wherein the nucleic acid sequence of the probe is selected from the group consisting of:
   (a) a nucleic acid sequence consisting of SEQ ID NO:64,
   (b) a nucleic acid sequence prepared using amplification and primer pairs having the nucleic acid sequence of SEQ ID NO:61 and SEQ ID NO:62,
   (c) a nucleic acid sequence of (a) or (b) wherein T can be U, and
   (d) a fragment of (a), (b) or (c);
17) a probe that specifically hybridizes to a nucleic acid sequence encoding FXR, wherein the nucleic acid sequence of the probe is selected from the group consisting of:
   (a) a nucleic acid sequence consisting of SEQ ID NO:68,
   (b) a nucleic acid sequence prepared using amplification and primer pairs having the nucleic acid sequence of SEQ ID NO:65 and SEQ ID NO:66,
   (c) a nucleic acid sequence of (a) or (b) wherein T can be U, and
   (d) a fragment of (a), (b) or (c);
18) a probe that specifically hybridizes to a nucleic acid sequence encoding LXR, wherein the nucleic acid sequence of the probe is selected from the group consisting of:
   (a) a nucleic acid sequence consisting of SEQ ID NO:72,
   (b) a nucleic acid sequence prepared using amplification and primer pairs having the nucleic acid sequence of SEQ ID NO:69 and SEQ ID NO:70,
   (c) a nucleic acid sequence of (a) or (b) wherein T can be U, and
   (d) a fragment of (a), (b) or (c);
19) a probe that specifically hybridizes to a nucleic acid sequence encoding PPARA, wherein the nucleic acid sequence of the probe is selected from the group consisting of:
   (a) a nucleic acid sequence consisting of SEQ ID NO:76,
   (b) a nucleic acid sequence prepared using amplification and primer pairs having the nucleic acid sequence of SEQ ID NO:73 and SEQ ID NO:74,
   (c) a nucleic acid sequence of (a) or (b) wherein T can be U, and
   (d) a fragment of (a), (b) or (c);
20) a probe that specifically hybridizes to a nucleic acid sequence encoding PPARD-B, wherein the nucleic acid sequence of the probe is selected from the group consisting of:
   (a) a nucleic acid sequence consisting of SEQ ID NO:80,
   (b) a nucleic acid sequence prepared using amplification and primer pairs having the nucleic acid sequence of SEQ ID NO:77 and SEQ ID NO:78,
   (c) a nucleic acid sequence of (a) or (b) wherein T can be U, and
   (d) a fragment of (a), (b) or (c);
21) a probe that specifically hybridizes to a nucleic acid sequence encoding PPARG, wherein the nucleic acid sequence of the probe is selected from the group consisting of:
   (a) a nucleic acid sequence consisting of SEQ ID NO:84,
   (b) a nucleic acid sequence prepared using amplification and primer pairs having the nucleic acid sequence of SEQ ID NO:81 and SEQ ID NO:82,
   (c) a nucleic acid sequence of (a) or (b) wherein T can be U, and
   (d) a fragment of (a), (b) or (c);
22) a probe that specifically hybridizes to a nucleic acid sequence encoding RXRA, wherein the nucleic acid sequence of the probe is selected from the group consisting of:
   (a) a nucleic acid sequence consisting of SEQ ID NO:88,
   (b) a nucleic acid sequence prepared using amplification and primer pairs having the nucleic acid sequence of SEQ ID NO:85 and SEQ ID NO:86,
   (c) a nucleic acid sequence of (a) or (b) wherein T can be U, and
   (d) a fragment of (a), (b) or (c);
23) a probe that specifically hybridizes to a nucleic acid sequence encoding RXRB, wherein the nucleic acid sequence of the probe is selected from the group consisting of:
   (a) a nucleic acid sequence consisting of SEQ ID NO:92,
   (b) a nucleic acid sequence prepared using amplification and primer pairs having the nucleic acid sequence of SEQ ID NO:89 and SEQ ID NO:90,
   (c) a nucleic acid sequence of (a) or (b) wherein T can be U, and
   (d) a fragment of (a), (b) or (c);
24) a probe that specifically hybridizes to a nucleic acid sequence encoding RXRG, wherein the nucleic acid sequence of the probe is selected from the group consisting of:
   (a) a nucleic acid sequence consisting of SEQ ID NO:96,
   (b) a nucleic acid sequence prepared using amplification and primer pairs having the nucleic acid sequence of SEQ ID NO:93 and SEQ ID NO:94,
   (c) a nucleic acid sequence of (a) or (b) wherein T can be U, and
   (d) a fragment of (a), (b) or (c);
25) a probe that specifically hybridizes to a nucleic acid sequence encoding SXR (PXR) transcript variant 1, wherein the nucleic acid sequence of the probe is selected from the group consisting of:
   (a) a nucleic acid sequence consisting of SEQ ID NO:100,
   (b) a nucleic acid sequence prepared using amplification and primer pairs having the nucleic acid sequence of SEQ ID NO:97 and SEQ ID NO:98,
   (c) a nucleic acid sequence of (a) or (b) wherein T can be U, and
   (d) a fragment of (a), (b) or (c);
26) a probe that specifically hybridizes to a nucleic acid sequence encoding SULT1A1, wherein the nucleic acid sequence of the probe is selected from the group consisting of:
   (a) a nucleic acid sequence consisting of SEQ ID NO:104,
   (b) a nucleic acid sequence prepared using amplification and primer pairs having the nucleic acid sequence of SEQ ID NO:101 and SEQ ID NO:102,
   (c) a nucleic acid sequence of (a) or (b) wherein T can be U, and
   (d) a fragment of (a), (b) or (c);
27) a probe that specifically hybridizes to a nucleic acid sequence encoding SULT1B1, wherein the nucleic acid sequence of the probe is selected from the group consisting of:
   (a) a nucleic acid sequence consisting of SEQ ID NO:108,
   (b) a nucleic acid sequence prepared using amplification and primer pairs having the nucleic acid sequence of SEQ ID NO:105 and SEQ ID NO:106,
   (c) a nucleic acid sequence of (a) or (b) wherein T can be U, and
   (d) a fragment of (a), (b) or (c);
28) a probe that specifically hybridizes to a nucleic acid sequence encoding SULT1C1, wherein the nucleic acid sequence of the probe is selected from the group consisting of:
   (a) a nucleic acid sequence consisting of SEQ ID NO:112,
   (b) a nucleic acid sequence prepared using amplification and primer pairs having the nucleic acid sequence of SEQ ID NO:109 and SEQ ID NO:110,
   (c) a nucleic acid sequence of (a) or (b) wherein T can be U, and
   (d) a fragment of (a), (b) or (c);
29) a probe that specifically hybridizes to a nucleic acid sequence encoding SULT1 E1, wherein the nucleic acid sequence of the probe is selected from the group consisting of:
   (a) a nucleic acid sequence consisting of SEQ ID NO:116,
   (b) a nucleic acid sequence prepared using amplification and primer pairs having the nucleic acid sequence of SEQ ID NO:113 and SEQ ID NO:114,
   (c) a nucleic acid sequence of (a) or (b) wherein T can be U, and
   (d) a fragment of (a), (b) or (c);
30) a probe that specifically hybridizes to a nucleic acid sequence encoding SULT2A1, wherein the nucleic acid sequence of the probe is selected from the group consisting of:
   (a) a nucleic acid sequence consisting of SEQ ID NO:120,
   (b) a nucleic acid sequence prepared using amplification and primer pairs having the nucleic acid sequence of SEQ ID NO:117 and SEQ ID NO:118,
   (c) a nucleic acid sequence of (a) or (b) wherein T can be U, and
   (d) a fragment of (a), (b) or (c);
31) a probe that specifically hybridizes to a nucleic acid sequence encoding SULT2B1b, wherein the nucleic acid sequence of the probe is selected from the group consisting of:
   (a) a nucleic acid sequence consisting of SEQ ID NO:124,
   (b) a nucleic acid sequence prepared using amplification and primer pairs having the nucleic acid sequence of SEQ ID NO:121 and SEQ ID NO:122,
   (c) a nucleic acid sequence of (a) or (b) wherein T can be U, and
   (d) a fragment of (a), (b) or (c);
32) a probe that specifically hybridizes to a nucleic acid sequence encoding UGT2A1, wherein the nucleic acid sequence of the probe is selected from the group consisting of:
   (a) a nucleic acid sequence consisting of SEQ ID NO:128,
   (b) a nucleic acid sequence prepared using amplification and primer pairs having the nucleic acid sequence of SEQ ID NO:125 and SEQ ID NO:126,
   (c) a nucleic acid sequence of (a) or (b) wherein T can be U, and
   (d) a fragment of (a), (b) or (c);
33) a probe that specifically hybridizes to a nucleic acid sequence encoding UGT2B4, wherein the nucleic acid sequence of the probe is selected from the group consisting of:
   (a) a nucleic acid sequence consisting of SEQ ID NO:132,
   (b) a nucleic acid sequence prepared using amplification and primer pairs having the nucleic acid sequence of SEQ ID NO:129 and SEQ ID NO:130,
   (c) a nucleic acid sequence of (a) or (b) wherein T can be U, and
   (d) a fragment of (a), (b) or (c);
34) a probe that specifically hybridizes to a nucleic acid sequence encoding UGT2B15, wherein the nucleic acid sequence of the probe is selected from the group consisting of:
   (a) a nucleic acid sequence consisting of SEQ ID NO:136,
   (b) a nucleic acid sequence prepared using amplification and primer pairs having the nucleic acid sequence of SEQ ID NO:133 and SEQ ID NO:134,
   (c) a nucleic acid sequence of (a) or (b) wherein T can be U, and
   (d) a fragment of (a), (b) or (c);
35) a probe that specifically hybridizes to a nucleic acid sequence encoding UGT2B17, wherein the nucleic acid sequence of the probe is selected from the group consisting of:
   (a) a nucleic acid sequence consisting of SEQ ID NO:140,
   (b) a nucleic acid sequence prepared using amplification and primer pairs having the nucleic acid sequence of SEQ ID NO:137 and SEQ ID NO:138,
   (c) a nucleic acid sequence of (a) or (b) wherein T can be U, and
   (d) a fragment of (a), (b) or (c);
36) a probe that specifically hybridizes to a nucleic acid sequence encoding UGT8, wherein the nucleic acid sequence of the probe is selected from the group consisting of:
   (a) a nucleic acid sequence consisting of SEQ ID NO:144,
   (b) a nucleic acid sequence prepared using amplification and primer pairs having the nucleic acid sequence of SEQ ID NO:141 and SEQ ID NO:142,
   (c) a nucleic acid sequence of (a) or (b) wherein T can be U, and
   (d) a fragment of (a), (b) or (c);
37) a probe that specifically hybridizes to a nucleic acid sequence encoding CNT1, wherein the nucleic acid sequence of the probe is selected from the group consisting of:
   (a) a nucleic acid sequence consisting of SEQ ID NO:148,
   (b) a nucleic acid sequence prepared using amplification and primer pairs having the nucleic acid sequence of SEQ ID NO:145 and SEQ ID NO:146,
   (c) a nucleic acid sequence of (a) or (b) wherein T can be U, and
   (d) a fragment of (a), (b) or (c);
38) a probe that specifically hybridizes to a nucleic acid sequence encoding CNT2, wherein the nucleic acid sequence of the probe is selected from the group consisting of:
   (a) a nucleic acid sequence consisting of SEQ ID NO:152,
   (b) a nucleic acid sequence prepared using amplification and primer pairs having the nucleic acid sequence of SEQ ID NO:149 and SEQ ID NO:150,
   (c) a nucleic acid sequence of (a) or (b) wherein T can be U, and
   (d) a fragment of (a), (b) or (c);
39) a probe that specifically hybridizes to a nucleic acid sequence encoding CNT3, wherein the nucleic acid sequence of the probe is selected from the group consisting of:
   (a) a nucleic acid sequence consisting of SEQ ID NO:156,
   (b) a nucleic acid sequence prepared using amplification and primer pairs having the nucleic acid sequence of SEQ ID NO:153 and SEQ ID NO:154,
   (c) a nucleic acid sequence of (a) or (b) wherein T can be U, and
   (d) a fragment of (a), (b) or (c);
40) a probe that specifically hybridizes to a nucleic acid sequence encoding ENT1, wherein the nucleic acid sequence of the probe is selected from the group consisting of:
   (a) a nucleic acid sequence consisting of SEQ ID NO:160,
   (b) a nucleic acid sequence prepared using amplification and primer pairs having the nucleic acid sequence of SEQ ID NO:157 and SEQ ID NO:158,
   (c) a nucleic acid sequence of (a) or (b) wherein T can be U, and
   (d) a fragment of (a), (b) or (c);
41) a probe that specifically hybridizes to a nucleic acid sequence encoding ENT2, wherein the nucleic acid sequence of the probe is selected from the group consisting of:
   (a) a nucleic acid sequence consisting of SEQ ID NO:164,
   (b) a nucleic acid sequence prepared using amplification and primer pairs having the nucleic acid sequence of SEQ ID NO:161 and SEQ ID NO:162,
   (c) a nucleic acid sequence of (a) or (b) wherein T can be U, and
   (d) a fragment of (a), (b) or (c);
42) a probe that specifically hybridizes to a nucleic acid sequence encoding ENT3, wherein the nucleic acid sequence of the probe is selected from the group consisting of:
   (a) a nucleic acid sequence consisting of SEQ ID NO:168,
   (b) a nucleic acid sequence prepared using amplification and primer pairs having the nucleic acid sequence of SEQ ID NO:165 and SEQ ID NO:166,
   (c) a nucleic acid sequence of (a) or (b) wherein T can be U, and
   (d) a fragment of (a), (b) or (c);
43) a probe that specifically hybridizes to a nucleic acid sequence encoding LST1, wherein the nucleic acid sequence of the probe is selected from the group consisting of:
   (a) a nucleic acid sequence consisting of SEQ ID NO:172,
   (b) a nucleic acid sequence prepared using amplification and primer pairs having the nucleic acid sequence of SEQ ID NO:169 and SEQ ID NO:170,
   (c) a nucleic acid sequence of (a) or (b) wherein T can be U, and
   (d) a fragment of (a), (b) or (c);
44) a probe that specifically hybridizes to a nucleic acid sequence encoding LST2, wherein the nucleic acid sequence of the probe is selected from the group consisting of:
   (a) a nucleic acid sequence consisting of SEQ ID NO:176,
   (b) a nucleic acid sequence prepared using amplification and primer pairs having the nucleic acid sequence of SEQ ID NO:173 and SEQ ID NO:174,
   (c) a nucleic acid sequence of (a) or (b) wherein T can be U, and
   (d) a fragment of (a), (b) or (c);
45) a probe that specifically hybridizes to a nucleic acid sequence encoding LST3, wherein the nucleic acid sequence of the probe is selected from the group consisting of:
   (a) a nucleic acid sequence consisting of SEQ ID NO:180,
   (b) a nucleic acid sequence prepared using amplification and primer pairs having the nucleic acid sequence of SEQ ID NO:177 and SEQ ID NO:178,
   (c) a nucleic acid sequence of (a) or (b) wherein T can be U, and
   (d) a fragment of (a), (b) or (c);
46) a probe that specifically hybridizes to a nucleic acid sequence encoding NTCP, wherein the nucleic acid sequence of the probe is selected from the group consisting of:
   (a) a nucleic acid sequence consisting of SEQ ID NO:184,
   (b) a nucleic acid sequence prepared using amplification and primer pairs having the nucleic acid sequence of SEQ ID NO:181 and SEQ ID NO:182,
   (c) a nucleic acid sequence of (a) or (b) wherein T can be U, and
   (d) a fragment of (a), (b) or (c);
47) a probe that specifically hybridizes to a nucleic acid sequence encoding NTCP2, wherein the nucleic acid sequence of the probe is selected from the group consisting of:
   (a) a nucleic acid sequence consisting of SEQ ID NO:188,
   (b) a nucleic acid sequence prepared using amplification and primer pairs having the nucleic acid sequence of SEQ ID NO:185 and SEQ ID NO:186,
   (c) a nucleic acid sequence of (a) or (b) wherein T can be U, and
   (d) a fragment of (a), (b) or (c);
48) a probe that specifically hybridizes to a nucleic acid sequence encoding OAT1, wherein the nucleic acid sequence of the probe is selected from the group consisting of:
   (a) a nucleic acid sequence consisting of SEQ ID NO:192,
   (b) a nucleic acid sequence prepared using amplification and primer pairs having the nucleic acid sequence of SEQ ID NO:189 and SEQ ID NO:190,
   (c) a nucleic acid sequence of (a) or (b) wherein T can be U, and
   (d) a fragment of (a), (b) or (c);
49) a probe that specifically hybridizes to a nucleic acid sequence encoding OAT2, wherein the nucleic acid sequence of the probe is selected from the group consisting of:
   (a) a nucleic acid sequence consisting of SEQ ID NO:196,
   (b) a nucleic acid sequence prepared using amplification and primer pairs having the nucleic acid sequence of SEQ ID NO:193 and SEQ ID NO:194,
   (c) a nucleic acid sequence of (a) or (b) wherein T can be U, and
   (d) a fragment of (a), (b) or (c);
50) a probe that specifically hybridizes to a nucleic acid sequence encoding OAT3, wherein the nucleic acid sequence of the probe is selected from the group consisting of:
   (a) a nucleic acid sequence consisting of SEQ ID NO:200,
   (b) a nucleic acid sequence prepared using amplification and primer pairs having the nucleic acid sequence of SEQ ID NO:197 and SEQ ID NO:198,
   (c) a nucleic acid sequence of (a) or (b) wherein T can be U, and
   (d) a fragment of (a), (b) or (c);
51) a probe that specifically hybridizes to a nucleic acid sequence encoding OAT4, wherein the nucleic acid sequence of the probe is selected from the group consisting of:
   (a) a nucleic acid sequence consisting of SEQ ID NO:204,
   (b) a nucleic acid sequence prepared using amplification and primer pairs having the nucleic acid sequence of SEQ ID NO:201 and SEQ ID NO:202,
   (c) a nucleic acid sequence of (a) or (b) wherein T can be U, and
   (d) a fragment of (a), (b) or (c);
52) a probe that specifically hybridizes to a nucleic acid sequence encoding OAT4L, wherein the nucleic acid sequence of the probe is selected from the group consisting of:
   (a) a nucleic acid sequence consisting of SEQ ID NO:208,
   (b) a nucleic acid sequence prepared using amplification and primer pairs having the nucleic acid sequence of SEQ ID NO:205 and SEQ ID NO:206,
   (c) a nucleic acid sequence of (a) or (b) wherein T can be U, and
   (d) a fragment of (a), (b) or (c);
53) a probe that specifically hybridizes to a nucleic acid sequence encoding OATP-A, wherein the nucleic acid sequence of the probe is selected from the group consisting of:
   (a) a nucleic acid sequence consisting of SEQ ID NO:212,
   (b) a nucleic acid sequence prepared using amplification and primer pairs having the nucleic acid sequence of SEQ ID NO:209 and SEQ ID NO:210,
   (c) a nucleic acid sequence of (a) or (b) wherein T can be U, and
   (d) a fragment of (a), (b) or (c);
54) a probe that specifically hybridizes to a nucleic acid sequence encoding OATP-B, wherein the nucleic acid sequence of the probe is selected from the group consisting of:
   (a) a nucleic acid sequence consisting of SEQ ID NO:216,
   (b) a nucleic acid sequence prepared using amplification and primer pairs having the nucleic acid sequence of SEQ ID NO:213 and SEQ ID NO:214,
   (c) a nucleic acid sequence of (a) or (b) wherein T can be U, and
   (d) a fragment of (a), (b) or (c);
55) a probe that specifically hybridizes to a nucleic acid sequence encoding OATP-C, wherein the nucleic acid sequence of the probe is selected from the group consisting of:
   (a) a nucleic acid sequence consisting of SEQ ID NO:220,
   (b) a nucleic acid sequence prepared using amplification and primer pairs having the nucleic acid sequence of SEQ ID NO:217 and SEQ ID NO:218,
   (c) a nucleic acid sequence of (a) or (b) wherein T can be U, and
   (d) a fragment of (a), (b) or (c);
56) a probe that specifically hybridizes to a nucleic acid sequence encoding OATP-D, wherein the nucleic acid sequence of the probe is selected from the group consisting of:
   (a) a nucleic acid sequence consisting of SEQ ID NO:224,
   (b) a nucleic acid sequence prepared using amplification and primer pairs having the nucleic acid sequence of SEQ ID NO:221 and SEQ ID NO:222,
   (c) a nucleic acid sequence of (a) or (b) wherein T can be U, and
   (d) a fragment of (a), (b) or (c);
57) a probe that specifically hybridizes to a nucleic acid sequence encoding OATP-E, wherein the nucleic acid sequence of the probe is selected from the group consisting of:
   (a) a nucleic acid sequence consisting of SEQ ID NO:228,
   (b) a nucleic acid sequence prepared using amplification and primer pairs having the nucleic acid sequence of SEQ ID NO:225 and SEQ ID NO:226,
   (c) a nucleic acid sequence of (a) or (b) wherein T can be U, and
   (d) a fragment of (a), (b) or (c);
58) a probe that specifically hybridizes to a nucleic acid sequence encoding OATP-F, wherein the nucleic acid sequence of the probe is selected from the group consisting of:
   (a) a nucleic acid sequence consisting of SEQ ID NO:232,
   (b) a nucleic acid sequence prepared using amplification and primer pairs having the nucleic acid sequence of SEQ ID NO:229 and SEQ ID NO:230,
   (c) a nucleic acid sequence of (a) or (b) wherein T can be U, and
   (d) a fragment of (a), (b) or (c);
59) a probe that specifically hybridizes to a nucleic acid sequence encoding OATP-RP1, wherein the nucleic acid sequence of the probe is selected from the group consisting of:
   (a) a nucleic acid sequence consisting of SEQ ID NO:236,
   (b) a nucleic acid sequence prepared using amplification and primer pairs having the nucleic acid sequence of SEQ ID NO:233 and SEQ ID NO:234,
   (c) a nucleic acid sequence of (a) or (b) wherein T can be U, and
   (d) a fragment of (a), (b) or (c);
60) a probe that specifically hybridizes to a nucleic acid sequence encoding OATP-RP2, wherein the nucleic acid sequence of the probe is selected from the group consisting of:
   (a) a nucleic acid sequence consisting of SEQ ID NO:240,
   (b) a nucleic acid sequence prepared using amplification and primer pairs having the nucleic acid sequence of SEQ ID NO:237 and SEQ ID NO:238,
   (c) a nucleic acid sequence of (a) or (b) wherein T can be U, and
   (d) a fragment of (a), (b) or (c);
61) a probe that specifically hybridizes to a nucleic acid sequence encoding OATP-RP4, wherein the nucleic acid sequence of the probe is selected from the group consisting of:
   (a) a nucleic acid sequence consisting of SEQ ID NO:244,
   (b) a nucleic acid sequence prepared using amplification and primer pairs having the nucleic acid sequence of SEQ ID NO:241 and SEQ ID NO:242,
   (c) a nucleic acid sequence of (a) or (b) wherein T can be U, and
   (d) a fragment of (a), (b) or (c);
62) a probe that specifically hybridizes to a nucleic acid sequence encoding OATP-RP5, wherein the nucleic acid sequence of the probe is selected from the group consisting of:
   (a) a nucleic acid sequence consisting of SEQ ID NO:248,
   (b) a nucleic acid sequence prepared using amplification and primer pairs having the nucleic acid sequence of SEQ ID NO:245 and SEQ ID NO:246,
   (c) a nucleic acid sequence of (a) or (b) wherein T can be U, and
   (d) a fragment of (a), (b) or (c);
63) a probe that specifically hybridizes to a nucleic acid sequence encoding OATP8, wherein the nucleic acid sequence of the probe is selected from the group consisting of:
   (a) a nucleic acid sequence consisting of SEQ ID NO:252,
   (b) a nucleic acid sequence prepared using amplification and primer pairs having the nucleic acid sequence of SEQ ID NO:249 and SEQ ID NO:250,
   (c) a nucleic acid sequence of (a) or (b) wherein T can be U, and
   (d) a fragment of (a), (b) or (c);
64) a probe that specifically hybridizes to a nucleic acid sequence encoding OCT1, wherein the nucleic acid sequence of the probe is selected from the group consisting of:
   (a) a nucleic acid sequence consisting of SEQ ID NO:256,
   (b) a nucleic acid sequence prepared using amplification and primer pairs having the nucleic acid sequence of SEQ ID NO:253 and SEQ ID NO:254,
   (c) a nucleic acid sequence of (a) or (b) wherein T can be U, and
   (d) a fragment of (a), (b) or (c);
65) a probe that specifically hybridizes to a nucleic acid sequence encoding OCT2, wherein the nucleic acid sequence of the probe is selected from the group consisting of:
   (a) a nucleic acid sequence consisting of SEQ ID NO:260,
   (b) a nucleic acid sequence prepared using amplification and primer pairs having the nucleic acid sequence of SEQ ID NO:257 and SEQ ID NO:258,
   (c) a nucleic acid sequence of (a) or (b) wherein T can be U, and
   (d) a fragment of (a), (b) or (c);
66) a probe that specifically hybridizes to a nucleic acid sequence encoding OCTN1, wherein the nucleic acid sequence of the probe is selected from the group consisting of:
   (a) a nucleic acid sequence consisting of SEQ ID NO:264,
   (b) a nucleic acid sequence prepared using amplification and primer pairs having the nucleic acid sequence of SEQ ID NO:261 and SEQ ID NO:262,
   (c) a nucleic acid sequence of (a) or (b) wherein T can be U, and
   (d) a fragment of (a), (b) or (c);
67) a probe that specifically hybridizes to a nucleic acid sequence encoding OCTN2, wherein the nucleic acid sequence of the probe is selected from the group consisting of:
   (a) a nucleic acid sequence consisting of SEQ ID NO:268,
   (b) a nucleic acid sequence prepared using amplification and primer pairs having the nucleic acid sequence of SEQ ID NO:265 and SEQ ID NO:266,
   (c) a nucleic acid sequence of (a) or (b) wherein T can be U, and
   (d) a fragment of (a), (b) or (c);
68) a probe that specifically hybridizes to a nucleic acid sequence encoding ORCTL3, wherein the nucleic acid sequence of the probe is selected from the group consisting of:
   (a) a nucleic acid sequence consisting of SEQ ID NO:272,
   (b) a nucleic acid sequence prepared using amplification and primer pairs having the nucleic acid sequence of SEQ ID NO:269 and SEQ ID NO:270,
   (c) a nucleic acid sequence of (a) or (b) wherein T can be U, and
   (d) a fragment of (a), (b) or (c);
69) a probe that specifically hybridizes to a nucleic acid sequence encoding ORCTL4, wherein the nucleic acid sequence of the probe is selected from the group consisting of:
   (a) a nucleic acid sequence consisting of SEQ ID NO:276,
   (b) a nucleic acid sequence prepared using amplification and primer pairs having the nucleic acid sequence of SEQ ID NO:273 and SEQ ID NO:274,
   (c) a nucleic acid sequence of (a) or (b) wherein T can be U, and
   (d) a fragment of (a), (b) or (c);
70) a probe that specifically hybridizes to a nucleic acid sequence encoding PGT, wherein the nucleic acid sequence of the probe is selected from the group consisting of:
   (a) a nucleic acid sequence consisting of SEQ ID NO:280,
   (b) a nucleic acid sequence prepared using amplification and primer pairs having the nucleic acid sequence of SEQ ID NO:277 and SEQ ID NO:278,
   (c) a nucleic acid sequence of (a) or (b) wherein T can be U, and
   (d) a fragment of (a), (b) or (c);
71) a probe that specifically hybridizes to a nucleic acid sequence encoding SLC22A1 L, wherein the nucleic acid sequence of the probe is selected from the group consisting of:
   (a) a nucleic acid sequence consisting of SEQ ID NO:284,
   (b) a nucleic acid sequence prepared using amplification and primer pairs having the nucleic acid sequence of SEQ ID NO:281 and SEQ ID NO:282,
   (c) a nucleic acid sequence of (a) or (b) wherein T can be U, and
   (d) a fragment of (a), (b) or (c); and
72) a probe that specifically hybridizes to a nucleic acid sequence encoding SLC22A3, wherein the nucleic acid sequence of the probe is selected from the group consisting of:
   (a) a nucleic acid sequence consisting of SEQ ID NO:288,
   (b) a nucleic acid sequence prepared using amplification and primer pairs having the nucleic acid sequence of SEQ ID NO:285 and SEQ ID NO:286,
   (c) a nucleic acid sequence of (a) or (b) wherein T can be U, and
   (d) a fragment of (a), (b) or (c).

In one embodiment, the array is used to determine a change in the gene expression profile in a subject in response to a drug or a combination of drugs. In another embodiment, the array is used to detect or determine drug-drug interactions in a subject exposed to one or more compounds or drugs.

In a further embodiment of the present invention, at least two different nucleic acid molecules of the invention, at least 10 different nucleic acid molecules of the invention, at least 20 different nucleic acid molecules of the invention, at least 30 different nucleic acid molecules of the invention, at least 40 different nucleic acid molecules of the invention, at least 50 different nucleic acid molecules of the invention, at least 60 different nucleic acid molecules of the invention, at least 70 different nucleic acid molecules of the invention or at least 72 different nucleic acid molecules of the invention are immobilized on the substrate.

An array used to detect gene expression typically includes one or more control nucleic acid molecules or probes. The control may be, for example, expression level controls (e.g. positive controls and background negative controls).

Background controls are elements printed on the substrate that contain no nucleic acids and thus measure the amount of non-specific hybridization of the labeled cDNA to elements on the substrate.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the beta-actin gene, the transferrin receptor gene, the glyceraldehyde-3-phosphate dehydrogenase (GAPDH) gene, and the like [Warrington J A et al., Physiol Genomics 2:143-147, 2000, Hsiao L L et al., Physiol Genomics 7:97-104, 2001, Whitfield M L et al., Mol Cell Biol 13:1977-2000, 2002].

### (V) Methods for Detecting Gene Expression

The nucleic acids and arrays of the invention can be used to detect and profile gene expression, particularly the expression of cytochrome p450 genes, nuclear X receptor genes, phase II transferase genes and solute carrier family uptake pumps genes.

Accordingly, the invention includes methods of detecting the expression of two or more genes, comprising the steps:
(a) providing two or more nucleic acid molecules, wherein the two or more nucleic acid molecules each comprise a nucleic acid sequence selected from:
   (i) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288,
   (ii) a nucleic acid sequence prepared using amplification and primer pairs, wherein the primer pairs are selected from the following pairs of nucleic acid sequences:
      SEQ ID NO:1 and SEQ ID NO:2;
      SEQ ID NO:5 and SEQ ID NO:6;
      SEQ ID NO:9 and SEQ ID NO:10;
      SEQ ID NO:13 and SEQ ID NO:14;
      SEQ ID NO:17 and SEQ ID NO:18;
      SEQ ID NO:21 and SEQ ID NO:22;
      SEQ ID NO:25 and SEQ ID NO:26;
      SEQ ID NO:29 and SEQ ID NO:30;
      SEQ ID NO:33 and SEQ ID NO:34;
      SEQ ID NO:37 and SEQ ID NO:38;
      SEQ ID NO:41 and SEQ ID NO:42;
      SEQ ID NO:45 and SEQ ID NO:46;
      SEQ ID NO:49 and SEQ ID NO:50;
      SEQ ID NO:53 and SEQ ID NO:54;
      SEQ ID NO:57 and SEQ ID NO:58;
      SEQ ID NO:61 and SEQ ID NO:62;
      SEQ ID NO:65 and SEQ ID NO:66;
      SEQ ID NO:69 and SEQ ID NO:70;
      SEQ ID NO:73 and SEQ ID NO:74;
      SEQ ID NO:77 and SEQ ID NO:78;
      SEQ ID NO:81 and SEQ ID NO:82;
      SEQ ID NO:85 and SEQ ID NO:86;
      SEQ ID NO:89 and SEQ ID NO:90;
      SEQ ID NO:93 and SEQ ID NO:94;
      SEQ ID NO:97 and SEQ ID NO:98;
      SEQ ID NO:101 and SEQ ID NO:102;
      SEQ ID NO:105 and SEQ ID NO:106;
      SEQ ID NO:109 and SEQ ID NO:110;
      SEQ ID NO:113 and SEQ ID NO:114;
      SEQ ID NO:117 and SEQ ID NO:118;
      SEQ ID NO:121 and SEQ ID NO:122;
      SEQ ID NO:125 and SEQ ID NO:126;
      SEQ ID NO:129 and SEQ ID NO:130;
      SEQ ID NO:133 and SEQ ID NO:134;
      SEQ ID NO:137 and SEQ ID NO: 138;
      SEQ ID NO:141 and SEQ ID NO:142;
      SEQ ID NO:145 and SEQ ID NO:146;
      SEQ ID NO:149 and SEQ ID NO:150;
      SEQ ID NO:153 and SEQ ID NO:154;
      SEQ ID NO:157 and SEQ ID NO:158;
      SEQ ID NO:161 and SEQ ID NO:162;
      SEQ ID NO:165 and SEQ ID NO:166;
      SEQ ID NO:169 and SEQ ID NO:170;
      SEQ ID NO:173 and SEQ ID NO:174;
      SEQ ID NO:177 and SEQ ID NO:178;
      SEQ ID NO:181 and SEQ ID NO:182;
      SEQ ID NO:185 and SEQ ID NO:186;
      SEQ ID NO:189 and SEQ ID NO:190;
      SEQ ID NO:193 and SEQ ID NO:194;
      SEQ ID NO:197 and SEQ ID NO:198;
      SEQ ID NO:201 and SEQ ID NO:202;
      SEQ ID NO:205 and SEQ ID NO:206;
      SEQ ID NO:209 and SEQ ID NO:210;
      SEQ ID NO:213 and SEQ ID NO:214;
      SEQ ID NO:217 and SEQ ID NO:218;
      SEQ ID NO:221 and SEQ ID NO:222;
      SEQ ID NO:225 and SEQ ID NO:226;
      SEQ ID NO:229 and SEQ ID NO:230;
      SEQ ID NO:233 and SEQ ID NO:234;
      SEQ ID NO:237 and SEQ ID NO:238;
      SEQ ID NO:241 and SEQ ID NO:242;
      SEQ ID NO:245 and SEQ ID NO:246;
      SEQ ID NO:249 and SEQ ID NO:250;
      SEQ ID NO:253 and SEQ ID NO:254;
      SEQ ID NO:257 and SEQ ID NO:258;
      SEQ ID NO:261 and SEQ ID NO:262;
      SEQ ID NO:265 and SEQ ID NO:266;
      SEQ ID NO:269 and SEQ ID NO:270;
      SEQ ID NO:273 and SEQ ID NO:274;
      SEQ ID NO:277 and SEQ ID NO:278;
      SEQ ID NO:281 and SEQ ID NO:282; or
      SEQ ID NO:285 and SEQ ID NO:286;
   (iii) a nucleic acid sequence in (i) or (ii) wherein T can also be U;
   (iv) a nucleic acid sequence complementary to (i), (ii) or (iii);
   (v) a nucleic acid sequence that has substantial sequence homology to (i), (ii), (iii) or (iv); or
   (vi) a fragment of (i) to (v).
(b) providing transcription indicators from a test sample;
(c) allowing the transcription indicators to hybridize with said two or more nucleic acid molecules; and
(d) detecting hybridization of said transcription indicators with said two or more nucleic acid molecules, wherein hybridization is indicative of the expression of the genes.

In a further embodiment of the present invention, at least two different nucleic acid molecules of the invention, at least 10 different nucleic acid molecules of the invention, at least 20 different nucleic acid molecules of the invention, at least 30 different nucleic acid molecules of the invention, at least 40 different nucleic acid molecules of the invention, at least 50 different nucleic acid molecules of the invention, at least 60 different nucleic acid molecules of the invention, at least 70 different nucleic acid molecules of the invention or at least 72 different nucleic acid molecules of the invention are used in the methods of the invention.

In another embodiment of the invention, control nucleic acid molecules, particularly expression level controls, are used in the methods of the invention.

### (A) Transcription Indicators

Transcription of genes into RNA is a critical step in gene expression. Therefore, gene expression can be monitored by monitoring various transcription indicators. There are a variety of techniques known in the art to analyze and quantify gene transcription. In an embodiment of the present invention gene expression is detected by monitoring or detecting the hybridization of transcription indicators from a test sample with the two or more nucleic acid molecules of the present invention. In an embodiment, gene expression is detected using reverse transcription. For example, RNA is extracted from a test sample using techniques known in the art. cDNA is then synthesized using known techniques, such as using either oligo(dT) or random primers. Gene expression is then detected using the said cDNA by allowing the cDNA to hybridize to the one or more nucleic acid molecules, then detecting the amount of hybridization of said cDNA with the one or more nucleic acid molecules.

One of skill in the art will appreciate that it is desirable to have transcription indicators from a test sample that contain suitable nucleic samples having target nucleic acid sequences that reflect the transcripts of interest. Therefore, suitable nucleic acid samples from the test sample may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable transcription indicators include, but are not limited to, transcripts of the gene or genes, cDNA reverse transcribed from the transcript, cRNA transcribed from the cDNA, DNA amplified from the genes, RNA transcribed from amplified DNA, and the like. In an embodiment the transcription indicator is cDNA.

Transcripts, as used herein, may include, but are not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

The term “test sample” refers to one or more cells, cell lines, tissues or organisms, or portions or homogenates thereof which contain transcription indicators. In one embodiment, the test sample is from a subject. In another embodiment, the test sample is from a human. In a further embodiment, the test sample is from an animal, such as a laboratory animal useful to study drug effects, such as a rodent, including a mouse or rat. In an embodiment of the present invention, the test sample is a homogenate of cells or tissues or other biological samples. For example, such sample can be a total RNA preparation of a biological sample or such a nucleic acid sample can be the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with a poly (dT) column contains RNA molecules with poly (A) tails. Those polyA+ RNA molecules could be mature mRNA, RNA processing intermediates, nascent transcripts or degradation intermediates. For use in studying the impact of a compound or drug on gene expression, the test sample is obtained from a source that has been exposed to that compound or drug.

In an embodiment of the present invention, the test sample is a clinical sample which is a sample derived from a patient. Typical clinical samples include, but are not limited to, sputum, blood, blood cells (e.g. white blood cells), tissue or fine needle biopsy samples, urine, peritoneal fluid and pleural fluid, or cells therefrom. In another embodiment of the present invention, the test sample is derived from a cell culture containing specific cell lines, for example, HepG2, Caco-2 or HEK 293.

One skilled in the art will appreciate that one can inhibit or destroy RNAse present in any sample before they are used in the methods of the invention. Methods of inhibiting or destroying nucleases, including RNAse, are well known in the art. For example, chaotropic agents may be used to inhibit nucleases or, alternatively, heat treatment followed by proteinase treatment may be used.

Methods of isolating total mRNA are also well known to those skilled in the art. For example, see Chapter 3 of Laboratory Techniques in Biochemistry and Molecular Biology: Hybridization with Nucleic Acid Probes, Part I: Theory and Nucleic Acid Preparation, Tijssen, ed. Elsevier Press (1993); Sambrook et al., Molecular Cloning: A Laboratory Manual (2nd ed.), Vols. 1-3, Cold Spring Harbour Laboratory (1989); or Current Protocols in Molecular Biology, F. Ausubel et al., ed. Greene Publishing and Wiley-Interscience, New York (1987). In an embodiment, the total RNA is isolated from a given test sample, for example, using TRIzol reagent (Cat. No. 15596-018, Invitrogen Life Technologies) according to the manufacturer's instructions.

In embodiments of the present invention, the transcription indicator, whether it be cDNA or mRNA, may need to be amplified prior to performing the hybridization assay. Methods for amplification, including “quantitative amplification” are well known to those skilled in the art.

In an embodiment the transcription indicator is labeled with a detectable label. The term “label” refers to any detectable moiety. A label may be used to distinguish a particular nucleic acid from others that are unlabeled, or labeled differently, or the label may be used to enhance detection.

Methods for labeling nucleic acids are well known to those skilled in the art. In an embodiment of the invention, the label is simultaneously incorporated during an amplification step in the preparation of the transcription indicators. Thus for example, PCR with labeled primers or labeled nucleotides (for example fluorescein-labeled UTP and/or CTP) will provide a labeled amplification product. Alternatively, a label may be added directly to the original nucleic acid sample or to the amplification product after the amplification is completed using methods known to those skilled in the art (for example nick translation and end-labeling).

Detectable labels that are suitable for use in the methods of the present invention include those that are detectable by spectroscopic, photochemical, biochemical, immunochemical, electrical, optical or other means. Some examples of useful labels include biotin staining with labeled streptavidin conjugate, magnetic beads, fluorescent dyes (e.g. fluorescein, rhodamine, green fluorescent protein and the like), radiolabels (e.g. 3H, 32P, 14C, 25S or 125I), enzymes (e.g. horseradish peroxidase, alkaline phosphatase and others commonly used in ELISA) and colorimetric labels such as colloidal gold or colored glass or plastic (e.g. polystyrene, polypropylene, latex and the like) beads. Patents teaching the use of such labels include U.S. Pat. Nos. 3,817,837, 3,850,752, 3,939,350, 3,996,345, 4,277,437, 4,275,149 and 4,366,241, the contents of all of which are incorporated herein by reference.

### (B) Assay Format

The method of detecting gene expression can be performed using any hybridization assay, including solution and solid phase. Typically a set containing two or more nucleic acid molecules of the invention are put together in a common container or on a common object. These may be on an array (such as the arrays disclosed herein) or in a kit together. They are typically separated, either spatially on a solid support such as an array, or in separate vessels, such as vials, tubes or wells in a microwell plate.

In an embodiment of the present invention, the method of detecting gene expression is performed in an array format, such as a microarray. One of skill in the art will appreciate that an enormous number of array designs are suitable for the practice of this invention. The array will typically include a number of nucleic acid molecules or probes that specifically hybridize to the sequences of the gene of interest. In addition, in an embodiment, the array will include one or more control nucleic acid molecules or probes. The control probes may be, for example, expression level controls (e.g. positive controls and background negative controls).

Transcription indicators (targets) from a test sample that have been subjected to particular stringency conditions hybridize to the nucleic acid molecules (probes) on the array. One of skill in the art will appreciate that hybridization conditions may be selected to provide any degree of stringency. In an embodiment, hybridization is performed at low stringency [15-18 hrs at 37° C. in 500 mM sodium phosphate pH 6.0, 1% SDS, 1% BSA, 1 mM EDTA] to ensure hybridization and then subsequent washes are performed at higher stringency [0.1×SSC; 0.1% SDS then 0.1×SSC then water] to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test nucleic acid sequences with hybridization to the various controls that can be present (e.g., expression level controls (positive and negative), etc.).

The nucleic acids that do not form hybrid duplexes are washed away leaving the hybridized nucleic acids to be detected, typically through detection of an attached detectable label. After hybridization, the arrays are inserted into a scanner that can detect patterns of hybridization. These hybridization patterns are captured by detecting the labeled transcription indicator now attached to the array, for e.g., if the transcription indicator is fluorescently labeled, the hybridization data are collected as light emitted from the labeled groups. Comparison of the absolute intensities of an array exposed to nucleic acids from a test sample with intensities produced from the various control samples provides a measure of the relative expression of the nucleic acids represented by each of the probes.

If the transcription indicator, for example cDNA, is fluorescently labeled, the fluorescence is detected and acquired using a confocal fluorescence scanner, for example, a GSI Lumonics ScanArray Lite Microarray Analysis System, and the fluorescence intensity analyzed with specific quantitation and data processing software on a dedicated computer, for example, QuantArray and GeneLinker Gold. In an embodiment, the intensity of fluorescence increases with increased gene expression. If the transcription indicator, for example cDNA, is radiolabeled, then detection can be carried out using an RU image scanner and such, and the intensity of the radiation can be analyzed with a computer. In an embodiment, the intensity of the radiation increases with increased gene expression.

In further embodiments of the present invention, the methods of the invention further comprise (a) generating a set of expression data from the detection of the amount of hybridization; (b) storing the data in a database; and (c) performing comparative analysis on the set of expression data, thereby analyzing gene expression.

The gene expression data generated using the materials and methods of the invention can be contained in a database. Accordingly, the present invention also relates to a computer system comprising (a) a database containing information identifying the expression level of two or more genes; and b) a user interface to view the information, wherein the information identifying the expression level of two or more genes is obtained using the method according to the invention.

In embodiments of the invention, the method of detecting gene expression in a test sample is performed once or more, over a set period of time and at specified intervals, to monitor and compare the levels of gene expression over that period of time.

### (VI) Drug Screening Assays

The materials and methods of the invention can been used in drug screening analysis. For example, a subject is exposed to a chemical compound or a drug, and then gene expression is detected in a test sample from the subject using the methods of the invention. In an embodiment of the invention, gene expression is detected at various time intervals after the subject is exposed to a compound or drug, for example, every 2 hours after exposure over a 24 hour period. In a further embodiment, after (and optionally before) the subject is exposed to the chemical or drug, mRNA is extracted from a test sample from the subject and then cDNA is produced using the extracted mRNA. The cDNA is labeled and allowed to hybridize with the two or more nucleic acid molecules of the invention. The amount of hybridization is detected and compared with the amount of hybridization obtained with the test sample taken either at a different point from the same subject, or taken from a different subject that was treated under the same conditions except that the subject has not been exposed to the compound or drug (i.e. a control sample). By performing this comparison, the effect of the drug or compound on the expression of each of genes (whether it be increased, decreased or the same) in the test sample from the subject is determined.

The term “subject” as used herein includes all members of the animal kingdom including mammals, preferably humans. The methods of the invention can also be used on cells, tissues and cell lines; thus the term “subject” as used herein also includes cells, tissues and cell lines, preferably derived from humans or laboratory animals, such as rodents including mice and rats.

The nucleic acid molecules and methods of the present invention can be used to perform drug-associated gene expression profiling. Such profiling can identify potential modulators of gene expression, of genes encoding cytochrome p450, nuclear X receptors, phase II transferases, and solute carrier family uptake pumps.

Accordingly, the invention includes a method for screening a compound for its effect on the expression of two or more genes, comprising the steps:
(a) providing a transcription indicator from a test sample from a subject exposed to the compound;
(b) providing two or more nucleic acid molecules, wherein the two or more nucleic acid molecules each comprise a nucleic acid sequence selected from:
   (i) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288,
   (ii) a nucleic acid sequence prepared using amplification and primer pairs, wherein the primer pairs are selected from the following pairs of nucleic acid sequences:
      SEQ ID NO:1 and SEQ ID NO:2;
      SEQ ID NO:5 and SEQ ID NO:6;
      SEQ ID NO:9 and SEQ ID NO:10;
      SEQ ID NO:13 and SEQ ID NO:14;
      SEQ ID NO:17 and SEQ ID NO:18;
      SEQ ID NO:21 and SEQ ID NO:22;
      SEQ ID NO:25 and SEQ ID NO:26;
      SEQ ID NO:29 and SEQ ID NO:30;
      SEQ ID NO:33 and SEQ ID NO:34;
      SEQ ID NO:37 and SEQ ID NO:38;
      SEQ ID NO:41 and SEQ ID NO:42;
      SEQ ID NO:45 and SEQ ID NO:46;
      SEQ ID NO:49 and SEQ ID NO:50;
      SEQ ID NO:53 and SEQ ID NO:54;
      SEQ ID NO:57 and SEQ ID NO:58;
      SEQ ID NO:61 and SEQ ID NO:62;
      SEQ ID NO:65 and SEQ ID NO:66;
      SEQ ID NO:69 and SEQ ID NO:70;
      SEQ ID NO:73 and SEQ ID NO:74;
      SEQ ID NO:77 and SEQ ID NO:78;
      SEQ ID NO:81 and SEQ ID NO:82;
      SEQ ID NO:85 and SEQ ID NO:86;
      SEQ ID NO:89 and SEQ ID NO:90;
      SEQ ID NO:93 and SEQ ID NO:94;
      SEQ ID NO:97 and SEQ ID NO:98;
      SEQ ID NO:101 and SEQ ID NO:102;
      SEQ ID NO:105 and SEQ ID NO:106;
      SEQ ID NO:109 and SEQ ID NO:110;
      SEQ ID NO:113 and SEQ ID NO:114;
      SEQ ID NO:117 and SEQ ID NO:118;
      SEQ ID NO:121 and SEQ ID NO:122;
      SEQ ID NO:125 and SEQ ID NO:126;
      SEQ ID NO:129 and SEQ ID NO:130;
      SEQ ID NO:133 and SEQ ID NO:134;
      SEQ ID NO:137 and SEQ ID NO: 138;
      SEQ ID NO:141 and SEQ ID NO:142;
      SEQ ID NO:145 and SEQ ID NO:146;
      SEQ ID NO:149 and SEQ ID NO:150;
      SEQ ID NO:153 and SEQ ID NO:154;
      SEQ ID NO:157 and SEQ ID NO:158;
      SEQ ID NO:161 and SEQ ID NO:162;
      SEQ ID NO:165 and SEQ ID NO:166;
      SEQ ID NO:169 and SEQ ID NO:170;
      SEQ ID NO:173 and SEQ ID NO:174;
      SEQ ID NO:177 and SEQ ID NO:178;
      SEQ ID NO:181 and SEQ ID NO:182;
      SEQ ID NO:185 and SEQ ID NO:186;
      SEQ ID NO:189 and SEQ ID NO:190;
      SEQ ID NO:193 and SEQ ID NO:194;
      SEQ ID NO:197 and SEQ ID NO:198;
      SEQ ID NO:201 and SEQ ID NO:202;
      SEQ ID NO:205 and SEQ ID NO:206;
      SEQ ID NO:209 and SEQ ID NO:210;
      SEQ ID NO:213 and SEQ ID NO:214;
      SEQ ID NO:217 and SEQ ID NO:218;
      SEQ ID NO:221 and SEQ ID NO:222;
      SEQ ID NO:225 and SEQ ID NO:226;
      SEQ ID NO:229 and SEQ ID NO:230;
      SEQ ID NO:233 and SEQ ID NO:234;
      SEQ ID NO:237 and SEQ ID NO:238;
      SEQ ID NO:241 and SEQ ID NO:242;
      SEQ ID NO:245 and SEQ ID NO:246;
      SEQ ID NO:249 and SEQ ID NO:250;
      SEQ ID NO:253 and SEQ ID NO:254;
      SEQ ID NO:257 and SEQ ID NO:258;
      SEQ ID NO:261 and SEQ ID NO:262;
      SEQ ID NO:265 and SEQ ID NO:266;
      SEQ ID NO:269 and SEQ ID NO:270;
      SEQ ID NO:273 and SEQ ID NO:274;
      SEQ ID NO:277 and SEQ ID NO:278;
      SEQ ID NO:281 and SEQ ID NO:282; or
      SEQ ID NO:285 and SEQ ID NO:286;
   (iii) a nucleic acid sequence in (i) or (ii) wherein T can also be U;
   (iv) a nucleic acid sequence complementary to (i), (ii) or (iii);
   (v) a nucleic acid sequence that has substantial sequence homology to (i), (ii), (iii) or (iv); or
   (vi) a fragment of (i) to (v).
(c) allowing said transcription indicator to hybridize with said two or more nucleic acid molecules; and
(d) detecting hybridization of said transcription indicator with said two or more nucleic acid molecules, wherein hybridization is indicative of the expression of the two or more genes.

In further embodiments of the invention, changes in the expression of the genes can be quantitatively or qualitatively determined by comparing the hybridization patterns of treated and untreated samples. In one embodiment, the change in the expression of the genes in a test sample from a subject is compared to a control sample.

The term “control sample” as used herein means a sample from a subject that has been treated under the same conditions as the test subject except that the control sample has not been exposed to one or more compounds, drugs or other conditions that is under investigation. The control can also be a predetermined standard.

The term “compound” as used herein means any agent, including drugs, which may have an effect on gene expression, particularly expression of genes encoding cytochrome p450, nuclear X receptors, phase II transferases, and solute carrier family uptake pumps, and includes, but is not limited to, small inorganic or organic molecules: peptides and proteins and fragments thereof; carbohydrates, and nucleic acid molecules and fragments thereof. The compound may be isolated from a natural source or be synthetic. The term compound also includes mixtures of compounds or agents such as, but not limited to, combinatorial libraries and extracts from an organism.

The term “exposed” as used herein means that the subject has been brought into contact with the compound(s) using any method known in the art. For example, cells lines may be exposed to a compound by adding the compound(s) to the media used for cell storage, growth and/or washing. In a further example, the exposure may be effected by administering the compound(s) to a test subject using any known methods for administration, and the test sample is obtained from the subject, again using any known means.

In a further embodiment of the present invention there is provided a method for screening a compound for its effect on the expression of two or more genes comprising:
(a) preparing a gene expression profile of a test sample from a subject that has been exposed to the compound using the method according to the invention;
(b) preparing a gene expression profile of a control sample using the method according to the invention; and
(c) quantitatively or qualitatively comparing the gene expression profiles from (a) and (b), wherein differential expression profiles in (a) and (b) is indicative of a compound having an effect on the expression of two or more genes

For example, if the expression of the genes is increased compared to the control sample, then the efficacy of the compound is decreased. For example, if the expression of the genes is decreased compared to the control sample, then the efficacy of the compound is increased.

In yet another embodiment of the invention, the expression of the genes in the test and/or control samples is monitored over a set period of time and at specified time intervals to determine the effect of the compound on the expression of the genes over that period of time.

In embodiments of the invention, the methods may be used to identify compounds or agents that stimulate, induce and/or up-regulate the transcription or expression of one or more cytochrome p450 genes, nuclear X receptor genes, phase II transferase genes, or solute carrier family uptake pump genes, or to down-regulate, suppress and/or counteract the transcription or expression of these genes, or that have no effect on transcription or expression of these genes, in a given system. According to the present invention, one can also compare the specificity of a compound's effect by looking at the expression profile of these genes. Typically, more specific compounds will have fewer transcriptional targets. Further, similar sets of results for two different compounds typically indicates a similarity of effects for the two compounds.

The gene expression profile data can be used to design or choose an effective drug or chemical for the treatment of disease, such as cancer. For example, by knowing which genes are modulated in the presence of the drug or compound, one can determine a cell's or patient's predisposition to drug toxicity and/or response to drug treatment

Accordingly the present invention further relates to a method of assessing the toxicity and/or efficacy of a compound in a subject comprising:
(a) preparing a gene expression profile of a test sample from a subject that has been exposed to the compound using the methods of the invention;
(b) preparing a gene expression profile of a control sample using the methods of the invention; and
(c) quantitatively or qualitatively comparing the gene expression profiles from (a) and (b), wherein a difference in the gene expression profiles in (a) and (b) is indicative of the toxicity and/or efficacy of the compound

In an embodiment of the invention, the compound is administered to a subject and gene expression is profiled in a test sample from the subject before and/or after administration of the compounds. Changes in gene expression are indicative of the toxicity and/or efficacy of the compound in the subject.

In a further embodiment, the nucleic acids and methods of the present invention are used to detect potential drug/drug interactions by virtue of their concomitant effect on the expression of cytochrome p450 genes, nuclear X receptor genes, phase II transferase genes, and solute carrier family uptake pump genes. When two or more drugs are administered together, for example in combination therapy, gene expression may be altered. This is particularly relevant if two or more drugs are transported by the same transporter. What might be a non-toxic dose of a drug when administered on its own, may be a toxic dose when that drug is administered along with another drug particularly when both drugs are transported by or substrates for the same transporter. Therefore it is important to determine a drug's effect on gene expression alone, as well as in the presence of one or more other drugs with which it may be co-administered.

Accordingly, in a further embodiment of the present invention there is provided a method for determining a change in gene expression profile for a compound in the presence of one or more different compounds comprising:
(a) preparing a gene expression profile of a test sample from a subject that has been exposed to the compound using the methods of the invention;
(b) preparing a gene expression profile of the test sample from a subject that has been exposed to the compound and one or more different compounds using the methods of the invention; and
(c) quantitatively or qualitatively comparing the gene expression profiles from (a) and (b), wherein differential expression in (a) and (b) indicates that the gene expression profile of the compound changes in the presence of the one or more different compounds.

In an embodiment of the invention, differential gene expression may indicate the presence of drug-drug interactions. If drug-drug interactions are found, then caution would need to be taken when determining effective drug therapies, including dosing, when the drugs are to be present in the body or cell at the same time.

The methods of the present invention may also be used to monitor the changes in the gene expression profile as a function of disease state. For example, a gene expression profile of a test sample from the subject may be obtained at one point in time and again at a later date. Changes in the gene expression profile may be indicative of changes in disease state, treatment response or treatment toxicity.

Another embodiment of the invention is the use of the gene expression information for population profiling. For example, gene expression profile data can be used to select or stratify clinical trial participants into non-responder and responder groups to a particular drug or chemical before initiation of the clinical trial.

### (VII) Databases

The present invention also includes relational databases containing gene expression profiles in various tissue samples and/or cell lines, particularly cytochrome p450 genes, nuclear X receptor genes, phase II transferase genes and solute carrier family uptake pump genes. The database may also contain sequence information as well as descriptive information about the gene associated with the sequence information, the clinical status of the test sample and/or its source. Methods of configuring and constructing such databases are known to those skilled in the art (see for example, Akerblom et al. U.S. Pat. No. 5,953,727).

The databases of the invention may be used in methods to identify the gene expression level in a test sample by comparing the expression level at least one of the genes in the test sample with the level of expression of the gene(s) in the database. Such methods may be used to assess the physiological state of a given test sample by comparing the level of expression of a gene(s) in the sample with that found in samples from normal, untreated samples or samples treated with other agents.

### (VIII) Kits

The present invention further includes kits combining, in different combinations, nucleic acid arrays or microarrays, reagents for use with the arrays, signal detection and array-processing instruments, gene expression databases and analysis and database management software described above. The kits may be used, for example, to predict or model the toxic or therapeutic response of a test compound, to monitor the progression of disease states, to identify genes that show promise as new drug targets and to screen known and newly designed drugs as discussed above.

The databases packaged with the kits are a compilation of expression patterns from human or laboratory animal genes, particularly including the genes targeted by the present methods and arrays. Data is collected from a repository of both normal and diseased animal tissues and provides reproducible, quantitative results, i.e., the degree to which a gene is up-regulated or down-regulated under a given condition.

The kits may used in the pharmaceutical industry, where the need for early drug testing is strong due to the high costs associated with drug development but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with microarrays is discussed in Balaban et al., U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

### (IX) Methods of Conducting Drug Discovery Businesses

Yet another aspect of the present invention provides a method of conducting a target discovery business comprising:
(a) providing one or more assay systems for identifying agents by their ability to modulate gene expression of cytochrome p450 genes, nuclear X receptor genes, phase II transferase genes, and solute carrier family uptake pump genes, said assay systems using a method of the invention;
(b) (optionally) conducting therapeutic profiling of agents identified in step (a) for efficacy and toxicity in animals; and
(c) licensing, to a third party, the rights for further drug development and/or sales or agents identified in step (a), or analogs thereof.

By assay systems, it is meant, the equipment, reagents and methods involved in conducting a screen of compounds for the ability to modulate gene expression using the method of the invention.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present invention:

### EXAMPLES

### Example 1

### Sets of Primers and Resulting PCR Products for Each Cytochrome P450 (CYP), Nuclear X Receptor (NXR), Solute Carrier Family Member (Nucleoside, Anion, Cation Transporters) [SCL] and Transferase (SULT; UGT] Gene

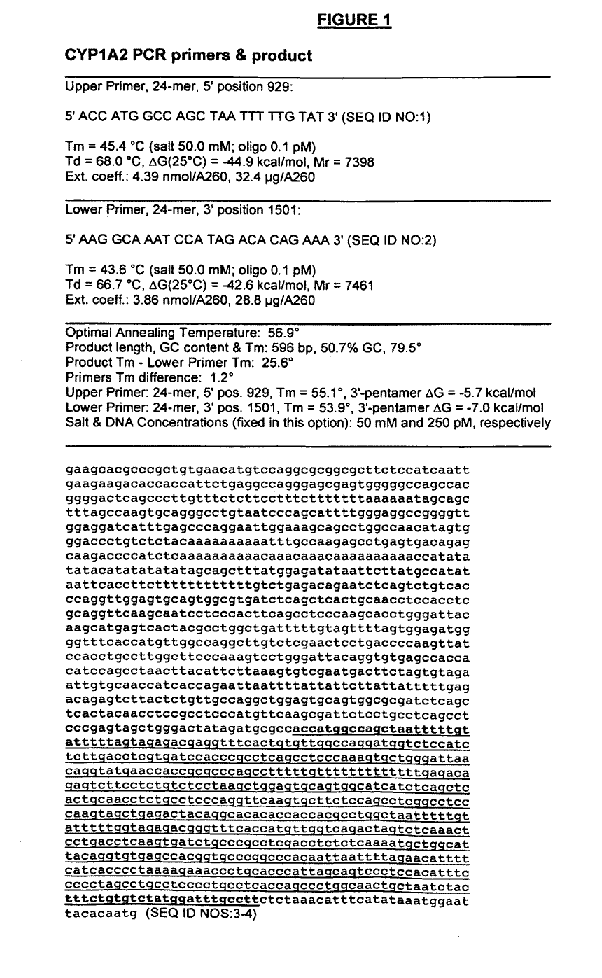

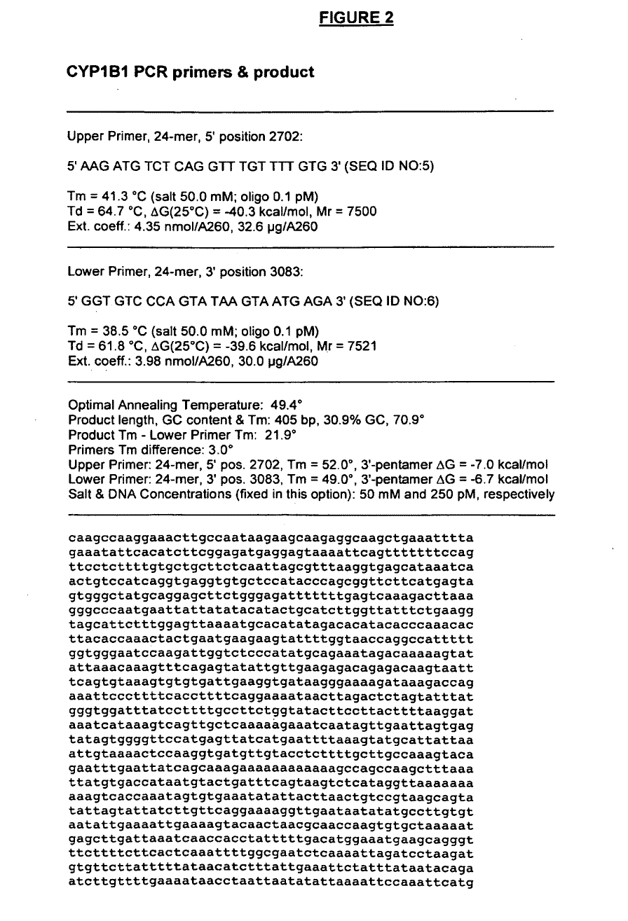

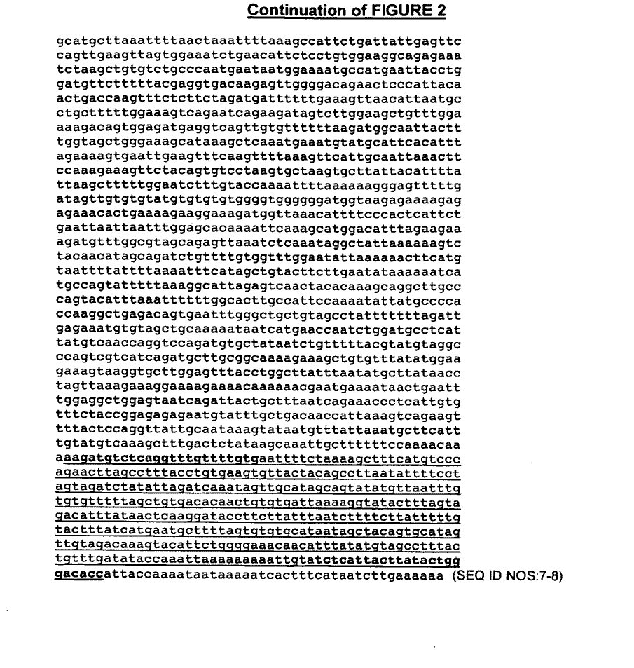

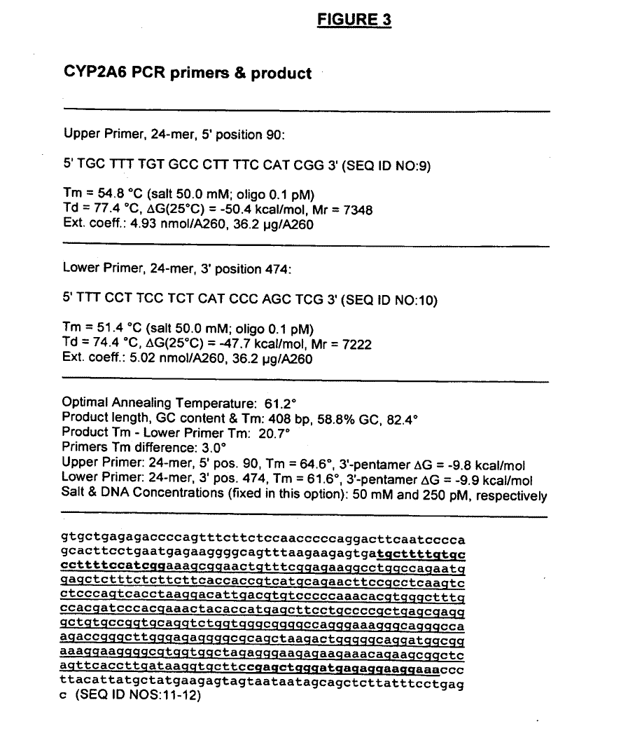

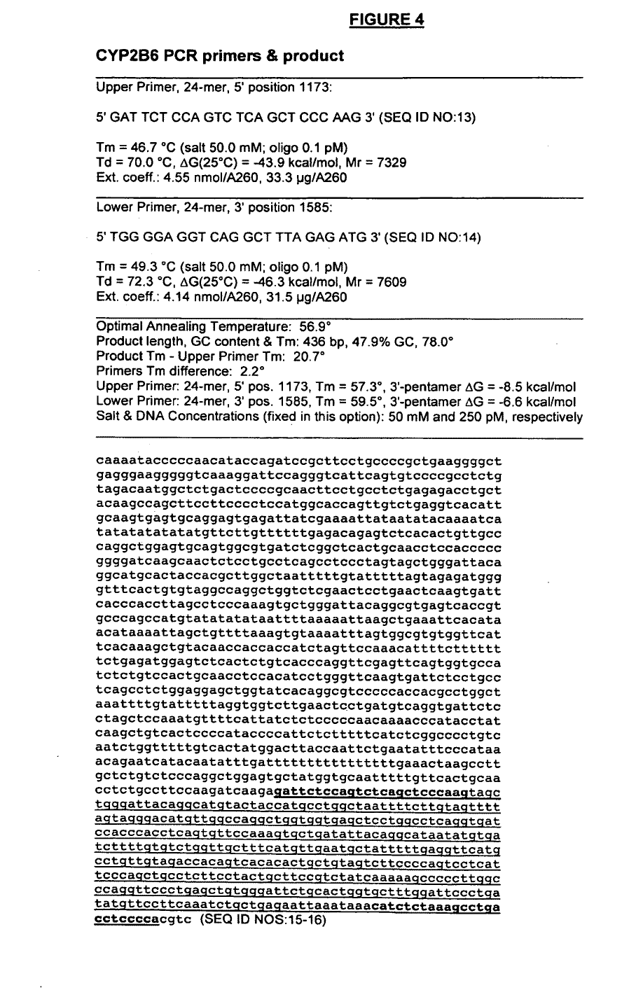

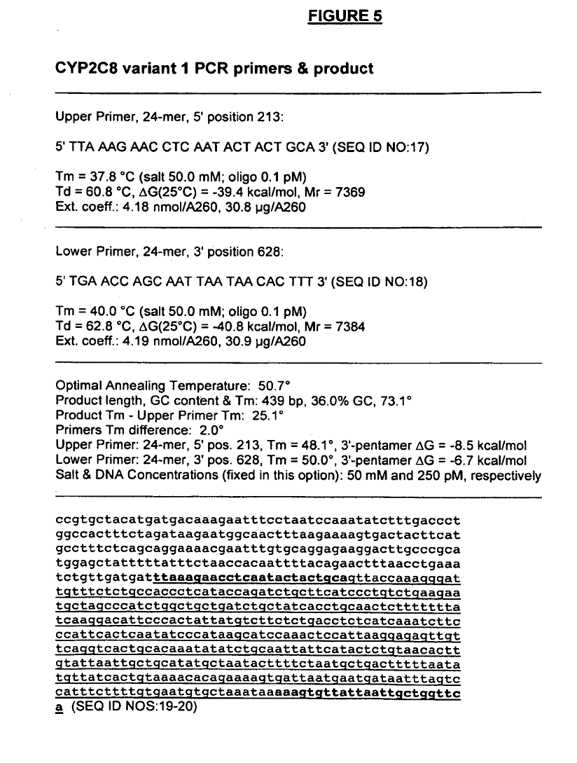

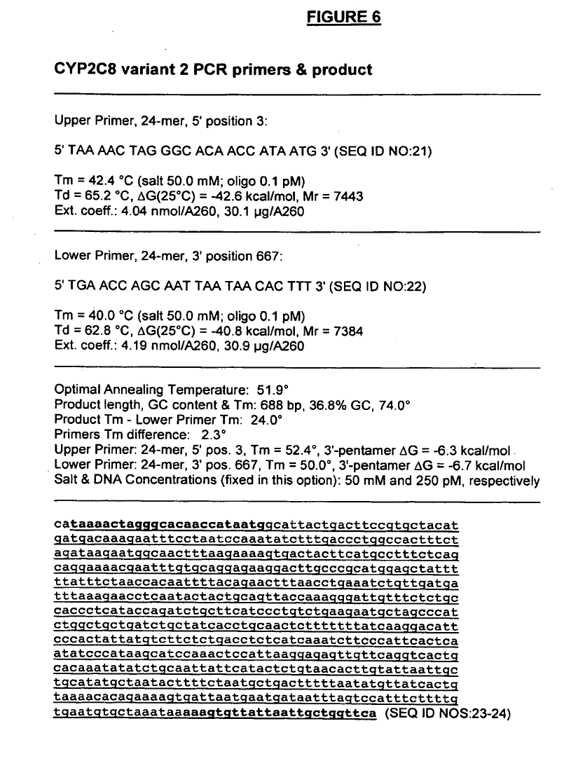

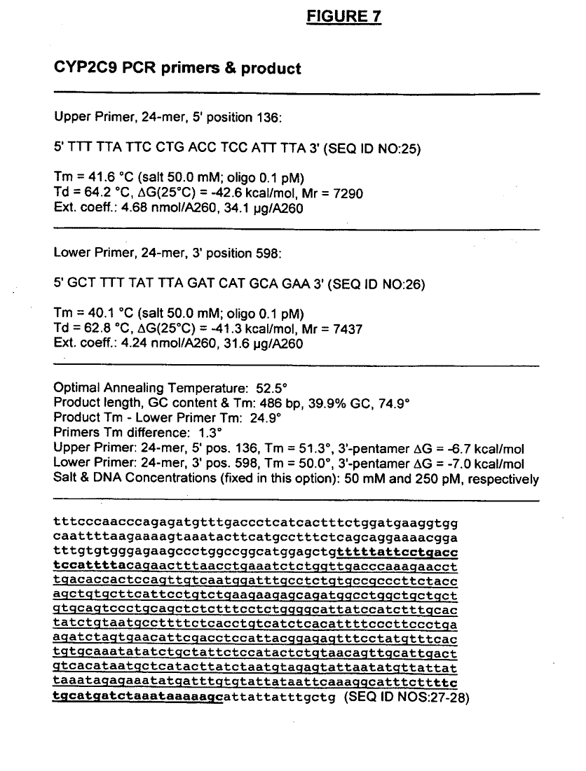

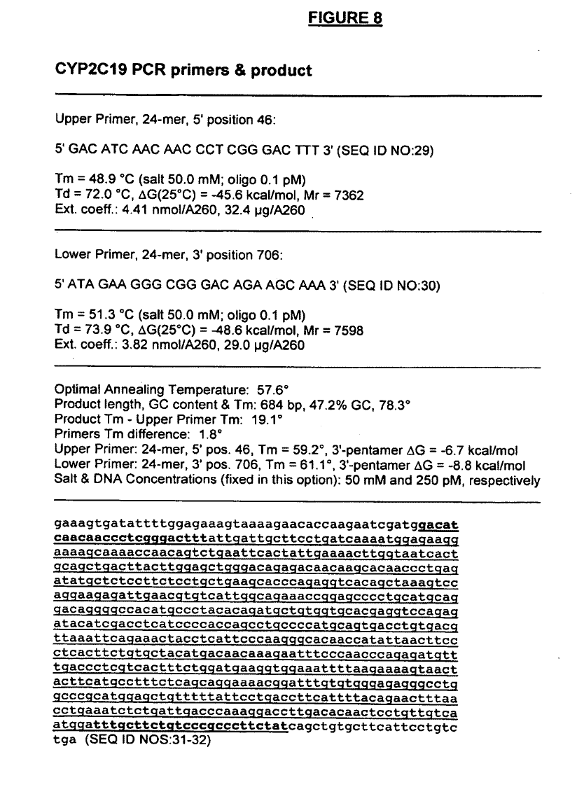

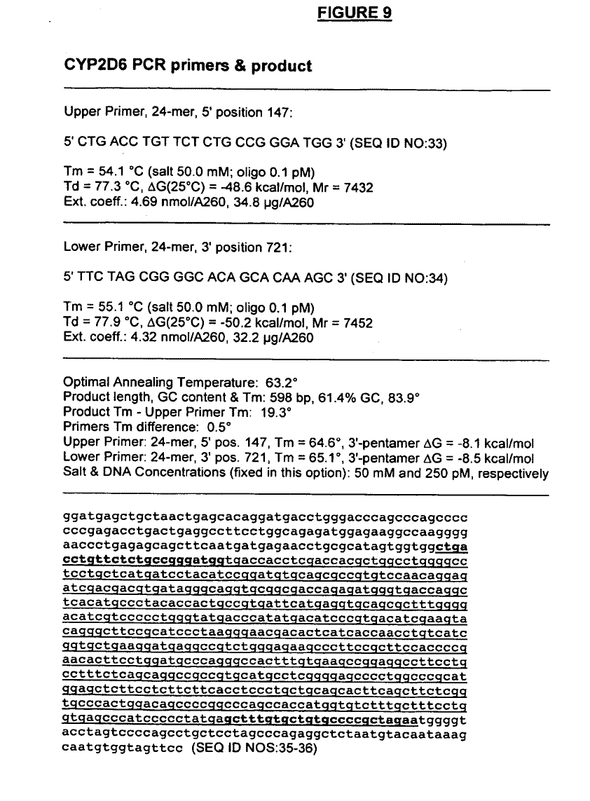

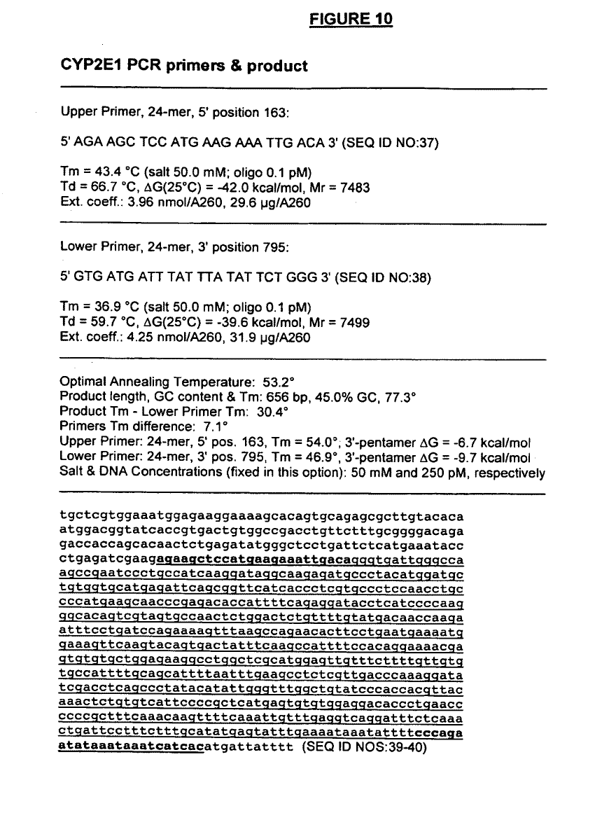

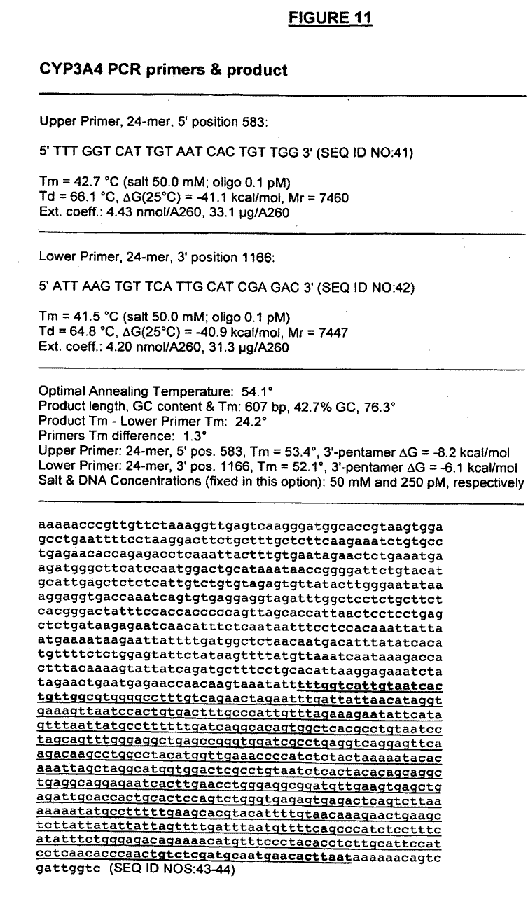

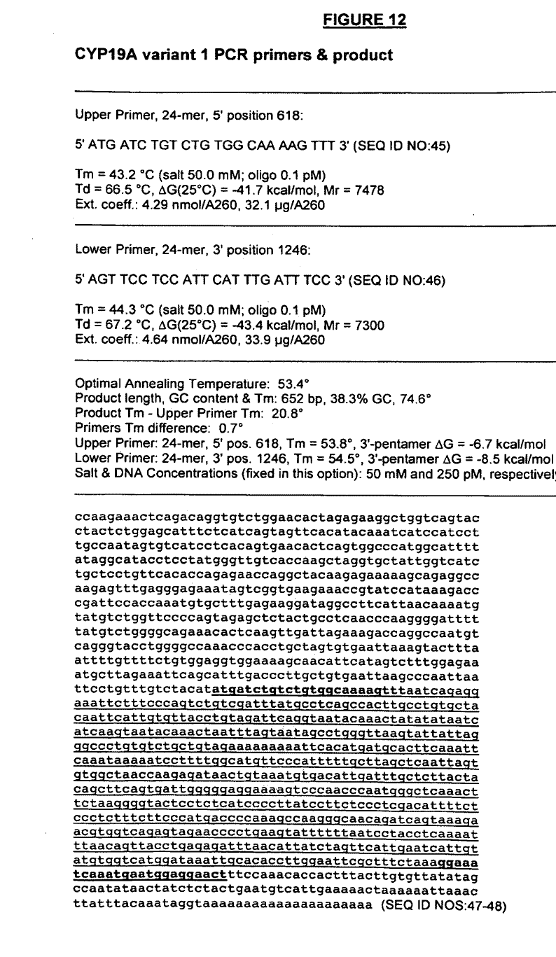

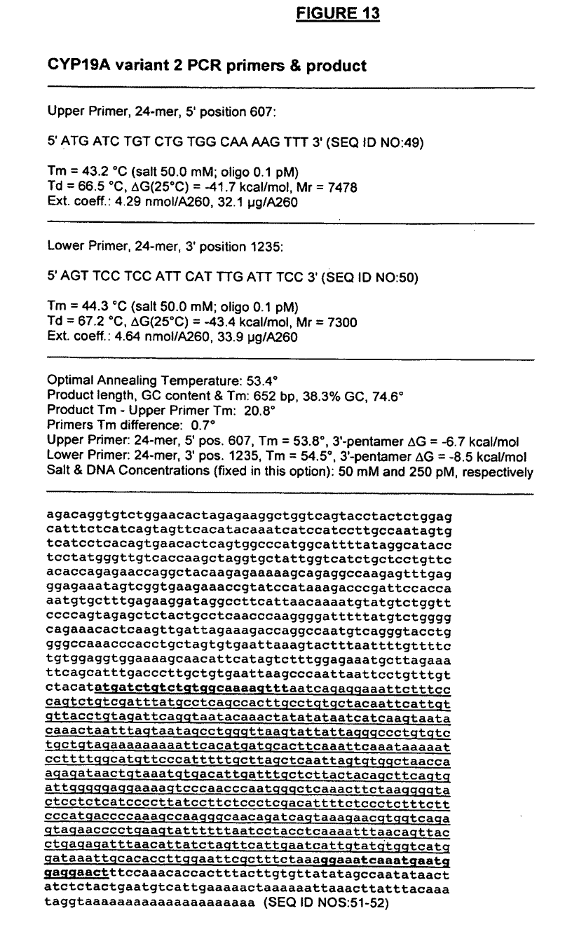

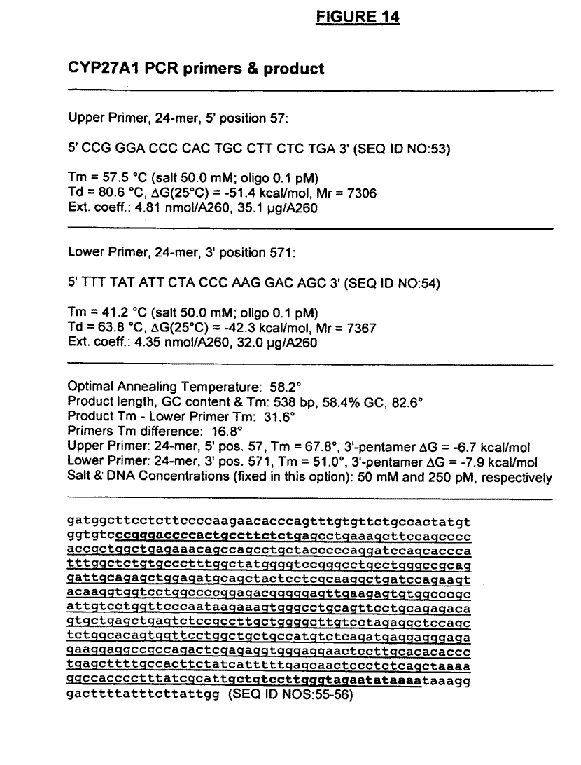

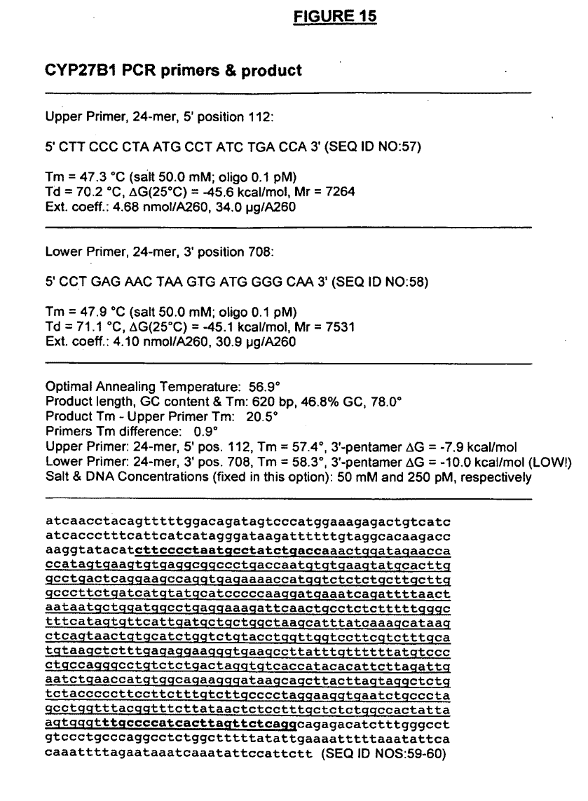

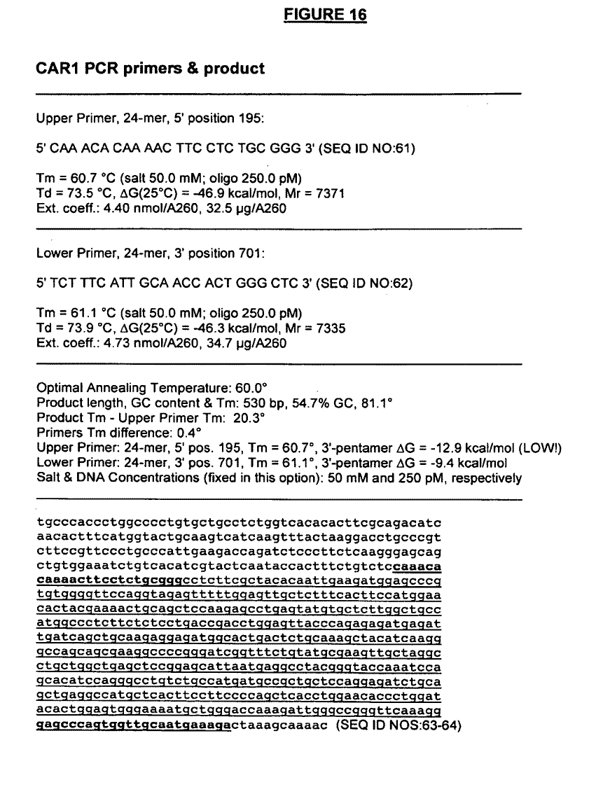

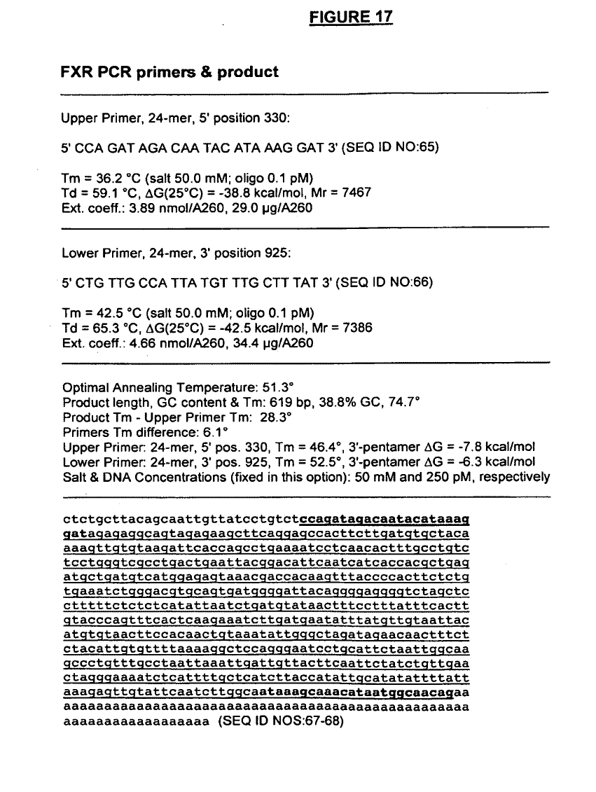

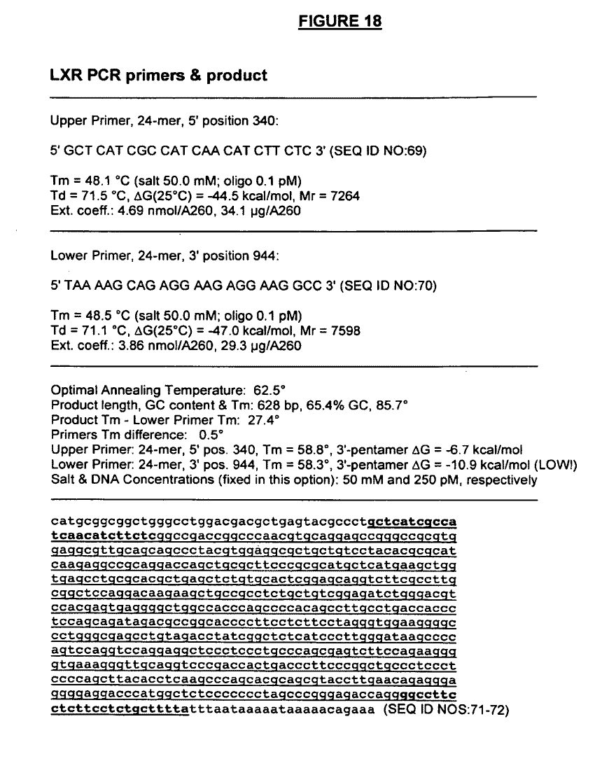

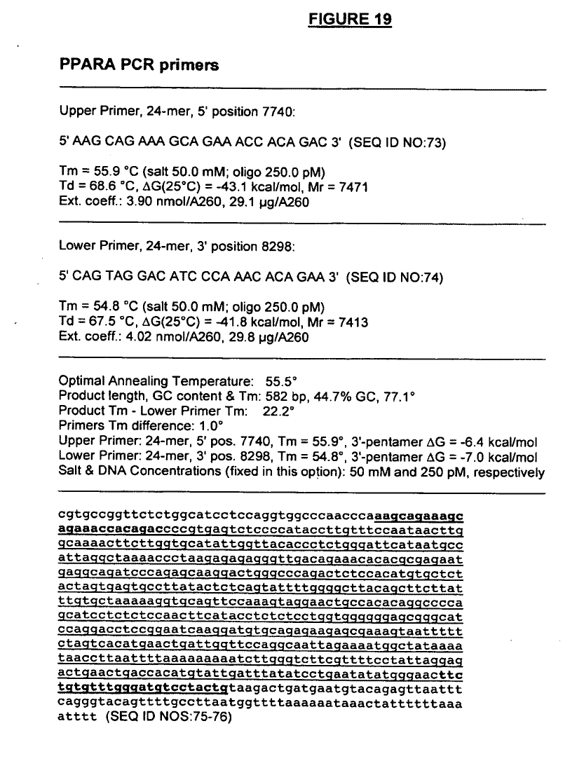

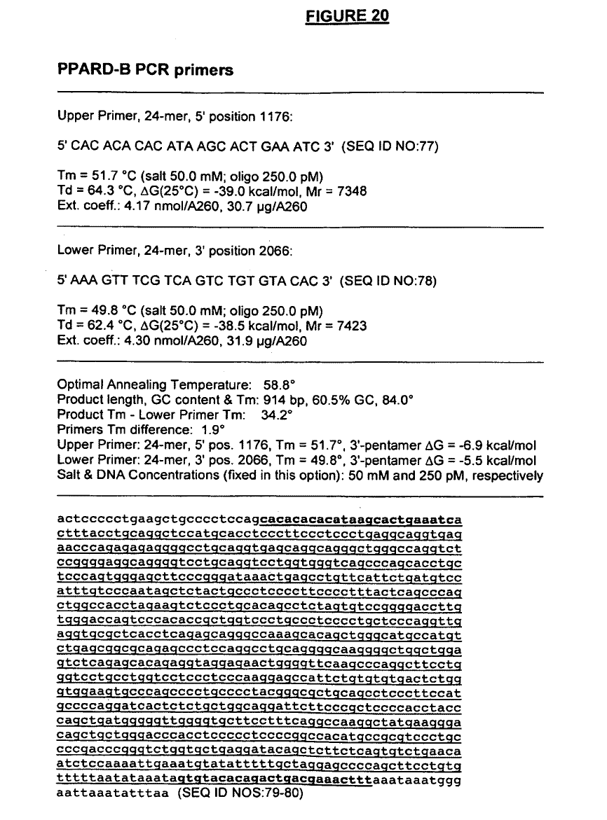

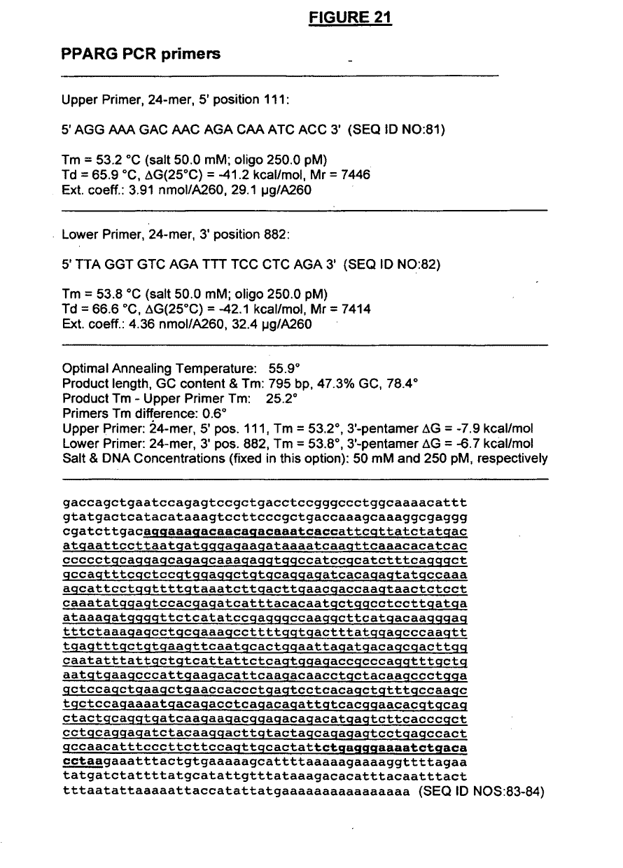

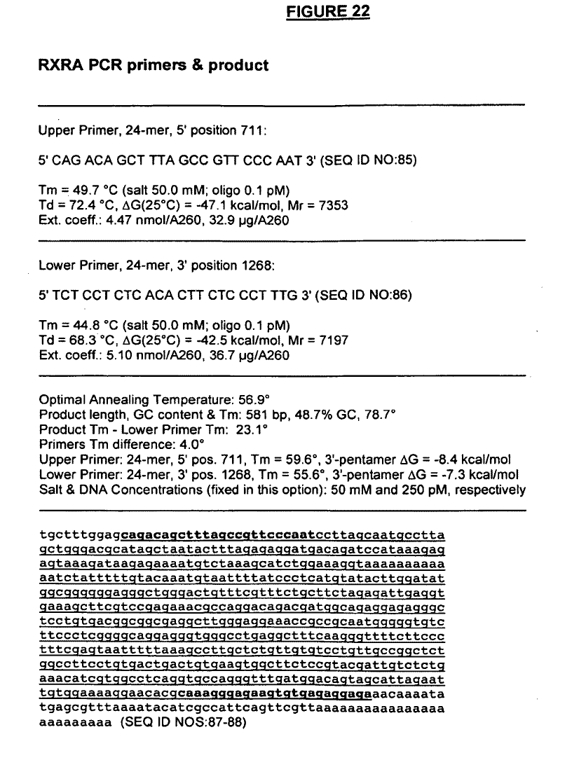

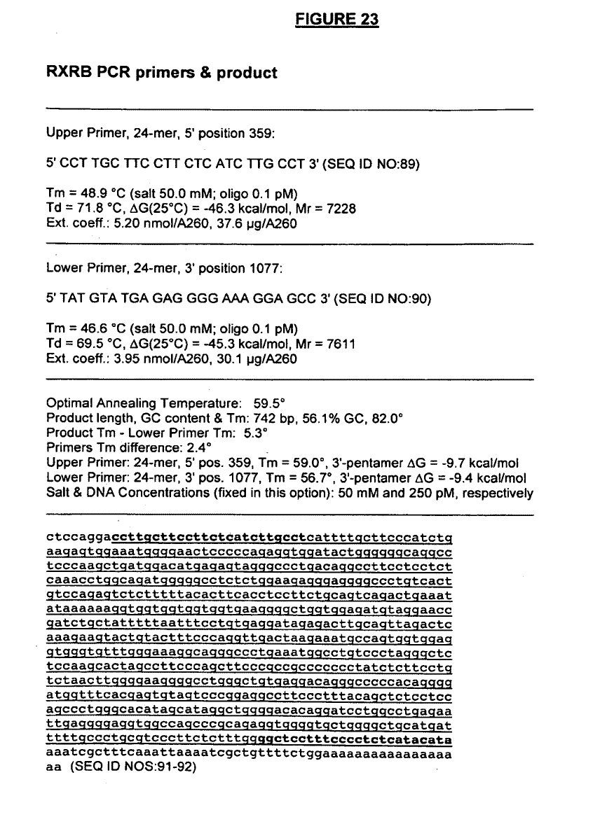

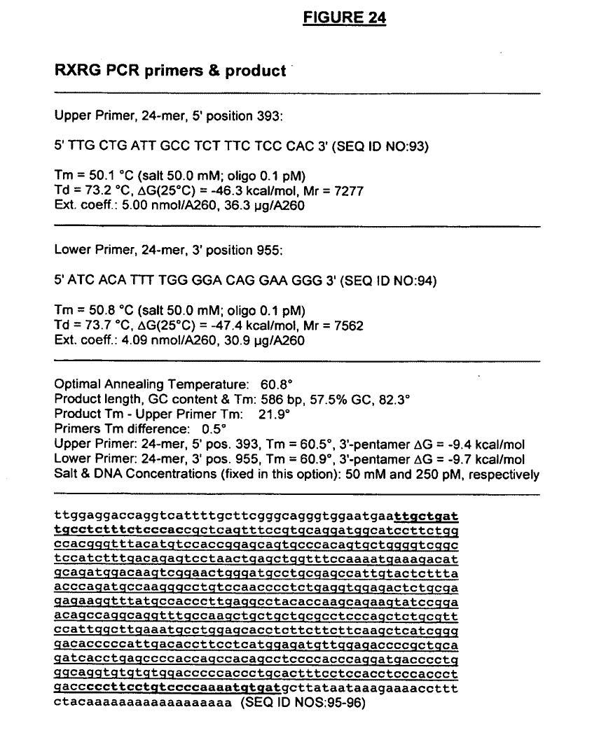

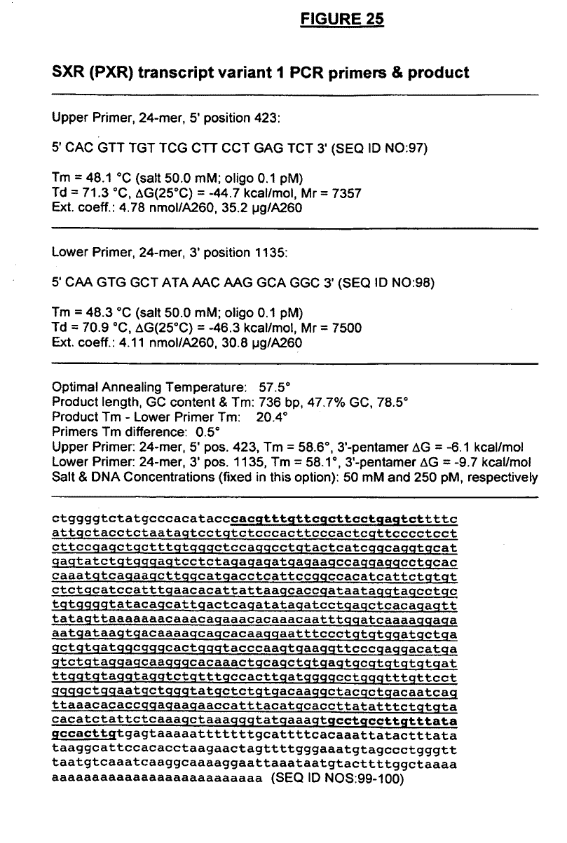

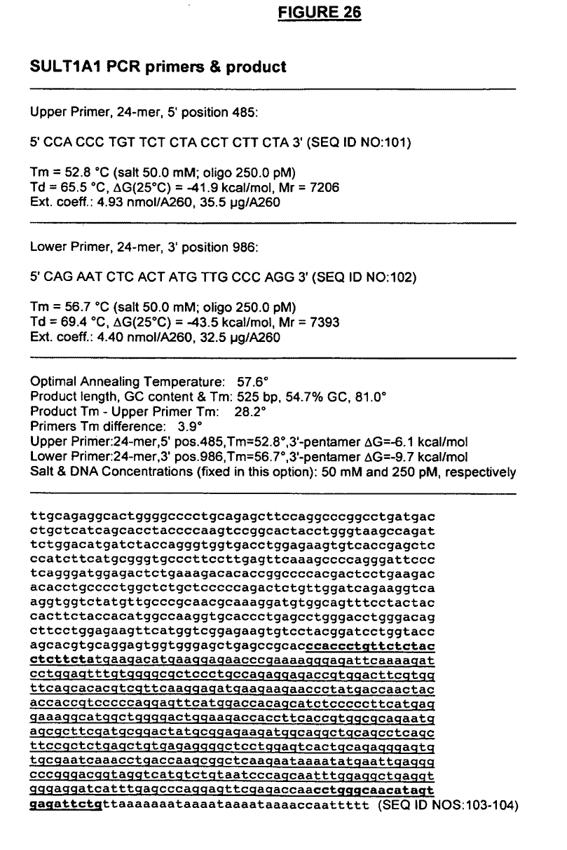

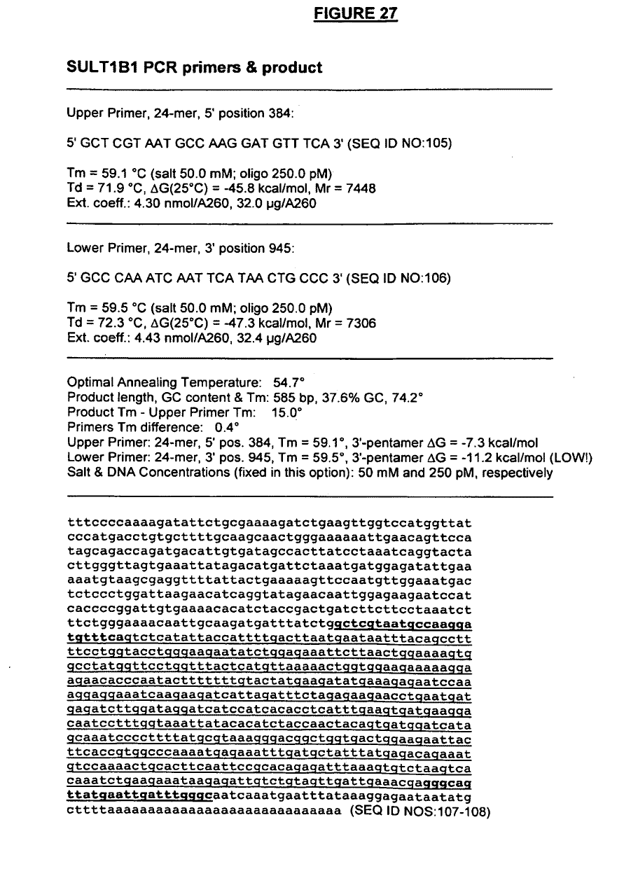

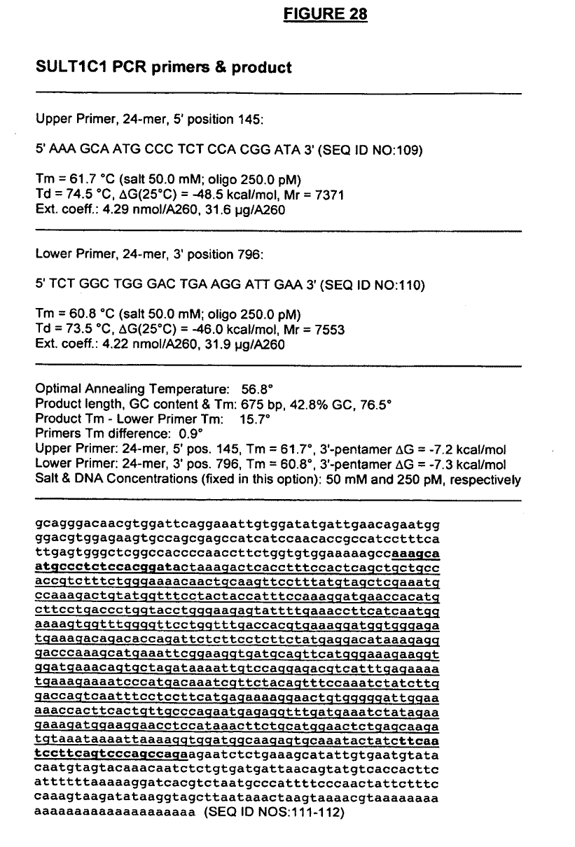

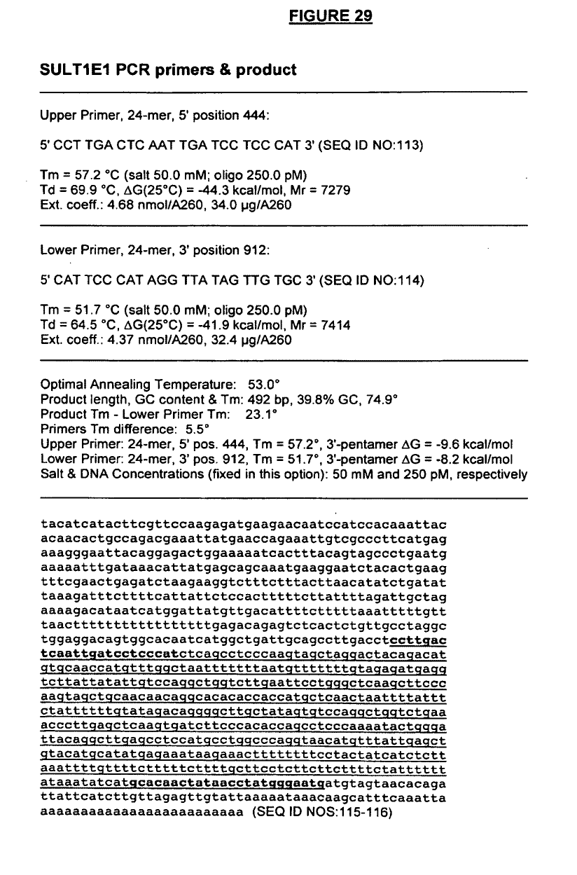

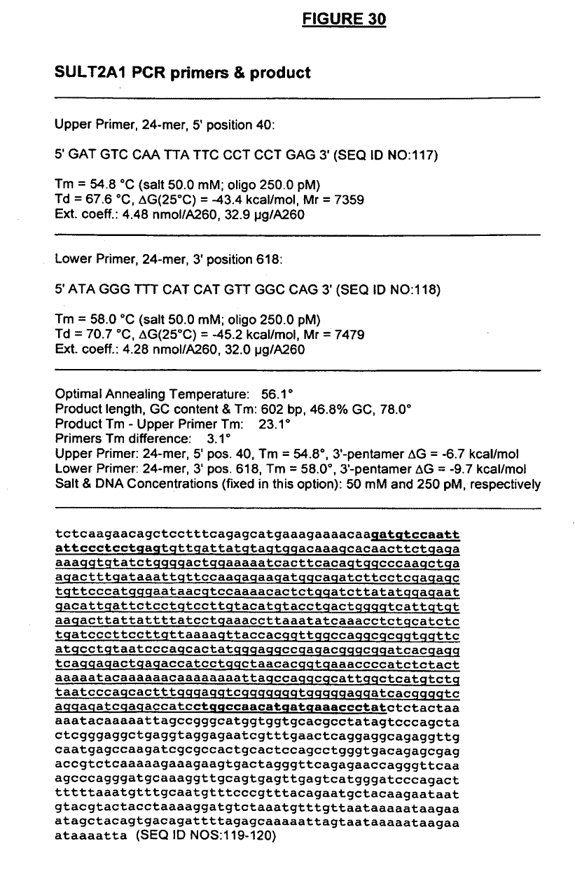

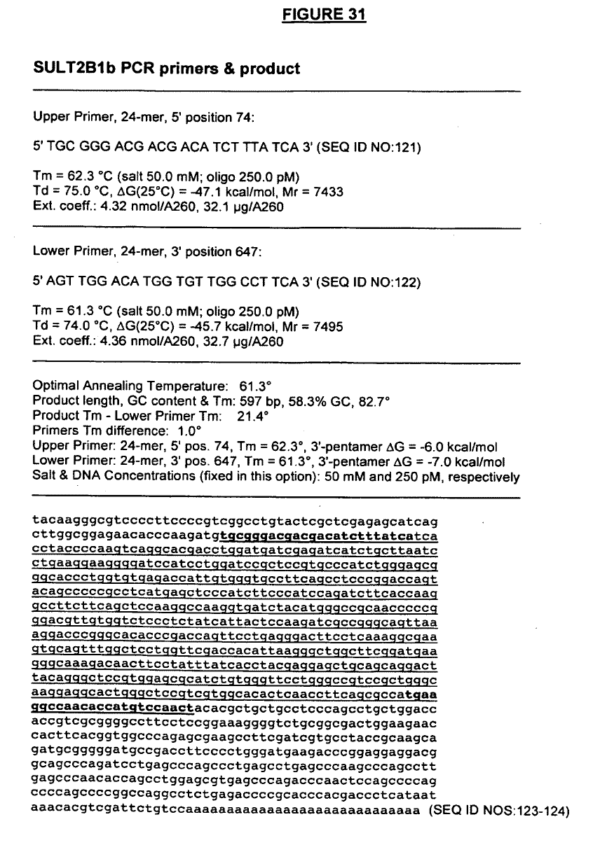

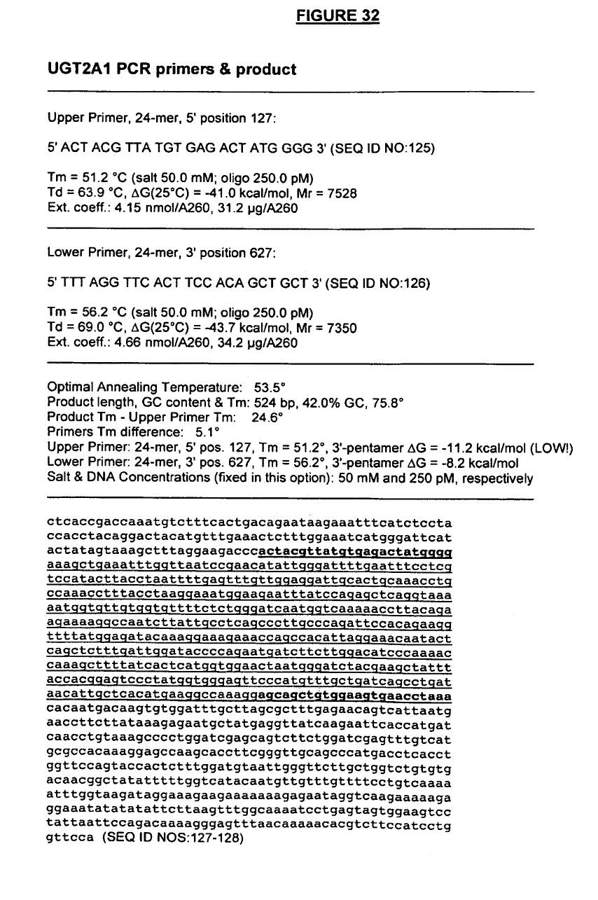

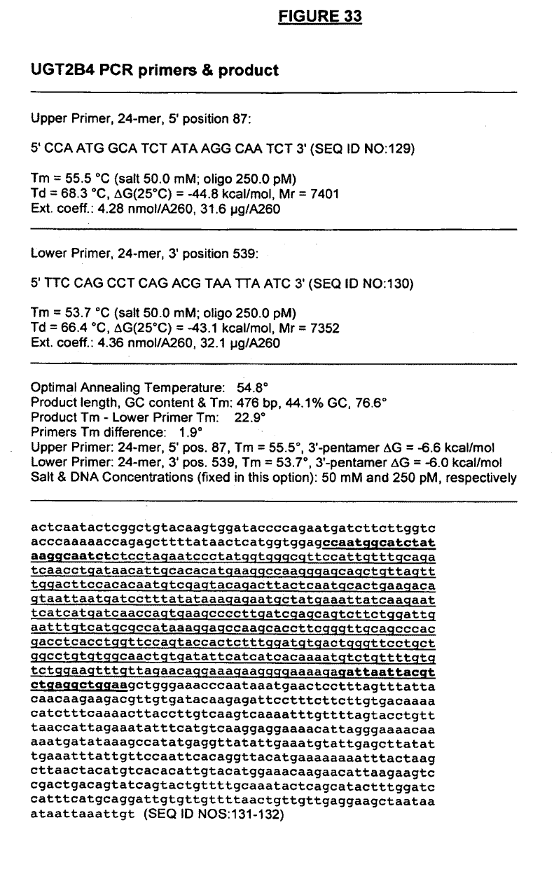

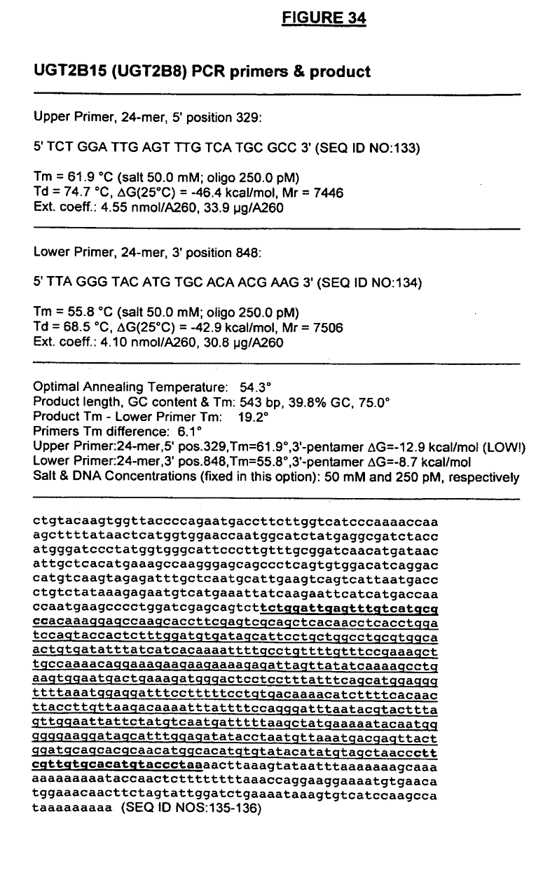

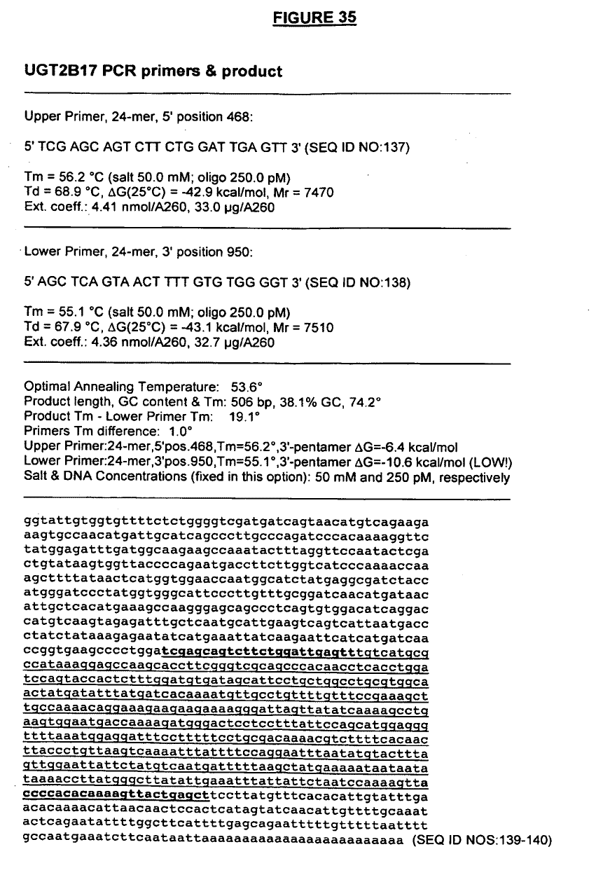

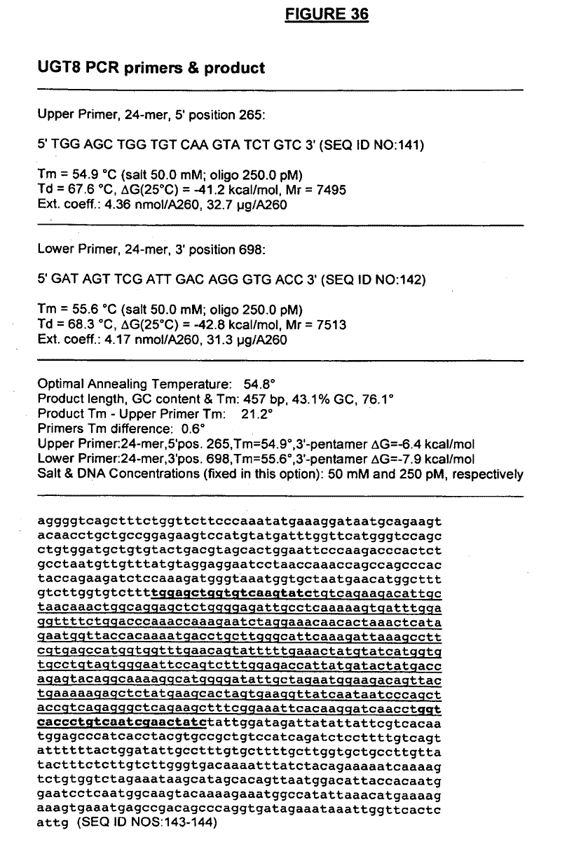

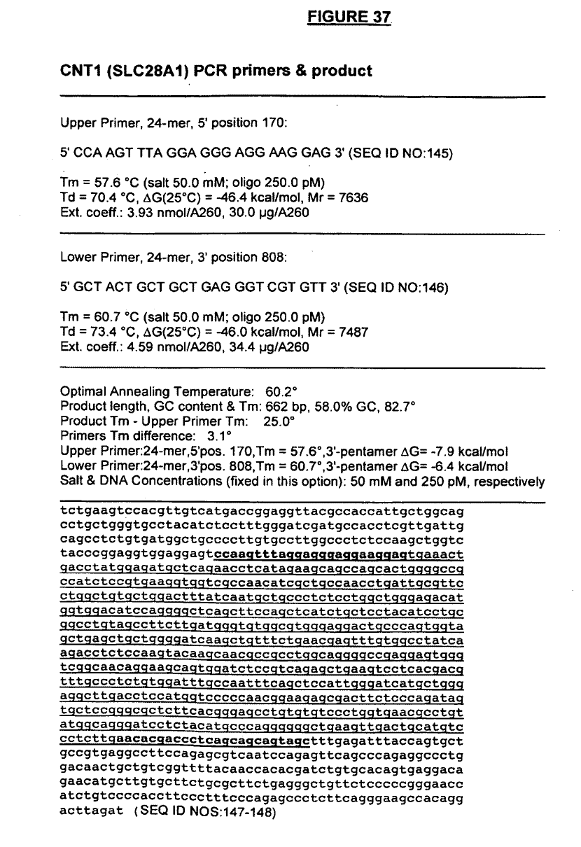

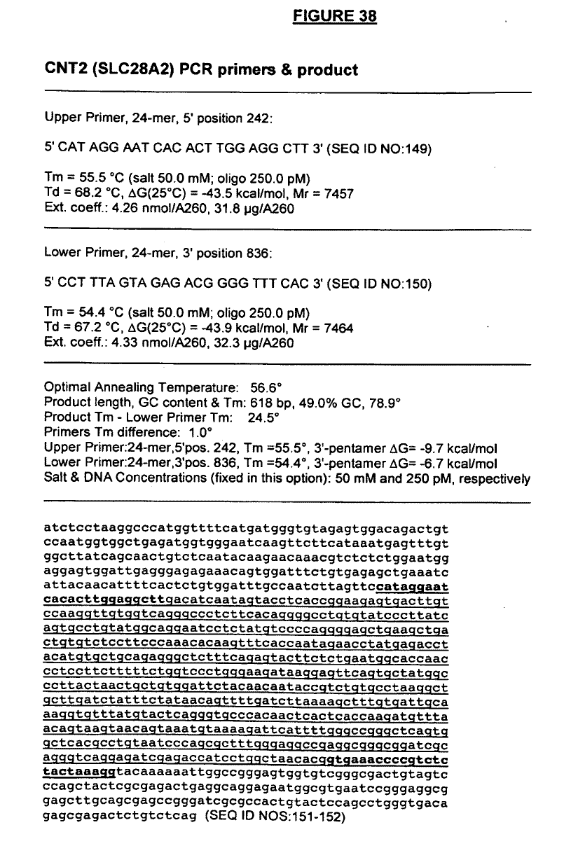

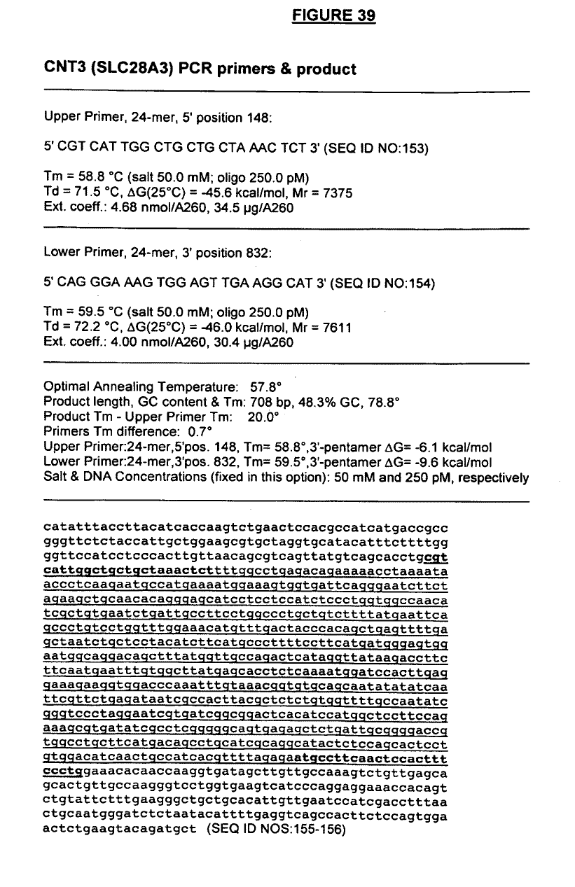

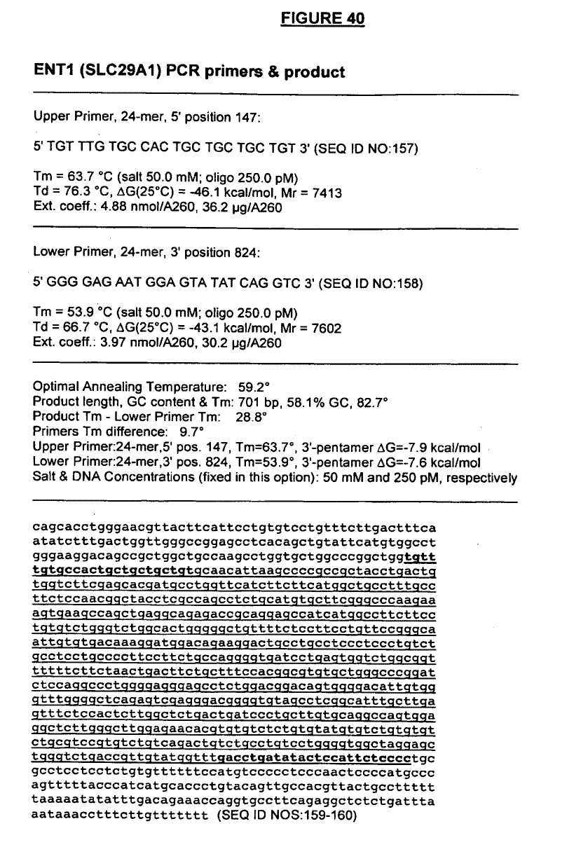

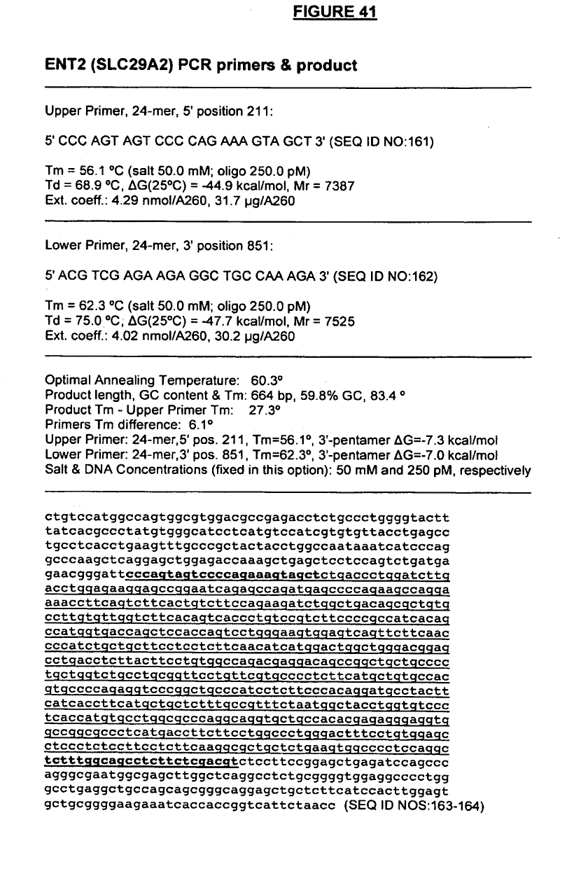

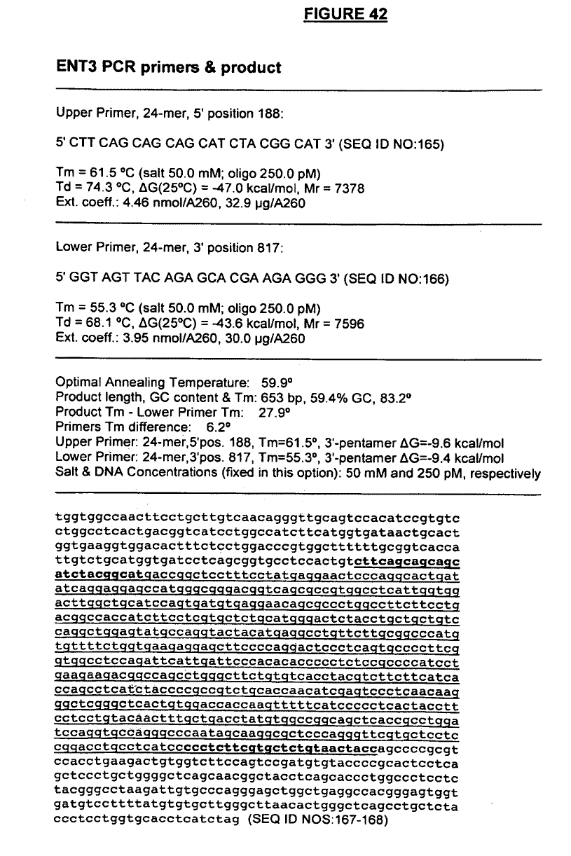

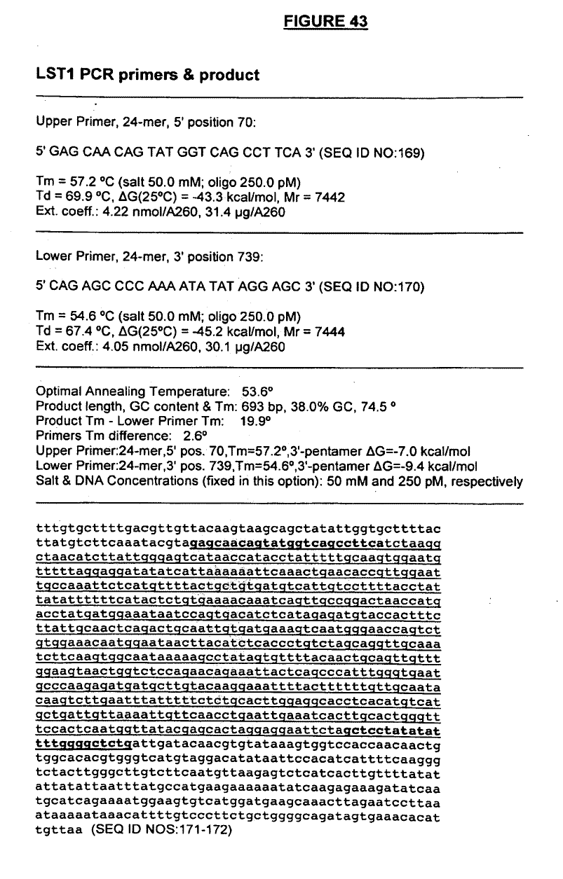

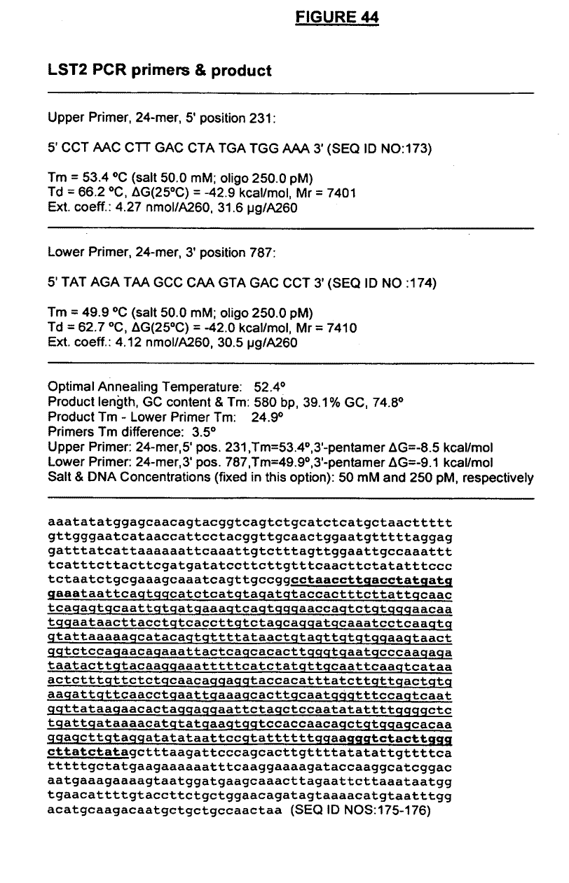

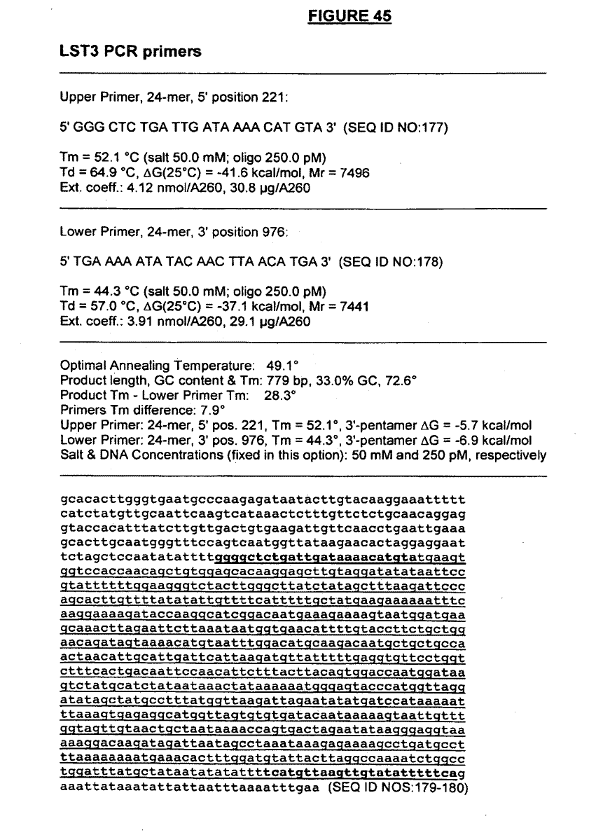

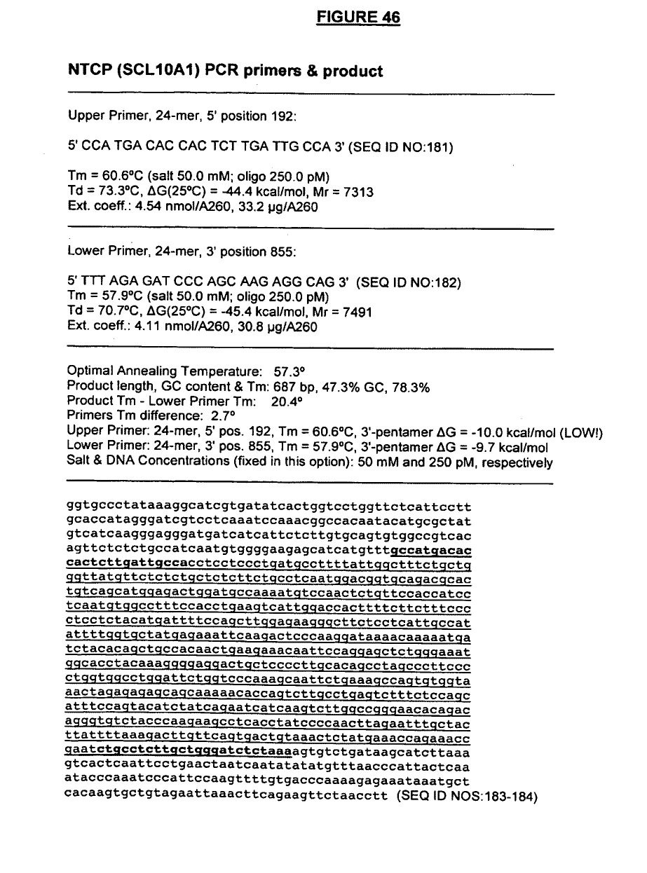

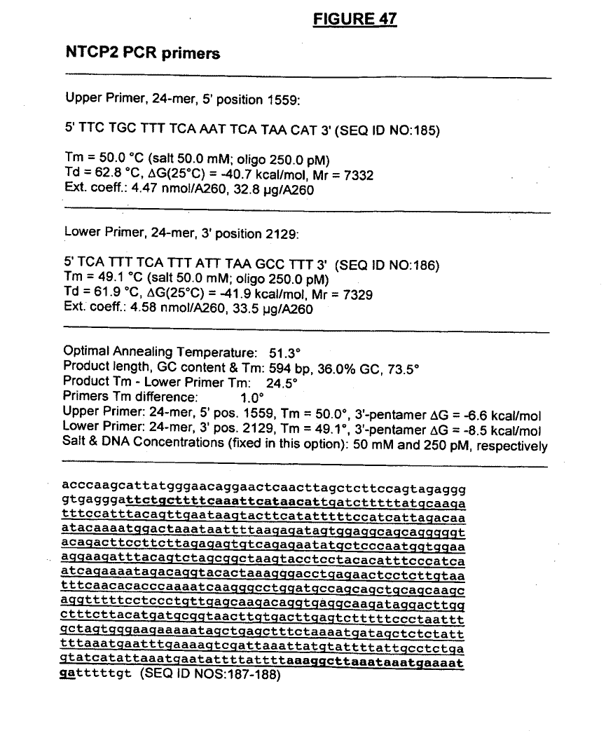

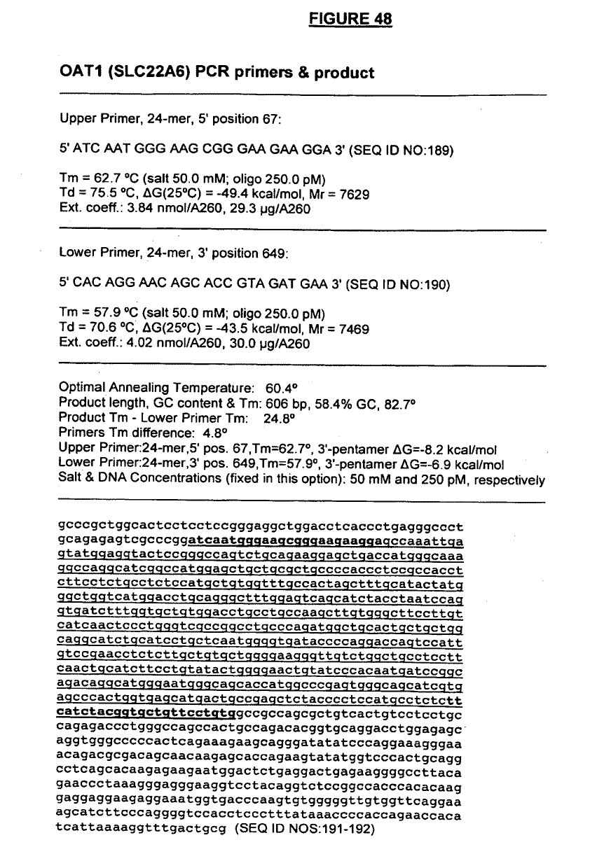

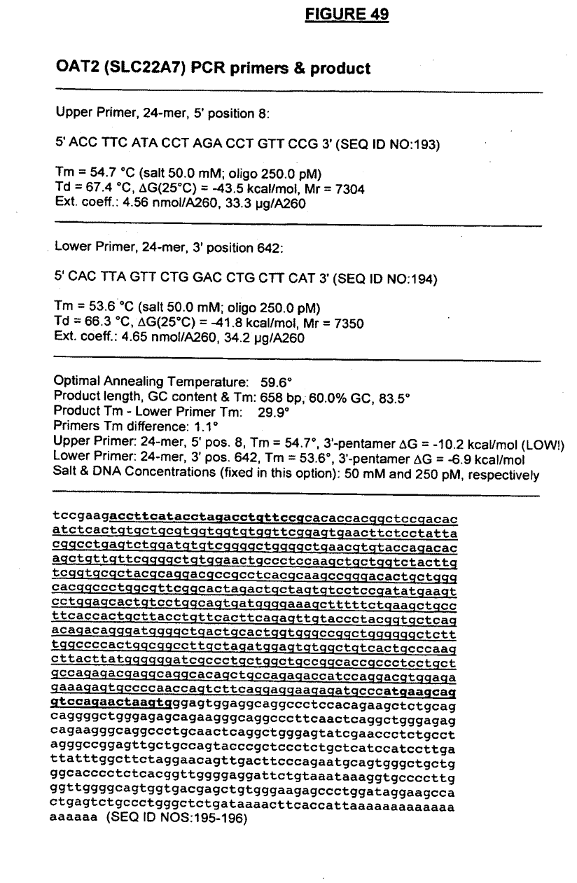

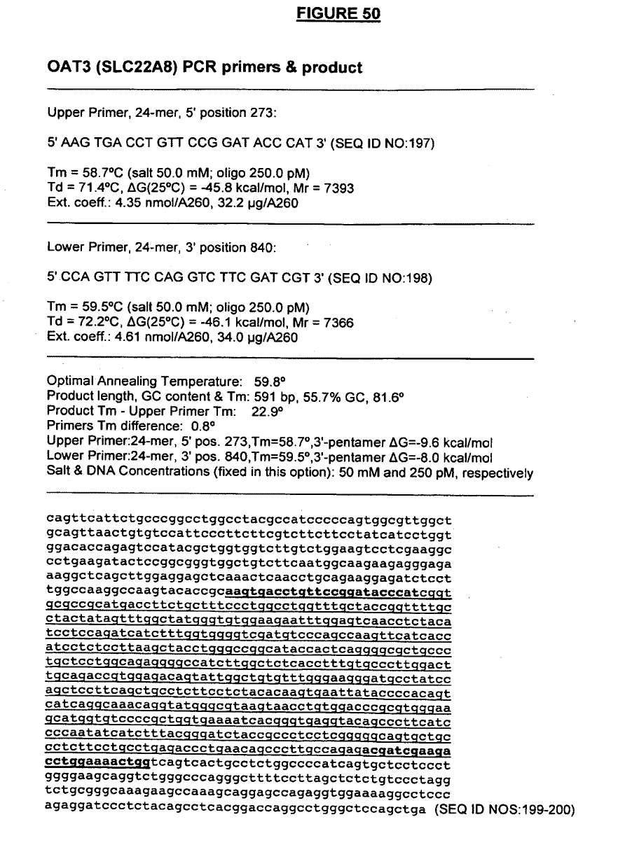

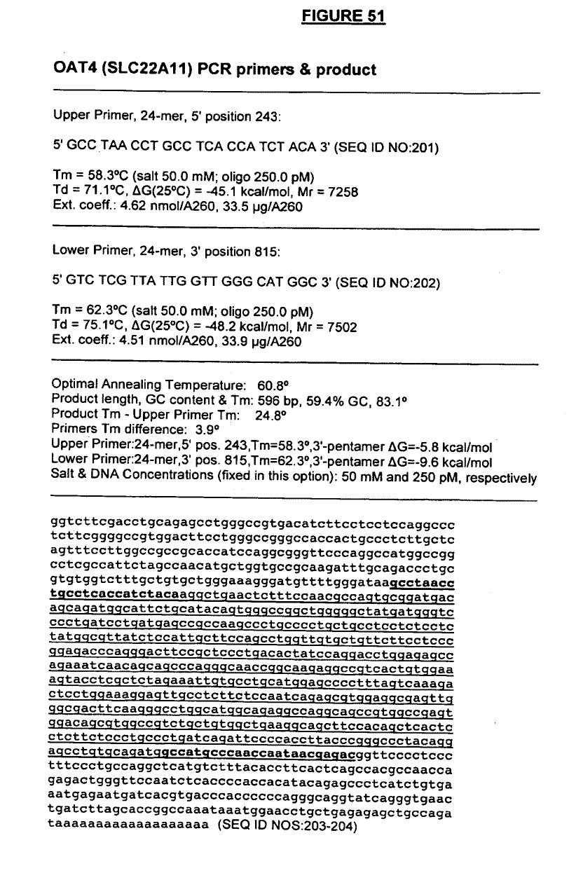

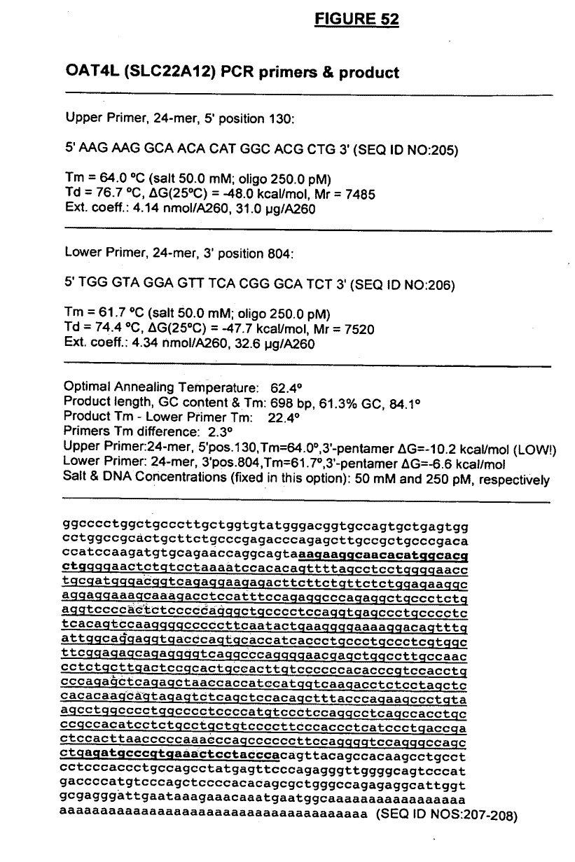

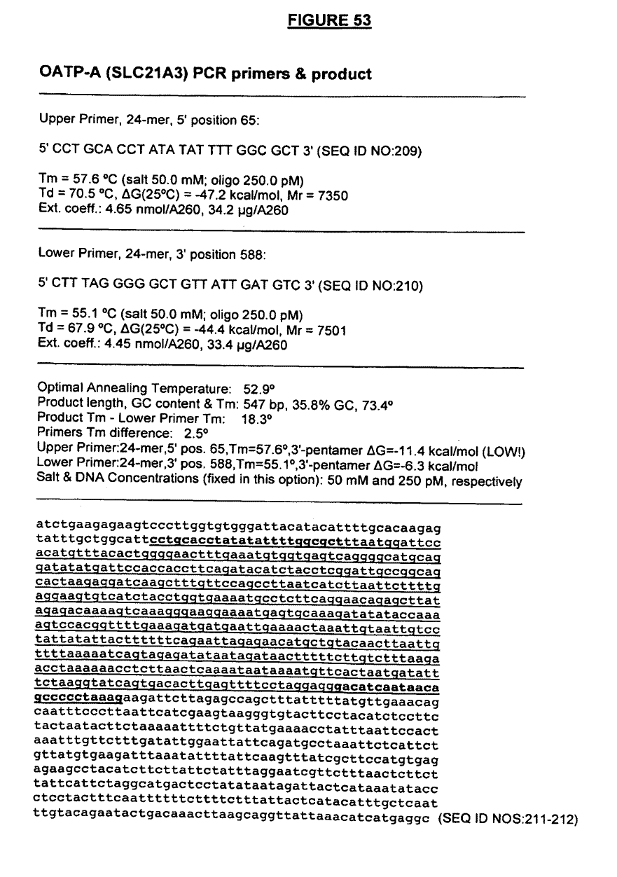

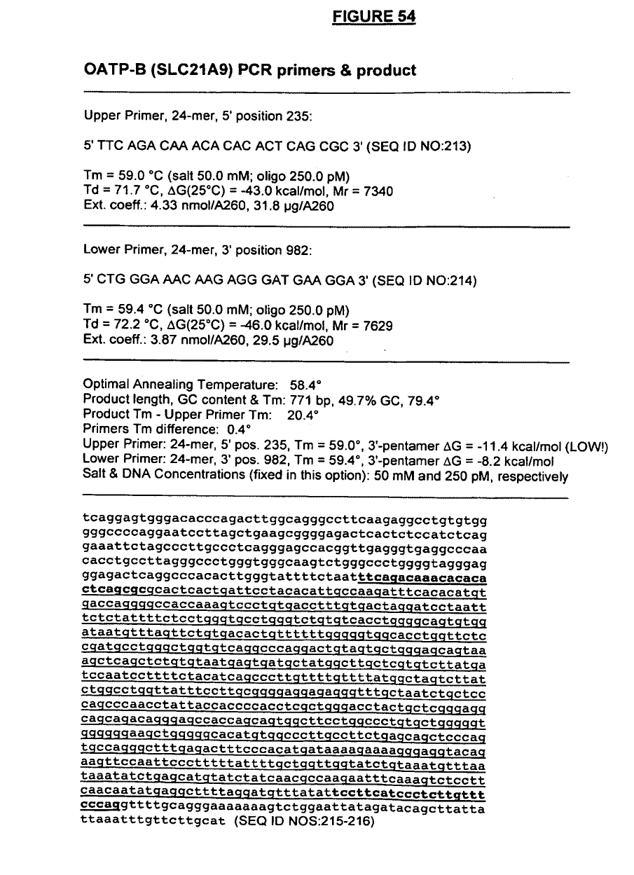

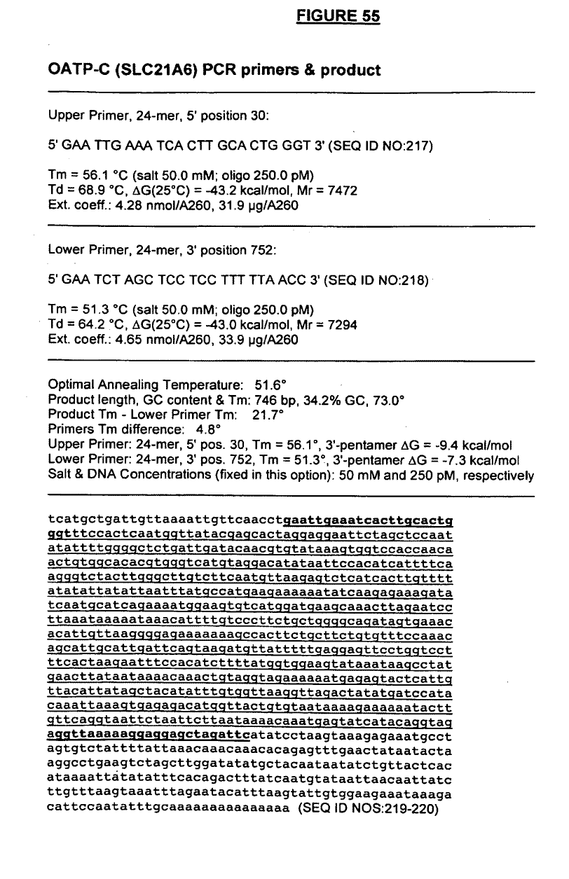

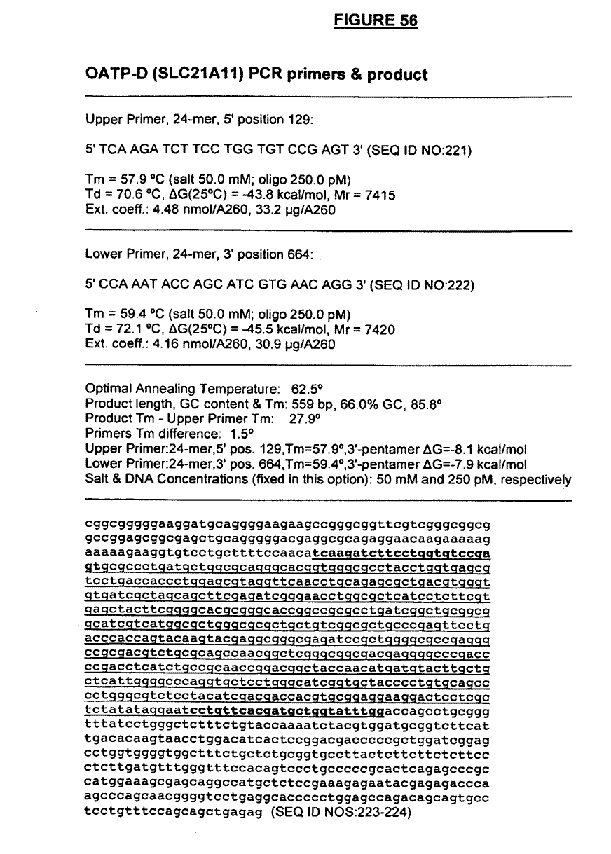

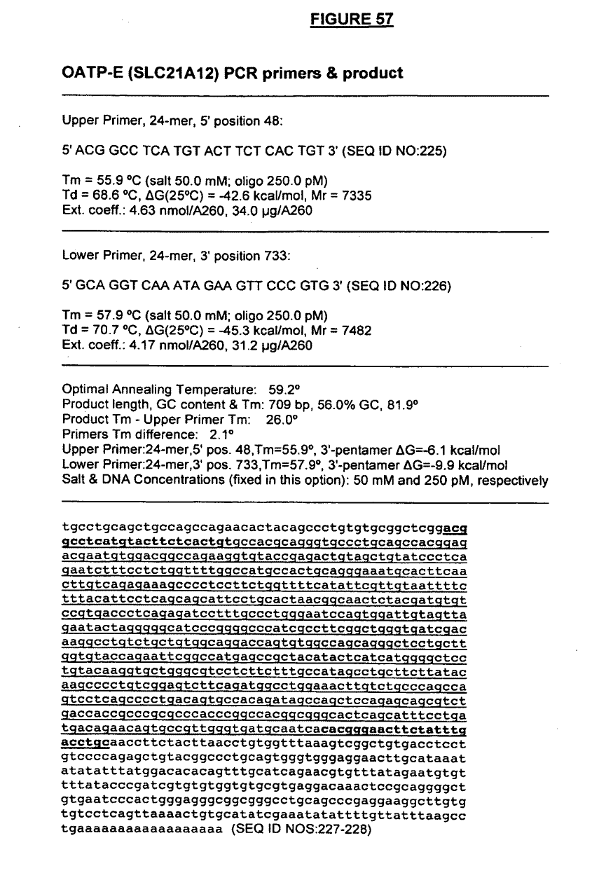

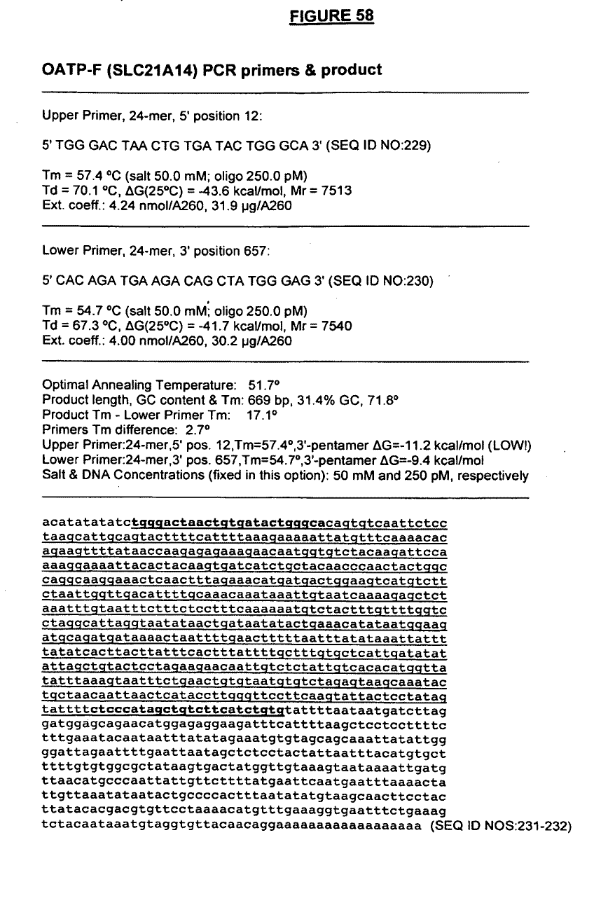

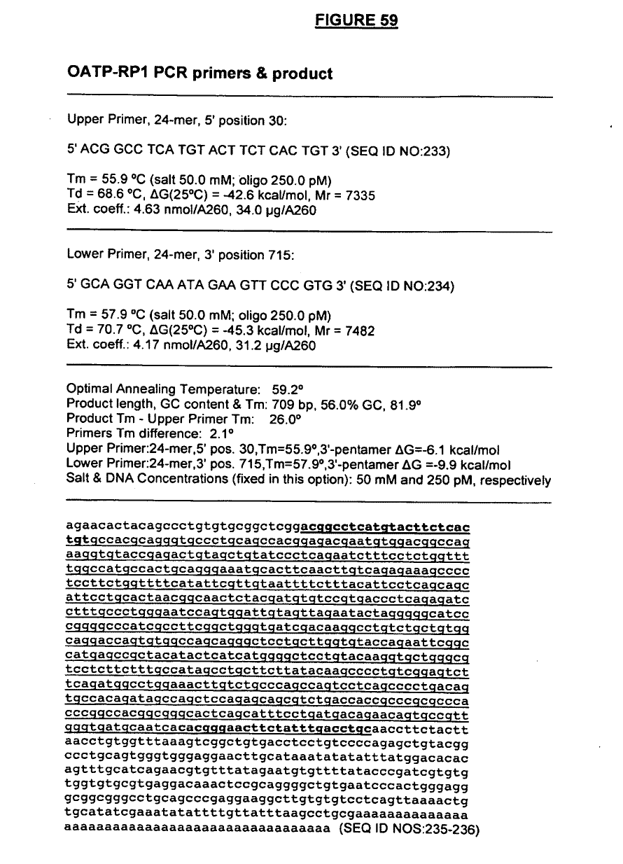

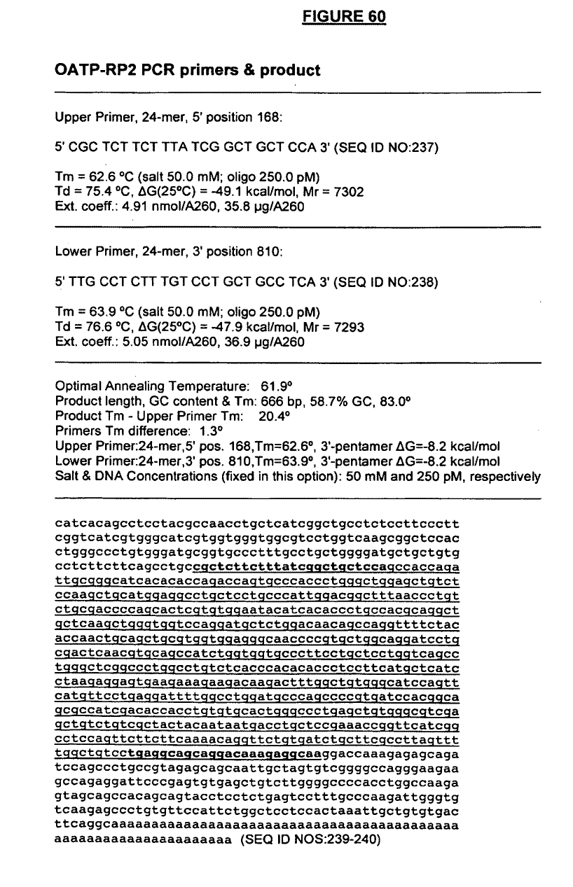

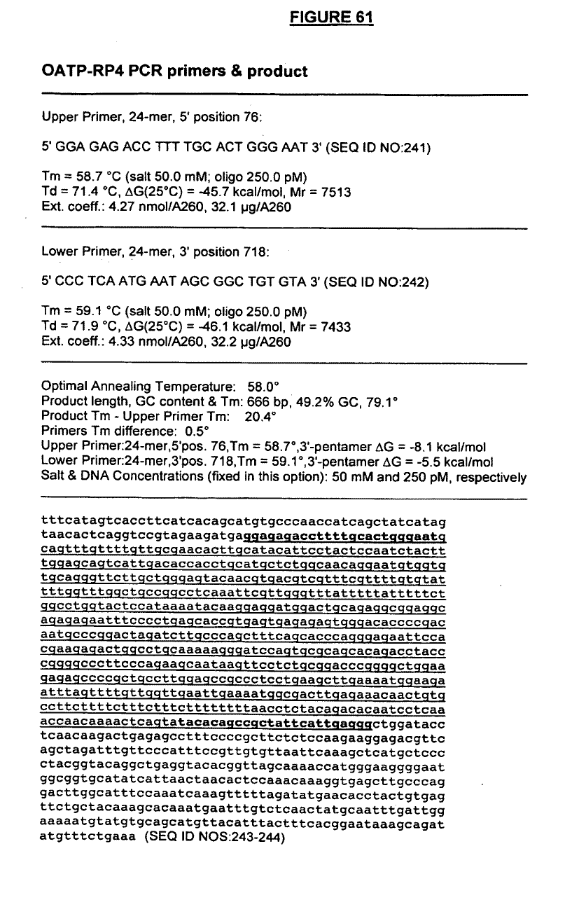

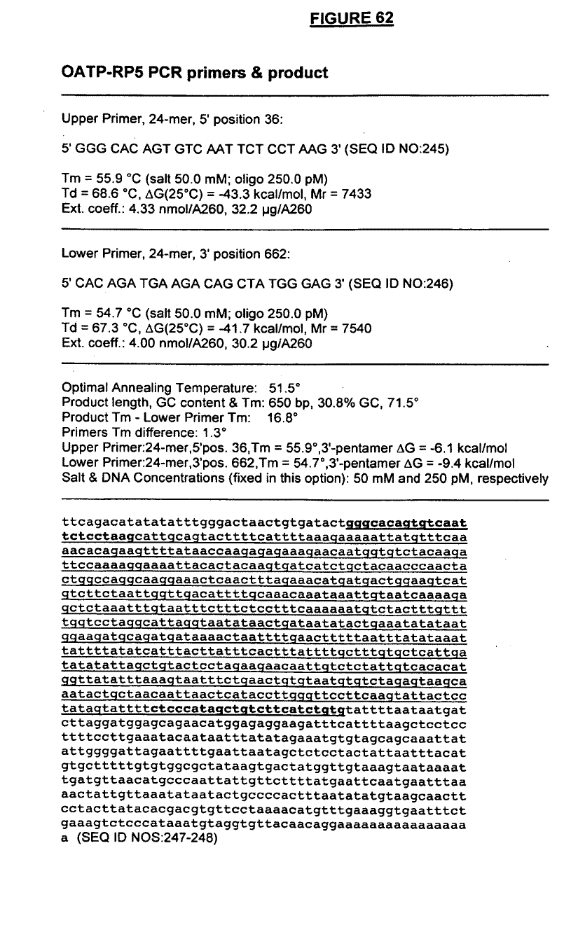

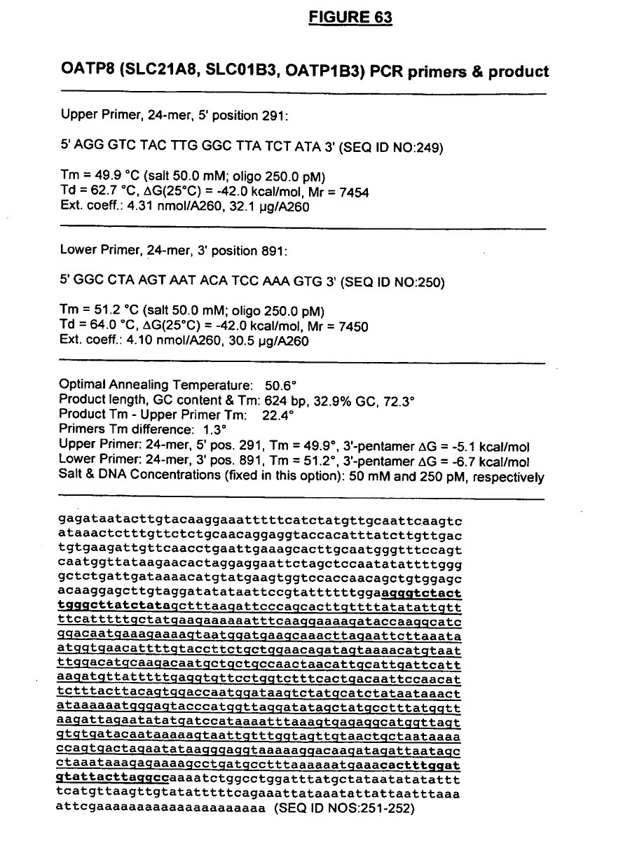

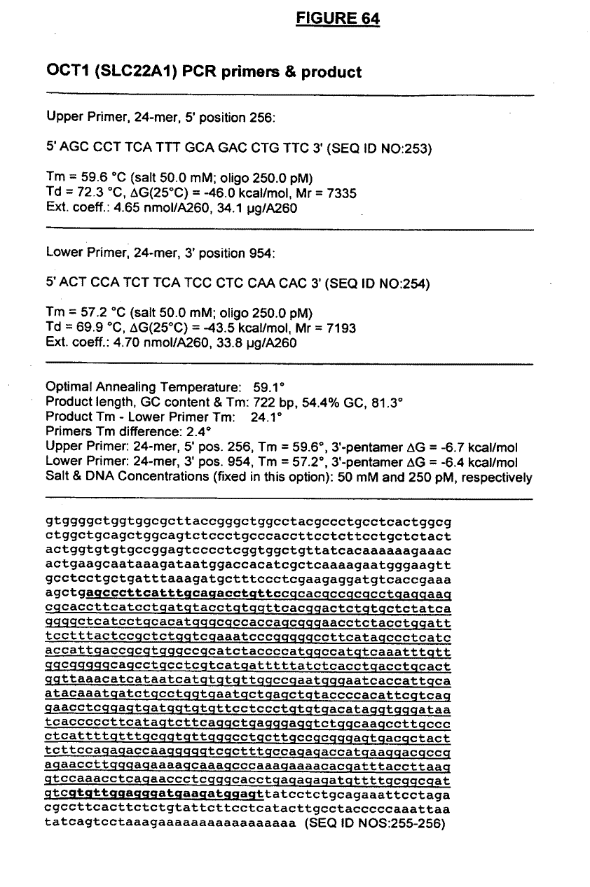

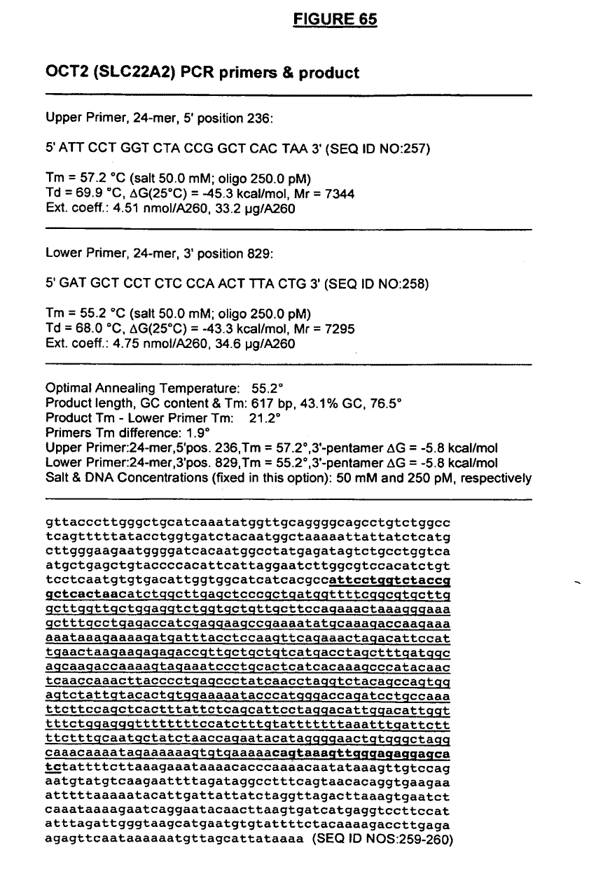

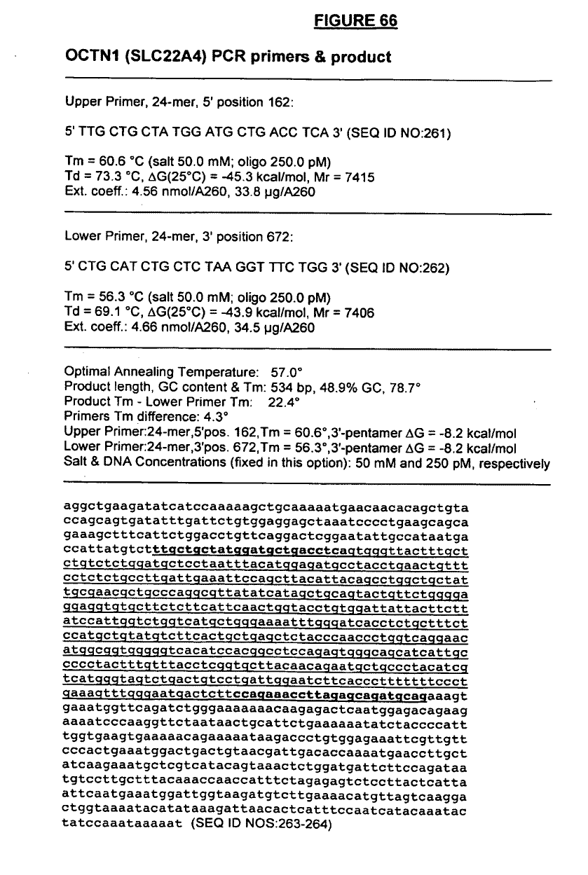

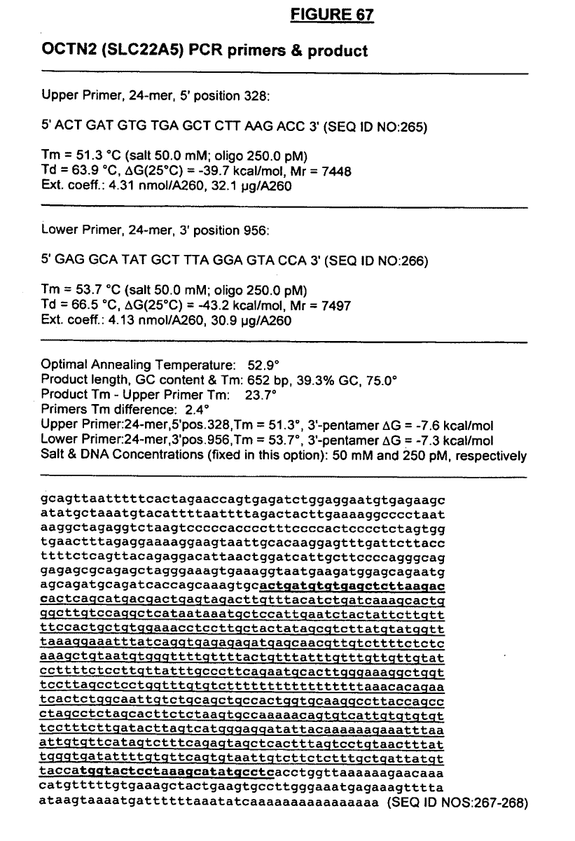

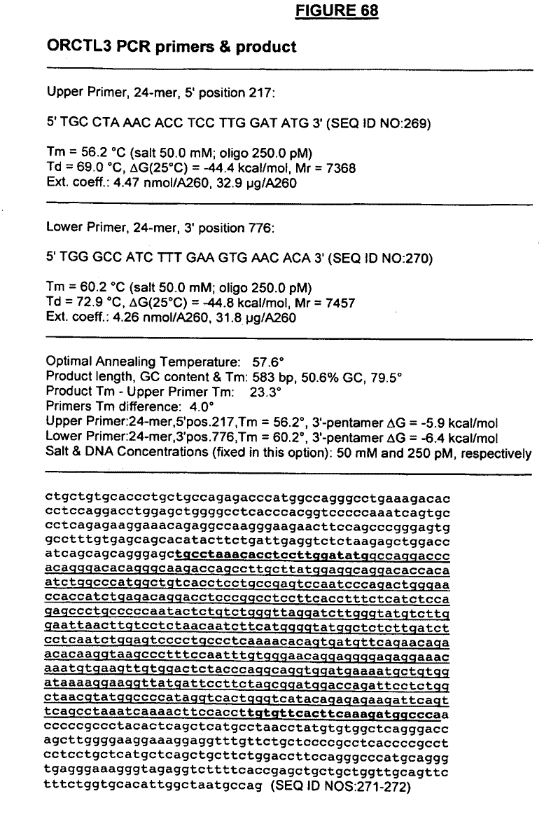

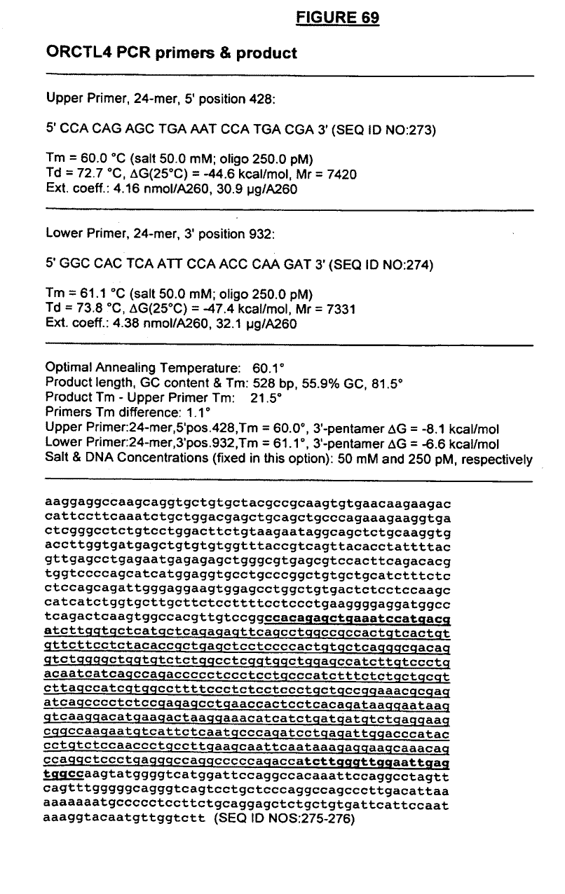

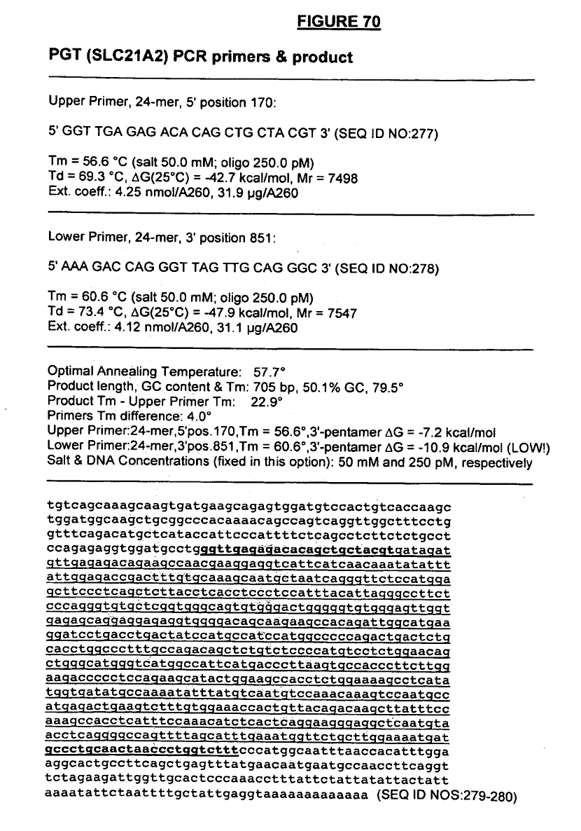

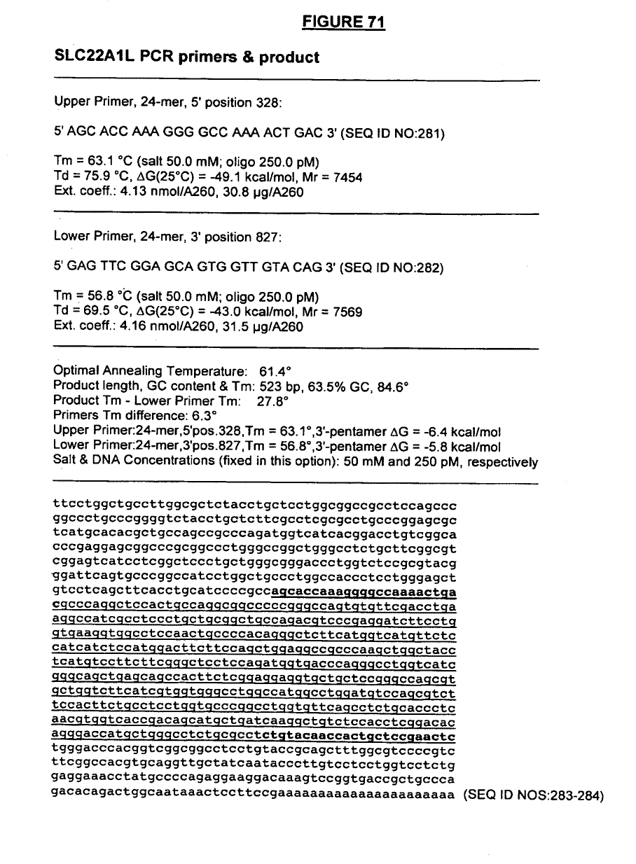

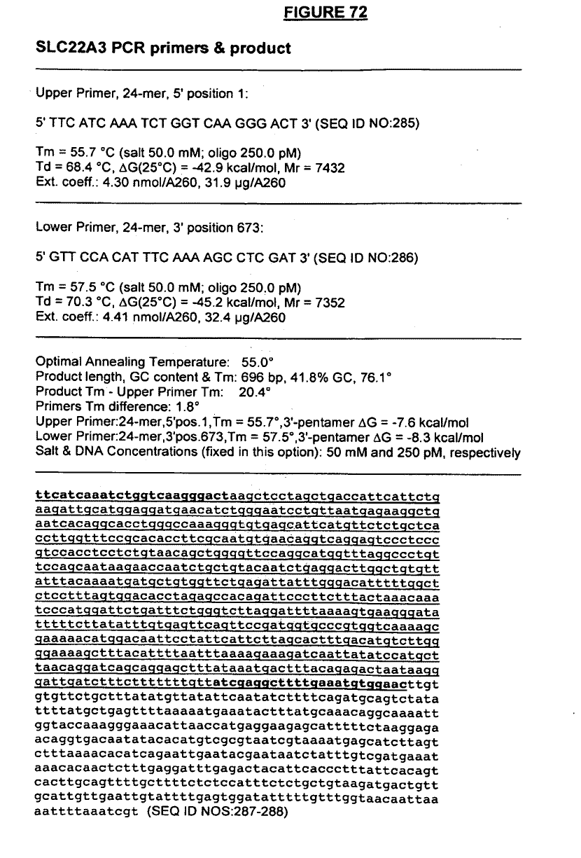

The sets of primers were designed such that the amplification product is a PCR amplicon that is a unique portion of a CYP, NXR, SCL transporter or SULT/UGT gene (See Table 1). FIGS. 1-72 show the nucleic acid sequences of each PCR amplicon (underlined). The primers are shown in bold. The Figures also show the PCR conditions used to generate the PCR amplicon.

The NCBI (www.ncbi.nlm.nig.gov) and BCM search launcher (www.searchlauncher.bcm.tme.edu) websites were used to verify PCR primer identity with the CYP, NXR, SLC transporter or SULT/UGT gene region of interest. BLAST sequence searches and alignment analyses were completed for each PCR primer pair and PCR amplicon to ensure minimum cross-hybridization with other known genes and other known CYP, NXR, SLC transporter or SULT/UGT genes.

### Total RNA Preparation

Cell lines were grown as adherent monolayers following the ATCC guidelines in Falcon T175 flasks until semi-confluent. Culture medium was removed. The adherent cells were washed twice with PBS (phosphate buffered saline) pH7.4. 1.5 ml TriZol reagent (Cat. No. 15596-018, Invitrogen Life Technologies) was added to each flask to lyse the cells and liberate the nucleic acids. The total RNA component of the nucleic acid lysate was isolated according to the manufacturer's instructions. Total RNA was quantitated by spectrophotometric analysis and OD₂₆₀ₙₘ:OD₂₈₀ₙₘ ratios.

cDNA Synthesis

cDNA was prepared from 20 μg of total RNA in a total volume of 40 μg of total RNA was added to a 200 μl RNase-free microtube and placed on ice. 4 μl of a 300 ng/μl solution of random primers (9 mers, 12 mers or 15 mers, MWG-Biotech) was added to the tube containing the total RNA and the final volume made up to 22 μl with RNase-free dH₂O. The microtube was capped and then heated at 65° C. for 10 min in a thermal cycler (PTC200 DNA Engine, MJ Research). The microtube was then removed from the thermal cycler and placed on ice for 3 min. The microtube was spun in a microfuge (C-1200, VWR Scientific Products) to collect the solution in the bottom of the microtube and placed on ice.

First-strand cDNA synthesis was accomplished with the SuperScript II RNase H-Reverse Transcriptase reagent set (Cat. No. 18064-014, Invitrogen Life Technologies). 8 μl 5× First-Strand Buffer [250 mM Tris-HCl pH 8.3, 375 mM KCl, 15 mM MgCl₂], 4 μl 100 mM DTT, 2 μl 10 mM dNTP Mix [10 mM each dATP, dCTP, dGTP, dTTP] were added to the microtube on ice. The microtube was capped and then heated at 25° C. for 10 min in a thermal cycler. The microtube was then heated at 42° C. for 2 min in a thermal cycler. The microtube was uncapped and left in the thermal cycler. 2 μl SuperScript II (200 U/μl) was added to the solution in the microtube and mixed with the micropipette tip. The microtube was recapped and incubated at 42° C. for 60 min in a thermal cycler. Subsequent to this incubation the microtube was heated at 70° C. for 15 min in a thermal cycler. The microtube was then removed from the thermal cycler and spun in a microfuge to collect the solution in the bottom of the microtube and then returned to the thermal cycler. 1 μl of RNase H (2 U/μl) was added to the cDNA synthesis reaction and incubated at 37° C. for 20 min in a thermal cycler. The first-strand cDNA synthesis reaction was then stored at −20° C. until required for RT-PCR.

### RT-PCR

RT-PCR was performed in a final volume of 25 μl. 2 μl of the first-strand cDNA synthesis reaction was added to a 200 μl microtube and placed on ice. 2 μl of a specific CYP, NXR, SLC transporter or SULT/UGT gene primer pair mix [10 μM each forward PCR primer and reverse PCR primer], 2.5 μl 10×PCR Buffer [200 mM Tris-HCl pH 8.4, 500 mM KCl], 0.75 μl 50 mM MgCl₂, 0.5 μl 10 mM dNTP Mix [10 mM each dATP, dCTP, dGTP, dTTP], 16.25 μl dH₂O and 1 μl Taq polymerase (5 U/ul) were added to the side of the microtube. The reagents were mixed and collected in the bottom of the microtube by spinning the capped microtube in a microfuge. The capped microtube was then placed in a thermal cycler block with a heated lid (PTC200 DNA Engine, MJ Research), both pre-heated to 95° C., and incubated at this temperature for 5 min. After this initial denaturation step 40 cycles of PCR amplification were performed as follows: Denature 95° C. for 30s, Anneal 60° C. for 30s, Extend 72° C. for 60s. Following the final 72° C. Extend step the PCR was incubated for an additional 10 min at 72° C. The PCR was then maintained at a temperature of 15° C. PCR products were stored at −20° C. until needed.

### PCR Amplicon Purification

CYP, NXR, SLC transporter or SULT/UGT gene RT-PCR amplification products (PCR amplicons) were analysed by electrophoresis at 150V for 20 min in 1×TAE running buffer in an agarose gel [0.8% agarose, 1×TAE, 0.5 μg/ml ethidium bromide] with 4 μl of a 250 bp DNA Ladder (Cat. No. 10596-013, Invitrogen Life Technologies) to permit size estimates of the PCR amplicons.

The CYP, NXR, SLC transporter or SULT/UGT gene RT-PCR amplification products (PCR amplicons) were visualised “in gel” with a UV transilluminator (UVP M-15, DiaMed Lab Supplies) and photographed with a photo-documentation camera and hood (FB-PDC-34, FB-PDH-1216, Fisher Biotech), a #15 Deep Yellow 40.5 mm screw-in optical glass filter (FB-PDF-15, Fisher Biotech) and Polaroid Polapan 667 film.

The CYP, NXR, SLC transporter or SULT/UGT gene RT-PCR amplification products (PCR amplicons) were isolated and purified from the CYP, NXR, SLC transporter or SULT/UGT gene RT-PCR using the QIAquick PCR purification kit (Cat. No. 28104, QIAGEN Inc.) according to the manufacturer's instructions. In some cases the entire PCR was analysed by electrophoresis on an agarose gel [see below], the PCR product of interest excised from the gel and the PCR product purified using the MinElute gel extraction kit (Cat. No. 28604, QIAGEN Inc.) according to the manufacturer's instructions. After purification, the CYP, NXR, SLC transporter or SULT/UGT gene RT-PCR amplification products (PCR amplicons) were analysed by electrophoresis at 150V for 20 min in 1×TAE running buffer in an agarose gel [0.8% agarose, 1×TAE, 0.5 ug/ml ethidium bromide] with 4 μl of a Low DNA Mass Ladder (Cat. No. 10068-013, Invitrogen Life Technologies) to permit PCR amplicon sizing and quantitation.

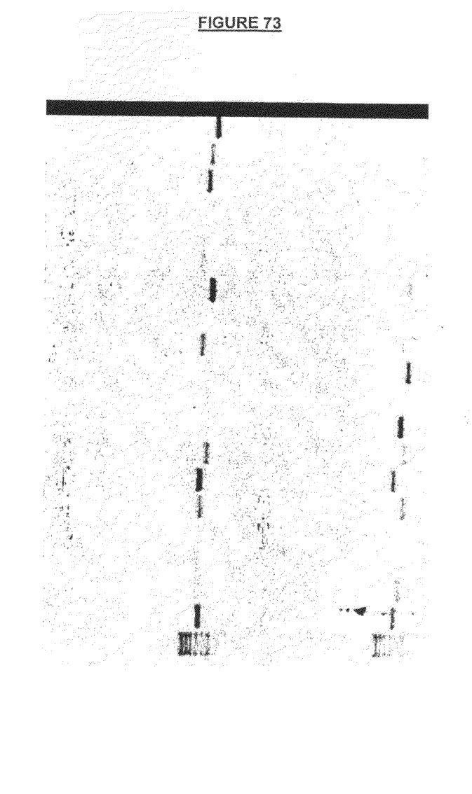

FIG. 73 shows the CYP, NXR, SLC transporter or SULT/UGT gene RT-PCR amplification products from various total RNA sources including cell lines (Caco-2, HEK293, HepG2) and human tissues (colon, kidney, liver).

### Example 2

### Verification of Human CYP, NXR, SLC Transporter or SULT/UGT Gene Close by DNA Sequencing

The sequences of the cloned PCR amplicons, which are each unique portions of each of the known human CYP, NXR, SLC transporter or SULT/UGT genes, are verified.

### CYP, NXR, SLC Transporter or SULT/UGT Gene PCR Amplicon Cloning and Sequencing

A number of the purified CYP, NXR, SLC transporter or SULT/UGT gene RT-PCR amplification products (PCR amplicons) were cloned into pCR4-TOPO vectors using the TOPO TA Cloning Kit for Sequencing (Cat. No. K4575-40, Invitrogen Life Technologies) according to the manufacturer's instructions to verify the sequence of the purified CYP, NXR, SLC transporter or SULT/UGT gene PCR amplicon.

DNA sequence analysis was performed by MWG-Biotech. Sequence files from each clone were verified by comparison to the NCBI nucleotide database.

### Example 3

### DNA Microarray

### CYP, NXR, SLC Transporter or SULT/UGT Gene Microarray (DT2 Microarray)

1-2 μg of each of the purified CYP, NXR, SLC transporter or SULT/UGT gene vector-PCR amplification products (PCR amplicons) and 5 purified positive control vector-PCR amplification products (PCR amplicons) were aliquoted into individual wells of a CoStar SeroCluster 96 well U-bottom polypropylene microwell plate (source plate). The source plate was placed in a Speed-Vac concentrator (SPD101B, Savant Instruments Inc.) and dried under vacuum for 1 hour at 45° C. The dry RT-PCR amplification products (PCR amplicons) in the source plate were resuspended in 20 μl 1× NoAb Print Buffer (150 mM sodium phosphate pH 8.5, Cat. No. UAS0001PB, NoAb BioDiscoveries Inc.), sealed with mylar sealing tape (Cat. No. T-2162, Sigma Chemical Company) and dissolved by shaking at 300 rpm for 1 hour at room temperature on a microplate shaker (EAS2/4, SLT Lab Instruments).

The source plate was then placed in a humidified (21-25° C., 45-60% RH) microarrayer cabinet (SDDC-2, ESI/Virtek Vision Corp./BioRad Laboratories Inc.). Each purified RT-PCR amplification product (PCR amplicon) was printed in quadruplicate on activated covalent-binding epoxy slides (Cat. No. UAS0005E, NoAb BioDiscoveries Inc.) using Stealth micro-spotting pins (Cat. No. SMP5, TeleChem International Inc.). The 384 element microarrays were air-dried in the microarrayer cabinet for at least 4 hours. Printed microarrays were stored in 20 slide racks under vacuum until needed.

### Example 4

### Method for Detecting CYP, NXR, SLC Transporter or SULT/UGT Gene Expression Using a DNA Microarray

The CYP, NXR, SLC transporter or SULT/UGT gene expression profile for several different cell lines was prepared using the DNA microarray.

### Total RNA Preparation

All cell lines (Caco-2, HEK293, HepG2) were grown as adherent monolayers following the ATCC guidelines in tissue culture flasks until semi-confluent. Culture medium was removed. The adherent cells were washed twice with PBS (phosphate buffered saline) pH7.4. 1.5 ml TriZol reagent (Cat. No. 15596-018, Invitrogen Life Technologies) was added to each flask to lyse the cells and liberate the nucleic acids. The total RNA component of the nucleic acid lysate was isolated according to the manufacturer's instructions. Total RNA was quantitated by spectrophotometric analysis and OD₂₆₀ₙₘ:OD₂₈₀ₙₘ ratios.

Fluorescent cDNA Target Preparation

Fluorescently labeled cDNA targets were prepared from each of the cell lines using 20 μg of total RNA in a total volume of 40 μl.

20 μg of total RNA was added to a 200 μl RNase-free microtube and placed on ice. 3 μl of a 1 nmole/μl solution of Cy5-labeled random primers (9 mers, 12 mers, 15 mers, MWG-Biotech) was added to the tube containing the total RNA and the final volume made up to 22 μl with RNase-free dH₂O. The microtube was capped and then heated at 65° C. for 10 min in a thermal cycler (PTC200 DNA Engine, MJ Research). The microtube was then removed from the thermal cycler and placed on ice for 3 min. The microtube was spun in a microfuge (C-1200, VWR Scientific Products) to collect the solution in the bottom of the microtube and placed on ice.

First-strand cDNA synthesis was accomplished with the SuperScript II RNase H-Reverse Transcriptase reagent set (Cat. No. 18064-014, Invitrogen Life Technologies). 8 μl 5× First-Strand Buffer [250 mM Tris-HCl pH 8.3, 375 mM KCl, 15 mM MgCl₂], 4 μl 100 mM DTT, 2 μl 10 mM dNTP Mix [10 mM each dATP, dCTP, dGTP, dTTP], were added to the microtube on ice. The microtube was capped and then heated at 25° C. for 10 min in a thermal cycler. The microtube was then heated at 42° C. for 2 min in a thermal cycler. The microtube was uncapped and left in the thermal cycler. 2 ul SuperScript II (200 U/μl) was added to the solution in the microtube and mixed with the micropipette tip. The microtube was recapped and incubated at 42° C. for 60 min in a thermal cycler. Subsequent to this incubation the microtube was heated at 70° C. for 15 min in a thermal cycler. The microtube was then removed from the thermal cycler and spun in a microfuge to collect the solution in the bottom of the microtube and then returned to the thermal cycler. 1 μl of RNase H (2 U/μl) was added to the cDNA synthesis reaction and incubated at 37° C. for 20 min in a thermal cycler. The fluorescently labeled cDNA targets were stored at −20° C. overnight before QIAquick column purification.

The fluorescently labeled cDNA targets were thawed and the total volume adjusted to 100 μl with dH₂O. Labeled cDNA targets were isolated and purified using the QIAquick PCR purification kit (Cat. No. 28104, QIAGEN Inc.) according to the manufacturer's instructions except that the final elution volume was adjusted to 150 μl. The purified cDNA target preparation was stored at −20° C. until required for microarray hybridization.

### DT2 Microarray Hybridization

The printed DT2 microarray(s) was removed from storage under vacuum and placed in a 20 slide rack. The DT2 microarray was then denatured by dipping the microarray slide into “boiled” dH₂O for 30s. The denatured DT2 microarray was then placed in a polypropylene 5 slide mailer (Cat. No. 240-3074-030, Evergreen Scientific) and blocked in 1× NoAb Pre-Hybridization Blocking Buffer (Cat. No. UAS0001 BB, NoAb BioDiscoveries Inc.) for 2 hours at room temperature. Pre-hybridized, blocked DT2 microarrays were removed from this solution and placed in a new polypropylene 5 slide mailer (Cat. No. 240-3074-030, Evergreen Scientific) containing a solution of denatured, labeled cDNA targets from a specific cell line.

The labeled cDNA target preparation was thawed and the 1500 added to 850 μl hybridization buffer (500 mM sodium Phosphate pH 6.0, 1% SDS, 1% BSA, 1 mM EDTA) in a 1.5 ml microtube and heated at 95° C. for 10 min. Following denaturation the microtube was spun briefly in a microcentrifuge to collect all the liquid. The denatured, labeled cDNA targets were then added to a polypropylene 5 slide mailer (Cat. No. 240-3074-030, Evergreen Scientific) that contained a pre-hybridized, blocked DT2 microarray placed “array-side” down in the bottom-most slot of the 5 slide mailer. In this orientation the entire surface of the microarray slide is bathed in the hybridization buffer. 5 slide mailers containing the DT2 microarrays were incubated on their sides, “array-side” down, in a 37° C. incubator for 15-18 h.

Hybridized DT2 microarrays were removed from the 5 slide mailers with forceps and placed directly into a 20 slide rack in a slide wash box containing a 0.1×SSC, 0.1% SDS solution. DT2 microarrays were incubated in this solution at 37° C. for 15 min. The slide rack containing the DT2 microarrays was then transferred to a slide wash box containing 0.1×SSC and incubated in this solution at 37° C. for 15 min. Following this step the DT2 microarrays were rinsed in dH2O and air-dried by centrifugation at 1200 rpm.

DT2 Microarray Image Acquisition and Data Analysis

Processed DT2 microarrays were scanned using ScanArray software in a ScanArray Lite MicroArray Analysis System (GSI Lumonics Inc.) at a scan resolution of 10 μm, a laser setting of 90 and a PMT gain of 80. Images were analysed using QuantArray software (GSI Lumonics Inc.). The data generated from QuantArray was exported to GeneLinker Gold (Molecular Mining Inc./Predictive Patterns Software) for bioinformatic analysis and data mining. Gene expression profiles and hierarchical clustering maps (“heat maps”) were also generated using GeneLinker Gold.

FIG. 74 shows the fluorescence intensity matrix plot for CYP, NXR, SLC transporter or SULT/UGT gene expression in normal colon, normal liver, the Caco-2 cell line and Caco-2 treated with doxorubicin.

### Example 5

### Method for Detecting Drug-Associated Changes in CYP, NXR, SLC Transporter or SULT/UGT Gene Expression Using a DNA Microarray (Drug Screening Assay)

Cell lines were treated with two chemotherapeutic agents, doxorubicin and vinblastine, at 2 hour intervals.

Total RNA Preparation from Drug-Treated HepG2 Cell Line

The HepG2 cell line was grown as an adherent monolayer in 8 Falcon T175 flasks following the ATCC guidelines until semi-confluent. Tissue culture flasks were then divided into pairs for each of four timepoints (0 h, 2 h, 4 h, 8 h).

For vinblastine sulfate treatment, 5 μl of a 1000× (5 mM in DMSO) stock solution of vinblastine sulfate was added to 10 Falcon T175 flasks containing the HepG2 monolayer in 10 mls of culture medium (25 nM final concentration), mixed gently by rocking, returned to the CO₂ incubator and harvested for total RNA at the indicated times. The 0 h timepoint flasks were processed immediately after the addition of 5 μl DMSO.

For doxorubicin HCl treatment, 5 μl of a 1000× (5 mM in DMSO) stock solution of doxorubicin HCl was added to 10 Falcon T175 flasks containing the HepG2 monolayer in 10 mls of culture medium (25 nM final concentration), mixed gently by rocking, returned to the CO₂ incubator and harvested for total RNA at the indicated times. The 0 h timepoint flasks were processed immediately after the addition of 5 μl DMSO.

Prior to cell lysis the tissue culture medium was removed. The adherent cells were washed twice with PBS (phosphate buffered saline) pH7.4. 1.5 ml TriZol reagent (Cat. No. 15596-018, Invitrogen Life Technologies) was added to each flask to lyse the cells and liberate the nucleic acids. The total RNA component of the nucleic acid lysate was isolated according to the manufacturer's instructions. Total RNA was quantitated by spectrophotometric analysis and OD₂₆₀ₙₘ:OD₂₈₀ₙₘ ratios.

Fluorescent cDNA Target Preparation

Fluorescently labeled cDNA targets were prepared from each of the 8 timepoint samples for the drug-treated HepG2 cell line (4× vinblastine sulfate, 4× doxorubicin HCl) using 20 μg of total RNA in a total volume of 40 μl.

20 μg of total RNA was added to a 200 μl RNase-free microtube and placed on ice. 3 μl of a 1 nmole/μl solution of Cy5-labeled random primers (9 mers, 12 mers, 15 mers, MWG-Biotech) was added to the tube containing the total RNA and the final volume made up to 22 μl with RNase-free dH₂O. The microtube was capped and then heated at 65° C. for 10 min in a thermal cycler (PTC200 DNA Engine, MJ Research). The microtube was then removed from the thermal cycler and placed on ice for 3 min. The microtube was spun in a microfuge (C-1200, VWR Scientific Products) to collect the solution in the bottom of the microtube and placed on ice.

First-strand cDNA synthesis was accomplished with the SuperScript II RNase H-Reverse Transcriptase reagent set (Cat. No. 18064-014, Invitrogen Life Technologies). 8 μl 5× First-Strand Buffer [250 mM Tris-HCl pH 8.3, 375 mM KCl, 15 mM MgCl₂], 4 μl 100 mM DTT, 2 μl 10 mM dNTP Mix [10 mM each dATP, dCTP, dGTP, dTTP], were added to the microtube on ice. The microtube was capped and then heated at 25° C. for 10 min in a thermal cycler. The microtube was then heated at 42° C. for 2 min in a thermal cycler. The microtube was uncapped and left in the thermal cycler. 2 ul SuperScript II (200 U/μl) was added to the solution in the microtube and mixed with the micropipette tip. The microtube was recapped and incubated at 42° C. for 60 min in a thermal cycler. Subsequent to this incubation the microtube was heated at 70° C. for 15 min in a thermal cycler. The microtube was then removed from the thermal cycler and spun in a microfuge to collect the solution in the bottom of the microtube and then returned to the thermal cycler. 1 μl of RNase H (2 U/μl) was added to the cDNA synthesis reaction and incubated at 37° C. for 20 min in a thermal cycler. The fluorescently labeled cDNA targets were stored at −20° C. overnight before QIAquick column purification.

The fluorescently labeled cDNA targets were thawed and the total volume adjusted to 100 μl with dH₂O. Labeled cDNA targets were isolated and purified using the QIAquick PCR purification kit (Cat. No. 28104, QIAGEN Inc.) according to the manufacturer's instructions except that the final elution volume was adjusted to 150 μl. The purified cDNA target preparation was stored at −20° C. until required for microarray hybridization.

### DT2 Microarray Hybridization

The printed DT2 microarray(s) was removed from storage under vacuum and placed in a 20 slide rack. The DT2 microarray was then denatured by dipping the microarray slide into “boiled” dH₂O for 30 s. The denatured DT2 microarray was then placed in a polypropylene 5 slide mailer (Cat. No. 240-3074-030, Evergreen Scientific) and blocked in 1× NoAb Pre-Hybridization Blocking Buffer (Cat. No. UAS0001 BB, NoAb BioDiscoveries Inc.) for 2 hours at room temperature. Pre-hybridized, blocked DT2 microarrays were removed from this solution and placed in a new polypropylene 5 slide mailer (Cat. No. 240-3074-030, Evergreen Scientific) containing a solution of denatured, labeled cDNA targets from a specific cell line.

The labeled cDNA target preparation was thawed and the 150 μl added to 850 ul hybridization buffer (500 mM sodium Phosphate pH 6.0, 1% SDS, 1% BSA, 1 mM EDTA) in a 1.5 ml microtube and heated at 95° C. for 10 min. Following denaturation the microtube was spun briefly in a microcentrifuge to collect all the liquid. The denatured, labeled cDNA targets were then added to a polypropylene 5 slide mailer (Cat. No. 240-3074-030, Evergreen Scientific) that contained a pre-hybridized, blocked DT2 microarray placed “array-side” down in the bottom-most slot of the 5 slide mailer. In this orientation the entire surface of the microarray slide is bathed in the hybridization buffer. 5 slide mailers containing the DT2 microarrays were incubated on their sides, “array-side” down, in a 37° C. incubator for 15-18 h.

Hybridized DT2 microarrays were removed from the 5 slide mailers with forceps and placed directly into a 20 slide rack in a slide wash box containing a 0.1×SSC, 0.1% SDS solution. DT2 microarrays were incubated in this solution at 37° C. for 15 min. The slide rack containing the DT2 microarrays was then transferred to a slide wash box containing 0.1×SSC and incubated in this solution at 37° C. for 15 min. Following this step the DT2 microarrays were rinsed in dH₂O and air-dried by centrifugation at 1200 rpm.

### DT2 Microarray Image Acquisition and Data Analysis

Processed DT2 microarrays were scanned using ScanArray software in a ScanArray Lite MicroArray Analysis System (GSI Lumonics Inc.) at a scan resolution of 10 μm, a laser setting of 90 and a PMT gain of 80. Images were analyzed using QuantArray software (GSI Lumonics Inc.). The data generated from QuantArray was exported to GeneLinker Gold (Molecular Mining Inc./Predictive Patterns Software) for bioinformatic analysis and data mining. Gene expression profiles and hierarchical clustering maps for drug treatment-related changes in CYP, NXR, SLC transporter or SULT/UGT gene expression were also generated using GeneLinker Gold.

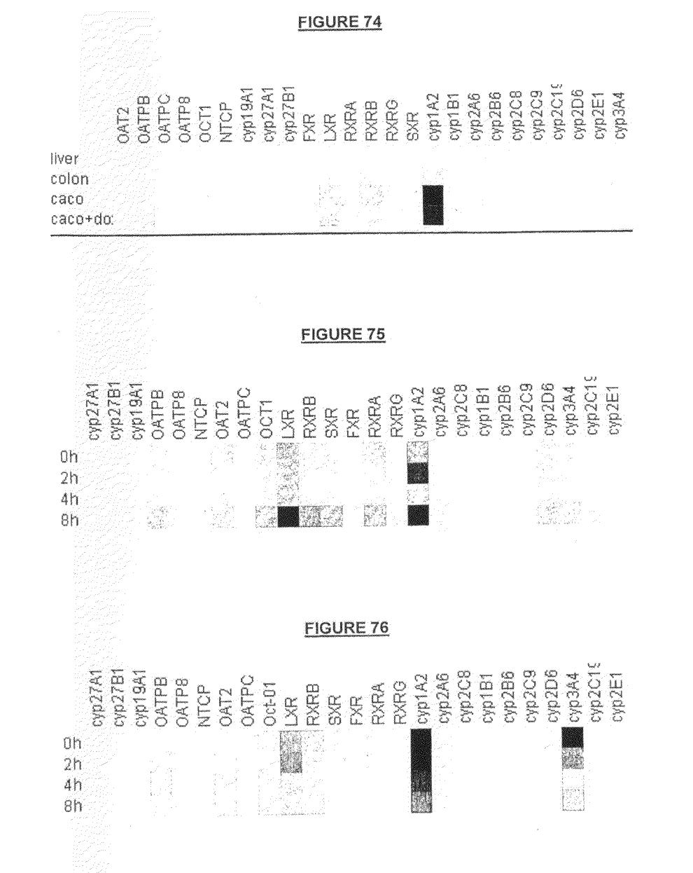

FIG. 75 shows the fluorescence intensity cluster plot for CYP, NXR, SLC transporter or SULT/UGT gene expression in the HepG2 cell line treated with doxorubicin at various time intervals.

FIG. 76 shows the fluorescence intensity cluster plot for CYP, NXR, SLC transporter or SULT/UGT gene expression in the HepG2 cell line treated with vinblastine at various time intervals.

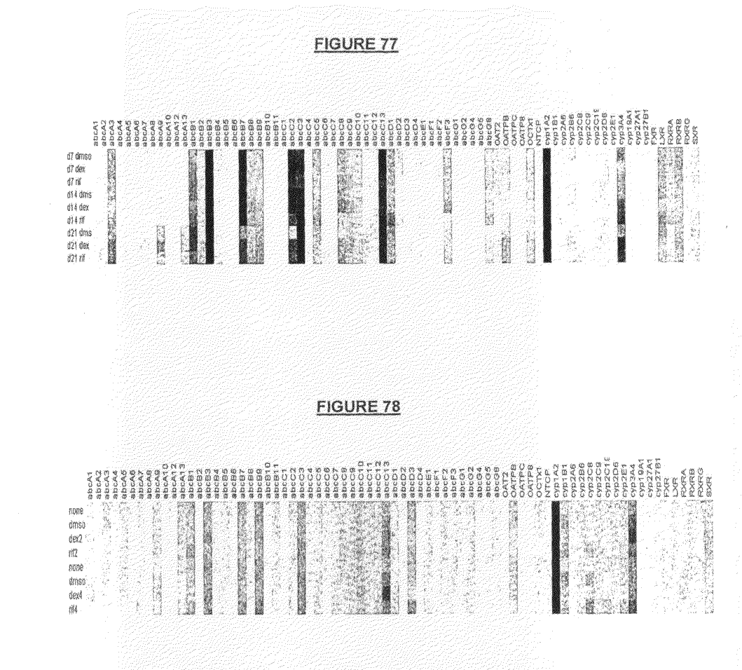

FIG. 77 shows drug transporter, drug metabolising enzyme and nuclear receptor-transcription factor gene expression profiles in Caco-2 cell monolayers. Total RNA isolated from untreated and drug-treated Caco-2 cells was labeled and hybridized to individual DTEx microarrays. Log 2-normalized fluorescence intensity values from each microarray hybridisation were used to generate the matrix plot. Gene expression values represent the normalized, log 2-transformed median value from 6 individual microarray hybridizations [n=24 for each gene]. The matrix plot displays the gene expression profiles for Caco-2 cells treated with dexamethasone [dex] and rifampin [rif] at day 7, day 14 and day 21.

FIG. 78 shows drug transporter, drug metabolising enzyme and nuclear receptor-transcription factor gene expression profiles in fresh human hepatocytes. Total RNA isolated from untreated and drug-treated human hepatocytes was labeled and hybridized to individual DTEx microarrays. Log 2-normalized fluorescence intensity values from each microarray hybridization were used to generate the matrix plot. Gene expression values represent the normalized, log 2-transformed median value from 6 individual microarray hybridizations [n=24 for each gene]. The matrix plot displays the gene expression profiles for human hepatocytes tested with dexamethasone [dex] and rifampin [rif].

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

| | | Primers | | | |
|---|---|---|---|---|---|
| | | primer | | | |
| | | location | | Primers | |
| | Product | 3′UTR or | Primers | sequence | |
| Gene | (bps) | CDNs | name | 5′ to 3′ direcetion | psuedonyms & comments |
| CYP1A2 | 696 | 3′UTR | CYP1A2For | ACCATGGCCAGCTAATTTTTGTAT | cytochrome P450, family 1, subfamily A, |
| | | | | | polypeptide 2, CP12, P3-450, P450(PA) |
| | | 3′UTR | CYP1A2ReV | AAGGCAAATCCATAGACACAGAAA | |
| CYP1B1 | 405 | 3′UTR | CYP1B1For | AAGATGTCTCAGGTTTGTTTTGTG | cytochrome P450, family 1, subfamily B, |
| | | | | | polypeptide 1, CP1B, GLC3A |
| | | 3′UTR | CYP1B1Rev | GGTGTCCCAGTATAAGTAATGAGA | |
| CYP2A6 | 408 | 3′UTR | CYP2A6For | TGCTTTTGTGCCCTTTTCCATCGG | cytochrome P450, family 2, subfamily A, |
| | | | | | polypeptide 6, CPA6, CYP2A, CYP2A3, |
| | | | | | P450PB, P450C2A |
| | | 3′UTR | CYP2A6Rev | TTTCCTTCCTCTCATCCCAGCTCG | |
| CYP2B6 | 436 | 3′UTR | CYP2B6For | GATTCTCCAGTCTCAGCTCCCAAG | cytochrome P450, family 2, subfamily B, |
| | | | | | polypeptide 6, CPB6, IIB1, P450, |
| | | | | | CYP2B, CYPIIB6 |
| | | 3′UTR | CYP2B8Rev | TGGGGAGGTCAGGCTTTAGAGATG | |
| CYP2C8 | 439 | 3′UTR | CYP2C8For | TTAAAGAACCTCAATACTACTGCA | cytochrome P450, family 2, subfamily C, |
| | | | | | polypeptide 8, CPC8, P450 MP-12/MP-20, |
| | | | | | variant transcripts differ in 3′UTR |
| | | 3′UTR | CYP2C8Rev | TGAACCAGCAATTAATAACACTTT | |
| CYP2C9 | 486 | 3′UTR | CYP2C9For | TTTTTATTCCTGACCTCCATTTTA | cytochrome P450, family 2, subfamily C, |
| | | | | | poyeptide 9, CPC9, CYP2C, CYP2C10, |
| | | | | | P450IIC9, P450 MP-4, P450 PB-1 |
| | | 3′UTR | CYP2C9Rev | GCTTTTTATTTAGATCATGCAGAA | |
| CYP2C19 | 684 | 3′UTR | CYP2C19For | GACATCAACAACCCTCGGGACTTT | cytochrome P450, family 2, subfamily C, |
| | | | | | polypeptide 19,CPCJ, CYP2C, CYP 2C, |
| | | | | | P450C2C, P450IIC19 |
| | | 3′UTR | CYP2C19Rev | ATAGAAGGGCGGCACAGAAGCAAA | |
| CYP2D6 | 598 | CDNs | CYP2D6For | CTGACCTGTTCTCTGCCGGGATGG | cytochrome P450, family 2, subfamily D, |
| | | | | | polypeptide 6, CPD6, CYP2D, CYP2D@, |
| | | | | | CYP2DL1, P450C2D, P450-DB1 |
| | | CDNs | CYP2D6Rev | TTCTAGCGGGGCACAGCACAAAGC | |
| CYP2E1 | 656 | CDNs | CYP2E1For | AGAAGCTCCATGAAGAAATTGACA | cytochrome P450, family 2, subfamily E, |
| | | | | | polypeptide 1, CPE1, CYP2E, P450-J, |
| | | | | | P450C2E |
| | | CDNs | CYP2E1Rev | GTGATGATTTATTTATATTCTGGG | |
| CYP3A4 | 607 | 3′UTR | CYP3A4For | TTTGGTCATTGTAATCACTGTTCG | cytochrome P450, family 3, subfamily A, |
| | | | | | polypeptide 4, HLP, CP33, CP34, CYP3A, |
| | | | | | NF-25, CYP3A3, P450C3, P450PCN1 |
| | | 3′UTR | CYP3A4Rev | ATTAACTGTTTATTGCATCGAGAC | |
| CYP19A1 | 652 | CDNs | CYP19A1For | ATGATCTGTCTGTGGCAAAAGTTT | cytochrome P450, family 19, subfamily A, |
| | | | | | polypeptide 1, ARO, ARO1, CPV1, CYAR, |
| | | | | | CYP19, P-450AROM, variant transcripts |
| | | | | | identical in 3′UTR |
| | | CDNs | CYP19A1Rev | AGTTCCTCCATTCATTTGATTTCC | |
| CYP27A1 | 538 | 3′UTR | CYP27A1For | CCGGGACCCCACTGCCTTCTCTGA | cytochrome P450, family 27, subfamily A, |
| | | | | | polypeptide 1, nuclear gene encoding |
| | | | | | mitochondrial protein, CTX, CP27, CYP27 |
| | | 3′UTR | CYP27A1Rev | TTTTATATTCTACCCAAGGACAGC | |
| CYP27B1 | 620 | 3′UTR | CYP27B1For | CTTCCCCTAATGCCTATCTGACCA | cytochrome P450, family 27, subfamily B, |
| | | | | | polypeptide 1, nuclear gene encoding |
| | | | | | mitochondrial protein, |
| | | 3′UTR | CYP27B1Rev | CCTGAGAACTAAGTGATGGGGCAA | VDR, CP2B, CYP1, PDDR, VDD1, VDDR, VDDRI, |
| | | | | | CYP27B, P450c1, CYP1alpha |
| CAR1 | 630 | CDNs | CARF | CAAACACAAAACTTCCTCTGCGGG | CAR = constitutive androstane receptor |
| | | | | | beta, NR1I3. Interacts with RAREs, |
| | | | | | transcriptional regulator of |
| | | | | | CYPs 3A4, 3A5, 2B6, 2B10, 2C9 |
| | | 3′UTR | CARR | TCTTTCATTGCAACCACTGCGCTC | NR1I3, nuclear receptor subfamily 1, |
| | | | | | group I, member 3, CAR, CAR1, MB67, |
| | | | | | CAR-SV1, CAR-BETA |
| FXR | 619 | CDNs | FXF | CCAGATAGACAATACATAAAGGAT | FXR = famesoid X receptor, represses |
| | | | | | CYP7A1, induces UGT2B4, NR1H4 |
| | | 3′UTR | FXR | CTGTTGCCATTATGTTTGCTTTAT | NR1H4, nuclear receptor subfamily 1, |
| | | | | | group H, member 4,BAR, FXR, HRR1, |
| | | | | | HRR-1, RIP14 |
| LXR | 626 | CDNs | LXF | GCTCATCGCCATCAACATCTTCTC | LXR = liver X receptor, encodes lxrb |
| | | | | | protein, NER, UNR, LXRB, LXR-b, |
| | | | | | NER-I, RIP15, NR1H2 |
| | | 3′UTR | LXR | TAAAAGCAGAGGAAGAGGAAGGCC | NR1H2, nuclear receptor subfamily 1, |
| | | | | | group H, member 2 |
| PPARA | 682 | 3′UTR | PARAF | AAGCAGAAAGCAGAAACCACAGAC | peroxisome proliferative activated |
| | | | | | receptor, alpha (PPARA), transcript |
| | | | | | variant 3, PPAR, NR1C1, hPPAR |
| | | 3′UTR | PARAR | CAGTAGGACATCCCAAACACAGAA | NR1C1, nuclear receptor subfamily 1, |
| | | | | | group C, member 1 |
| PPARD | 914 | 3′UTR | PARDF | CACACACACATAAGCACTGAAATC | peroxisome proliferative activated |
| | | | | | receptor, delta (PPARD), FAAR, NUC1, |
| | | | | | NUCI, NR1C2, NUCII, PPARB, PPAR-beta |
| | | 3′UTR | PARDR | AAAGTTTCGTCAGTCTGTGTACAC | NR1C2, nuclear receptor subfamily 1, |
| | | | | | group C, member 2 |
| PPARG | 795 | CDNs | PARGF | AGGAAAGACAACAGACAAATCACC | peroxisome proliferative activated |
| | | | | | receptor, gamma (PPARG), transcript |
| | | | | | variant 1, NR1C3, PPARG1, PPARG2, |
| | | | | | HUMPPARG |
| | | 3′UTR | PARGR | TTAGGTGTCAGATTTTCCCTCAGA | NR1C3, nuclear receptor subfamily 1, |
| | | | | | group C, member 3 |
| RXRA | 581 | 3′UTR | RXAF | CAGACAGCTTTAGCCGTTCCCAAT | RXRA = retinoid X receptor alpha, |
| | | | | | NR2B1 |
| | | 3′UTR | RXAR | TCTCCTCTCACACTTCTCCCTTTG | NR2B1, nuclear receptor subfamily 2, |
| | | | | | group B, member 1 |
| RXRB | 742 | 3′UTR | RXBF | CCTTGCTTCCITCTCATCTTGCCT | RXRB = retinoid X receptor beta, |
| | | | | | NR2B2, DAUDI6, RCoR-1, MGC1831, |
| | | | | | H-2RIIBP |
| | | 3′UTR | RXBR | TATGTATGAGAGGGGAAAGGAGCC | NR2B2, nuclear receptor subfamily 2, |
| | | | | | group B, member 2 |
| RXRG | 586 | CDNs | RXGF | TTGCTGATTGCCTCTTTCTCCCAC | RXRG = retinoid X receptor gamma, |
| | | | | | RXRC, NR2B3 |
| | | 3′UTR | RXGR | ATCACATTTTGGGGACAGGAAGGG | NR2B3, nuclear receptor subfamily 2, |
| | | | | | group B, member 3 |
| SXR | 736 | 3′UTR | SXF | CACGTTTGTTCGCTTCCTGAGTCT | PXR = pregnane X receptor, SXR = |
| | | | | | steroid and xenobiotic receptor, NR1I2, |
| | | | | | transcriptional regulator of CYP3A4, |
| | | | | | variant transcripts identical in 3′UTR. |
| | | 3′UTR | SXR | CAAGTGGCTATAAACAAGGCAGGC | NR1I2, nuclear receptor subfamily 1, |
| | | | | | group I, member 2, transcript |
| | | | | | variant 1BXR, PAR, PRR, PXR, |
| | | | | | SAR, SXR, ONR1, PAR1, PAR2, PARg |
| CNT1 | 662 | CDNs | CNT1F | CCAAGTTTAGGAGGGAGGAAGGAG | SLC28A1, solute carrier family 28 |
| | | | | | (sodium-coupled nucleoside trans- |
| | | | | | porter), member 1, HCNT1, concentrative |
| | | | | | Na+-nucleoside cotransporter 1 |
| | | 3′UTR | CNT1R | GCTACTGCTGCTGAGGGTCGTGTT | |
| CNT2 | 618 | 3′UTR | CNT2F | CATAGGAATCACACTTGGAGGCTT | SLC28A2, solute carrier family 28 |
| | | | | | (sodium-coupled nucleoside trans- |
| | | | | | porter), member 2, HCNT2, concentrative |
| | | | | | Na+-nucleosidecotransporter 2, SPNT1 |
| | | 3′UTR | CNT2R | CCTTTAGTAGAGACGGGGTTTCAC | |
| CNT3 | 708 | CDNs | CNT3F | CGTCATTGGCTGCTGCTAAACTCT | SLC28A3, solute carrier family 28 |
| | | | | | (sodium-coupled nucleoside trans- |
| | | | | | porter), member 3, HCNT3, concentrative |
| | | | | | Na+-nucleoside cotransporter 3 |
| | | 3′UTR | CNT3R | CAGGGAAAGTGGAGTTGAAGGCAT | |
| ENT1 | 701 | 3′UTR | ENT1F | TGTTTGTGCCACTGCTGCTGCTGT | SLC29A1, solute carrier family 29 |
| | | | | | (nucleoside transporters), |
| | | | | | member 1, hENT1, equilibrative |
| | | | | | nucleoside tranporter 1 |
| | | 3′UTR | ENT1R | GGGGAGAATGGAGTATATCAGGTC | |
| ENT2 | 684 | CDNs | ENT2F | CCCAGTAGTCCCCAGAAAGTAGCT | SLC29A2, solute carrier family 29 |
| | | | | | (nucleoside transporters), |
| | | | | | member 2, hENT2, equilibrative |
| | | | | | nucleoside tranporter 2, DER12, HNP36 |
| | | 3′UTR | ENT2R | ACGTCGAGAAGAGGCTGCCAAAGA | |
| ENT3 | 653 | CDNs | ENT3F | CTTCAGCAGCAGCATCTACGGCAT | SLC29A3, solute carrier family 29 |
| | | | | | (nucleoside transporters), |
| | | | | | member 3, hENT3, equilibrative |
| | | | | | nucleoside tranporter 3 |
| | | CDNs | EN73R | GGTAGTTACAGAGCACGAAGAGGG | |
| LST1 | 693 | CDNs | LST1F | GAGCAACAGTATGGTCAGCCTTCA | SLCO1B1, solute carrier organic anion |
| | | | | | transporter family, member |
| | | | | | 1B1, LST-1, OATP-C, OATP1B1 |
| | | CDNs | LST1R | CAGAGCCCCAAAATATATAGGAGC | |
| LST2 | 580 | CDNs | LST2F | CCTAACCTTGACCTATGATGGAAA | LST-2, liver specific organic anion |
| | | | | | transporter |
| | | CDNs | LST2R | TATAGATAAGCCCAAGTAGACCCT | |
| LST3 | 779 | CDNs | LST3F | GGGCTCTGATTGATAAAACATGTA | SLCO1B3, OATP8, OATPIB3, SLC21A8, |
| | | | | | LST-3TM13 |
| | | 3′UTR | LST3R | TGAAAAATATACAACTTAACATGA | |
| NTCP | 687 | CDNs | NTCPF | CCATGACACCACTCTTGATTGCCA | SLC10A1, solute carrier family 10 |
| | | | | | (sodium/bile acid cotransporter |
| | | | | | family), member 1, NTCP1 |
| | | CDNs | NTCPR | TTTAGAGATCCCAGCAAGAGGCAG | |
| NTCP2 | 594 | 3′UTR | NTCP2F | TTCTGCTTTTCAAATTCATAACAT | SLC10A2, solute carrier family 10 |
| | | | | | (sodium/bile acid cotransporter |
| | | | | | family), member 2, ASBT, ISBT, NTCP2 |
| | | 3′UTR | NTCP2R | TCATTTTCATTTATTTAAGCCTTT | |
| OAT1 | 606 | CDNs | OAT1F | ATCAATGGGAAGCGGGAAGAAGGA | SLC22A6, solute carrier family 22 |
| | | | | | (organic anion transporter), |
| | | | | | member 6, PAHT, HOAT1, ROAT1 |
| | | CDNs | OAT1R | CACAGGAACAGCACCGTAGATGAA | |
| OAT2 | 658 | CDNs | OAT2F | ACCTTCATACCTAGACCTGTTCCG | SLC22A7, solute cerrier family 22 |
| | | | | | (organic anion transporter), |
| | | | | | member 7, NLT |
| | | CDNs | OAT2R | CACTTAGTTCTGGACCTGCTTCAT | |
| OAT3 | 691 | CDNs | OAT3F | AAGTGACCTGTTCCGGATACCCAT | SLC22A8, solute carrier family 22 |
| | | | | | (organic anion transporter), |
| | | | | | member 8 |
| | | CDNs | OAT3R | CCAGTTTTCCAGGTCTTCGATCGT | |
| OAT4 | 596 | 3′UTR | OAT4F | GCCTAACCTGCCTCACCATCTACA | SLC22A11, solute carrier family 22 |
| | | | | | (organic anion/cation transporter), |
| | | | | | member 11, hOAT4 |
| | | 3′UTR | OAT4R | GTCTCGTTATTGGTTGGGCATGGC | |
| OAT4L | 698 | 3′UTR | OAT4LF | AAGAAGGCAACACATGGCACGCTG | SLC22A12, solute carrier family 22 |
| | | | | | (organic anion/cation transporter), |
| | | | | | member 12, RST, URAT1 |
| | | 3′UTR | OAT4LR | TGGGTAGGAGTTTCACGGGCATCT | |
| OATPA | 547 | 3′UTR | OATPAF | CCTGCACCTATATATTTTGGCGCT | SLCO1A2, solute carrier organic |
| | | | | | anion transporter family, member |
| | | | | | 1A2, SLC21A3, OATP, OATP-A, OATP1A2 |
| | | 3′UTR | OATPAR | CTTTAGGGGGCTGTTATTGATGTC | |
| OATPB | 771 | 3′UTR | OATPBF | TTCAGACAAACACACACTCAGCGC | SLCO2B1, solute carrier organic |
| | | | | | anion transporter family, member |
| | | | | | 2B1, SLC21A9, OATPB, OATP-B, OATP2B1 |
| | | 3′UTR | OATPBR | CTGGGAAACAAGAGGGATGAAGGA | |
| OATPC | 746 | CDNs | OATPCF | GAATTGAAATCACTTGCACTGGGT | SLCO1B1, solute carrier organic |
| | | | | | anion transporter family, member |
| | | | | | 1B1, SLC21A6, OATP2, OATP-C, OATP1B1 |
| | | CDNs | OATPCR | GAATCTAGCTCCTCCTTTTTAACC | |
| OATPD | 559 | 3′UTR | OATPDF | TCAAGATCTTCCTGGTGTCCGAGT | SLCO3A1, solute carrier organic |
| | | | | | anion transporter family, member |
| | | | | | 3A1, SLC21A11, OATP-D, OATP3A1 |
| | | 3′UTR | OATPDR | CCAAATACCAGCATCGTGAACAGG | |
| OATPE | 709 | 3′UTR | OATPEF | ACGGCCTCATGTACTTCTCACTGT | SLCO4A1, solute carrier organic |
| | | | | | anion transporter family, member |
| | | | | | 4A1, SLC21A12, POAT, OATP1, |
| | | | | | OATP-E, OATP4A1, OATPRP1 |
| | | 3′UTR | OATPER | GCAGGTCAAATAGAAGTTCCCGTG | |
| OATPF | 689 | 3′UTR | OATPFF | TGGGACTAACTGTGATACTGGGCA | SLCO1C1, solute carrier organic |
| | | | | | anion transporter family, member |
| | | | | | 1C1, SLC21A14, OATP1, OATP-F, OATP1C1 |
| | | 3′UTR | OATPFR | CACAGATGAAGACAGCTATGGGAG | |
| OATPRP4 | 666 | CDNs | OATPRP4F | GGAGAGACCTTTTGCACTGGGAAT | SLCO5A1, solute carrier organic |
| | | | | | anion transporter family, member |
| | | | | | 5A1, OATP-J, OATP5A1, SLC21A15 |
| | | CDNs | OATPRP4R | CCCTCAATGAATAGCGGCTGTGTA | |
| OATPRP5 | 650 | 3′UTR | OATPRP5F | GGGCACAGTGTCAATTCTCCTAAG | OATPRP5, organic anion transporter |
| | | | | | polypeptide-related protein 5 |
| | | 3′UTR | OATPRP5R | CACAGATGAAGACAGCTATGGGAG | |
| OATP8 | 624 | CDNs | OATP8F | AGGGTCTACTTGGGCTTATCTATA | SLC21A8, SLCO1B3, solute carrier |
| | | | | | organic anion transporter |
| | | | | | family, member 1B3, OATP1B3 |
| | | CDNs | OATP8R | GGCCTAAGTAATACATCCAAAGTG | |
| OCT1 | 722 | CDNs | OCT1F | AGCCCTTCATTTGCAGACCTGTTC | SLC22A1, solute carrier family 22 |
| | | | | | (organic cation transporter), |
| | | | | | member 1 |
| | | CDNs | OCT1R | ACTCCATCTTCATCCCTCCAACAC | |
| OCT2 | 617 | 3′UTR | OCT2F | ATTCCTGGTCTACCGGCTCACTAA | SLC22A2, solute carrier family 22 |
| | | | | | (organic cation transporter), |
| | | | | | member 2 |
| | | 3′UTR | OCT2R | GATGCTCCTCTCCCAACTTTACTG | |
| OCTN1 | 634 | 3′UTR | OCTN1F | TTGCTGCTATGGATGCTGACCTCA | SLC22A4, solute carrier family 22 |
| | | | | | (organic cation transporter), |
| | | | | | member 4 |
| | | 3′UTR | OCTN1R | CTGCATCTGCTCTAAGGTTTCTGG | |
| OCTN2 | 652 | 3′UTR | OCTN2F | ACTGATGTGTGAGCTCTTAAGACC | SLC22A5, solute carrier family 22 |
| | | | | | (organic cation transporter), |
| | | | | | member 5 |
| | | 3′UTR | OCTN2R | GAGGCATATGCTTTAGGAGTACCA | |
| ORCTL3 | 583 | 3′UTR | ORCTL3F | TGCCTAAACACCTCCTTGGATATG | SLC22A13, solute carrier family 22 |
| | | | | | (organic cation transporter), |
| | | | | | member 13, OCTL1, OCTL3 |
| | | 3′UTR | ORCTL3R | TGGGCCATCTTTGAAGTGAACACA | |
| ORCTL4 | 528 | CDNs | ORCTL4F | CCACAGAGCTGAAATCCATGACGA | SLC22A14, solute carrier family 22 |
| | | | | | (organic cation transporter), |
| | | | | | member 14, OCTL2, OCTL4 |
| | | 3′UTR | ORCTL4R | GGCCACTCAATTCCAACCCAAGAT | |
| PGT | 705 | 3′UTR | PGTF | GGTTGAGAGACACAGCTGCTACGT | SLCO2A1, solute carrier organic |
| | | | | | anion transporter family, |
| | | | | | member 2A1, SLC21A2, OATP2A1 |
| | | 3′UTR | PGTR | AAAGACCAGGGTTAGTTGCAGGGC | |
| SLC22A1L | 523 | CDNs | SLC22A1LF | AGCACCAAAGGGGCCAAAACTGAC | SLC22A18, solute carrier family 22 |
| | | | | | (organic cation transporter), |
| | | | | | member 18, ORCTL2 |
| | | 3′UTR | SLC22A1LR | GAGTTCGGAGCAGTGGTTGTACAG | |
| SLC22A3 | 696 | 3′UTR | SLC22A3F | TTCATCAAATCTGGTCAAGGGACT | solute carrier family 22 |
| | | | | | (extraneuronal monoamine |
| | | | | | transporter), member 3 |
| | | 3′UTR | SLC22A3R | GTTCCACATTTCAAAAGCCTCGAT | |
| SULT1A1 | 625 | CDNs | SULT1A1F | CCACCCTGTTCTCTACCTCTTCTA | sulfotransferase family, cytosolic, |
| | | | | | 1A, phenol-preferring, member 1 |
| | | 3′UTR | SULT1A1R | CAGAATCTCACTATGTTGCCCAGG | |
| SULT1B1 | 585 | CDNs | SULT1B1F | GCTCGTAATGCCAAGGATGTTTCA | sulfotransferase family, cytosolic, |
| | | | | | 1B, member 1 |
| | | 3′UTR | SULT1B1R | GCCCAAATCAATTCATAACTGCCC | |
| SULT1C1 | 675 | CDNs | SULT1C1F | AAAGCAATGCCCTCTCCACGGATA | sulfotransferase family, cytosolic, |
| | | | | | 1C, member 1 |
| | | 3′UTR | SULT1C1R | TCTGGCTGGGACTGAAGGATTGAA | |
| SULT1E1 | 492 | 3′UTR | SULT1E1F | CCTTGACTCAATTGATCCTCCCAT | sulfotransferase, estrogen- |
| | | | | | preferring (STE) |
| | | 3′UTR | SULT1E1R | CATTCCCATAGGTTATAGTTGTGC | |
| SULT2A1 | 602 | CDNs | SULT2A1F | GATGTCCAATTATTCCCTCCTGAG | sulfotransferase family, cytosolic, |
| | | | | | 2A, dehydroepiandrosterone |
| | | | | | (DHEA)-preferring, member 1 |
| | | 3′UTR | SULT2A1R | ATAGGGTTTCATCATGTTGGCCAG | |
| SULT2B1B | 597 | CDNs | SULT2B1BF | TGCGGGACGACGACATCTTTATCA | sulfotransferase family, cytosolic, |
| | | | | | 2B, member 1 |
| | | CDNs | SULT2B1BR | AGTTGGACATGGTGTTGGCCTTCA | |
| UGT2A1 | 524 | CDNs | UGT2A1F | ACTACGTTATGTGAGACTATGGGG | UDP glycosyltransferase 2 family, |
| | | | | | polypeptide A1 |
| | | CDNs | UGT2A1R | TTTAGGTTCACTTCCACAGCTGCT | |
| UGT2B4 | 476 | CDNs | UGT2B4F | CCAATGGCATCTATAAGGCAATCT | UDP glycosyltransferase 2 family, |
| | | | | | polypeptide B4 |
| | | CDNs | UGT2B4R | TTCCAGCCTCAGACGTAATTAATC | |
| UGT2B8 | 543 | CDNs | UGT2B8F | TCTGGATTGAGTTTGTCATGCGCC | UGT2B15, UDP glycosyltransferase 2 |
| | | | | | family, polypeptide B15 |
| | | 3′UTR | UGT2B8R | TTAGGGTACATGTGCACAACGAAG | |
| UGT2B17 | 506 | CDNs | UGT2B17F | TCGAGCAGTCTTCTGGATTGAGTT | UDP glycosyltransferase 2 family, |
| | | | | | polypeptide B17 |
| | | 3′UTR | UGT2B17R | AGCTCAGTAACTTTTCTGTGGGGT | |
| UGT8 | 457 | CDNs | UGT8F | TGGAGCTGGTGTCAAGTATCTGTC | UDP glycosyltransferase 8 |
| | | | | | (UDP-galactose ceramide |
| | | | | | galactosyltransferese) |
| | | CDNs | UGT8R | GATAGTTCGATTGACAGGGTGACC | |

We claim:

1. An array comprising two or more nucleic acid molecules immobilized on a substrate, wherein at least two of the nucleic acid molecules have a nucleic acid sequence consisting of:
(a) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288;
(b) a nucleic acid sequence prepared using amplification and primer pairs, wherein the primer pairs are selected from the following pairs of nucleic acid sequences:
SEQ ID NO:1 and SEQ ID NO:2;
SEQ ID NO:5 and SEQ ID NO:6;
SEQ ID NO:9 and SEQ ID NO:10;
SEQ ID NO:13 and SEQ ID NO:14;
SEQ ID NO:17 and SEQ ID NO:18;
SEQ ID NO:21 and SEQ ID NO:22;
SEQ ID NO:25 and SEQ ID NO:26;
SEQ ID NO:29 and SEQ ID NO:30;
SEQ ID NO:33 and SEQ ID NO:34;
SEQ ID NO:37 and SEQ ID NO:38;
SEQ ID NO:41 and SEQ ID NO:42;
SEQ ID NO:45 and SEQ ID NO:46;
SEQ ID NO:49 and SEQ ID NO:50;
SEQ ID NO:53 and SEQ ID NO:54;
SEQ ID NO:57 and SEQ ID NO:58;
SEQ ID NO:61 and SEQ ID NO:62;
SEQ ID NO:65 and SEQ ID NO:66;
SEQ ID NO:69 and SEQ ID NO:70;
SEQ ID NO:73 and SEQ ID NO:74;
SEQ ID NO:77 and SEQ ID NO:78;
SEQ ID NO:81 and SEQ ID NO:82;
SEQ ID NO:85 and SEQ ID NO:86;
SEQ ID NO:89 and SEQ ID NO:90;
SEQ ID NO:93 and SEQ ID NO:94;
SEQ ID NO:97 and SEQ ID NO:98;
SEQ ID NO:101 and SEQ ID NO:102;
SEQ ID NO:105 and SEQ ID NO:106;
SEQ ID NO:109 and SEQ ID NO:110;
SEQ ID NO:113 and SEQ ID NO:114;
SEQ ID NO:117 and SEQ ID NO:118;
SEQ ID NO:121 and SEQ ID NO:122;
SEQ ID NO:125 and SEQ ID NO:126;
SEQ ID NO:129 and SEQ ID NO:130;
SEQ ID NO:133 and SEQ ID NO:134;
SEQ ID NO:137 and SEQ ID NO: 138;
SEQ ID NO:141 and SEQ ID NO:142;
SEQ ID NO:145 and SEQ ID NO:146;
SEQ ID NO:149 and SEQ ID NO:150;
SEQ ID NO:153 and SEQ ID NO:154;
SEQ ID NO:157 and SEQ ID NO:158;
SEQ ID NO:161 and SEQ ID NO:162;
SEQ ID NO:165 and SEQ ID NO:166;
SEQ ID NO:169 and SEQ ID NO:170;
SEQ ID NO:173 and SEQ ID NO:174;
SEQ ID NO:177 and SEQ ID NO:178;
SEQ ID NO:181 and SEQ ID NO:182;
SEQ ID NO:185 and SEQ ID NO:186;
SEQ ID NO:189 and SEQ ID NO:190;
SEQ ID NO:193 and SEQ ID NO:194;
SEQ ID NO:197 and SEQ ID NO:198;
SEQ ID NO:201 and SEQ ID NO:202;
SEQ ID NO:205 and SEQ ID NO:206;
SEQ ID NO:209 and SEQ ID NO:210;
SEQ ID NO:213 and SEQ ID NO:214;
SEQ ID NO:217 and SEQ ID NO:218;
SEQ ID NO:221 and SEQ ID NO:222;
SEQ ID NO:225 and SEQ ID NO:226;
SEQ ID NO:229 and SEQ ID NO:230;
SEQ ID NO:233 and SEQ ID NO:234;
SEQ ID NO:237 and SEQ ID NO:238;
SEQ ID NO:241 and SEQ ID NO:242;
SEQ ID NO:245 and SEQ ID NO:246;
SEQ ID NO:249 and SEQ ID NO:250;
SEQ ID NO:253 and SEQ ID NO:254;
SEQ ID NO:257 and SEQ ID NO:258;
SEQ ID NO:261 and SEQ ID NO:262;
SEQ ID NO:265 and SEQ ID NO:266;
SEQ ID NO:269 and SEQ ID NO:270;
SEQ ID NO:273 and SEQ ID NO:274;
SEQ ID NO:277 and SEQ ID NO:278;
SEQ ID NO:281 and SEQ ID NO:282; or
SEQ ID NO:285 and SEQ ID NO:286;
(c) a nucleic acid sequence in (a) or (b) wherein T can also be U; or
(d) a fragment of (a) to (c).

2. The array according to claim 1, comprising at least 10 different nucleic acid molecules according to claim 1.

3. The array according to claim 1, comprising at least 20 different nucleic acid molecules according to claim 1.

4. The array according to claim 1, comprising at least 30 different nucleic acid molecules according to claim 1.

5. The array according to claim 1, comprising at least 40 different nucleic acid molecules according to claim 1.

6. The array according to claim 1, comprising at least 50 different nucleic acid molecules according to claim 1.

7. The array according to claim 1, comprising at least 60 different nucleic acid molecules according to claim 1.

8. The array according to claim 1, comprising at least 72 different nucleic acid molecules according to claim 1.

9. The array according to any one of claims 1 to 8, further comprising one or more control nucleic acid molecules.

10. The array according to claim 9, wherein the one or more control nucleic acid molecules comprise one or more expression level controls.

11. The array according to any one of claims 1 to 10, wherein the array is a microarray.

12. An array for screening a sample for the presence of nucleic acid molecules that encode cytochrome P450 enzymes, uptake transporters and/or nuclear xenoreceptors, the array comprising a substrate having immobilized in distinct spots thereon at least 2 nucleic acid probes selected from the group consisting of:
1) a probe that specifically hybridizes to a nucleic acid sequence encoding CYP1A2, wherein the nucleic acid sequence of the probe is selected from the group consisting of:
(a) a nucleic acid sequence consisting of SEQ ID NO:4,
(b) a nucleic acid sequence prepared using amplification and primer pairs having the nucleic acid sequence of SEQ ID NO:1 and SEQ ID NO:2,
(c) a nucleic acid sequence of (a) or (b) wherein T can be U, and
(d) a fragment of (a), (b) or (c);
2) a probe that specifically hybridizes to a nucleic acid sequence encoding CYP1B1, wherein the nucleic acid sequence of the probe is selected from the group consisting of:
(a) a nucleic acid sequence consisting of SEQ ID NO:8,
(b) a nucleic acid sequence prepared using amplification and primer pairs having the nucleic acid sequence of SEQ ID NO:5 and SEQ ID NO:6,
(c) a nucleic acid sequence of (a) or (b) wherein T can be U, and
(d) a fragment of (a), (b) or (c);
3) a probe that specifically hybridizes to a nucleic acid sequence encoding CYP2A6, wherein the nucleic acid sequence of the probe is selected from the group consisting of:
(a) a nucleic acid sequence consisting of SEQ ID NO:12,
(b) a nucleic acid sequence prepared using amplification and primer pairs having the nucleic acid sequence of SEQ ID NO:9 and SEQ ID NO:10,
(c) a nucleic acid sequence of (a) or (b) wherein T can be U, and
(d) a fragment of (a), (b) or (c);
4) a probe that specifically hybridizes to a nucleic acid sequence encoding CYP2B6, wherein the nucleic acid sequence of the probe is selected from the group consisting of:
(a) a nucleic acid sequence consisting of SEQ ID NO:16,
(b) a nucleic acid sequence prepared using amplification and primer pairs having the nucleic acid sequence of SEQ ID NO:13 and SEQ ID NO:14,
(c) a nucleic acid sequence of (a) or (b) wherein T can be U, and
(d) a fragment of (a), (b) or (c);
5) a probe that specifically hybridizes to a nucleic acid sequence encoding CYP2C8 variant 1, wherein the nucleic acid sequence of the probe is selected from the group consisting of:
(a) a nucleic acid sequence consisting of SEQ ID NO:20,
(b) a nucleic acid sequence prepared using amplification and primer pairs having the nucleic acid sequence of SEQ ID NO:17 and SEQ ID NO:18,
(c) a nucleic acid sequence of (a) or (b) wherein T can be U, and
(d) a fragment of (a), (b) or (c);
6) a probe that specifically hybridizes to a nucleic acid sequence encoding CYP2C8 variant 2, wherein the nucleic acid sequence of the probe is selected from the group consisting of:
(a) a nucleic acid sequence consisting of SEQ ID NO:24,
(b) a nucleic acid sequence prepared using amplification and primer pairs having the nucleic acid sequence of SEQ ID NO:21 and SEQ ID NO:22,
(c) a nucleic acid sequence of (a) or (b) wherein T can be U, and
(d) a fragment of (a), (b) or (c);
7) a probe that specifically hybridizes to a nucleic acid sequence encoding CYP2C9, wherein the nucleic acid sequence of the probe is selected from the group consisting of:
(a) a nucleic acid sequence consisting of SEQ ID NO:28,
(b) a nucleic acid sequence prepared using amplification and primer pairs having the nucleic acid sequence of SEQ ID NO:25 and SEQ ID NO:26,
(c) a nucleic acid sequence of (a) or (b) wherein T can be U, and
(d) a fragment of (a), (b) or (c);
8) a probe that specifically hybridizes to a nucleic acid sequence encoding CYP2C19, wherein the nucleic acid sequence of the probe is selected from the group consisting of:
(a) a nucleic acid sequence consisting of SEQ ID NO:32,
(b) a nucleic acid sequence prepared using amplification and primer pairs having the nucleic acid sequence of SEQ ID NO:29 and SEQ ID NO:30,
(c) a nucleic acid sequence of (a) or (b) wherein T can be U, and
(d) a fragment of (a), (b) or (c);
9) a probe that specifically hybridizes to a nucleic acid sequence encoding CYP2D6, wherein the nucleic acid sequence of the probe is selected from the group consisting of:
(a) a nucleic acid sequence consisting of SEQ ID NO:36,
(b) a nucleic acid sequence prepared using amplification and primer pairs having the nucleic acid sequence of SEQ ID NO:33 and SEQ ID NO:34,
(c) a nucleic acid sequence of (a) or (b) wherein T can be U, and
(d) a fragment of (a), (b) or (c);
10) a probe that specifically hybridizes to a nucleic acid sequence encoding CYP2E1, wherein the nucleic acid sequence of the probe is selected from the group consisting of:
(a) a nucleic acid sequence consisting of SEQ ID NO:40,
(b) a nucleic acid sequence prepared using amplification and primer pairs having the nucleic acid sequence of SEQ ID NO:37 and SEQ ID NO:38,
(c) a nucleic acid sequence of (a) or (b) wherein T can be U, and
(d) a fragment of (a), (b) or (c);
11) a probe that specifically hybridizes to a nucleic acid sequence encoding CYP3A4, wherein the nucleic acid sequence of the probe is selected from the group consisting of:
(a) a nucleic acid sequence consisting of SEQ ID NO:44,
(b) a nucleic acid sequence prepared using amplification and primer pairs having the nucleic acid sequence of SEQ ID NO:41 and SEQ ID NO:42,
(c) a nucleic acid sequence of (a) or (b) wherein T can be U, and
(d) a fragment of (a), (b) or (c);
12) a probe that specifically hybridizes to a nucleic acid sequence encoding CYP19A variant 1, wherein the nucleic acid sequence of the probe is selected from the group consisting of:
(a) a nucleic acid sequence consisting of SEQ ID NO:48,
(b) a nucleic acid sequence prepared using amplification and primer pairs having the nucleic acid sequence of SEQ ID NO:45 and SEQ ID NO:46,
(c) a nucleic acid sequence of (a) or (b) wherein T can be U, and
(d) a fragment of (a), (b) or (c);
13) a probe that specifically hybridizes to a nucleic acid sequence encoding CYP19A variant 2, wherein the nucleic acid sequence of the probe is selected from the group consisting of:
(a) a nucleic acid sequence consisting of SEQ ID NO:52,
(b) a nucleic acid sequence prepared using amplification and primer pairs having the nucleic acid sequence of SEQ ID NO:49 and SEQ ID NO:50,
(c) a nucleic acid sequence of (a) or (b) wherein T can be U, and
(d) a fragment of (a), (b) or (c);
14) a probe that specifically hybridizes to a nucleic acid sequence encoding CYP27A1, wherein the nucleic acid sequence of the probe is selected from the group consisting of:
(a) a nucleic acid sequence consisting of SEQ ID NO:56,
(b) a nucleic acid sequence prepared using amplification and primer pairs having the nucleic acid sequence of SEQ ID NO:53 and SEQ ID NO:54,
(c) a nucleic acid sequence of (a) or (b) wherein T can be U, and
(d) a fragment of (a), (b) or (c);
15) a probe that specifically hybridizes to a nucleic acid sequence encoding CYP27B1, wherein the nucleic acid sequence of the probe is selected from the group consisting of:
(a) a nucleic acid sequence consisting of SEQ ID NO:60,
(b) a nucleic acid sequence prepared using amplification and primer pairs having the nucleic acid sequence of SEQ ID NO:57 and SEQ ID NO:58,
(c) a nucleic acid sequence of (a) or (b) wherein T can be U, and
(d) a fragment of (a), (b) or (c);
16) a probe that specifically hybridizes to a nucleic acid sequence encoding CAR1, wherein the nucleic acid sequence of the probe is selected from the group consisting of:
(a) a nucleic acid sequence consisting of SEQ ID NO:64,
(b) a nucleic acid sequence prepared using amplification and primer pairs having the nucleic acid sequence of SEQ ID NO:61 and SEQ ID NO:62,
(c) a nucleic acid sequence of (a) or (b) wherein T can be U, and
(d) a fragment of (a), (b) or (c);
17) a probe that specifically hybridizes to a nucleic acid sequence encoding FXR, wherein the nucleic acid sequence of the probe is selected from the group consisting of:
(a) a nucleic acid sequence consisting of SEQ ID NO:68,
(b) a nucleic acid sequence prepared using amplification and primer pairs having the nucleic acid sequence of SEQ ID NO:65 and SEQ ID NO:66,
(c) a nucleic acid sequence of (a) or (b) wherein T can be U, and
(d) a fragment of (a), (b) or (c);
18) a probe that specifically hybridizes to a nucleic acid sequence encoding LXR, wherein the nucleic acid sequence of the probe is selected from the group consisting of:
(a) a nucleic acid sequence consisting of SEQ ID NO:72,
(b) a nucleic acid sequence prepared using amplification and primer pairs having the nucleic acid sequence of SEQ ID NO:69 and SEQ ID NO:70,
(c) a nucleic acid sequence of (a) or (b) wherein T can be U, and
(d) a fragment of (a), (b) or (c);
19) a probe that specifically hybridizes to a nucleic acid sequence encoding PPARA, wherein the nucleic acid sequence of the probe is selected from the group consisting of:
(a) a nucleic acid sequence consisting of SEQ ID NO:76,
(b) a nucleic acid sequence prepared using amplification and primer pairs having the nucleic acid sequence of SEQ ID NO:73 and SEQ ID NO:74,
(c) a nucleic acid sequence of (a) or (b) wherein T can be U, and
(d) a fragment of (a), (b) or (c);
20) a probe that specifically hybridizes to a nucleic acid sequence encoding PPARD-B, wherein the nucleic acid sequence of the probe is selected from the group consisting of:
(a) a nucleic acid sequence consisting of SEQ ID NO:80,
(b) a nucleic acid sequence prepared using amplification and primer pairs having the nucleic acid sequence of SEQ ID NO:77 and SEQ ID NO:78,
(c) a nucleic acid sequence of (a) or (b) wherein T can be U, and
(d) a fragment of (a), (b) or (c);
21) a probe that specifically hybridizes to a nucleic acid sequence encoding PPARG, wherein the nucleic acid sequence of the probe is selected from the group consisting of:
(a) a nucleic acid sequence consisting of SEQ ID NO:84,
(b) a nucleic acid sequence prepared using amplification and primer pairs having the nucleic acid sequence of SEQ ID NO:81 and SEQ ID NO:82,
(c) a nucleic acid sequence of (a) or (b) wherein T can be U, and
(d) a fragment of (a), (b) or (c);
22) a probe that specifically hybridizes to a nucleic acid sequence encoding RXRA, wherein the nucleic acid sequence of the probe is selected from the group consisting of:
(a) a nucleic acid sequence consisting of SEQ ID NO:88,
(b) a nucleic acid sequence prepared using amplification and primer pairs having the nucleic acid sequence of SEQ ID NO:85 and SEQ ID NO:86,
(c) a nucleic acid sequence of (a) or (b) wherein T can be U, and
(d) a fragment of (a), (b) or (c);
23) a probe that specifically hybridizes to a nucleic acid sequence encoding RXRB, wherein the nucleic acid sequence of the probe is selected from the group consisting of:
(a) a nucleic acid sequence consisting of SEQ ID NO:92,
(b) a nucleic acid sequence prepared using amplification and primer pairs having the nucleic acid sequence of SEQ ID NO:89 and SEQ ID NO:90,
(c) a nucleic acid sequence of (a) or (b) wherein T can be U, and
(d) a fragment of (a), (b) or (c);
24) a probe that specifically hybridizes to a nucleic acid sequence encoding RXRG, wherein the nucleic acid sequence of the probe is selected from the group consisting of:
(a) a nucleic acid sequence consisting of SEQ ID NO:96,
(b) a nucleic acid sequence prepared using amplification and primer pairs having the nucleic acid sequence of SEQ ID NO:93 and SEQ ID NO:94,
(c) a nucleic acid sequence of (a) or (b) wherein T can be U, and
(d) a fragment of (a), (b) or (c);
25) a probe that specifically hybridizes to a nucleic acid sequence encoding SXR (PXR) transcript variant 1, wherein the nucleic acid sequence of the probe is selected from the group consisting of:
(a) a nucleic acid sequence consisting of SEQ ID NO:100,
(b) a nucleic acid sequence prepared using amplification and primer pairs having the nucleic acid sequence of SEQ ID NO:97 and SEQ ID NO:98,
(c) a nucleic acid sequence of (a) or (b) wherein T can be U, and
(d) a fragment of (a), (b) or (c);
26) a probe that specifically hybridizes to a nucleic acid sequence encoding SULT1A1, wherein the nucleic acid sequence of the probe is selected from the group consisting of:
(a) a nucleic acid sequence consisting of SEQ ID NO:104,
(b) a nucleic acid sequence prepared using amplification and primer pairs having the nucleic acid sequence of SEQ ID NO:101 and SEQ ID NO:102,
(c) a nucleic acid sequence of (a) or (b) wherein T can be U, and
(d) a fragment of (a), (b) or (c);
27) a probe that specifically hybridizes to a nucleic acid sequence encoding SULT1B1, wherein the nucleic acid sequence of the probe is selected from the group consisting of:
(a) a nucleic acid sequence consisting of SEQ ID NO:108,
(b) a nucleic acid sequence prepared using amplification and primer pairs having the nucleic acid sequence of SEQ ID NO:105 and SEQ ID NO:106,
(c) a nucleic acid sequence of (a) or (b) wherein T can be U, and
(d) a fragment of (a), (b) or (c);
28) a probe that specifically hybridizes to a nucleic acid sequence encoding SULT1C1, wherein the nucleic acid sequence of the probe is selected from the group consisting of:
(a) a nucleic acid sequence consisting of SEQ ID NO:112,
(b) a nucleic acid sequence prepared using amplification and primer pairs having the nucleic acid sequence of SEQ ID NO:109 and SEQ ID NO:110,
(c) a nucleic acid sequence of (a) or (b) wherein T can be U, and
(d) a fragment of (a), (b) or (c);
29) a probe that specifically hybridizes to a nucleic acid sequence encoding SULT1E1, wherein the nucleic acid sequence of the probe is selected from the group consisting of:
(a) a nucleic acid sequence consisting of SEQ ID NO:116,
(b) a nucleic acid sequence prepared using amplification and primer pairs having the nucleic acid sequence of SEQ ID NO:113 and SEQ ID NO:114,
(c) a nucleic acid sequence of (a) or (b) wherein T can be U, and
(d) a fragment of (a), (b) or (c);
30) a probe that specifically hybridizes to a nucleic acid sequence encoding SULT2A1, wherein the nucleic acid sequence of the probe is selected from the group consisting of:
(a) a nucleic acid sequence consisting of SEQ ID NO:120,
(b) a nucleic acid sequence prepared using amplification and primer pairs having the nucleic acid sequence of SEQ ID NO:117 and SEQ ID NO:118,
(c) a nucleic acid sequence of (a) or (b) wherein T can be U, and
(d) a fragment of (a), (b) or (c);
31) a probe that specifically hybridizes to a nucleic acid sequence encoding SULT2B1b, wherein the nucleic acid sequence of the probe is selected from the group consisting of:
(a) a nucleic acid sequence consisting of SEQ ID NO:124,
(b) a nucleic acid sequence prepared using amplification and primer pairs having the nucleic acid sequence of SEQ ID NO:121 and SEQ ID NO:122,
(c) a nucleic acid sequence of (a) or (b) wherein T can be U, and
(d) a fragment of (a), (b) or (c);
32) a probe that specifically hybridizes to a nucleic acid sequence encoding UGT2A1, wherein the nucleic acid sequence of the probe is selected from the group consisting of:
(a) a nucleic acid sequence consisting of SEQ ID NO:128,
(b) a nucleic acid sequence prepared using amplification and primer pairs having the nucleic acid sequence of SEQ ID NO:125 and SEQ ID NO:126,
(c) a nucleic acid sequence of (a) or (b) wherein T can be U, and
(d) a fragment of (a), (b) or (c);
33) a probe that specifically hybridizes to a nucleic acid sequence encoding UGT2B4, wherein the nucleic acid sequence of the probe is selected from the group consisting of:
(a) a nucleic acid sequence consisting of SEQ ID NO:132,
(b) a nucleic acid sequence prepared using amplification and primer pairs having the nucleic acid sequence of SEQ ID NO:129 and SEQ ID NO:130,
(c) a nucleic acid sequence of (a) or (b) wherein T can be U, and
(d) a fragment of (a), (b) or (c);
34) a probe that specifically hybridizes to a nucleic acid sequence encoding UGT2B15, wherein the nucleic acid sequence of the probe is selected from the group consisting of:
(a) a nucleic acid sequence consisting of SEQ ID NO:136,
(b) a nucleic acid sequence prepared using amplification and primer pairs having the nucleic acid sequence of SEQ ID NO:133 and SEQ ID NO:134,
(c) a nucleic acid sequence of (a) or (b) wherein T can be U, and
(d) a fragment of (a), (b) or (c);
35) a probe that specifically hybridizes to a nucleic acid sequence encoding UGT2B17, wherein the nucleic acid sequence of the probe is selected from the group consisting of:
(a) a nucleic acid sequence consisting of SEQ ID NO:140,
(b) a nucleic acid sequence prepared using amplification and primer pairs having the nucleic acid sequence of SEQ ID NO:137 and SEQ ID NO:138,
(c) a nucleic acid sequence of (a) or (b) wherein T can be U, and
(d) a fragment of (a), (b) or (c);
36) a probe that specifically hybridizes to a nucleic acid sequence encoding UGT8, wherein the nucleic acid sequence of the probe is selected from the group consisting of:
(a) a nucleic acid sequence consisting of SEQ ID NO:144,
(b) a nucleic acid sequence prepared using amplification and primer pairs having the nucleic acid sequence of SEQ ID NO:141 and SEQ ID NO:142,
(c) a nucleic acid sequence of (a) or (b) wherein T can be U, and
(d) a fragment of (a), (b) or (c);
37) a probe that specifically hybridizes to a nucleic acid sequence encoding CNT1, wherein the nucleic acid sequence of the probe is selected from the group consisting of:
(a) a nucleic acid sequence consisting of SEQ ID NO:148,
(b) a nucleic acid sequence prepared using amplification and primer pairs having the nucleic acid sequence of SEQ ID NO:145 and SEQ ID NO:146,
(c) a nucleic acid sequence of (a) or (b) wherein T can be U, and
(d) a fragment of (a), (b) or (c);
38) a probe that specifically hybridizes to a nucleic acid sequence encoding CNT2, wherein the nucleic acid sequence of the probe is selected from the group consisting of:
(a) a nucleic acid sequence consisting of SEQ ID NO:152,
(b) a nucleic acid sequence prepared using amplification and primer pairs having the nucleic acid sequence of SEQ ID NO:149 and SEQ ID NO:150,
(c) a nucleic acid sequence of (a) or (b) wherein T can be U, and
(d) a fragment of (a), (b) or (c);
39) a probe that specifically hybridizes to a nucleic acid sequence encoding CNT3, wherein the nucleic acid sequence of the probe is selected from the group consisting of:
(a) a nucleic acid sequence consisting of SEQ ID NO:156,
(b) a nucleic acid sequence prepared using amplification and primer pairs having the nucleic acid sequence of SEQ ID NO:153 and SEQ ID NO:154,
(c) a nucleic acid sequence of (a) or (b) wherein T can be U, and
(d) a fragment of (a), (b) or (c);
40) a probe that specifically hybridizes to a nucleic acid sequence encoding ENT1, wherein the nucleic acid sequence of the probe is selected from the group consisting of:
(a) a nucleic acid sequence consisting of SEQ ID NO:160,
(b) a nucleic acid sequence prepared using amplification and primer pairs having the nucleic acid sequence of SEQ ID NO:157 and SEQ ID NO:158,
(c) a nucleic acid sequence of (a) or (b) wherein T can be U, and
(d) a fragment of (a), (b) or (c);
41) a probe that specifically hybridizes to a nucleic acid sequence encoding ENT2, wherein the nucleic acid sequence of the probe is selected from the group consisting of:
(a) a nucleic acid sequence consisting of SEQ ID NO:164,
(b) a nucleic acid sequence prepared using amplification and primer pairs having the nucleic acid sequence of SEQ ID NO:161 and SEQ ID NO:162,
(c) a nucleic acid sequence of (a) or (b) wherein T can be U, and
(d) a fragment of (a), (b) or (c);
42) a probe that specifically hybridizes to a nucleic acid sequence encoding ENT3, wherein the nucleic acid sequence of the probe is selected from the group consisting of:
(a) a nucleic acid sequence consisting of SEQ ID NO:168,
(b) a nucleic acid sequence prepared using amplification and primer pairs having the nucleic acid sequence of SEQ ID NO:165 and SEQ ID NO:166,
(c) a nucleic acid sequence of (a) or (b) wherein T can be U, and
(d) a fragment of (a), (b) or (c);
43) a probe that specifically hybridizes to a nucleic acid sequence encoding LST1, wherein the nucleic acid sequence of the probe is selected from the group consisting of:
(a) a nucleic acid sequence consisting of SEQ ID NO:172,
(b) a nucleic acid sequence prepared using amplification and primer pairs having the nucleic acid sequence of SEQ ID NO:169 and SEQ ID NO:170,
(c) a nucleic acid sequence of (a) or (b) wherein T can be U, and
(d) a fragment of (a), (b) or (c);
44) a probe that specifically hybridizes to a nucleic acid sequence encoding LST2, wherein the nucleic acid sequence of the probe is selected from the group consisting of:
(a) a nucleic acid sequence consisting of SEQ ID NO:176,
(b) a nucleic acid sequence prepared using amplification and primer pairs having the nucleic acid sequence of SEQ ID NO:173 and SEQ ID NO:174,
(c) a nucleic acid sequence of (a) or (b) wherein T can be U, and
(d) a fragment of (a), (b) or (c);
45) a probe that specifically hybridizes to a nucleic acid sequence encoding LST3, wherein the nucleic acid sequence of the probe is selected from the group consisting of:
(a) a nucleic acid sequence consisting of SEQ ID NO:180,
(b) a nucleic acid sequence prepared using amplification and primer pairs having the nucleic acid sequence of SEQ ID NO:177 and SEQ ID NO:178,
(c) a nucleic acid sequence of (a) or (b) wherein T can be U, and
(d) a fragment of (a), (b) or (c);
46) a probe that specifically hybridizes to a nucleic acid sequence encoding NTCP, wherein the nucleic acid sequence of the probe is selected from the group consisting of:
(a) a nucleic acid sequence consisting of SEQ ID NO:184,
(b) a nucleic acid sequence prepared using amplification and primer pairs having the nucleic acid sequence of SEQ ID NO:181 and SEQ ID NO:182,
(c) a nucleic acid sequence of (a) or (b) wherein T can be U, and
(d) a fragment of (a), (b) or (c);
47) a probe that specifically hybridizes to a nucleic acid sequence encoding NTCP2, wherein the nucleic acid sequence of the probe is selected from the group consisting of:
(a) a nucleic acid sequence consisting of SEQ ID NO:188,
(b) a nucleic acid sequence prepared using amplification and primer pairs having the nucleic acid sequence of SEQ ID NO:185 and SEQ ID NO:186,
(c) a nucleic acid sequence of (a) or (b) wherein T can be U, and
(d) a fragment of (a), (b) or (c);
48) a probe that specifically hybridizes to a nucleic acid sequence encoding OAT1, wherein the nucleic acid sequence of the probe is selected from the group consisting of:
(a) a nucleic acid sequence consisting of SEQ ID NO:192,
(b) a nucleic acid sequence prepared using amplification and primer pairs having the nucleic acid sequence of SEQ ID NO:189 and SEQ ID NO:190,
(c) a nucleic acid sequence of (a) or (b) wherein T can be U, and
(d) a fragment of (a), (b) or (c);
49) a probe that specifically hybridizes to a nucleic acid sequence encoding OAT2, wherein the nucleic acid sequence of the probe is selected from the group consisting of:
(a) a nucleic acid sequence consisting of SEQ ID NO:196,
(b) a nucleic acid sequence prepared using amplification and primer pairs having the nucleic acid sequence of SEQ ID NO:193 and SEQ ID NO:194,
(c) a nucleic acid sequence of (a) or (b) wherein T can be U, and
(d) a fragment of (a), (b) or (c);
50) a probe that specifically hybridizes to a nucleic acid sequence encoding OAT3, wherein the nucleic acid sequence of the probe is selected from the group consisting of:
(a) a nucleic acid sequence consisting of SEQ ID NO:200,
(b) a nucleic acid sequence prepared using amplification and primer pairs having the nucleic acid sequence of SEQ ID NO:197 and SEQ ID NO:198,
(c) a nucleic acid sequence of (a) or (b) wherein T can be U, and
(d) a fragment of (a), (b) or (c);
51) a probe that specifically hybridizes to a nucleic acid sequence encoding OAT4, wherein the nucleic acid sequence of the probe is selected from the group consisting of:
(a) a nucleic acid sequence consisting of SEQ ID NO:204,
(b) a nucleic acid sequence prepared using amplification and primer pairs having the nucleic acid sequence of SEQ ID NO:201 and SEQ ID NO:202,
(c) a nucleic acid sequence of (a) or (b) wherein T can be U, and
(d) a fragment of (a), (b) or (c);
52) a probe that specifically hybridizes to a nucleic acid sequence encoding OAT4L, wherein the nucleic acid sequence of the probe is selected from the group consisting of:
(a) a nucleic acid sequence consisting of SEQ ID NO:208,
(b) a nucleic acid sequence prepared using amplification and primer pairs having the nucleic acid sequence of SEQ ID NO:205 and SEQ ID NO:206,
(c) a nucleic acid sequence of (a) or (b) wherein T can be U, and
(d) a fragment of (a), (b) or (c);
53) a probe that specifically hybridizes to a nucleic acid sequence encoding OATP-A, wherein the nucleic acid sequence of the probe is selected from the group consisting of:
(a) a nucleic acid sequence consisting of SEQ ID NO:212,
(b) a nucleic acid sequence prepared using amplification and primer pairs having the nucleic acid sequence of SEQ ID NO:209 and SEQ ID NO:210,
(c) a nucleic acid sequence of (a) or (b) wherein T can be U, and
(d) a fragment of (a), (b) or (c);
54) a probe that specifically hybridizes to a nucleic acid sequence encoding OATP-B, wherein the nucleic acid sequence of the probe is selected from the group consisting of:
(a) a nucleic acid sequence consisting of SEQ ID NO:216,
(b) a nucleic acid sequence prepared using amplification and primer pairs having the nucleic acid sequence of SEQ ID NO:213 and SEQ ID NO:214,
(c) a nucleic acid sequence of (a) or (b) wherein T can be U, and
(d) a fragment of (a), (b) or (c);
55) a probe that specifically hybridizes to a nucleic acid sequence encoding OATP-C, wherein the nucleic acid sequence of the probe is selected from the group consisting of:
(a) a nucleic acid sequence consisting of SEQ ID NO:220,
(b) a nucleic acid sequence prepared using amplification and primer pairs having the nucleic acid sequence of SEQ ID NO:217 and SEQ ID NO:218,
(c) a nucleic acid sequence of (a) or (b) wherein T can be U, and
(d) a fragment of (a), (b) or (c);
56) a probe that specifically hybridizes to a nucleic acid sequence encoding OATP-D, wherein the nucleic acid sequence of the probe is selected from the group consisting of:
(a) a nucleic acid sequence consisting of SEQ ID NO:224,
(b) a nucleic acid sequence prepared using amplification and primer pairs having the nucleic acid sequence of SEQ ID NO:221 and SEQ ID NO:222,
(c) a nucleic acid sequence of (a) or (b) wherein T can be U, and
(d) a fragment of (a), (b) or (c);
57) a probe that specifically hybridizes to a nucleic acid sequence encoding OATP-E, wherein the nucleic acid sequence of the probe is selected from the group consisting of:
(a) a nucleic acid sequence consisting of SEQ ID NO:228,
(b) a nucleic acid sequence prepared using amplification and primer pairs having the nucleic acid sequence of SEQ ID NO:225 and SEQ ID NO:226,
(c) a nucleic acid sequence of (a) or (b) wherein T can be U, and
(d) a fragment of (a), (b) or (c);
58) a probe that specifically hybridizes to a nucleic acid sequence encoding OATP-F, wherein the nucleic acid sequence of the probe is selected from the group consisting of:
(a) a nucleic acid sequence consisting of SEQ ID NO:232,
(b) a nucleic acid sequence prepared using amplification and primer pairs having the nucleic acid sequence of SEQ ID NO:229 and SEQ ID NO:230,
(c) a nucleic acid sequence of (a) or (b) wherein T can be U, and
(d) a fragment of (a), (b) or (c);
59) a probe that specifically hybridizes to a nucleic acid sequence encoding OATP-RP1, wherein the nucleic acid sequence of the probe is selected from the group consisting of:
(a) a nucleic acid sequence consisting of SEQ ID NO:236,
(b) a nucleic acid sequence prepared using amplification and primer pairs having the nucleic acid sequence of SEQ ID NO:233 and SEQ ID NO:234,
(c) a nucleic acid sequence of (a) or (b) wherein T can be U, and
(d) a fragment of (a), (b) or (c);
60) a probe that specifically hybridizes to a nucleic acid sequence encoding OATP-RP2, wherein the nucleic acid sequence of the probe is selected from the group consisting of:
(a) a nucleic acid sequence consisting of SEQ ID NO:240,
(b) a nucleic acid sequence prepared using amplification and primer pairs having the nucleic acid sequence of SEQ ID NO:237 and SEQ ID NO:238,
(c) a nucleic acid sequence of (a) or (b) wherein T can be U, and
(d) a fragment of (a), (b) or (c);
61) a probe that specifically hybridizes to a nucleic acid sequence encoding OATP-RP4, wherein the nucleic acid sequence of the probe is selected from the group consisting of:
(a) a nucleic acid sequence consisting of SEQ ID NO:244,
(b) a nucleic acid sequence prepared using amplification and primer pairs having the nucleic acid sequence of SEQ ID NO:241 and SEQ ID NO:242,
(c) a nucleic acid sequence of (a) or (b) wherein T can be U, and
(d) a fragment of (a), (b) or (c);
62) a probe that specifically hybridizes to a nucleic acid sequence encoding OATP-RP5, wherein the nucleic acid sequence of the probe is selected from the group consisting of:
(a) a nucleic acid sequence consisting of SEQ ID NO:248,
(b) a nucleic acid sequence prepared using amplification and primer pairs having the nucleic acid sequence of SEQ ID NO:245 and SEQ ID NO:246,
(c) a nucleic acid sequence of (a) or (b) wherein T can be U, and
(d) a fragment of (a), (b) or (c);
63) a probe that specifically hybridizes to a nucleic acid sequence encoding OATP8, wherein the nucleic acid sequence of the probe is selected from the group consisting of:
(a) a nucleic acid sequence consisting of SEQ ID NO:252,
(b) a nucleic acid sequence prepared using amplification and primer pairs having the nucleic acid sequence of SEQ ID NO:249 and SEQ ID NO:250,
(c) a nucleic acid sequence of (a) or (b) wherein T can be U, and
(d) a fragment of (a), (b) or (c);
64) a probe that specifically hybridizes to a nucleic acid sequence encoding OCT1, wherein the nucleic acid sequence of the probe is selected from the group consisting of:
(a) a nucleic acid sequence consisting of SEQ ID NO:256,
(b) a nucleic acid sequence prepared using amplification and primer pairs having the nucleic acid sequence of SEQ ID NO:253 and SEQ ID NO:254,
(c) a nucleic acid sequence of (a) or (b) wherein T can be U, and
(d) a fragment of (a), (b) or (c);
65) a probe that specifically hybridizes to a nucleic acid sequence encoding OCT2, wherein the nucleic acid sequence of the probe is selected from the group consisting of:
(a) a nucleic acid sequence consisting of SEQ ID NO:260,
(b) a nucleic acid sequence prepared using amplification and primer pairs having the nucleic acid sequence of SEQ ID NO:257 and SEQ ID NO:258,
(c) a nucleic acid sequence of (a) or (b) wherein T can be U, and
(d) a fragment of (a), (b) or (c);
66) a probe that specifically hybridizes to a nucleic acid sequence encoding OCTN1, wherein the nucleic acid sequence of the probe is selected from the group consisting of:
(a) a nucleic acid sequence consisting of SEQ ID NO:264,
(b) a nucleic acid sequence prepared using amplification and primer pairs having the nucleic acid sequence of SEQ ID NO:261 and SEQ ID NO:262,
(c) a nucleic acid sequence of (a) or (b) wherein T can be U, and
(d) a fragment of (a), (b) or (c);
67) a probe that specifically hybridizes to a nucleic acid sequence encoding OCTN2, wherein the nucleic acid sequence of the probe is selected from the group consisting of:
(a) a nucleic acid sequence consisting of SEQ ID NO:268,
(b) a nucleic acid sequence prepared using amplification and primer pairs having the nucleic acid sequence of SEQ ID NO:265 and SEQ ID NO:266,
(c) a nucleic acid sequence of (a) or (b) wherein T can be U, and
(d) a fragment of (a), (b) or (c);
68) a probe that specifically hybridizes to a nucleic acid sequence encoding ORCTL3, wherein the nucleic acid sequence of the probe is selected from the group consisting of:
(a) a nucleic acid sequence consisting of SEQ ID NO:272,
(b) a nucleic acid sequence prepared using amplification and primer pairs having the nucleic acid sequence of SEQ ID NO:269 and SEQ ID NO:270,
(c) a nucleic acid sequence of (a) or (b) wherein T can be U, and
(d) a fragment of (a), (b) or (c);
69) a probe that specifically hybridizes to a nucleic acid sequence encoding ORCTL4, wherein the nucleic acid sequence of the probe is selected from the group consisting of:
(a) a nucleic acid sequence consisting of SEQ ID NO:276,
(b) a nucleic acid sequence prepared using amplification and primer pairs having the nucleic acid sequence of SEQ ID NO:273 and SEQ ID NO:274,
(c) a nucleic acid sequence of (a) or (b) wherein T can be U, and
(d) a fragment of (a), (b) or (c);
70) a probe that specifically hybridizes to a nucleic acid sequence encoding PGT, wherein the nucleic acid sequence of the probe is selected from the group consisting of:
(a) a nucleic acid sequence consisting of SEQ ID NO:280,
(b) a nucleic acid sequence prepared using amplification and primer pairs having the nucleic acid sequence of SEQ ID NO:277 and SEQ ID NO:278,
(c) a nucleic acid sequence of (a) or (b) wherein T can be U, and
(d) a fragment of (a), (b) or (c);
71) a probe that specifically hybridizes to a nucleic acid sequence encoding SLC22A1 L, wherein the nucleic acid sequence of the probe is selected from the group consisting of:
(a) a nucleic acid sequence consisting of SEQ ID NO:284,
(b) a nucleic acid sequence prepared using amplification and primer pairs having the nucleic acid sequence of SEQ ID NO:281 and SEQ ID NO:282,
(c) a nucleic acid sequence of (a) or (b) wherein T can be U, and
(d) a fragment of (a), (b) or (c); and
72) a probe that specifically hybridizes to a nucleic acid sequence encoding SLC22A3, wherein the nucleic acid sequence of the probe is selected from the group consisting of:
(a) a nucleic acid sequence consisting of SEQ ID NO:288,
(b) a nucleic acid sequence prepared using amplification and primer pairs having the nucleic acid sequence of SEQ ID NO:285 and SEQ ID NO:286,
(c) a nucleic acid sequence of (a) or (b) wherein T can be U, and
(d) a fragment of (a), (b) or (c).

13. The array of claim 12, comprising at least 10 different probes according to claim 12.

14. The array of claim 12, comprising at least 20 different probes according to claim 12.

15. The array of claim 12, comprising at least 30 different probes according to claim 12.

16. The array of claim 12, comprising at least 40 different probes according to claim 12.

17. The array of claim 12, comprising at least 50 different probes according to claim 12.

18. The array of claim 12, comprising at least 60 different probes according to claim 12.

19. The array of claim 12, comprising at least 72 different probes according to claim 12.

20. The array according to any one of claims 12 to 19, further comprising one or more control nucleic acid molecules.

21. The array according to claim 20, wherein the one or more control nucleic acid molecules comprise one or more expression level controls.

22. The array according to any one of claims 12 to 21, wherein the array is a microarray.

23. A method of detecting the expression of two or more genes, comprising the steps:
(a) providing two or more nucleic acid molecules, wherein two of the nucleic acid molecules have a nucleic acid sequence consisting of:
(i) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288;
(ii) a nucleic acid sequence prepared using amplification and primer pairs, wherein the primer pairs are selected from the following pairs of nucleic acid sequences:
SEQ ID NO:1 and SEQ ID NO:2;
SEQ ID NO:5 and SEQ ID NO:6;
SEQ ID NO:9 and SEQ ID NO:10;
SEQ ID NO:13 and SEQ ID NO:14;
SEQ ID NO:17 and SEQ ID NO:18;
SEQ ID NO:21 and SEQ ID NO:22;
SEQ ID NO:25 and SEQ ID NO:26;
SEQ ID NO:29 and SEQ ID NO:30;
SEQ ID NO:33 and SEQ ID NO:34;
SEQ ID NO:37 and SEQ ID NO:38;
SEQ ID NO:41 and SEQ ID NO:42;
SEQ ID NO:45 and SEQ ID NO:46;
SEQ ID NO:49 and SEQ ID NO:50;
SEQ ID NO:53 and SEQ ID NO:54;
SEQ ID NO:57 and SEQ ID NO:58;
SEQ ID NO:61 and SEQ ID NO:62;
SEQ ID NO:65 and SEQ ID NO:66;
SEQ ID NO:69 and SEQ ID NO:70;
SEQ ID NO:73 and SEQ ID NO:74;
SEQ ID NO:77 and SEQ ID NO:78;
SEQ ID NO:81 and SEQ ID NO:82;
SEQ ID NO:85 and SEQ ID NO:86;
SEQ ID NO:89 and SEQ ID NO:90;
SEQ ID NO:93 and SEQ ID NO:94;
SEQ ID NO:97 and SEQ ID NO:98;
SEQ ID NO:101 and SEQ ID NO:102;
SEQ ID NO:105 and SEQ ID NO:106;
SEQ ID NO:109 and SEQ ID NO:110;
SEQ ID NO:113 and SEQ ID NO:114;
SEQ ID NO:117 and SEQ ID NO:118;
SEQ ID NO:121 and SEQ ID NO:122;
SEQ ID NO:125 and SEQ ID NO:126;
SEQ ID NO:129 and SEQ ID NO:130;
SEQ ID NO:133 and SEQ ID NO:134;
SEQ ID NO:137 and SEQ ID NO: 138;
SEQ ID NO:141 and SEQ ID NO:142;
SEQ ID NO:145 and SEQ ID NO:146;
SEQ ID NO:149 and SEQ ID NO:150;
SEQ ID NO:153 and SEQ ID NO:154;
SEQ ID NO:157 and SEQ ID NO:158;
SEQ ID NO:161 and SEQ ID NO:162;
SEQ ID NO:165 and SEQ ID NO:166;
SEQ ID NO:169 and SEQ ID NO:170;
SEQ ID NO:173 and SEQ ID NO:174;
SEQ ID NO:177 and SEQ ID NO:178;
SEQ ID NO:181 and SEQ ID NO:182;
SEQ ID NO:185 and SEQ ID NO:186;
SEQ ID NO:189 and SEQ ID NO:190;
SEQ ID NO:193 and SEQ ID NO:194;
SEQ ID NO:197 and SEQ ID NO:198;
SEQ ID NO:201 and SEQ ID NO:202;
SEQ ID NO:205 and SEQ ID NO:206;
SEQ ID NO:209 and SEQ ID NO:210;
SEQ ID NO:213 and SEQ ID NO:214;
SEQ ID NO:217 and SEQ ID NO:218;
SEQ ID NO:221 and SEQ ID NO:222;
SEQ ID NO:225 and SEQ ID NO:226;
SEQ ID NO:229 and SEQ ID NO:230;
SEQ ID NO:233 and SEQ ID NO:234;
SEQ ID NO:237 and SEQ ID NO:238;
SEQ ID NO:241 and SEQ ID NO:242;
SEQ ID NO:245 and SEQ ID NO:246;
SEQ ID NO:249 and SEQ ID NO:250;
SEQ ID NO:253 and SEQ ID NO:254;
SEQ ID NO:257 and SEQ ID NO:258;
SEQ ID NO:261 and SEQ ID NO:262;
SEQ ID NO:265 and SEQ ID NO:266;
SEQ ID NO:269 and SEQ ID NO:270;
SEQ ID NO:273 and SEQ ID NO:274;
SEQ ID NO:277 and SEQ ID NO:278;
SEQ ID NO:281 and SEQ ID NO:282; or
SEQ ID NO:285 and SEQ ID NO:286;
(iii) a nucleic acid sequence in (i) or (ii) wherein T can also be U; or
(iv) a fragment of (i) to (iii);
(b) providing transcription indicators from a test sample;
(c) allowing the transcription indicators to hybridize with said two or more nucleic acid molecules; and
(d) detecting hybridization of said transcription indicators with said two or more nucleic acid molecules, wherein hybridization is indicative of the expression of the genes.

24. The method according to claim 23, wherein at least 10 different nucleic acid molecules according to claim 23 are provided.

25. The method according to claim 23, wherein at least 20 different nucleic acid molecules according to claim 23 are provided.

26. The method according to claim 23, wherein at least 30 different nucleic acid molecules according to claim 23 are provided.

27. The method according to claim 23, wherein at least 40 different nucleic acid molecules according to claim 23 are provided.

28. The method according to claim 23, wherein at least 50 different nucleic acid molecules according to claim 23 are provided.

29. The method according to claim 23, wherein at least 60 different nucleic acid molecules according to claim 23 are provided.

30. The method according to claim 23, wherein at least 72 different nucleic acid molecules according to claim 23 are provided.

31. The method according to any one of claims 23 to 30, wherein one or more control nucleic acid molecules are provided in step (a).

32. The method according to claim 31, wherein one or more control nucleic acid molecules comprise one or more expression level controls.

33. The method according to any one of claims 23-32, wherein the transcription indicators are selected from the group consisting of: transcripts of the gene or genes; cDNA reverse transcribed from the transcript; cRNA transcribed from the cDNA; DNA amplified from the genes; and RNA transcribed from amplified DNA.

34. The method according to claim 33, wherein the transcription indicator is cDNA.

35. The method according to any one of claims 23-34, wherein the transcription indicator is labeled.

36. The method according to any one of claims 23-35, wherein the test sample is from a human.

37. The method according to any one of claims 23-35, wherein the test sample is selected from one or more of cells, cell lines, tissues or organisms.

38. The method according to any one of claims 23-35, wherein the test sample is a clinical sample.

39. The method according to any one of claims 23-38 performed in a microarray format.

40. The method according to any one of claims 23-39, further comprising the steps of:
a) generating a set of expression data;
b) storing the data in a database; and
c) performing comparative analysis on the set of expression data, thereby analyzing gene expression.

41. A computer system comprising (a) a database containing information identifying the expression level of two or more genes; and b) a user interface to view the information, wherein the information identifying the expression level of two or more genes is obtained using the method according to any one of claims 23-40.

42. A method for screening a compound for its effect on the expression of two or more genes, comprising the steps:
(a) providing a transcription indicator from a test sample from a subject exposed to the compound;
(b) providing two or more nucleic acid molecules, wherein two of the nucleic acid molecules have a nucleic acid sequence consisting of:
(i) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288;
(ii) a nucleic acid sequence prepared using amplification and primer pairs, wherein the primer pairs are selected from the following pairs of nucleic acid sequences:
SEQ ID NO:1 and SEQ ID NO:2;
SEQ ID NO:5 and SEQ ID NO:6;
SEQ ID NO:9 and SEQ ID NO:10;
SEQ ID NO:13 and SEQ ID NO:14;
SEQ ID NO:17 and SEQ ID NO:18;
SEQ ID NO:21 and SEQ ID NO:22;
SEQ ID NO:25 and SEQ ID NO:26;
SEQ ID NO:29 and SEQ ID NO:30;
SEQ ID NO:33 and SEQ ID NO:34;
SEQ ID NO:37 and SEQ ID NO:38;
SEQ ID NO:41 and SEQ ID NO:42;
SEQ ID NO:45 and SEQ ID NO:46;
SEQ ID NO:49 and SEQ ID NO:50;
SEQ ID NO:53 and SEQ ID NO:54;
SEQ ID NO:57 and SEQ ID NO:58;
SEQ ID NO:61 and SEQ ID NO:62;
SEQ ID NO:65 and SEQ ID NO:66;
SEQ ID NO:69 and SEQ ID NO:70;
SEQ ID NO:73 and SEQ ID NO:74;
SEQ ID NO:77 and SEQ ID NO:78;
SEQ ID NO:81 and SEQ ID NO:82;
SEQ ID NO:85 and SEQ ID NO:86;
SEQ ID NO:89 and SEQ ID NO:90;
SEQ ID NO:93 and SEQ ID NO:94;
SEQ ID NO:97 and SEQ ID NO:98;
SEQ ID NO:101 and SEQ ID NO:102;
SEQ ID NO:105 and SEQ ID NO:106;
SEQ ID NO:109 and SEQ ID NO:110;
SEQ ID NO:113 and SEQ ID NO:114;
SEQ ID NO:117 and SEQ ID NO:118;
SEQ ID NO:121 and SEQ ID NO:122;
SEQ ID NO:125 and SEQ ID NO:126;
SEQ ID NO:129 and SEQ ID NO:130;
SEQ ID NO:133 and SEQ ID NO:134;
SEQ ID NO:137 and SEQ ID NO: 138;
SEQ ID NO:141 and SEQ ID NO:142;
SEQ ID NO:145 and SEQ ID NO:146;
SEQ ID NO:149 and SEQ ID NO:150;
SEQ ID NO:153 and SEQ ID NO:154;
SEQ ID NO:157 and SEQ ID NO:158;
SEQ ID NO:161 and SEQ ID NO:162;
SEQ ID NO:165 and SEQ ID NO:166;
SEQ ID NO:169 and SEQ ID NO:170;
SEQ ID NO:173 and SEQ ID NO:174;
SEQ ID NO:177 and SEQ ID NO:178;
SEQ ID NO:181 and SEQ ID NO:182;
SEQ ID NO:185 and SEQ ID NO:186;
SEQ ID NO:189 and SEQ ID NO:190;
SEQ ID NO:193 and SEQ ID NO:194;
SEQ ID NO:197 and SEQ ID NO:198;
SEQ ID NO:201 and SEQ ID NO:202;
SEQ ID NO:205 and SEQ ID NO:206;
SEQ ID NO:209 and SEQ ID NO:210;
SEQ ID NO:213 and SEQ ID NO:214;
SEQ ID NO:217 and SEQ ID NO:218;
SEQ ID NO:221 and SEQ ID NO:222;
SEQ ID NO:225 and SEQ ID NO:226;
SEQ ID NO:229 and SEQ ID NO:230;
SEQ ID NO:233 and SEQ ID NO:234;
SEQ ID NO:237 and SEQ ID NO:238;
SEQ ID NO:241 and SEQ ID NO:242;
SEQ ID NO:245 and SEQ ID NO:246;
SEQ ID NO:249 and SEQ ID NO:250;
SEQ ID NO:253 and SEQ ID NO:254;
SEQ ID NO:257 and SEQ ID NO:258;
SEQ ID NO:261 and SEQ ID NO:262;
SEQ ID NO:265 and SEQ ID NO:266;
SEQ ID NO:269 and SEQ ID NO:270;
SEQ ID NO:273 and SEQ ID NO:274;
SEQ ID NO:277 and SEQ ID NO:278;
SEQ ID NO:281 and SEQ ID NO:282; or
SEQ ID NO:285 and SEQ ID NO:286;
(iii) a nucleic acid sequence in (i) or (ii) wherein T can also be U; or
(iv) a fragment of (i) to (iii);
(c) allowing said transcription indicator to hybridize with said two or more nucleic acid molecules; and
(d) detecting hybridization of said transcription indicator with said two or more nucleic acid molecules, wherein hybridization is indicative of the expression of the two or more genes.

43. The method according to claim 42, further comprising the step of quantitatively or qualitatively comparing the hybridization detected in step (d) with the hybridization of transcription indicators from a control sample.

44. A method for screening a compound for its effect on the expression of two or more genes comprising:
(a) preparing a gene expression profile of a test sample from a subject that has been exposed to the compound using the method according to any one of claims 23 to 40;
(b) preparing a gene expression profile of a control sample using the method according to any one of claims 23 to 40; and
(c) quantitatively or qualitatively comparing the gene expression profiles from (a) and (b), wherein differential expression profiles in (a) and (b) is indicative of a compound having an effect on the expression of two or more genes.

45. The method according to claim 44, wherein the differential expression of two or more of the genes in the test sample when compared to the control sample is indicative of the efficacy of the compound.

46. The method according to claim 44, wherein the differential expression of two or more of the genes in the test sample when compared to the control sample is indicative of the toxicity of the compound.

47. A method of assessing the toxicity and/or efficacy of a compound in a subject comprising:
(a) preparing a gene expression profile of a test sample from a subject that has been exposed to the compound using the method according to any one of claims 23 to 40;
(b) preparing a gene expression profile of a control sample using the method according to any one of claims 23 to 40; and
(c) quantitatively or qualitatively comparing the gene expression profiles from (a) and (b), wherein a difference in the gene expression profiles in (a) and (b) is indicative of the toxicity and/or efficacy of the compound.

48. A method for determining a change in gene expression profile for a compound in the presence of one or more different compounds comprising:
(a) preparing a gene expression profile of a test sample from a subject that has been exposed to the compound using the method according to any one of claims 23 to 40;
(b) preparing a gene expression profile of the test sample that has been exposed to the compound and one or more different compounds using the method according to any one of claims 23 to 40; and
(c) quantitatively or qualitatively comparing the gene expression profiles from (a) and (b), wherein differential expression in (a) and (b) indicates that the gene expression profile of the compound changes in the presence of the one or more different compounds.

49. The method according to claim 48, wherein changes in the gene expression profile indicate the presence of drug-drug interactions.

50. The method according to any one of claims 42-49 wherein the hybridization is detected over a period of time at specified time intervals.

51. A kit comprising the array according to any one of claims 1-22 and one or more of the following: reagents for use with the array; signal detection and array-processing instruments; gene expression databases; or analysis and database management software.

52. A relational database comprising gene expression profiles obtained using the method according to any one of claim 23-40 or 42-50.

53. The database according to claim 52, further comprising information selected from the group consisting of: sequence information; descriptive information about the gene associated with the sequence information; and the clinical status of the test sample and/or its source.

54. An isolated nucleic acid molecule having a nucleic acid sequence consisting of:
(a) a nucleic acid sequence as shown in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, or 288;
(b) a nucleic acid sequence prepared using amplification and primer pairs, wherein the primer pairs are selected from the following pairs of nucleic acid sequences:
SEQ ID NO:1 and SEQ ID NO:2;
SEQ ID NO:5 and SEQ ID NO:6;
SEQ ID NO:9 and SEQ ID NO:10;
SEQ ID NO:13 and SEQ ID NO:14;
SEQ ID NO:17 and SEQ ID NO:18;
SEQ ID NO:21 and SEQ ID NO:22;
SEQ ID NO:25 and SEQ ID NO:26;
SEQ ID NO:29 and SEQ ID NO:30;
SEQ ID NO:33 and SEQ ID NO:34;
SEQ ID NO:37 and SEQ ID NO:38;
SEQ ID NO:41 and SEQ ID NO:42;
SEQ ID NO:45 and SEQ ID NO:46;
SEQ ID NO:49 and SEQ ID NO:50;
SEQ ID NO:53 and SEQ ID NO:54;
SEQ ID NO:57 and SEQ ID NO:58;
SEQ ID NO:61 and SEQ ID NO:62;
SEQ ID NO:65 and SEQ ID NO:66;
SEQ ID NO:69 and SEQ ID NO:70;
SEQ ID NO:73 and SEQ ID NO:74;
SEQ ID NO:77 and SEQ ID NO:78;
SEQ ID NO:81 and SEQ ID NO:82;
SEQ ID NO:85 and SEQ ID NO:86;
SEQ ID NO:89 and SEQ ID NO:90;
SEQ ID NO:93 and SEQ ID NO:94;
SEQ ID NO:97 and SEQ ID NO:98;
SEQ ID NO:101 and SEQ ID NO:102;
SEQ ID NO:105 and SEQ ID NO:106;
SEQ ID NO:109 and SEQ ID NO:110;
SEQ ID NO:113 and SEQ ID NO:114;
SEQ ID NO:117 and SEQ ID NO:118;
SEQ ID NO:121 and SEQ ID NO:122;
SEQ ID NO:125 and SEQ ID NO:126;
SEQ ID NO:129 and SEQ ID NO:130;
SEQ ID NO:133 and SEQ ID NO:134;
SEQ ID NO:137 and SEQ ID NO: 138;
SEQ ID NO:141 and SEQ ID NO:142;
SEQ ID NO:145 and SEQ ID NO:146;
SEQ ID NO:149 and SEQ ID NO:150;
SEQ ID NO:153 and SEQ ID NO:154;
SEQ ID NO:157 and SEQ ID NO:158;
SEQ ID NO:161 and SEQ ID NO:162;
SEQ ID NO:165 and SEQ ID NO:166;
SEQ ID NO:169 and SEQ ID NO:170;
SEQ ID NO:173 and SEQ ID NO:174;
SEQ ID NO:177 and SEQ ID NO:178;
SEQ ID NO:181 and SEQ ID NO:182;
SEQ ID NO:185 and SEQ ID NO:186;
SEQ ID NO:189 and SEQ ID NO:190;
SEQ ID NO:193 and SEQ ID NO:194;
SEQ ID NO:197 and SEQ ID NO:198;
SEQ ID NO:201 and SEQ ID NO:202;
SEQ ID NO:205 and SEQ ID NO:206;
SEQ ID NO:209 and SEQ ID NO:210;
SEQ ID NO:213 and SEQ ID NO:214;
SEQ ID NO:217 and SEQ ID NO:218;
SEQ ID NO:221 and SEQ ID NO:222;
SEQ ID NO:225 and SEQ ID NO:226;
SEQ ID NO:229 and SEQ ID NO:230;
SEQ ID NO:233 and SEQ ID NO:234;
SEQ ID NO:237 and SEQ ID NO:238;
SEQ ID NO:241 and SEQ ID NO:242;
SEQ ID NO:245 and SEQ ID NO:246;
SEQ ID NO:249 and SEQ ID NO:250;
SEQ ID NO:253 and SEQ ID NO:254;
SEQ ID NO:257 and SEQ ID NO:258;
SEQ ID NO:261 and SEQ ID NO:262;
SEQ ID NO:265 and SEQ ID NO:266;
SEQ ID NO:269 and SEQ ID NO:270;
SEQ ID NO:273 and SEQ ID NO:274;
SEQ ID NO:277 and SEQ ID NO:278;
SEQ ID NO:281 and SEQ ID NO:282; or
SEQ ID NO:285 and SEQ ID NO:286;
(c) a nucleic acid sequence in (a) or (b) wherein T can also be U; or
(d) a fragment of (a) to (c).

55. A pair of primers for preparing the nucleic acid molecule according to claim 54.

56. The pair of primers according to claim 55, wherein the pair of primers is selected from the following pairs of nucleic acid sequences:
SEQ ID NO:1 and SEQ ID NO:2;
SEQ ID NO:5 and SEQ ID NO:6;
SEQ ID NO:9 and SEQ ID NO:10;
SEQ ID NO:13 and SEQ ID NO:14;
SEQ ID NO:17 and SEQ ID NO:18;
SEQ ID NO:21 and SEQ ID NO:22;
SEQ ID NO:25 and SEQ ID NO:26;
SEQ ID NO:29 and SEQ ID NO:30;
SEQ ID NO:33 and SEQ ID NO:34;
SEQ ID NO:37 and SEQ ID NO:38;
SEQ ID NO:41 and SEQ ID NO:42;
SEQ ID NO:45 and SEQ ID NO:46;
SEQ ID NO:49 and SEQ ID NO:50;
SEQ ID NO:53 and SEQ ID NO:54;
SEQ ID NO:57 and SEQ ID NO:58;
SEQ ID NO:61 and SEQ ID NO:62;
SEQ ID NO:65 and SEQ ID NO:66;
SEQ ID NO:69 and SEQ ID NO:70;
SEQ ID NO:73 and SEQ ID NO:74;
SEQ ID NO:77 and SEQ ID NO:78;
SEQ ID NO:81 and SEQ ID NO:82;
SEQ ID NO:85 and SEQ ID NO:86;
SEQ ID NO:89 and SEQ ID NO:90;
SEQ ID NO:93 and SEQ ID NO:94;
SEQ ID NO:97 and SEQ ID NO:98;
SEQ ID NO:101 and SEQ ID NO:102;
SEQ ID NO:105 and SEQ ID NO:106;
SEQ ID NO:109 and SEQ ID NO:110;
SEQ ID NO:113 and SEQ ID NO:114;
SEQ ID NO:117 and SEQ ID NO:118;
SEQ ID NO:121 and SEQ ID NO:122;
SEQ ID NO:125 and SEQ ID NO:126;
SEQ ID NO:129 and SEQ ID NO:130;
SEQ ID NO:133 and SEQ ID NO:134;
SEQ ID NO:137 and SEQ ID NO: 138;
SEQ ID NO:141 and SEQ ID NO:142;
SEQ ID NO:145 and SEQ ID NO:146;
SEQ ID NO:149 and SEQ ID NO:150;
SEQ ID NO:153 and SEQ ID NO:154;
SEQ ID NO:157 and SEQ ID NO:158;
SEQ ID NO:161 and SEQ ID NO:162;
SEQ ID NO:165 and SEQ ID NO:166;
SEQ ID NO:169 and SEQ ID NO:170;
SEQ ID NO:173 and SEQ ID NO:174;
SEQ ID NO:177 and SEQ ID NO:178;
SEQ ID NO:181 and SEQ ID NO:182;
SEQ ID NO:185 and SEQ ID NO:186;
SEQ ID NO:189 and SEQ ID NO:190;
SEQ ID NO:193 and SEQ ID NO:194;
SEQ ID NO:197 and SEQ ID NO:198;
SEQ ID NO:201 and SEQ ID NO:202;
SEQ ID NO:205 and SEQ ID NO:206;
SEQ ID NO:209 and SEQ ID NO:210;
SEQ ID NO:213 and SEQ ID NO:214;
SEQ ID NO:217 and SEQ ID NO:218;
SEQ ID NO:221 and SEQ ID NO:222;
SEQ ID NO:225 and SEQ ID NO:226;
SEQ ID NO:229 and SEQ ID NO:230;
SEQ ID NO:233 and SEQ ID NO:234;
SEQ ID NO:237 and SEQ ID NO:238;
SEQ ID NO:241 and SEQ ID NO:242;
SEQ ID NO:245 and SEQ ID NO:246;
SEQ ID NO:249 and SEQ ID NO:250;
SEQ ID NO:253 and SEQ ID NO:254;
SEQ ID NO:257 and SEQ ID NO:258;
SEQ ID NO:261 and SEQ ID NO:262;
SEQ ID NO:265 and SEQ ID NO:266;
SEQ ID NO:269 and SEQ ID NO:270;
SEQ ID NO:273 and SEQ ID NO:274;
SEQ ID NO:277 and SEQ ID NO:278;
SEQ ID NO:281 and SEQ ID NO:282; or
SEQ ID NO:285 and SEQ ID NO:286;
wherein T can also be U.
